(19) United States
(12) Patent Application Publication
(10) Pub. No.: US 2017/0369444 A1
(43) Pub. Date: Dec. 28, 2017

(54) INHIBITORS OF HISTONE DEMETHYLASES

(71) Applicants: Marc LABELLE, Basking Ridge, NJ (US); Gilead Sciences, Inc., Foster City, CA (US)

(72) Inventors: Marc Labelle, Basking Ridge, NJ (US); Rui Zhang, Winnipeg, Manitoba (CA); Cuthbert D. Martyr, Winnipeg, Manitoba (CA); Neerja Saraswat, Winnipeg (CA); Thomas Boesen, Kobenhavn (DK)

(21) Appl. No.: 15/129,351

(22) PCT Filed: Mar. 30, 2015
(86) PCT No.: PCT/US2015/023407

§ 371 Date: Sep. 26, 2016

Related U.S. Application Data
(60) Provisional application No. 61/972,972, filed on Mar. 31, 2014.

(51) Int. Cl. C07D 213/79; C07D 471/04; C07D 417/12; C07D 405/12; C07D 237/24; C07D 401/12; C07D 401/06; C07D 239/28; C07D 487/04; C07D 409/12

(52) U.S. Cl. CPC C07D 213/79; C07D 487/04; C07D 471/04; C07D 417/12; C07D 409/12; C07D 237/24; C07D 401/12; C07D 401/06; C07D 239/28; C07D 405/12

(57) ABSTRACT

Compounds of the form in which Q is selected from —COOH—CH═NR¹², —W, —CH₂NHR¹³, —CH=0 and —CH(OR¹⁷)₂ capable of modulating the activity of histone demethylases (HDMEs), which are useful for prevention and/or treatment of diseases in which genomic dysregulation is involved in the pathogenesis, such as e.g. cancer and formulations and methods of use of such compounds.

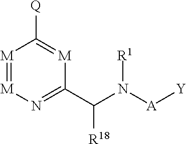

### RELATED APPLICATION

This application claims priority to, and the benefits of, U.S. Provisional Application Ser. No. 61/972,972, filed Mar. 31, 2014. The entire content of which is incorporated by reference herein.

### FIELD OF THE DISCLOSURE

The present disclosure relates to compounds capable of modulating the activity of histone demethylases (HDMEs), which compounds are useful for the prevention and/or the treatment of diseases in which genomic dysregulation is involved in the pathogenesis, such as e.g. cancer.

### BACKGROUND OF THE DISCLOSURE

The DNA of eukaryotic cells is packaged into chromatin by winding of the DNA around histone proteins to form nucleosomes, the basic unit of chromatin. One of the important functions of chromatin is to determine regions of active and silenced transcription by changing the ordered chromatin structure. Such changes have profound effects on cellular function since they affect fundamental processes as differentiation, proliferation and apoptosis, and are often referred collectively to as “epigenetic” since they can lead to heritable changes that do not involve changes in gene sequences (Quina, A. S. et al. (2006), Biochem. Pharmacol. 72; 1563-1569)

These highly controlled chromatin changes are mediated by alterations histone proteins associated with DNA in the nucleosome. Most notably, the N-terminal histone tail of Histone H3 and histone H4 are subject to such covalent changes, which include changes in methylation, acetylation, phosphorylation and ubiquitination. The addition or removal of these groups on histones is mediated by specific enzymes, e.g. histone methyl transferases and histone demethylases for methyl groups, histone acetyltransferases and histone deacetylases for acetyl groups, etc. In the event that the activity or expression of these “epigenetic” enzymes is not correctly controlled and regulated it may lead to disease. Cancer, in particular, is an area of high importance in relation to dysregulated epigenetic enzyme activity due to the role of epigenetics in cell differentiation, proliferation and apoptosis, but epigenetics may also play a role in other diseases like metabolic, inflammatory, neurodegenerative and cardiovascular diseases. Therefore the selective modulation of aberrant action of epigenetic enzymes may hold great promise for the treatment of human disease (Kelly, T. K. et al. (2010), Nat. Biotechnol. 28; 1069-1078, and Cloos, P.a.C. et al. (2008), Genes. Dev. 22; 1115-1140).

Methylation and demethylation of lysine residues on the histone H3 tail constitute important epigenetic marks delineating transcriptionally active and inactive chromatin. For example, methylation of lysine 9 on histone H3 (H3K9) is usually associated with epigenetically silenced chromatin (Fischle, W., et. al. (2003), Curr. Opinion Cell Biol. 15, 172-183; Margueron, R., et al. (2005), Curr. Opinion Genet. Dev. 15, 163-176) while methylation of lysine 4 on histone 3 is associated with transcriptionally active chromatin. Similarly, the lysine 27 histone H3 (H3K27) mark is repressive in its di- and tri-methylated states whereas the lysine 36 histone H3 mark is found in association with gene activation (Barski, A. et al. (2007), Cell, 129, 823-837; Vakoc, C. et al. (2006) Mol. Cell. Biol. 26, 9185-9195; Wagner, E. J. & Carpenter, P. B. (2012) Nature Mol. Cell Biol 13, 115-126). There are, however, many exemptions from these general rules of association between methylation states of epigenetic marks and the effect they have on transcription.

As documented by studies of the SUV39H1 knockout mouse, loss of the tri-methyl variant of the H3K9 mark results in chromosomal aberrations and predisposes to cancer (Peters, A. H. et al., Cell 107, 323-337, 2001). The JMJD2C protein (KDM4C, GASC1) has been identified as an eraser of the H3K9 mark (a histone demethylase) and may therefore promote cancer if its expression and activity is not tightly controlled (Cloos, P. et al. (2006), Nature 442, 307-311; Klose, R. J. et al. (2006), Nature 442, 312-316; Liu, G. et al. (2009), Oncogene 28, 4491-4500). For example, JMJD2C has been shown to induce transformed phenotypes like growth factor independent growth, anchorage independent growth and mammosphere formation, if it is overexpressed in cells (Liu, G. et al. (2009), Oncogene 28, 4491-4500). These findings are supported by the overexpression of JMJD2C in a range of human tumours like squamous cell carcinoma, metastatic lung carcinoma, prostate cancer, breast cancer and several others (Yang, Z. Q. et al. (2000) Cancer Res. 60, 4735-4739; Yang, Z. Q. et al. (2001) Jpn. J. Cancer Res. 92, 423-428; Hu, N. et al. (2005) Cancer Res. 65, 2542-2546; Liu, G. et al. (2009) Oncogene 28, 4491-4500; Wissmann, M. et al. (2007) Nat. Cell Biol. 9, 347-353), indicating the potential importance of JMJD2C as an oncogene.

The JMJD2A protein (KDM4A, JHDM3A) shows similar properties to JMJD2C. JMJD2A shows high sequence identity to JMJD2C in its JmjC catalytic domain, is an eraser of the H3K9 mark and has also been shown to be overexpressed in prostate cancer (Cloos, P. Et al., Nature 442, 307-311, 2006). JMJD2A has been shown to interact with the estrogen receptor alpha (ER-alpha) and overexpression of JMJD2A enhances estrogen-dependent transcription and the down-regulation of JMJD2A reduced transcription of a seminal ER-alpha target gene, cyclin Dl (Kawazu et al., (2011) PLoS One 6; Berry et al., (2012) Int J Oncol 41). Additionally, it has been shown that catalytically inactive JMJD2A is compromised in its ability to stimulate ER-alpha mediated transcription, suggesting that inhibitors of JMJD2A may be beneficial for the treatment of ER-alpha positive breast tumours (Berry et al., (2012) Int J Oncol 41).

Likewise, an eraser of the tri-methyl variant of the H3K4 mark, JARID1B (KDM5B, PLU1) has also been identified as potential oncogene. In cancer JARID1B most likely acts as a repressor of tumour repressor genes via removal of the H3K4 tri-methylation leading to decreased transcriptional activation in the affected chromatin regions. The oncogenic potential of JARID1B is demonstrated by its stimulation of proliferation in cell lines and further validated by shRNA knockdown studies of JARID1B expression showing inhibition of proliferation in MCF7 human breast cancer cells, in SW780 and RT4 bladder cancer cells, in A549 and LC319 lung cancer cells and in 4T1 mouse tumour cells in vitro and/or in mouse xenograft experiments (Yamane K. et al. (2007), Mol. Cell 25, 801-812; Hayami S. et al. (2010) Mol. Cancer 9, 59; Catchpole S et al. (2011), Int. J. Oncol. 38, 1267-1277). Finally, JARID1B is overexpressed in prostate cancer and is associated with malignancy and poor prognosis (Xiang Y. et al. (2007) PNAS 104).

JARID1A (KDM5A, RBP2) is also an eraser of the tri- and di-methyl variant of the H3K4 mark. JARID1A is overexpressed in gastric cancer (Zeng et al., (2010) Gastroenterology 138) and its gene is amplified in cervix carcinoma (Hidalgo et al, (2005) BMC Cancer 5). It has been suggested that JARID1A is fine-tuning progesterone receptor expression control by estrogens (Stratmann and Haendler (2011) FEBS J 278). Together with JARID1B, JARID1A has been implicated in the maintenance of a slow-growing population of cancer cells that are required for continuous tumor growth and that are resistant to cytotoxic and targeted therapy (Roesch, et al, (2010) Cell 141; Sharma, et al., (2010) Cell 141). JARID1A is required for the tumor initiation and progression in Rb+/− and Men1-defective mice (Lin, et al., (2011) PNAS 108). Data from Pasini show that JARID1A binds to Polycomb group protein target genes which are involved in regulating important cellular processes such as embryogenesis, cell proliferation, and stem cell self-renewal through the transcriptional repression of genes determining cell fate decisions (Pasini et al., (2008) Genes & Dev 22). Additionally, JARID1A were also shown to binds the PRC2 complex and being regulator of PRC2 target genes (Pasini et al., (2008) Genes & Dev 22).

Another potential oncogene, an eraser of the di-methyl variant of the H3K36 mark, JHDM1B (KDM2B, FBXL10) has been shown to be highly expressed in human cancers (Tzatsos A et al. (2009), PNAS 106 (8), 2641-2646; He, J. et al. (2011), Blood 117 (14), 3869-3880). Knock-down of FBXL10 causes senescence in mouse embryonic fibroblasts (MEFs), which can be rescued by expression of catalytic active (but not catalytic inactive) JHDM1B (Pfau, R et al. (2008), PNAS 105(6), 1907-1912; He, J et al. (2008), Nat Struct Mol Biol 15, 1169-1175). JHDM1B demethylates H3K36me2 on the tumor-suppressor gene Ink4b (p15^{Ink4b}), and thereby silences the expression of this senescence-mediating gene in MEFs and in leukemic cells (He, J. et al. (2008), Nat Struct Mol Biol 15, 1169-1175; He, J. et al. (2011), Blood 117 (14), 3869-3880). The catalytic dependency of JHDM1B is further shown by He et al. as catalytic activity is required for development of leukemia in a mouse AML model.

Inhibitors of the histone demethylase class of epigenetic enzymes, and in particular the potential oncogenes JARID1B, JARID1A, JMJD2C, JMJD2A, and JHDM1B, would present a novel approach for intervention in cancers and other proliferative diseases. Being one of the most devastating diseases, affecting millions of people worldwide, there remains a high need for efficacious and specific compounds against cancer.

PCT/EP2013/070457 and PCT/EP2014/053674 disclose histone demethylase (HDME) inhibitors or activity modulators.

Embodiments of the disclosure provide novel series of compounds capable of modulating the activity of histone demethylases, at least some of which compounds are useful for the prevention and/or the treatment of diseases in which genomic disregulation is involved in the pathogenesis, such as, e.g., cancer.

The inventors have surprisingly found that novel compounds of Formula (I) as defined herein can be used in the treatment of HDME dependent diseases by inhibiting HDMEs. Inhibiting HDMEs would provide a novel approach to the prevention and treatment of cancer and other proliferative diseases. Accordingly, it is an object of the present disclosure to provide compounds that when administered alone or optionally in combination with anti-neoplastic compounds, increases the efficacy of the treatment of HDME dependent diseases.

Accordingly, a first aspect of the present disclosure relates to a compound of the Formula (Ia)

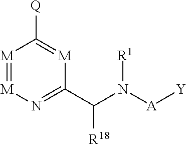

wherein
Q is selected from CO₂H, —CH═NR¹², —W, —CHR²⁰NR²¹R¹³, —CH═O and —CH(OR¹⁷)₂;
A is selected from —C(R^{2a})₂C(O)—, —C(R²)₂C(R²)₂C(O)—, —Z′—C₃₋₁₀ cycloalkylene, —Z′-heterocyclylene, —Z′-heteroarylene and —Z′-arylene, which —Z′-cycloalkylene, —Z′-heterocyclylene, —Z′-heteroarylene and —Z′-arylene may optionally be substituted with one or more R³ and may form a cyclic or heterocyclic structure with Y, wherein said cyclic or heterocyclic structure formed with Y is optionally fused to an optionally substituted aryl or heteroaryl group;
Z′ is selected from C₁₋₄ alkylene, C₂₋₅ alkenene, C₂₋₅ alkynene, heterocyclylene and C₃₋₆ cycloalkylene;
Each M is independently selected from CH or N;
Y is selected from —H, —NR⁶R⁷, —OR⁷, C₁₋₈ alkyl, C₂₋₈ alkenyl, C₂₋₈ alkynyl, C₃₋₁₀ cycloalkyl, heterocyclyl, heteroaryl and aryl, which alkyl, alkenyl, alkynyl, cycloalkyl, heterocyclyl, heteroaryl and aryl may optionally be substituted with one or more R³;
R¹ is selected from —H, C₁₋₈ alkyl, C₂₋₈ alkenyl, C₂₋₈ alkynyl, C₃₋₁₀ cycloalkyl, which alkyl, alkenyl, alkynyl and cycloalkyl may be optionally substituted with one or more selected from —OH, aryl, C₁₋₆ alkoxy, heteroaryl, aryloxy, heteroaryloxy, —NR⁶R⁷, F, and C₃₋₆ cycloalkyl; or more preferably is selected from —H and C₁₋₄ alkyl; or with -A-Y forms a nitrogen containing optionally substituted heterocyclic group where the optional substitution may be C₁₋₈ alkyl, C₂₋₈ alkenyl, C₂₋₈ alkynyl, C₃₋₁₀ cycloalkyl, C₁₋₆ alkoxy, C₁₋₆ hydroxyalkyl, aryl, —C(═O)NR⁶R⁷, —NR⁶R⁷, —OH, and halogen, which alkyl, alkenyl, alkynyl, cycloalkyl and aryl may be optionally substituted with one or more selected from —OH, aryl, C₁₋₆ alkoxy, heteroaryl, aryloxy, heteroaryloxy, —NR⁶R⁷, F, and C₃₋₆ cycloalkyl, or said nitrogen containing optionally substituted heterocyclic group formed with -A-Y is optionally fused to an optionally substituted aryl or heteroaryl group; or with R¹⁸ forms a nitrogen containing optionally substituted heterocyclic group where the optional substitution may be C₁₋₈ alkyl, C₂₋₈ alkenyl, C₂₋₈ alkynyl, or C₃₋₁₀ cycloalkyl, which alkyl, alkenyl, alkynyl and cycloalkyl may be optionally substituted with one or more selected from —OH, aryl, C₁₋₆ alkoxy, heteroaryl, aryloxy, heteroaryloxy, —NR⁶R⁷, F, and C₃₋₆ cycloalkyl;
R² is selected from —H, C₁₋₈ alkyl, C₂₋₈ alkenyl, C₂₋₈ alkynyl, and C₃₋₁₀ cycloalkyl, which alkyl, alkenyl, alkynyl and cycloalkyl may be optionally substituted with one or more selected from —OH, aryl, C₁₋₆ alkoxy, heteroaryl, aryloxy, heteroaryloxy, F, and C₃₋₆ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—C(═O)—R⁷, —Z—OR⁷, halogen, —Z—SR⁷, —Z—SOR⁷, —Z—SO₂R⁷, —Z—SO₂NR⁶R⁷ and —Z—COOR⁷; alternatively, R² may form a cyclic or heterocyclic structure with another R², R¹ R¹⁸ or Y;
R^{2a} is selected from —H, C₁₋₈ alkyl, C₂₋₈ alkenyl, C₂₋₈ alkynyl, and C₃₋₁₀ cycloalkyl, which alkyl, alkenyl, alkynyl and cycloalkyl may be optionally substituted with one or more selected from —OH, aryl, C₁₋₆ alkoxy, heteroaryl, aryloxy, heteroaryloxy, F, and C₃₋₆ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—NR⁶—C(═O)—OR⁷, —Z—C(═O)—OR⁷, —Z—OR⁷, halogen, —Z—SR⁷, —Z—SOR⁷, —Z—SO₂R⁷, —Z—SO₂NR⁶R⁷ and —Z—COOR⁷; with the proviso that the two R^{2a} groups are either both non-hydrogen, or that one of the R^{2a} forms a ring with R¹ or R¹⁸;
each R³ is independently selected from C₁₋₆ alkyl, C₁₋₄ fluoroalkyl, C₁₋₄ hydroxyalkyl, C₂₋₆ alkenyl, C₂₋₆ alkynyl, C₃₋₁₀ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—NR⁶—C(═O)—OR⁷, —Z—C(═O)—R⁷, —Z—OR⁷, halogen, —Z—SR⁷, —Z—SOR⁷, —Z—SO₂R⁷, —Z—SO₂NR⁶R⁷, and —Z—COOR⁷, wherein any heterocyclyl may be substituted with one or more R⁴, and wherein any heteroaryl and any aryl may be substituted with one or more R⁵;
Z is selected from a single bond, C₁₋₄ alkylene, heterocyclylene and C₃₋₆ cycloalkylene;
each R⁴ is independently selected from C₁₋₆ alkyl, C₁₋₄ fluoroalkyl, C₁₋₄ hydroxyalkyl, C₁₋₄ alkoxy, C₃₋₁₀ cycloalkyl, NR⁶R⁷, C(═O)—NR⁶R⁷, NR⁶—C(═O)—R⁷, Z—C(═O)—R⁷, —Z—C(═O)—H, OR⁷, halogen, SR⁷, SOR⁷, SO₂R⁷, SO₂NR⁶R⁷ and COOR⁷ and —OH;
each R⁵ is independently selected from C₁₋₆ alkyl, C₁₋₄ fluoroalkyl, C₁₋₄ hydroxyalkyl, C₁₋₄ alkoxy, C₃₋₆ cycloalkyl, —Z—C(═O)—NR⁶R⁷, —Z—NR⁶—C(═O)—R⁷, —Z—C(═O)—R⁷, —Z—C(═O)OR⁷, —Z—NR⁶C(═O)OR⁷, OR⁷, —CN and halogen;
each of R⁶ and R⁷ is independently selected from hydrogen, optionally not both being hydrogen, C₁₋₈ alkyl, C₁₋₄ fluoroalkyl, C₁₋₄ perfluoroalkyl, C₁₋₄ hydroxyalkyl, C₂₋₈ alkenyl, C₂₋₈ alkynyl, C₃₋₁₀ cycloalkyl, —Z-heterocyclyl, —Z-heteroaryl and —Z-aryl, which alkyl, alkenyl, alkynyl, cycloalkyl, heterocyclyl, heteroaryl and aryl may optionally be substituted with one or more independently selected R⁸; or, alternatively, R⁶ and R⁷ may together with the N-atom to which they are attached form an N-heterocyclic ring optionally substituted with one or more independently selected R⁸;
each R⁸ is independently selected from C₁₋₆ alkyl, C₁₋₄ fluoroalkyl, C₁₋₄ hydroxyalkyl, C₂₋₆ alkenyl, C₂₋₆ alkynyl, C₃₋₁₀ cycloalkyl, —Z-heterocyclyl, —Z-heteroaryl, —Z-aryl, —Z—NR¹⁰R¹¹, —Z—C(═O)—NR¹⁰R¹¹, —Z—OR⁹, halogen, —CN, —Z—SR⁹, —Z—SOR⁹, —Z—SO₂R⁹ and —Z—COOR⁹, which alkyl, alkenyl, alkynyl, cycloalkyl, heterocyclics, heteroaryl and aryl may optionally be substituted with one or more selected from C₁₋₄ alkyl, C₁₋₄ fluoroalkyl, C₁₋₄ hydroxyalkyl, C₃₋₆ cycloalkyl, —Z-heterocyclyl, —Z-heteroaryl, —Z-aryl, —Z—NR¹⁰R¹¹, —Z—C(═O)—NR¹⁰R¹¹, —Z—OR⁹, halogen, —CN, —Z—SR⁹, —Z—SOR⁹, —Z—SO₂R⁹ and —Z—COOR⁹; wherein any heterocyclyl may be further substituted with one or more R⁴ as defined above, and wherein any heteroaryl and any aryl may be further substituted with one or more R⁵ as defined above;
each R⁹ is independently selected from —H, C₁₋₈ alkyl, C₁₋₄ fluoroalkyl, C₁₋₄ hydroxyalkyl, C₂₋₈ alkenyl, C₂₋₈ alkynyl, C₃₋₁₀ cycloalkyl, —Z-heterocyclyl, —Z-aryl, and —Z-heteroaryl, wherein any heterocyclyl may be substituted with one or more R⁴ as defined above, and wherein any heteroaryl and any aryl may be substituted with one or more R⁵ as defined above;
each of R¹⁰ and R¹¹ is independently selected from —H, C₁₋₆ alkyl, C₁₋₄ fluoroalkyl, C₁₋₄ hydroxyalkyl, C₂₋₈ alkenyl, C₂₋₈ alkynyl, C₃₋₁₀ cycloalkyl, heterocyclyl, heteroaryl, and aryl, wherein any heterocyclyl may be substituted with one or more R⁴ as defined above, and wherein any heteroaryl and any aryl may be substituted with one or more R⁵ as defined above, or, alternatively, R¹⁰ and R¹¹ may together with the N-atom to which they are attached form an optionally 5 to 7 membered, N-heterocyclic ring optionally substituted with one or more R⁴ as defined above;
when Q is —CH═NR¹², R¹² is selected from C₁₋₁₀ alkyl, C₂₋₁₀ alkenyl, C₂₋₁₀ alkynyl, C₃₋₁₀ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—C(═O)—R⁷, —Z—OR⁷, halogen, —Z—SR⁷, —Z—SOR⁷, —Z—SO₂R⁷ and —Z—COOR⁷, which alkyl, alkenyl, alkynyl, cycloalkyl, heterocyclyl, heteroaryl and aryl may optionally be substituted with one or more R³;
when Q is —CHR²⁰NR²¹R¹³, R¹³ is selected from hydrogen, —C(O)R⁷, —C(O)C(O)R⁷, —C(O)C(O)OR⁷, C₁₋₈ alkyl, C₁₋₄ fluoroalkyl, C₁₋₄ perfluoroalkyl, C₁₋₄ hydroxyalkyl, C₂₋₈ alkenyl, C₂₋₈ alkynyl, C₃₋₁₀ cycloalkyl, —Z-heterocyclyl, —Z-aryl and —Z-heteroaryl, which alkyl, alkenyl, alkynyl, cycloalkyl, heterocyclyl, aryl and heteroaryl may optionally be substituted with one or more independently selected R⁸, or is —CR¹⁴R¹⁵—NR⁶R⁷, —CR¹⁴R¹⁵CN, or —CR¹⁴R¹⁵OR⁷, wherein each of R¹⁴ and R¹⁵ is independently selected from —H, C₁₋₈ alkyl, C₂₋₈ alkenyl, C₂₋₈ alkynyl, C₃₋₁₀ cycloalkyl, heterocyclyl, heteroaryl and aryl, and wherein R¹⁴ and R¹⁵ together with the intervening carbon atom may designate a C₃₋₁₀ cycloalkyl or C₅₋₁₀-cycloalkenyl ring, which alkyl, alkenyl, alkynyl, cycloalkyl (ring), cycloalkenyl ring, heterocyclyl, heteroaryl and aryl may optionally be substituted with one or more R³;
R²⁰ and R²¹ are hydrogen, or together form a 1,3-diaza-C₅₋₇-cycloalk-2-yl group which is N-substituted with R¹⁶ and optionally further substituted with one or more R³, and optionally containing one or two oxo groups; a 1,3-thiaza-C₅₋₇-cycloalk-2-yl group which is N-substituted with R¹⁶ and optionally further substituted with one or more R³ and optionally containing one or two oxo groups; an 1,3-oxaza-C₅₋₇-cycloalk-2-yl group which is N-substituted with R¹⁶ and optionally further substituted with one or more R³, and optionally containing one or two oxo groups, wherein in all three instances two R³'s on the same carbon atom may together form a spiro group;
when Q is W, W is selected from an 1,3-diaza-C₅₋₇-cycloalk-2-yl group which is N-substituted with R¹⁶ and optionally further substituted with one or more R³, and optionally containing one or two oxo groups; a 1,3-thiaza-C₅₋₇-cycloalk-2-yl group which is N-substituted with R¹⁶ and optionally further substituted with one or more R³ and optionally containing one or two oxo groups; an 1,3-oxaza-C₅₋₇-cycloalk-2-yl group which is N-substituted with R¹⁶ and optionally further substituted with one or more R³, and optionally containing one or two oxo groups, wherein in all three instances two R³'s on the same carbon atom may together form a spiro group;
R¹⁶ is selected from hydrogen, —C(O)R⁷, —C(O)C(O)R⁷, —C(O)C(O)OR⁷, and —C(O)C(O)NR⁶R⁷;
when Q is —CH(OR¹⁷)₂, each R¹⁷ independently is R³, or wherein two R¹⁷ substituents together with the intervening —O—CH(−)—O— may form a heterocyclyl optionally substituted with one or more R³ and containing up to two oxo groups;
R¹⁸ is selected from hydrogen, C₁₋₆ alkyl, C₁₋₆ fluoroalkyl, C₁₋₆ hydroxyalkyl, C₂₋₇ alkenyl, C₂₋₇ alkynyl, C₃₋₇ cycloalkyl, C₃₋₇ oxyalkyl and may form a cyclic or heterocyclic structure with A, Y or R¹;
or an isomer or a mixture of isomers thereof, or a pharmaceutically acceptable salt, or solvate or prodrug thereof.

A second aspect of the present disclosure relates to a compound of the Formula (Ib)

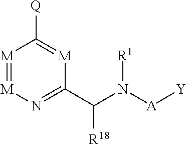

wherein
Q is selected from CO₂H, —CH═NR¹², —W, —CHR²⁰NR²¹R¹³, —CH═O and —CH(OR¹⁷)₂;
A is selected from —C(R^{2a})₂C(O)—, —C(R²)₂C(R²)₂C(O)—, C₁₋₈ alkylene, C₂₋₈ alkenylene, C₂₋₈ alkynylene, —Z′—C₃₋₁₀ cycloalkylene, —Z′-heterocyclylene, —Z′-heteroarylene and —Z′-arylene, which alkylene, alkenylene, alkynylene, —Z′-cycloalkylene, —Z′-heterocyclylene, —Z′-heteroarylene and —Z′-arylene may optionally be substituted with one or more R³ and may form a cyclic or heterocyclic structure with Y; with the proviso that when Q is —CH═O, A is not alkynylene;
Z′ is selected from C₁₋₄ alkylene, C₂₋₅ alkenene, C₂₋₅ alkynene, heterocyclylene and C₃₋₆ cycloalkylene;
Each M is independently selected from CH or N, with the proviso that at least one M is N;
Y is selected from —H, —NR⁶R⁷, C₁₋₈ alkyl, C₂₋₈ alkenyl, C₂₋₈ alkynyl, C₃₋₁₀ cycloalkyl, heterocyclyl, heteroaryl and aryl, which alkyl, alkenyl, alkynyl, cycloalkyl, heterocyclyl, heteroaryl and aryl may optionally be substituted with one or more R³;
R¹ is selected from —H, C₁₋₈ alkyl, C₂₋₈ alkenyl, C₂₋₈ alkynyl, C₃₋₁₀ cycloalkyl, which alkyl, alkenyl, alkynyl and cycloalkyl may be optionally substituted with one or more selected from —OH, aryl, C₁₋₆ alkoxy, heteroaryl, aryloxy, heteroaryloxy, —NR⁶R⁷, F, and C₃₋₆ cycloalkyl; or more preferably is selected from —H and C₁₋₄ alkyl; or with -A-Y forms a nitrogen containing optionally substituted heterocyclic group where the optional substitution may be C₁₋₈ alkyl, C₂₋₈ alkenyl, C₂₋₈ alkynyl, or C₃₋₁₀ cycloalkyl, which alkyl, alkenyl, alkynyl and cycloalkyl may be optionally substituted with one or more selected from —OH, aryl, C₁₋₆ alkoxy, heteroaryl, aryloxy, heteroaryloxy, —NR⁶R⁷, F, and C₃₋₆ cycloalkyl; or with R¹⁸ forms a nitrogen containing optionally substituted heterocyclic group where the optional substitution may be C₁₋₈ alkyl, C₂₋₈ alkenyl, C₂₋₈ alkynyl, or C₃₋₁₀ cycloalkyl, which alkyl, alkenyl, alkynyl and cycloalkyl may be optionally substituted with one or more selected from —OH, aryl, C₁₋₆ alkoxy, heteroaryl, aryloxy, heteroaryloxy, —NR⁶R⁷, F, and C₃₋₆ cycloalkyl;
R² is selected from —H, C₁₋₈ alkyl, C₂₋₈ alkenyl, C₂₋₈ alkynyl, and C₃₋₁₀ cycloalkyl, which alkyl, alkenyl, alkynyl and cycloalkyl may be optionally substituted with one or more selected from —OH, aryl, C₁₋₆ alkoxy, heteroaryl, aryloxy, heteroaryloxy, F, and C₃₋₆ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—C(═O)—R⁷, —Z—OR⁷, halogen, —Z—SR⁷, —Z—SOR⁷, —Z—SO₂R⁷, —Z—SO₂NR⁶R⁷ and —Z—COOR^{7;} alternatively, R² may form a cyclic or heterocyclic structure with another R², R¹ R¹⁸ or Y;
R^{2a} is selected from —H, C₁₋₈ alkyl, C₂₋₈ alkenyl, C₂₋₈ alkynyl, and C₃₋₁₀ cycloalkyl, which alkyl, alkenyl, alkynyl and cycloalkyl may be optionally substituted with one or more selected from —OH, aryl, C₁₋₆ alkoxy, heteroaryl, aryloxy, heteroaryloxy, F, and C₃₋₆ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—NR⁶—C(═O)—OR⁷, —Z—C(═O)—OR⁷, —Z—OR⁷, halogen, —Z—SR⁷, —Z—SOR⁷, —Z—SO₂R⁷, —Z—SO₂NR⁶R⁷ and —Z—COOR^{7;} with the proviso that the two R^{2a} groups are either both non-hydrogen, or that one of the R^{2a} forms a ring with R¹ or R¹⁸;
each R³ is independently selected from C₁₋₆ alkyl, C₁₋₄ fluoroalkyl, C₁₋₄ hydroxyalkyl, C₂₋₆ alkenyl, C₂₋₆ alkynyl, C₃₋₁₀ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—NR⁶—C(═O)—OR⁷, —Z—C(═O)—R⁷, —Z—OR⁷, halogen, —Z—SR⁷, —Z—SOR⁷, —Z—SO₂R⁷, —Z—SO₂NR⁶R⁷, and —Z—COOR⁷, wherein any heterocyclyl may be substituted with one or more R⁴, and wherein any heteroaryl and any aryl may be substituted with one or more R⁵;
Z is selected from a single bond, C₁₋₄ alkylene, heterocyclylene and C₃₋₆ cycloalkylene;
each R⁴ is independently selected from C₁₋₆ alkyl, C₁₋₄ fluoroalkyl, C₁₋₄ hydroxyalkyl, C₁₋₄ alkoxy, C₃₋₁₀ cycloalkyl, NR⁶R⁷, C(═O)—NR⁶R⁷, NR⁶—C(═O)—R⁷, Z—C(═O)—R⁷, —Z—C(═O)—H, OR⁷, halogen, SR⁷, SOR⁷, SO₂R⁷, SO₂NR⁶R⁷ and COOR⁷ and —OH;
each R⁵ is independently selected from C₁₋₆ alkyl, C₁₋₄ fluoroalkyl, C₁₋₄ hydroxyalkyl, C₁₋₄ alkoxy, C₃₋₆ cycloalkyl, —Z—C(═O)—NR⁶R⁷, —Z—NR⁶—C(═O)—R⁷, —Z—C(═O)—R⁷, —Z—C(═O)OR⁷, —Z—NR⁶C(═O)OR⁷, OR⁷, —CN and halogen each of R⁶ and R⁷ is independently selected from hydrogen, C₁₋₈ alkyl, C₁₋₄ fluoroalkyl, C₁₋₄ perfluoroalkyl, C₁₋₄ hydroxyalkyl, C₂₋₈ alkenyl, C₂₋₈ alkynyl, C₃₋₁₀ cycloalkyl, —Z-heterocyclyl, —Z-heteroaryl and —Z-aryl, which alkyl, alkenyl, alkynyl, cycloalkyl, heterocyclyl, heteroaryl and aryl may optionally be substituted with one or more independently selected R⁸; or, alternatively, R⁶ and R⁷ may together with the N-atom to which they are attached form an N-heterocyclic ring optionally substituted with one or more independently selected R⁸;
each R⁸ is independently selected from C₁₋₆ alkyl, C₁₋₄ fluoroalkyl, C₁₋₄ hydroxyalkyl, C₂₋₆ alkenyl, C₂₋₆ alkynyl, C₃₋₁₀ cycloalkyl, —Z-heterocyclyl, —Z-heteroaryl, —Z-aryl, —Z—NR¹⁰R¹¹, —Z—C(═O)—NR¹⁰R¹¹, —Z—OR⁹, halogen, —CN, —Z—SR⁹, —Z—SOR⁹, —Z—SO₂R⁹ and —Z—COOR⁹, which alkyl, alkenyl, alkynyl, cycloalkyl, heterocyclics, heteroaryl and aryl may optionally be substituted with one or more selected from C₁₋₄ alkyl, C₁₋₄ fluoroalkyl, C₁₋₄ hydroxyalkyl, C₃₋₆ cycloalkyl, —Z-heterocyclyl, —Z-heteroaryl, —Z-aryl, —Z—NR¹⁰R¹¹, —Z—C(═O)—NR¹⁰R¹¹, —Z—OR⁹, halogen, —CN, —Z—SR⁹, —Z—SOR⁹, —Z—SO₂R⁹ and —Z—COOR⁹; wherein any heterocyclyl may be further substituted with one or more R⁴ as defined above, and wherein any heteroaryl and any aryl may be further substituted with one or more R⁵ as defined above;
each R⁹ is independently selected from —H, C₁₋₈ alkyl, C₁₋₄ fluoroalkyl, C₁₋₄ hydroxyalkyl, C₂₋₈ alkenyl, C₂₋₈ alkynyl, C₃₋₁₀ cycloalkyl, —Z-heterocyclyl, —Z-aryl, and —Z-heteroaryl, wherein any heterocyclyl may be substituted with one or more R⁴ as defined above, and wherein any heteroaryl and any aryl may be substituted with one or more R⁵ as defined above;
each of R¹⁰ and R¹¹ is independently selected from —H, C₁₋₆ alkyl, C₁₋₄ fluoroalkyl, C₁₋₄ hydroxyalkyl, C₂₋₈ alkenyl, C₂₋₈ alkynyl, C₃₋₁₀ cycloalkyl, heterocyclyl, heteroaryl, and aryl, wherein any heterocyclyl may be substituted with one or more R⁴ as defined above, and wherein any heteroaryl and any aryl may be substituted with one or more R⁵ as defined above, or, alternatively, R¹⁰ and R¹¹ may together with the N-atom to which they are attached form an optionally 5 to 7 membered, N-heterocyclic ring optionally substituted with one or more R⁴ as defined above;
when Q is —CH═NR¹², R¹² selected from C₁₋₁₀ alkyl, C₂₋₁₀ alkenyl, C₂₋₁₀ alkynyl, C₃₋₁₀ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—C(═O)—R⁷, —Z—OR⁷, halogen, —Z—SR⁷, —Z—SOR⁷, —Z—SO₂R⁷ and —Z—COOR⁷, which alkyl, alkenyl, alkynyl, cycloalkyl, heterocyclyl, heteroaryl and aryl may optionally be substituted with one or more R³;
when Q is —CHR²⁰NR²¹R¹³, R¹³ is selected from hydrogen, —C(O)R⁷, —C(O)C(O)R⁷, C₁₋₈ alkyl, C₁₋₄ fluoroalkyl, C₁₋₄ perfluoroalkyl, C₁₋₄ hydroxyalkyl, C₂₋₈ alkenyl, C₂₋₈ alkynyl, C₃₋₁₀ cycloalkyl, —Z-heterocyclyl, —Z-heteroaryl and —Z-aryl, which alkyl, alkenyl, alkynyl, cycloalkyl, heterocyclyl, heteroaryl and aryl may optionally be substituted with one or more independently selected R⁸, or is —CR¹⁴R¹⁵—NR⁶R⁷, —CR¹⁴R¹⁵CN, or —CR¹⁴R¹⁵OR⁷, wherein each of R¹⁴ and R¹⁵ is independently selected from —H, C₁₋₈ alkyl, C₂₋₈ alkenyl, C₂₋₈ alkynyl, C₃₋₁₀ cycloalkyl, heterocyclyl, heteroaryl and aryl, and wherein R¹⁴ and R¹⁵ together with the intervening carbon atom may designate a C₃₋₁₀ cycloalkyl or C₅₋₁₀-cycloalkenyl ring, which alkyl, alkenyl, alkynyl, cycloalkyl (ring), cycloalkenyl ring, heterocyclyl, heteroaryl and aryl may optionally be substituted with one or more R³;
R²⁰ and R²¹ are hydrogen, or together form a 1,3-diaza-C₅₋₇-cycloalk-2-yl group which is N-substituted with R¹⁶ and optionally further substituted with one or more R³, and optionally containing one or two oxo groups; a 1,3-thiaza-C₅₋₇-cycloalk-2-yl group which is N-substituted with R¹⁶ and optionally further substituted with one or more R³ and optionally containing one or two oxo groups; an 1,3-oxaza-C₅₋₇-cycloalk-2-yl group which is N-substituted with R¹⁶ and optionally further substituted with one or more R³, and optionally containing one or two oxo groups, wherein in all three instances two R³'s on the same carbon atom may together form a spiro group;
when Q is W, W is selected from an 1,3-diaza-C₅₋₇-cycloalk-2-yl group which is N-substituted with R¹⁶ and optionally further substituted with one or more R³, and optionally containing one or two oxo groups; a 1,3-thiaza-C₅₋₇-cycloalk-2-yl group which is N-substituted with R¹⁶ and optionally further substituted with one or more R³ and optionally containing one or two oxo groups; an 1,3-oxaza-C₅₋₇-cycloalk-2-yl group which is N-substituted with R¹⁶ and optionally further substituted with one or more R³, and optionally containing one or two oxo groups, wherein in all three instances two R³'s on the same carbon atom may together form a spiro group;
R¹⁶ is selected from hydrogen, —C(O)R⁷, —C(O)C(O)R⁷, —C(O)C(O)OR⁷, and —C(O)C(O)NR⁶R⁷;
when Q is —CH(OR¹⁷)₂, each R¹⁷ independently is R³, or wherein two R¹⁷ substituents together with the intervening —O—CH(−)—O— may form a heterocyclyl optionally substituted with one or more R³ and containing up to two oxo groups;
R¹⁸ is selected from hydrogen, C₁₋₆ alkyl, C₁₋₆ fluoroalkyl, C₁₋₆ hydroxyalkyl, C₂₋₇ alkenyl, C₂₋₇ alkynyl, C₃₋₇ cycloalkyl, C₃₋₇ oxyalkyl and may form a cyclic or heterocyclic structure with A, Y or R¹;
or an isomer or a mixture of isomers thereof, or a pharmaceutically acceptable salt, or solvate or prodrug thereof.

A third aspect of the present disclosure relates to a compound of the Formula (Ic)

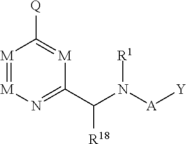

wherein
Q is selected from CO₂H, —CH═NR¹², —W, —CHR²⁰NR²¹R¹³, —CH═O and —CH(OR¹⁷)₂;
A is selected from —C(R^{2a})₂C(O)—, —C(R²)₂C(R²)₂C(O)—, C₁₋₈ alkylene, C₂₋₈ alkenylene, C₂₋₈ alkynylene, —Z′—C₃₋₁₀ cycloalkylene, —Z′-heterocyclylene, —Z′-heteroarylene and —Z′-arylene, which alkylene, alkenylene, alkynylene, —Z′-cycloalkylene, —Z′-heterocyclylene, —Z′-heteroarylene and —Z′-arylene may optionally be substituted with one or more R³ and may form a cyclic or heterocyclic structure with Y; with the proviso that when Q is —CH═O, A is not alkynylene;
Z′ is selected from C₁₋₄ alkylene, C₂₋₅ alkenene, C₂₋₅ alkynene, heterocyclylene and C₃₋₆ cycloalkylene;
Each M is independently selected from CH or N;
Y is selected from —H, —NR⁶R⁷, —OR⁷, C₁₋₈ alkyl, C₂₋₈ alkenyl, C₂₋₈ alkynyl, C₃₋₁₀ cycloalkyl, heterocyclyl, heteroaryl and aryl, which alkyl, alkenyl, alkynyl, cycloalkyl, heterocyclyl, heteroaryl and aryl may optionally be substituted with one or more R³;
R¹ is selected from C₁₋₈ alkyl, C₂₋₈ alkenyl, C₂₋₈ alkynyl, C₃₋₁₀ cycloalkyl, which alkyl, alkenyl, alkynyl and cycloalkyl are substituted with one or more selected from —Z—C(═O)—NR⁶R⁷, —Z—NR⁶C(═O)—NR⁶R⁷, —Z—NR⁶—C(═O)—R⁷, —Z—C(═O)—R⁷, —Z—NR⁶C(═O)OR⁷, —Z—C(═O)OR⁷; or more preferably is selected from —H and C₁₋₄ alkyl; or with -A-Y forms a nitrogen containing substituted heterocyclic group where the substitution is C₁₋₈ alkyl, C₂₋₈ alkenyl, C₂₋₈ alkynyl, or C₃₋₁₀ cycloalkyl, which alkyl, alkenyl, alkynyl and cycloalkyl is substituted with one or more selected from —NR⁶R⁷, —Z—C(═O)—NR⁶R⁷, —Z—NR⁶C(═O)—NR⁶R⁷, —Z—NR⁶—C(═O)—R⁷, —Z—C(═O)—R⁷, —Z—NR⁶C(═O)OR⁷, —Z—C(═O)OR⁷; or with R¹⁸ forms a nitrogen containing optionally substituted heterocyclic group where the optional substitution may be C₁₋₈ alkyl, C₂₋₈ alkenyl, C₂₋₈ alkynyl, or C₃₋₁₀ cycloalkyl, which alkyl, alkenyl, alkynyl and cycloalkyl may be optionally substituted with one or more selected from —OH, aryl, C₁₋₆ alkoxy, heteroaryl, aryloxy, heteroaryloxy, —NR⁶R⁷, F, —Z—C(═O)—NR⁶R⁷, —Z—NR⁶C(═O)—NR⁶R⁷, —Z—NR⁶—C(═O)—R⁷, —Z—C(═O)—R⁷, —Z—NR⁶C(═O)OR⁷, —Z—C(═O)OR⁷ and C₃₋₆ cycloalkyl;
R² is selected from —H, C₁₋₈ alkyl, C₂₋₈ alkenyl, C₂₋₈ alkynyl, and C₃₋₁₀ cycloalkyl, which alkyl, alkenyl, alkynyl and cycloalkyl may be optionally substituted with one or more selected from —OH, aryl, C₁₋₆ alkoxy, heteroaryl, aryloxy, heteroaryloxy, F, and C₃₋₆ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—C(═O)—R⁷, —Z—OR⁷, halogen, —Z—SR⁷, —Z—SOR⁷, —Z—SO₂R⁷, —Z—SO₂NR⁶R⁷ and —Z—COOR⁷; alternatively, R² may form a cyclic or heterocyclic structure with another R², R¹ R¹⁸ or Y;
R^{2a} is selected from —H, C₁₋₈ alkyl, C₂₋₈ alkenyl, C₂₋₈ alkynyl, and C₃₋₁₀ cycloalkyl, which alkyl, alkenyl, alkynyl and cycloalkyl may be optionally substituted with one or more selected from —OH, aryl, C₁₋₆ alkoxy, heteroaryl, aryloxy, heteroaryloxy, F, and C₃₋₆ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—NR⁶—C(═O)—OR⁷, —Z—C(═O)—OR⁷, —Z—OR⁷, halogen, —Z—SR⁷, —Z—SOR⁷, —Z—SO₂R⁷, —Z—SO₂NR⁶R⁷ and —Z—COOR⁷; with the proviso that the two R^{2a} groups are either both non-hydrogen, or that one of the R^{2a} forms a ring with R¹ or R¹⁸;
each R³ is independently selected from C₁₋₆ alkyl, C₁₋₄ fluoroalkyl, C₁₋₄ hydroxyalkyl, C₂₋₆ alkenyl, C₂₋₆ alkynyl, C₃₋₁₀ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—NR⁶—C(═O)—OR⁷, —Z—C(═O)—R⁷, —Z—OR⁷, halogen, —Z—SR⁷, —Z—SOR⁷, —Z—SO₂R⁷, —Z—SO₂NR⁶R⁷, and —Z—COOR⁷, wherein any heterocyclyl may be substituted with one or more R⁴, and wherein any heteroaryl and any aryl may be substituted with one or more R⁵;
Z is selected from a single bond, C₁₋₄ alkylene, heterocyclylene and C₃₋₆ cycloalkylene;
each R⁴ is independently selected from C₁₋₆ alkyl, C₁₋₄ fluoroalkyl, C₁₋₄ hydroxyalkyl, C₁₋₄ alkoxy, C₃₋₁₀ cycloalkyl, NR⁶R⁷, C(═O)—NR⁶R⁷, NR⁶—C(═O)—R⁷, Z—C(═O)—R⁷, —Z—C(═O)—H, OR⁷, halogen, SR⁷, SOR⁷, SO₂R⁷, SO₂NR⁶R⁷ and COOR⁷ and —OH;
each R⁵ is independently selected from C₁₋₆ alkyl, C₁₋₄ fluoroalkyl, C₁₋₄ hydroxyalkyl, C₁₋₄ alkoxy, C₃₋₆ cycloalkyl, —Z—C(═O)—NR⁶R⁷, —Z—NR⁶—C(═O)—R⁷, —Z—C(═O)—R⁷, —Z—NR⁶C(═O)OR⁷, —Z—C(═O)OR⁷, OR⁷, —CN and halogen each of R⁶ and R⁷ is independently selected from hydrogen, C₁₋₈ alkyl, C₁₋₄ fluoroalkyl, C₁₋₄ perfluoroalkyl, C₁₋₄ hydroxyalkyl, C₂₋₈ alkenyl, C₂₋₈ alkynyl, C₃₋₁₀ cycloalkyl, —Z-heterocyclyl, —Z-heteroaryl and —Z-aryl, which alkyl, alkenyl, alkynyl, cycloalkyl, heterocyclyl, heteroaryl and aryl may optionally be substituted with one or more independently selected R⁸; or, alternatively, R⁶ and R⁷ may together with the N-atom to which they are attached form an N-heterocyclic ring optionally substituted with one or more independently selected R⁸;
each R⁸ is independently selected from C₁₋₆ alkyl, C₁₋₄ fluoroalkyl, C₁₋₄ hydroxyalkyl, C₂₋₆ alkenyl, C₂₋₆ alkynyl, C₃₋₁₀ cycloalkyl, —Z-heterocyclyl, —Z-heteroaryl, —Z-aryl, —Z—NR¹⁰R¹¹, —Z—C(═O)—NR¹⁰R¹¹, —Z—OR⁹, halogen, —CN, —Z—SR⁹, —Z—SOR⁹, —Z—SO₂R⁹ and —Z—COOR⁹, which alkyl, alkenyl, alkynyl, cycloalkyl, heterocyclics, heteroaryl and aryl may optionally be substituted with one or more selected from C₁₋₄ alkyl, C₁₋₄ fluoroalkyl, C₁₋₄ hydroxyalkyl, C₃₋₆ cycloalkyl, —Z-heterocyclyl, —Z-heteroaryl, —Z-aryl, —Z—NR¹⁰R¹¹, —Z—C(═O)—NR¹⁰R¹¹, —Z—OR⁹, halogen, —CN, —Z—SR⁹, —Z—SOR⁹, —Z—SO₂R⁹ and —Z—COOR⁹; wherein any heterocyclyl may be further substituted with one or more R⁴ as defined above, and wherein any heteroaryl and any aryl may be further substituted with one or more R⁵ as defined above;
each R⁹ is independently selected from —H, C₁₋₈ alkyl, C₁₋₄ fluoroalkyl, C₁₋₄ hydroxyalkyl, C₂₋₈ alkenyl, C₂₋₈ alkynyl, C₃₋₁₀ cycloalkyl, —Z-heterocyclyl, —Z-aryl, and —Z-heteroaryl, wherein any heterocyclyl may be substituted with one or more R⁴ as defined above, and wherein any heteroaryl and any aryl may be substituted with one or more R⁵ as defined above;
each of R¹⁰ and R¹¹ is independently selected from —H, C₁₋₆ alkyl, C₁₋₄ fluoroalkyl, C₁₋₄ hydroxyalkyl, C₂₋₈ alkenyl, C₂₋₈ alkynyl, C₃₋₁₀ cycloalkyl, heterocyclyl, heteroaryl, and aryl, wherein any heterocyclyl may be substituted with one or more R⁴ as defined above, and wherein any heteroaryl and any aryl may be substituted with one or more R⁵ as defined above, or, alternatively, R¹⁰ and R¹¹ may together with the N-atom to which they are attached form an optionally 5 to 7 membered, N-heterocyclic ring optionally substituted with one or more R⁴ as defined above;
with the proviso that Y is not H when A is —CH₂—;
when Q is —CH═NR¹², R¹² is selected from C₁₋₁₀ alkyl, C₂₋₁₀ alkenyl, C₂₋₁₀ alkynyl, C₃₋₁₀ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—C(═O)—R⁷, —Z—OR⁷, halogen, —Z—SR⁷, —Z—SOR⁷, —Z—SO₂R⁷ and —Z—COOR⁷, which alkyl, alkenyl, alkynyl, cycloalkyl, heterocyclyl, heteroaryl and aryl may optionally be substituted with one or more R³;
when Q is —CHR²⁰NR²¹R¹³, R¹³ is selected from hydrogen, —C(O)R⁷, —C(O)C(O)R⁷, —C(O)C(O)OR⁷, C₁₋₈ alkyl, C₁₋₄ fluoroalkyl, C₁₋₄ perfluoroalkyl, C₁₋₄ hydroxyalkyl, C₂₋₈ alkenyl, C₂₋₈ alkynyl, C₃₋₁₀ cycloalkyl, —Z-heterocyclyl, and —Z-monocyclic-heteroaryl, which alkyl, alkenyl, alkynyl, cycloalkyl, heterocyclyl, and monocyclic-heteroaryl may optionally be substituted with one or more independently selected R⁸, or is —CR¹⁴R¹⁵—NR⁶R⁷, —CR¹⁴R¹⁵CN, or —CR¹⁴R¹⁵OR⁷, wherein each of R¹⁴ and R¹⁵ is independently selected from —H, C₁₋₈ alkyl, C₂₋₈ alkenyl, C₂₋₈ alkynyl, C₃₋₁₀ cycloalkyl, heterocyclyl, heteroaryl and aryl, and wherein R¹⁴ and R¹⁵ together with the intervening carbon atom may designate a C₃₋₁₀ cycloalkyl or C₅₋₁₀-cycloalkenyl ring, which alkyl, alkenyl, alkynyl, cycloalkyl (ring), cycloalkenyl ring, heterocyclyl, heteroaryl and aryl may optionally be substituted with one or more R³;
R²⁰ and R²¹ are hydrogen, or together form a 1,3-diaza-C₅₋₇-cycloalk-2-yl group which is N-substituted with R¹⁶ and optionally further substituted with one or more R³, and optionally containing one or two oxo groups; a 1,3-thiaza-C₅₋₇-cycloalk-2-yl group which is N-substituted with R¹⁶ and optionally further substituted with one or more R³ and optionally containing one or two oxo groups; an 1,3-oxaza-C₅₋₇-cycloalk-2-yl group which is N-substituted with R¹⁶ and optionally further substituted with one or more R³, and optionally containing one or two oxo groups, wherein in all three instances two R³'s on the same carbon atom may together form a spiro group;
when Q is W, W is selected from an 1,3-diaza-C₅₋₇-cycloalk-2-yl group which is N-substituted with R¹⁶ and optionally further substituted with one or more R³, and optionally containing one or two oxo groups; a 1,3-thiaza-C₅₋₇-cycloalk-2-yl group which is N-substituted with R¹⁶ and optionally further substituted with one or more R³ and optionally containing one or two oxo groups; an 1,3-oxaza-C₅₋₇-cycloalk-2-yl group which is N-substituted with R¹⁶ and optionally further substituted with one or more R³, and optionally containing one or two oxo groups, wherein in all three instances two R³'s on the same carbon atom may together form a spiro group;
R¹⁶ is selected from hydrogen, —C(O)R⁷, —C(O)C(O)R⁷, —C(O)C(O)OR⁷, and —C(O)C(O)NR⁶R⁷;
when Q is —CH(OR¹⁷)₂, each R¹⁷ independently is R³, or wherein two R¹⁷ substituents together with the intervening —O—CH(−)—O— may form a heterocyclyl optionally substituted with one or more R³ and containing up to two oxo groups;
R¹⁸ is selected from hydrogen, C₁₋₆ alkyl, C₁₋₆ fluoroalkyl, C₁₋₆ hydroxyalkyl, C₂₋₇ alkenyl, C₂₋₇ alkynyl, C₃₋₇ cycloalkyl, C₃₋₇ oxyalkyl and may form a cyclic or heterocyclic structure with A, Y or R¹;
or an isomer or a mixture of isomers thereof, or a pharmaceutically acceptable salt, or solvate or prodrug thereof.

A fourth aspect of the present disclosure relates to a compound of the Formula (Id)

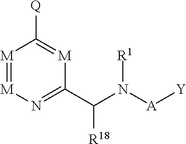

wherein
Q is selected from CO₂H, —CH═NR¹², —W, —CHR²⁰NR²¹R¹³, —CH═O and —CH(OR¹⁷)₂;
A is selected from —C(R^{2a})₂C(O)—, —C(R²)₂C(R²)₂C(O)—, C₁₋₈ alkylene, C₂₋₈ alkenylene, C₂₋₈ alkynylene, —Z′—C₃₋₁₀ cycloalkylene, —Z′-heterocyclylene, —Z′-heteroarylene and —Z′-arylene, which alkylene, alkenylene, alkynylene, —Z′-cycloalkylene, —Z′-heterocyclylene, —Z′-heteroarylene and —Z′-arylene may optionally be substituted with one or more R³ and may form a cyclic or heterocyclic structure with Y; with the proviso that when Q is —CH═O, A is not alkynylene;
Z′ is selected from C₁₋₄ alkylene, C₂₋₅ alkenene, C₂₋₅ alkynene, heterocyclylene and C₃₋₆ cycloalkylene;
Each M is independently selected from CH or N;
Y is selected from —H, —NR⁶R⁷, —OR⁷, C₁₋₈ alkyl, C₂₋₈ alkenyl, C₂₋₈ alkynyl, C₃₋₁₀ cycloalkyl, heterocyclyl, heteroaryl and aryl, which alkyl, alkenyl, alkynyl, cycloalkyl, heterocyclyl, heteroaryl and aryl may optionally be substituted with one or more R³; or may form a cyclic or heterocyclic structure with R²;
R¹ is selected from —H, C₁₋₈ alkyl, C₂₋₈ alkenyl, C₂₋₈ alkynyl, C₃₋₁₀ cycloalkyl, which alkyl, alkenyl, alkynyl and cycloalkyl may be optionally substituted with one or more selected from —OH, aryl, C₁₋₆ alkoxy, heteroaryl, aryloxy, heteroaryloxy, —NR⁶R⁷, F, and C₃₋₆ cycloalkyl; or more preferably is selected from —H and C₁₋₄ alkyl; or with -A-Y forms a nitrogen containing optionally substituted heterocyclic group where the optional substitution may be C₁₋₈ alkyl, C₂₋₈ alkenyl, C₂₋₈ alkynyl, or C₃₋₁₀ cycloalkyl, which alkyl, alkenyl, alkynyl and cycloalkyl may be optionally substituted with one or more selected from —OH, aryl, C₁₋₆ alkoxy, heteroaryl, aryloxy, heteroaryloxy, —NR⁶R⁷, F, and C₃₋₆ cycloalkyl; or with R¹⁸ forms a nitrogen containing optionally substituted heterocyclic group where the optional substitution may be C₁₋₈ alkyl, C₂₋₈ alkenyl, C₂₋₈ alkynyl, or C₃₋₁₀ cycloalkyl, which alkyl, alkenyl, alkynyl and cycloalkyl may be optionally substituted with one or more selected from —OH, aryl, C₁₋₆ alkoxy, heteroaryl, aryloxy, heteroaryloxy, —NR⁶R⁷, F, and C₃₋₆ cycloalkyl;
R² is selected from C₁₋₈ alkyl, C₂₋₈ alkenyl, C₂₋₈ alkynyl, and C₃₋₁₀ cycloalkyl, which alkyl, alkenyl, alkynyl and cycloalkyl are substituted with one or more selected from —Z′-aryl, —Z′-heteroaryl, —Z′—NR⁶R⁷, —Z′—C(═O)—NR⁶R⁷, —Z′—NR⁶C(═O)—NR⁶R⁷, —Z′—NR⁶—C(═O)—R⁷, —Z′—C(═O)—R⁷, —Z′—C(═O)OR⁷, —Z′—OR⁷, halogen, —Z′—SR⁷, —Z′—SOR⁷, —Z′—SO₂R⁷, —Z′—SO₂NR⁶R⁷ and —Z′—COOR⁷; R² may form a ring with R¹, R¹⁸, another R² or Y;
each R³ is independently selected from C₁₋₆ alkyl, C₁₋₄ fluoroalkyl, C₁₋₄ hydroxyalkyl, C₂₋₆ alkenyl, C₂₋₆ alkynyl, C₃₋₁₀ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—C(═O)—R⁷, —Z—OR⁷, halogen, —Z—SR⁷, —Z—SOR⁷, —Z—SO₂R⁷, —Z—SO₂NR⁶R⁷ and —Z—COOR⁷, wherein any heterocyclyl may be substituted with one or more R⁴, and wherein any heteroaryl and any aryl may be substituted with one or more R⁵;
Z is selected from a single bond, C₁₋₄ alkylene, heterocyclylene and C₃₋₆ cycloalkylene;
each R⁴ is independently selected from C₁₋₆ alkyl, C₁₋₄ fluoroalkyl, C₁₋₄ hydroxyalkyl, C₁₋₄ alkoxy, C₃₋₁₀ cycloalkyl, NR⁶R⁷, C(═O)—NR⁶R⁷, NR⁶—C(═O)—R⁷, Z—C(═O)—R⁷, —Z—C(═O)—H, OR⁷, halogen, SR⁷, SOR⁷, SO₂R⁷, SO₂NR⁶R⁷ and COOR⁷ and —OH;
each R⁵ is independently selected from C₁₋₆ alkyl, C₁₋₄ fluoroalkyl, C₁₋₄ hydroxyalkyl, C₁₋₄ alkoxy, C₃₋₆ cycloalkyl, —Z—C(═O)—NR⁶R⁷, —Z—NR⁶—C(═O)—R⁷, —Z—C(═O)—R⁷, —Z—NR⁶C(═O)OR⁷, —Z—C(═O)OR⁷, OR⁷, —CN and halogen each of R⁶ and R⁷ is independently selected from hydrogen, C₁₋₈ alkyl, C₁₋₄ fluoroalkyl, C₁₋₄ perfluoroalkyl, C₁₋₄ hydroxyalkyl, C₂₋₈ alkenyl, C₂₋₈ alkynyl, C₃₋₁₀ cycloalkyl, —Z-heterocyclyl, —Z-heteroaryl and —Z-aryl, which alkyl, alkenyl, alkynyl, cycloalkyl, heterocyclyl, heteroaryl and aryl may optionally be substituted with one or more independently selected R⁸; or, alternatively, R⁶ and R⁷ may together with the N-atom to which they are attached form an N-heterocyclic ring optionally substituted with one or more independently selected R⁸;
each R⁸ is independently selected from C₁₋₆ alkyl, C₁₋₄ fluoroalkyl, C₁₋₄ hydroxyalkyl, C₂₋₆ alkenyl, C₂₋₆ alkynyl, C₃₋₁₀ cycloalkyl, —Z-heterocyclyl, —Z-heteroaryl, —Z-aryl, —Z—NR¹⁰R¹¹, —Z—C(═O)—NR¹⁰R¹¹, —Z—OR⁹, halogen, —CN, —Z—SR⁹, —Z—SOR⁹, —Z—SO₂R⁹ and —Z—COOR⁹, which alkyl, alkenyl, alkynyl, cycloalkyl, heterocyclics, heteroaryl and aryl may optionally be substituted with one or more selected from C₁₋₄ alkyl, C₁₋₄ fluoroalkyl, C₁₋₄ hydroxyalkyl, C₃₋₆ cycloalkyl, —Z-heterocyclyl, —Z-heteroaryl, —Z-aryl, —Z—NR¹⁰R¹¹, —Z—C(═O)—NR¹⁰R¹¹, —Z—OR⁹, halogen, —CN, —Z—SR⁹, —Z—SOR⁹, —Z—SO₂R⁹ and —Z—COOR⁹; wherein any heterocyclyl may be further substituted with one or more R⁴ as defined above, and wherein any heteroaryl and any aryl may be further substituted with one or more R⁵ as defined above;
each R⁹ is independently selected from —H, C₁₋₈ alkyl, C₁₋₄ fluoroalkyl, C₁₋₄ hydroxyalkyl, C₂₋₈ alkenyl, C₂₋₈ alkynyl, C₃₋₁₀ cycloalkyl, —Z-heterocyclyl, —Z-aryl, and —Z-heteroaryl, wherein any heterocyclyl may be substituted with one or more R⁴ as defined above, and wherein any heteroaryl and any aryl may be substituted with one or more R⁵ as defined above;
each of R¹⁰ and R¹¹ is independently selected from —H, C₁₋₆ alkyl, C₁₋₄ fluoroalkyl, C₁₋₄ hydroxyalkyl, C₂₋₈ alkenyl, C₂₋₈ alkynyl, C₃₋₁₀ cycloalkyl, heterocyclyl, heteroaryl, and aryl, wherein any heterocyclyl may be substituted with one or more R⁴ as defined above, and wherein any heteroaryl and any aryl may be substituted with one or more R⁵ as defined above, or, alternatively, R¹⁰ and R¹¹ may together with the N-atom to which they are attached form an optionally 5 to 7 membered, N-heterocyclic ring optionally substituted with one or more R⁴ as defined above;
when Q is —CH═NR¹², R¹² is selected from C₁₋₁₀ alkyl, C₂₋₁₀ alkenyl, C₂₋₁₀ alkynyl, C₃₋₁₀ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—C(═O)—R⁷, —Z—OR⁷, halogen, —Z—SR⁷, —Z—SOR⁷, —Z—SO₂R⁷ and —Z—COOR⁷, which alkyl, alkenyl, alkynyl, cycloalkyl, heterocyclyl, heteroaryl and aryl may optionally be substituted with one or more R³;
when Q is —CHR²⁰NR²¹R¹³, R¹³ is selected from hydrogen, —C(O)R⁷, —C(O)C(O)R⁷, C₁₋₈ alkyl, C₁₋₄ fluoroalkyl, C₁₋₄ perfluoroalkyl, C₁₋₄ hydroxyalkyl, C₂₋₈ alkenyl, C₂₋₈ alkynyl, C₃₋₁₀ cycloalkyl, —Z-heterocyclyl, —Z-heteroaryl and —Z-aryl, which alkyl, alkenyl, alkynyl, cycloalkyl, heterocyclyl, heteroaryl and aryl may optionally be substituted with one or more independently selected R⁸, or is —CR¹⁴R¹⁵—NR⁶R⁷, —CR¹⁴R¹⁵CN, or —CR¹⁴R¹⁵OR⁷, wherein each of R¹⁴ and R¹⁵ is independently selected from —H, C₁₋₈ alkyl, C₂₋₈ alkenyl, C₂₋₈ alkynyl, C₃₋₁₀ cycloalkyl, heterocyclyl, heteroaryl and aryl, and wherein R¹⁴ and R¹⁵ together with the intervening carbon atom may designate a C₃₋₁₀ cycloalkyl or C₅₋₁₀-cycloalkenyl ring, which alkyl, alkenyl, alkynyl, cycloalkyl (ring), cycloalkenyl ring, heterocyclyl, heteroaryl and aryl may optionally be substituted with one or more R³;
R²⁰ and R²¹ are hydrogen, or together form a 1,3-diaza-C₅₋₇-cycloalk-2-yl group which is N-substituted with R¹⁶ and optionally further substituted with one or more R³, and optionally containing one or two oxo groups; a 1,3-thiaza-C₅₋₇-cycloalk-2-yl group which is N-substituted with R¹⁶ and optionally further substituted with one or more R³ and optionally containing one or two oxo groups; an 1,3-oxaza-C₅₋₇-cycloalk-2-yl group which is N-substituted with R¹⁶ and optionally further substituted with one or more R³, and optionally containing one or two oxo groups, wherein in all three instances two R³'s on the same carbon atom may together form a spiro group;
when Q is W, W is selected from an 1,3-diaza-C₅₋₇-cycloalk-2-yl group which is N-substituted with R¹⁶ and optionally further substituted with one or more R³, and optionally containing one or two oxo groups; a 1,3-thiaza-C₅₋₇-cycloalk-2-yl group which is N-substituted with R¹⁶ and optionally further substituted with one or more R³ and optionally containing one or two oxo groups; an 1,3-oxaza-C₅₋₇-cycloalk-2-yl group which is N-substituted with R¹⁶ and optionally further substituted with one or more R³, and optionally containing one or two oxo groups, wherein in all three instances two R³'s on the same carbon atom may together form a spiro group;
R¹⁶ is selected from hydrogen, —C(O)R⁷, —C(O)C(O)R⁷, —C(O)C(O)OR⁷, and —C(O)C(O)NR⁶R⁷;
when Q is —CH(OR¹⁷)₂, each R¹⁷ independently is R³, or wherein two R¹⁷ substituents together with the intervening —O—CH(−)—O— may form a heterocyclyl optionally substituted with one or more R³ and containing up to two oxo groups;
R¹⁸ is selected from hydrogen, C₁₋₆ alkyl, C₁₋₆ fluoroalkyl, C₁₋₆ hydroxyalkyl, C₂₋₇ alkenyl, C₂₋₇ alkynyl, C₃₋₇ cycloalkyl, C₃₋₇ oxyalkyl and may form a cyclic or heterocyclic structure with A, Y or R¹;
or an isomer or a mixture of isomers thereof, or a pharmaceutically acceptable salt, or solvate or prodrug thereof.

A fifth aspect of the present disclosure relates to a compound of the Formula (Ie)

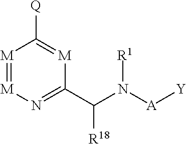

wherein
Q is selected from CO₂H, —CH═NR¹², —W, —CHR²⁰NR²¹R¹³, —CH═O and —CH(OR¹⁷)₂;
A is selected from —C(R^{2a})₂C(O)—, —C(R²)₂C(R²)₂C(O)—, C₁₋₈ alkylene, C₂₋₈ alkenylene, C₂₋₈ alkynylene, —Z′—C₃₋₁₀ cycloalkylene, —Z′-heterocyclylene, —Z′-heteroarylene and —Z′-arylene, which alkylene, alkenylene, alkynylene, —Z′-cycloalkylene, —Z′-heterocyclylene, —Z′-heteroarylene and —Z′-arylene may optionally be substituted with one or more R³ and may form a cyclic or heterocyclic structure with Y; with the proviso that when Q is —CH═O, A is not alkynylene;
Z′ is selected from C₁₋₄ alkylene, C₂₋₅ alkenene, C₂₋₅ alkynene, heterocyclylene and C₃₋₆ cycloalkylene;
Each M is independently selected from CH or N;
Y is selected from —H, —NR⁶R⁷, —OR⁷, C₁₋₈ alkyl, C₂₋₈ alkenyl, C₂₋₈ alkynyl, C₃₋₁₀ cycloalkyl, heterocyclyl, heteroaryl and aryl, which alkyl, alkenyl, alkynyl, cycloalkyl, heterocyclyl, heteroaryl and aryl may optionally be substituted with one or more R³;
R¹ is selected from —H, C₁₋₈ alkyl, C₂₋₈ alkenyl, C₂₋₈ alkynyl, C₃₋₁₀ cycloalkyl, which alkyl, alkenyl, alkynyl and cycloalkyl may be optionally substituted with one or more selected from —OH, aryl, C₁₋₆ alkoxy, heteroaryl, aryloxy, heteroaryloxy, —NR⁶R⁷, F, and C₃₋₆ cycloalkyl; or more preferably is selected from —H and C₁₋₄ alkyl; or with -A-Y forms a nitrogen containing optionally substituted heterocyclic group where the optional substitution may be C₁₋₈ alkyl, C₂₋₈ alkenyl, C₂₋₈ alkynyl, or C₃₋₁₀ cycloalkyl, which alkyl, alkenyl, alkynyl and cycloalkyl may be optionally substituted with one or more selected from —OH, aryl, C₁₋₆ alkoxy, heteroaryl, aryloxy, heteroaryloxy, —NR⁶R⁷, F, and C₃₋₆ cycloalkyl; or with R¹⁸ forms a nitrogen containing optionally substituted heterocyclic group where the optional substitution may be C₁₋₈ alkyl, C₂₋₈ alkenyl, C₂₋₈ alkynyl, or C₃₋₁₀ cycloalkyl, which alkyl, alkenyl, alkynyl and cycloalkyl may be optionally substituted with one or more selected from —OH, aryl, C₁₋₆ alkoxy, heteroaryl, aryloxy, heteroaryloxy, —NR⁶R⁷, F, and C₃₋₆ cycloalkyl;
R² is selected from —H, C₁₋₈ alkyl, C₂₋₈ alkenyl, C₂₋₈ alkynyl, and C₃₋₁₀ cycloalkyl, which alkyl, alkenyl, alkynyl and cycloalkyl may be optionally substituted with one or more selected from —OH, aryl, C₁₋₆ alkoxy, heteroaryl, aryloxy, heteroaryloxy, F, and C₃₋₆ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—C(═O)—R⁷, —Z—OR⁷, halogen, —Z—SR⁷, —Z—SOR⁷, —Z—SO₂R⁷, —Z—SO₂NR⁶R⁷ and —Z—COOR⁷; alternatively, R² may form a cyclic or heterocyclic structure with another R², R¹ R¹⁸ or Y;
R^{2a} is selected from —H, C₁₋₈ alkyl, C₂₋₈ alkenyl, C₂₋₈ alkynyl, and C₃₋₁₀ cycloalkyl, which alkyl, alkenyl, alkynyl and cycloalkyl may be optionally substituted with one or more selected from —OH, aryl, C₁₋₆ alkoxy, heteroaryl, aryloxy, heteroaryloxy, F, and C₃₋₆ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—NR⁶—C(═O)—OR⁷, —Z—C(═O)—OR⁷, —Z—OR⁷, halogen, —Z—SR⁷, —Z—SOR⁷, —Z—SO₂R⁷, —Z—SO₂NR⁶R⁷ and —Z—COOR⁷; with the proviso that the two R^{2a} groups are either both non-hydrogen, or that one of the R^{2a} forms a ring with R¹ or R¹⁸;
each R³ is independently selected from C₁₋₆ alkyl, C₁₋₄ fluoroalkyl, C₁₋₄ hydroxyalkyl, C₂₋₆ alkenyl, C₂₋₆ alkynyl, C₃₋₁₀ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—C(═O)—R⁷, —Z—OR⁷, halogen, —Z—SR⁷, —Z—SOR⁷, —Z—SO₂R⁷, —Z—SO₂NR⁶R⁷ and —Z—COOR⁷, wherein any heterocyclyl may be substituted with one or more R⁴, and wherein any heteroaryl and any aryl may be substituted with one or more R⁵;
Z is selected from a single bond, C₁₋₄ alkylene, heterocyclylene and C₃₋₆ cycloalkylene;
each R⁴ is independently selected from, —Z′—NR⁶C(═O)—NR⁶R⁷, —Z′—NR⁶—C(═O)—R⁷, —Z′—C(═O)—R⁷, —Z′—C(═O)OR⁷, OR⁷ (with the proviso that OR⁷ is not C₁₋₆ alkyl), halogen, SR⁷, SOR⁷, SO₂R⁷, SO₂NR⁶R⁷ and COOR⁷;
each R⁵ is independently selected from C₁₋₆ alkyl, C₁₋₄ fluoroalkyl, C₁₋₄ hydroxyalkyl, C₁₋₄ alkoxy, C₃₋₆ cycloalkyl, —Z—C(═O)—NR⁶R⁷, —Z—NR⁶—C(═O)—R⁷, —Z—C(═O)—R⁷, —Z—NR⁶C(═O)OR⁷, —Z—C(═O)OR⁷, OR⁷, —CN and halogen each of R⁶ and R⁷ is independently selected from hydrogen, C₁₋₈ alkyl, C₁₋₄ fluoroalkyl, C₁₋₄ perfluoroalkyl, C₁₋₄ hydroxyalkyl, C₂₋₈ alkenyl, C₂₋₈ alkynyl, C₃₋₁₀ cycloalkyl, —Z-heterocyclyl, —Z-heteroaryl and —Z-aryl, which alkyl, alkenyl, alkynyl, cycloalkyl, heterocyclyl, heteroaryl and aryl may optionally be substituted with one or more independently selected R⁸; or, alternatively, R⁶ and R⁷ may together with the N-atom to which they are attached form an N-heterocyclic ring optionally substituted with one or more independently selected R⁸;
each R⁸ is independently selected from C₁₋₆ alkyl, C₁₋₄ fluoroalkyl, C₁₋₄ hydroxyalkyl, C₂₋₆ alkenyl, C₂₋₆ alkynyl, C₃₋₁₀ cycloalkyl, —Z-heterocyclyl, —Z-heteroaryl, —Z-aryl, —Z—NR¹⁰R¹¹, —Z—C(═O)—NR¹⁰R¹¹, —Z—OR⁹, halogen, —CN, —Z—SR⁹, —Z—SOR⁹, —Z—SO₂R⁹ and —Z—COOR⁹, which alkyl, alkenyl, alkynyl, cycloalkyl, heterocyclics, heteroaryl and aryl may optionally be substituted with one or more selected from C₁₋₄ alkyl, C₁₋₄ fluoroalkyl, C₁₋₄ hydroxyalkyl, C₃₋₆ cycloalkyl, —Z-heterocyclyl, —Z-heteroaryl, —Z-aryl, —Z—NR¹⁰R¹¹, —Z—C(═O)—NR¹⁰R¹¹, —Z—OR⁹, halogen, —CN, —Z—SR⁹, —Z—SOR⁹, —Z—SO₂R⁹ and —Z—COOR⁹; wherein any heterocyclyl may be further substituted with one or more R⁴ as defined above, and wherein any heteroaryl and any aryl may be further substituted with one or more R⁵ as defined above;
each R⁹ is independently selected from —H, C₁₋₈ alkyl, C₁₋₄ fluoroalkyl, C₁₋₄ hydroxyalkyl, C₂₋₈ alkenyl, C₂₋₈ alkynyl, C₃₋₁₀ cycloalkyl, —Z-heterocyclyl, —Z-aryl, and —Z-heteroaryl, wherein any heterocyclyl may be substituted with one or more R⁴ as defined above, and wherein any heteroaryl and any aryl may be substituted with one or more R⁵ as defined above;
each of R¹⁰ and R¹¹ is independently selected from —H, C₁₋₆ alkyl, C₁₋₄ fluoroalkyl, C₁₋₄ hydroxyalkyl, C₂₋₈ alkenyl, C₂₋₈ alkynyl, C₃₋₁₀ cycloalkyl, heterocyclyl, heteroaryl, and aryl, wherein any heterocyclyl may be substituted with one or more R⁴ as defined above, and wherein any heteroaryl and any aryl may be substituted with one or more R⁵ as defined above, or, alternatively, R¹⁰ and R¹¹ may together with the N-atom to which they are attached form an optionally 5 to 7 membered, N-heterocyclic ring optionally substituted with one or more R⁴ as defined above;
when Q is —CH═NR¹², R¹² is selected from C₁₋₁₀ alkyl, C₂₋₁₀ alkenyl, C₂₋₁₀ alkynyl, C₃₋₁₀ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—C(═O)—R⁷, —Z—OR⁷, halogen, —Z—SR⁷, —Z—SOR⁷, —Z—SO₂R⁷ and —Z—COOR⁷, which alkyl, alkenyl, alkynyl, cycloalkyl, heterocyclyl, heteroaryl and aryl may optionally be substituted with one or more R³;
when Q is —CHR²⁰NR²¹R¹³, R¹³ is selected from hydrogen, —C(O)R⁷, —C(O)C(O)R⁷, C₁₋₈ alkyl, C₁₋₄ fluoroalkyl, C₁₋₄ perfluoroalkyl, C₁₋₄ hydroxyalkyl, C₂₋₈ alkenyl, C₂₋₈ alkynyl, C₃₋₁₀ cycloalkyl, —Z-heterocyclyl, —Z-heteroaryl and —Z-aryl, which alkyl, alkenyl, alkynyl, cycloalkyl, heterocyclyl, heteroaryl and aryl may optionally be substituted with one or more independently selected R⁸, or is —CR¹⁴R¹⁵—NR⁶R⁷, —CR¹⁴R¹⁵CN, or —CR¹⁴R¹⁵OR⁷, wherein each of R¹⁴ and R¹⁵ is independently selected from —H, C₁₋₈ alkyl, C₂₋₈ alkenyl, C₂₋₈ alkynyl, C₃₋₁₀ cycloalkyl, heterocyclyl, heteroaryl and aryl, and wherein R¹⁴ and R¹⁵ together with the intervening carbon atom may designate a C₃₋₁₀ cycloalkyl or C₅₋₁₀-cycloalkenyl ring, which alkyl, alkenyl, alkynyl, cycloalkyl (ring), cycloalkenyl ring, heterocyclyl, heteroaryl and aryl may optionally be substituted with one or more R³;
R²⁰ and R²¹ are hydrogen, or together form a 1,3-diaza-C₅₋₇-cycloalk-2-yl group which is N-substituted with R¹⁶ and optionally further substituted with one or more R³, and optionally containing one or two oxo groups; a 1,3-thiaza-C₅₋₇-cycloalk-2-yl group which is N-substituted with R¹⁶ and optionally further substituted with one or more R³ and optionally containing one or two oxo groups; an 1,3-oxaza-C₅₋₇-cycloalk-2-yl group which is N-substituted with R¹⁶ and optionally further substituted with one or more R³, and optionally containing one or two oxo groups, wherein in all three instances two R³'s on the same carbon atom may together form a spiro group;
when Q is W, W is selected from an 1,3-diaza-C₅₋₇-cycloalk-2-yl group which is N-substituted with R¹⁶ and optionally further substituted with one or more R³, and optionally containing one or two oxo groups; a 1,3-thiaza-C₅₋₇-cycloalk-2-yl group which is N-substituted with R¹⁶ and optionally further substituted with one or more R³ and optionally containing one or two oxo groups; an 1,3-oxaza-C₅₋₇-cycloalk-2-yl group which is N-substituted with R¹⁶ and optionally further substituted with one or more R³, and optionally containing one or two oxo groups, wherein in all three instances two R³'s on the same carbon atom may together form a spiro group;
R¹⁶ is selected from hydrogen, —C(O)R⁷, —C(O)C(O)R⁷, —C(O)C(O)OR⁷, and —C(O)C(O)NR⁶R⁷;
when Q is —CH(OR¹⁷)₂, each R¹⁷ independently is R³, or wherein two R¹⁷ substituents together with the intervening —O—CH(−)—O— may form a heterocyclyl optionally substituted with one or more R³ and containing up to two oxo groups;
R¹⁸ is selected from hydrogen, C₁₋₆ alkyl, C₁₋₆ fluoroalkyl, C₁₋₆ hydroxyalkyl, C₂₋₇ alkenyl, C₂₋₇ alkynyl, C₃₋₇ cycloalkyl, C₃₋₇ oxyalkyl and may form a cyclic or heterocyclic structure with A, Y or R¹;
or an isomer or a mixture of isomers thereof, or a pharmaceutically acceptable salt, or solvate or prodrug thereof.

A sixth aspect of the present disclosure relates to a compound of the Formula (If)

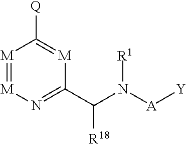

wherein
Q is selected from CO₂H, —CH═NR¹², —W, —CHR²⁰NR²¹R¹³, —CH═O and —CH(OR¹⁷)₂;
A is selected from —C(R^{2a})₂C(O)—, —C(R²)₂C(R²)₂C(O)—, C₁₋₈ alkylene, C₂₋₈ alkenylene, C₂₋₈ alkynylene, —Z′—C₃₋₁₀ cycloalkylene, —Z′-heterocyclylene, —Z′-heteroarylene and —Z′-arylene, which alkylene, alkenylene, alkynylene, —Z′-cycloalkylene, —Z′-heterocyclylene, —Z′-heteroarylene and —Z′-arylene may optionally be substituted with one or more R³ and may form a cyclic or heterocyclic structure with Y; with the proviso that when Q is —CH═O, A is not alkynylene;
Z′ is selected from C₁₋₄ alkylene, C₂₋₅ alkenene, C₂₋₅ alkynene, heterocyclylene and C₃₋₆ cycloalkylene;
Each M is independently selected from CH or N;
Y is selected from —H, —NR⁶R⁷, —OR⁷, C₁₋₈ alkyl, C₂₋₈ alkenyl, C₂₋₈ alkynyl, C₃₋₁₀ cycloalkyl, heterocyclyl, heteroaryl and aryl, which alkyl, alkenyl, alkynyl, cycloalkyl, heterocyclyl, heteroaryl and aryl may optionally be substituted with one or more R³;
R¹ is selected from —H, C₁₋₈ alkyl, C₂₋₈ alkenyl, C₂₋₈ alkynyl, C₃₋₁₀ cycloalkyl, which alkyl, alkenyl, alkynyl and cycloalkyl may be optionally substituted with one or more selected from —OH, aryl, C₁₋₆ alkoxy, heteroaryl, aryloxy, heteroaryloxy, —NR⁶R⁷, F, and C₃₋₆ cycloalkyl; or more preferably is selected from —H and C₁₋₄ alkyl; or with -A-Y forms a nitrogen containing optionally substituted heterocyclic group where the optional substitution may be C₁₋₈ alkyl, C₂₋₈ alkenyl, C₂₋₈ alkynyl, or C₃₋₁₀ cycloalkyl, which alkyl, alkenyl, alkynyl and cycloalkyl may be optionally substituted with one or more selected from —OH, aryl, C₁₋₆ alkoxy, heteroaryl, aryloxy, heteroaryloxy, —NR⁶R⁷, F, and C₃₋₆ cycloalkyl; or with R¹⁸ forms a nitrogen containing optionally substituted heterocyclic group where the optional substitution may be C₁₋₈ alkyl, C₂₋₈ alkenyl, C₂₋₈ alkynyl, or C₃₋₁₀ cycloalkyl, which alkyl, alkenyl, alkynyl and cycloalkyl may be optionally substituted with one or more selected from —OH, aryl, C₁₋₆ alkoxy, heteroaryl, aryloxy, heteroaryloxy, —NR⁶R⁷, F, and C₃₋₆ cycloalkyl;
R² is selected from —H, C₁₋₈ alkyl, C₂₋₈ alkenyl, C₂₋₈ alkynyl, and C₃₋₁₀ cycloalkyl, which alkyl, alkenyl, alkynyl and cycloalkyl may be optionally substituted with one or more selected from —OH, aryl, C₁₋₆ alkoxy, heteroaryl, aryloxy, heteroaryloxy, F, and C₃₋₆ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—C(═O)—R⁷, —Z—OR⁷, halogen, —Z—SR⁷, —Z—SOR⁷, —Z—SO₂R⁷, —Z—SO₂NR⁶R⁷ and —Z—COOR⁷; alternatively, R² may form a cyclic or heterocyclic structure with another R², R¹ R¹⁸ or Y;
R^{2a} is selected from —H, C₁₋₈ alkyl, C₂₋₈ alkenyl, C₂₋₈ alkynyl, and C₃₋₁₀ cycloalkyl, which alkyl, alkenyl, alkynyl and cycloalkyl may be optionally substituted with one or more selected from —OH, aryl, C₁₋₆ alkoxy, heteroaryl, aryloxy, heteroaryloxy, F, and C₃₋₆ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—NR⁶—C(═O)—OR⁷, —Z—C(═O)—OR⁷, —Z—OR⁷, halogen, —Z—SR⁷, —Z—SOR⁷, —Z—SO₂R⁷, —Z—SO₂NR⁶R⁷ and —Z—COOR⁷; with the proviso that the two R^{2a} groups are either both non-hydrogen, or that one of the R^{2a} forms a ring with R¹ or R¹⁸;
each R³ is independently selected from C₁₋₆ alkyl, C₁₋₄ fluoroalkyl, C₁₋₄ hydroxyalkyl, C₂₋₆ alkenyl, C₂₋₆ alkynyl, C₃₋₁₀ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—C(═O)—R⁷, —Z—OR⁷, halogen, —Z—SR⁷, —Z—SOR⁷, —Z—SO₂R⁷, —Z—SO₂NR⁶R⁷ and —Z—COOR⁷, wherein any heterocyclyl may be substituted with one or more R⁴, and wherein any heteroaryl and any aryl may be substituted with one or more R⁵;
Z is selected from a single bond, C₁₋₄ alkylene, heterocyclylene and C₃₋₆ cycloalkylene; each R⁴ is independently selected from C₁₋₆ alkyl, C₁₋₄ fluoroalkyl, C₁₋₄ hydroxyalkyl, C₁₋₄ alkoxy, C₃₋₁₀ cycloalkyl, NR⁶R⁷, C(═O)—NR⁶R⁷, NR⁶—C(═O)—R⁷, Z—C(═O)—R⁷, —Z—C(═O)—H, OR⁷, halogen, SR⁷, SOR⁷, SO₂R⁷, SO₂NR⁶R⁷ and COOR⁷ and —OH;
each R⁵ is independently selected from C₁₋₆ alkyl, C₁₋₄ fluoroalkyl, C₁₋₄ hydroxyalkyl, C₁₋₄ alkoxy, C₃₋₆ cycloalkyl, —Z—C(═O)—NR⁶R⁷, —Z—NR⁶—C(═O)—R⁷, —Z—C(═O)—R⁷, —Z—NR⁶C(═O)OR⁷, —Z—C(═O)OR⁷, OR⁷, —CN and halogen each of R⁶ and R⁷ is independently selected from hydrogen, C₁₋₈ alkyl, C₁₋₄ fluoroalkyl, C₁₋₄ perfluoroalkyl, C₁₋₄ hydroxyalkyl, C₂₋₈ alkenyl, C₂₋₈ alkynyl, C₃₋₁₀ cycloalkyl, —Z-heterocyclyl, —Z-heteroaryl and —Z-aryl, which alkyl, alkenyl, alkynyl, cycloalkyl, heterocyclyl, heteroaryl and aryl may optionally be substituted with one or more independently selected R⁸; or, alternatively, R⁶ and R⁷ may together with the N-atom to which they are attached form an N-heterocyclic ring optionally substituted with one or more independently selected R⁸;
each R⁸ is independently selected from C₁₋₆ alkyl, C₁₋₄ fluoroalkyl, C₁₋₄ hydroxyalkyl, C₂₋₆ alkenyl, C₂₋₆ alkynyl, C₃₋₁₀ cycloalkyl, —Z-heterocyclyl, —Z-heteroaryl, —Z-aryl, —Z—NR¹⁰R¹¹, —Z—C(═O)—NR¹⁰R¹¹, —Z—OR⁹, halogen, —CN, —Z—SR⁹, —Z—SOR⁹, —Z—SO₂R⁹ and —Z—COOR⁹, which alkyl, alkenyl, alkynyl, cycloalkyl, heterocyclics, heteroaryl and aryl may optionally be substituted with one or more selected from C₁₋₄ alkyl, C₁₋₄ fluoroalkyl, C₁₋₄ hydroxyalkyl, C₃₋₆ cycloalkyl, —Z-heterocyclyl, —Z-heteroaryl, —Z-aryl, —Z—NR¹⁰R¹¹, —Z—C(═O)—NR¹⁰R¹¹, —Z—OR⁹, halogen, —CN, —Z—SR⁹, —Z—SOR⁹, —Z—SO₂R⁹ and —Z—COOR⁹; wherein any heterocyclyl may be further substituted with one or more R⁴ as defined above, and wherein any heteroaryl and any aryl may be further substituted with one or more R⁵ as defined above;
each R⁹ is independently selected from —H, C₁₋₈ alkyl, C₁₋₄ fluoroalkyl, C₁₋₄ hydroxyalkyl, C₂₋₈ alkenyl, C₂₋₈ alkynyl, C₃₋₁₀ cycloalkyl, —Z-heterocyclyl, —Z-aryl, and —Z-heteroaryl, wherein any heterocyclyl may be substituted with one or more R⁴ as defined above, and wherein any heteroaryl and any aryl may be substituted with one or more R⁵ as defined above;
each of R¹⁰ and R¹¹ is independently selected from —H, C₁₋₆ alkyl, C₁₋₄ fluoroalkyl, C₁₋₄ hydroxyalkyl, C₂₋₈ alkenyl, C₂₋₈ alkynyl, C₃₋₁₀ cycloalkyl, heterocyclyl, heteroaryl, and aryl, wherein any heterocyclyl may be substituted with one or more R⁴ as defined above, and wherein any heteroaryl and any aryl may be substituted with one or more R⁵ as defined above, or, alternatively, R¹⁰ and R¹¹ may together with the N-atom to which they are attached form an optionally 5 to 7 membered, N-heterocyclic ring optionally substituted with one or more R⁴ as defined above;
when Q is —CH═NR¹², R¹² is selected from C₁₋₁₀ alkyl, C₂₋₁₀ alkenyl, C₂₋₁₀ alkynyl, C₃₋₁₀ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—C(═O)—R⁷, —Z—OR⁷, halogen, —Z—SR⁷, —Z—SOR⁷, —Z—SO₂R⁷ and —Z—COOR⁷, which alkyl, alkenyl, alkynyl, cycloalkyl, heterocyclyl, heteroaryl and aryl may optionally be substituted with one or more R³;
when Q is —CHR²⁰NR²¹R¹³, R¹³ is selected from hydrogen, —C(O)R⁷, —C(O)C(O)R⁷, C₁₋₈ alkyl, C₁₋₄ fluoroalkyl, C₁₋₄ perfluoroalkyl, C₁₋₄ hydroxyalkyl, C₂₋₈ alkenyl, C₂₋₈ alkynyl, C₃₋₁₀ cycloalkyl, —Z-heterocyclyl, —Z-heteroaryl and —Z-aryl, which alkyl, alkenyl, alkynyl, cycloalkyl, heterocyclyl, heteroaryl and aryl may optionally be substituted with one or more independently selected R⁸, or is —CR¹⁴R¹⁵—NR⁶R⁷, —CR¹⁴R¹⁵CN, or —CR¹⁴R¹⁵OR⁷, wherein each of R¹⁴ and R¹⁵ is independently selected from —H, C₁₋₈ alkyl, C₂₋₈ alkenyl, C₂₋₈ alkynyl, C₃₋₁₀ cycloalkyl, heterocyclyl, heteroaryl and aryl, and wherein R¹⁴ and R¹⁵ together with the intervening carbon atom may designate a C₃₋₁₀ cycloalkyl or C₅₋₁₀-cycloalkenyl ring, which alkyl, alkenyl, alkynyl, cycloalkyl (ring), cycloalkenyl ring, heterocyclyl, heteroaryl and aryl may optionally be substituted with one or more R³;
R²⁰ and R²¹ are hydrogen, or together form a 1,3-diaza-C₅₋₇-cycloalk-2-yl group which is N-substituted with R¹⁶ and optionally further substituted with one or more R³, and optionally containing one or two oxo groups; a 1,3-thiaza-C₅₋₇-cycloalk-2-yl group which is N-substituted with R¹⁶ and optionally further substituted with one or more R³ and optionally containing one or two oxo groups; an 1,3-oxaza-C₅₋₇-cycloalk-2-yl group which is N-substituted with R¹⁶ and optionally further substituted with one or more R³, and optionally containing one or two oxo groups, wherein in all three instances two R³'s on the same carbon atom may together form a spiro group;
when Q is W, W is selected from an 1,3-diaza-C₅₋₇-cycloalk-2-yl group which is N-substituted with R¹⁶ and optionally further substituted with one or more R³, and optionally containing one or two oxo groups; a 1,3-thiaza-C₅₋₇-cycloalk-2-yl group which is N-substituted with R¹⁶ and optionally further substituted with one or more R³ and optionally containing one or two oxo groups; an 1,3-oxaza-C₅₋₇-cycloalk-2-yl group which is N-substituted with R¹⁶ and optionally further substituted with one or more R³, and optionally containing one or two oxo groups, wherein in all three instances two R³'s on the same carbon atom may together form a spiro group;
R¹⁶ is selected from hydrogen, —C(O)R⁷, —C(O)C(O)R⁷, —C(O)C(O)OR⁷, and —C(O)C(O)NR⁶R⁷;
when Q is —CH(OR¹⁷)₂, each R¹⁷ independently is R³, or wherein two R¹⁷ substituents together with the intervening —O—CH(−)—O— may form a heterocyclyl optionally substituted with one or more R³ and containing up to two oxo groups;
R¹⁸ is selected from C₁₋₆ alkyl, C₁₋₆ fluoroalkyl, C₁₋₆ hydroxyalkyl, C₂₋₇ alkenyl, C₂₋₇ alkynyl, C₃₋₇ cycloalkyl, C₃₋₇ oxyalkyl and may form a cyclic or heterocyclic structure with A, Y or R¹;
or an isomer or a mixture of isomers thereof, or a pharmaceutically acceptable salt, or solvate or prodrug thereof.

A seventh aspect of the present disclosure relates to a compound of the Formula (Ig)

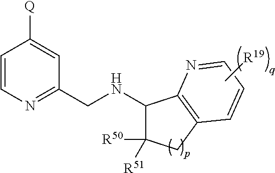

wherein
Q is selected from CO₂H, —CH═NR¹², —W, —CHR²⁰NR²¹R¹³, —CH═O and —CH(OR¹⁷)₂;
each R³ is independently selected from C₁₋₆ alkyl, C₁₋₄ fluoroalkyl, C₁₋₄ hydroxyalkyl, C₂₋₆ alkenyl, C₂₋₆ alkynyl, C₃₋₁₀ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—NR⁶—C(═O)—OR⁷, —Z—C(═O)—R⁷, —Z—OR⁷, halogen, —Z—SR⁷, —Z—SOR⁷, —Z—SO₂R⁷, —Z—SO₂NR⁶R⁷, and —Z—COOR⁷, wherein any heterocyclyl may be substituted with one or more R⁴, and wherein any heteroaryl and any aryl may be substituted with one or more R⁵;
each R⁴ is independently selected from C₁₋₆ alkyl, C₁₋₄ fluoroalkyl, C₁₋₄ hydroxyalkyl, C₁₋₄ alkoxy, C₃₋₁₀ cycloalkyl, NR⁶R⁷, C(═O)—NR⁶R⁷, NR⁶—C(═O)—R⁷, Z—C(═O)—R⁷, —Z—C(═O)—H, OR⁷, halogen, SR⁷, SOR⁷, SO₂R⁷, SO₂NR⁶R⁷ and COOR⁷ and —OH;
each R⁵ is independently selected from C₁₋₆ alkyl, C₁₋₄ fluoroalkyl, C₁₋₄ hydroxyalkyl, C₁₋₄ alkoxy, C₃₋₆ cycloalkyl, —Z—C(═O)—NR⁶R⁷, —Z—NR⁶—C(═O)—R⁷, —Z—C(═O)—R⁷, —Z—C(═O)OR⁷, —Z—NR⁶C(═O)OR⁷, OR⁷, —CN and halogen;
each of R⁶ and R⁷ is independently selected from hydrogen, optionally not both being hydrogen, C₁₋₈ alkyl, C₁₋₄ fluoroalkyl, C₁₋₄ perfluoroalkyl, C₁₋₄ hydroxyalkyl, C₂₋₈ alkenyl, C₂₋₈ alkynyl, C₃₋₁₀ cycloalkyl, —Z-heterocyclyl, —Z-heteroaryl and —Z-aryl, which alkyl, alkenyl, alkynyl, cycloalkyl, heterocyclyl, heteroaryl and aryl may optionally be substituted with one or more independently selected R⁸; or, alternatively, R⁶ and R⁷ may together with the N-atom to which they are attached form an N-heterocyclic ring optionally substituted with one or more independently selected R⁸;
each R⁸ is independently selected from C₁₋₆ alkyl, C₁₋₄ fluoroalkyl, C₁₋₄ hydroxyalkyl, C₂₋₆ alkenyl, C₂₋₆ alkynyl, C₃₋₁₀ cycloalkyl, —Z-heterocyclyl, —Z-heteroaryl, —Z-aryl, —Z—NR¹⁰R¹¹, —Z—C(═O)—NR¹⁰R¹¹, —Z—OR⁹, halogen, —CN, —Z—SR⁹, —Z—SOR⁹, —Z—SO₂R⁹ and —Z—COOR⁹, which alkyl, alkenyl, alkynyl, cycloalkyl, heterocyclics, heteroaryl and aryl may optionally be substituted with one or more selected from C₁₋₄ alkyl, C₁₋₄ fluoroalkyl, C₁₋₄ hydroxyalkyl, C₃₋₆ cycloalkyl, —Z-heterocyclyl, —Z-heteroaryl, —Z-aryl, —Z—NR¹⁰R¹¹, —Z—C(═O)—NR¹⁰R¹¹, —Z—OR⁹, halogen, —CN, —Z—SR⁹, —Z—SOR⁹, —Z—SO₂R⁹ and —Z—COOR⁹; wherein any heterocyclyl may be further substituted with one or more R⁴ as defined above, and wherein any heteroaryl and any aryl may be further substituted with one or more R⁵ as defined above;
each R⁹ is independently selected from —H, C₁₋₈ alkyl, C₁₋₄ fluoroalkyl, C₁₋₄ hydroxyalkyl, C₂₋₈ alkenyl, C₂₋₈ alkynyl, C₃₋₁₀ cycloalkyl, —Z-heterocyclyl, —Z-aryl, and —Z-heteroaryl, wherein any heterocyclyl may be substituted with one or more R⁴ as defined above, and wherein any heteroaryl and any aryl may be substituted with one or more R⁵ as defined above;
each of R¹⁰ and R¹¹ is independently selected from —H, C₁₋₆ alkyl, C₁₋₄ fluoroalkyl, C₁₋₄ hydroxyalkyl, C₂₋₈ alkenyl, C₂₋₈ alkynyl, C₃₋₁₀ cycloalkyl, heterocyclyl, heteroaryl, and aryl, wherein any heterocyclyl may be substituted with one or more R⁴ as defined above, and wherein any heteroaryl and any aryl may be substituted with one or more R⁵ as defined above, or, alternatively, R¹⁰ and R¹¹ may together with the N-atom to which they are attached form an optionally 5 to 7 membered, N-heterocyclic ring optionally substituted with one or more R⁴ as defined above;
when Q is —CH═NR¹², R¹² is selected from C₁₋₁₀ alkyl, C₂₋₁₀ alkenyl, C₂₋₁₀ alkynyl, C₃₋₁₀ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—C(═O)—R⁷, —Z—OR⁷, halogen, —Z—SR⁷, —Z—SOR⁷, —Z—SO₂R⁷ and —Z—COOR⁷, which alkyl, alkenyl, alkynyl, cycloalkyl, heterocyclyl, heteroaryl and aryl may optionally be substituted with one or more R³;
when Q is —CHR²⁰NR²¹R¹³, R¹³ is selected from hydrogen, —C(O)R⁷, —C(O)C(O)R⁷, —C(O)C(O)OR⁷, C₁₋₈ alkyl, C₁₋₄ fluoroalkyl, C₁₋₄ perfluoroalkyl, C₁₋₄ hydroxyalkyl, C₂₋₈ alkenyl, C₂₋₈ alkynyl, C₃₋₁₀ cycloalkyl, —Z-heterocyclyl, —Z-aryl and —Z-heteroaryl, which alkyl, alkenyl, alkynyl, cycloalkyl, heterocyclyl, aryl and heteroaryl may optionally be substituted with one or more independently selected R⁸, or is —CR¹⁴R¹⁵—NR⁶R⁷, —CR¹⁴R¹⁵CN, or —CR¹⁴R¹⁵OR⁷, wherein each of R¹⁴ and R¹⁵ is independently selected from —H, C₁₋₈ alkyl, C₂₋₈ alkenyl, C₂₋₈ alkynyl, C₃₋₁₀ cycloalkyl, heterocyclyl, heteroaryl and aryl, and wherein R¹⁴ and R¹⁵ together with the intervening carbon atom may designate a C₃₋₁₀ cycloalkyl or C₅₋₁₀-cycloalkenyl ring, which alkyl, alkenyl, alkynyl, cycloalkyl (ring), cycloalkenyl ring, heterocyclyl, heteroaryl and aryl may optionally be substituted with one or more R³;
R²⁰ and R²¹ are hydrogen, or together form a 1,3-diaza-C₅₋₇-cycloalk-2-yl group which is N-substituted with R¹⁶ and optionally further substituted with one or more R³, and optionally containing one or two oxo groups; a 1,3-thiaza-C₅₋₇-cycloalk-2-yl group which is N-substituted with R¹⁶ and optionally further substituted with one or more R³ and optionally containing one or two oxo groups; an 1,3-oxaza-C₅₋₇-cycloalk-2-yl group which is N-substituted with R¹⁶ and optionally further substituted with one or more R³, and optionally containing one or two oxo groups, wherein in all three instances two R³'s on the same carbon atom may together form a spiro group;
when Q is W, W is selected from an 1,3-diaza-C₅₋₇-cycloalk-2-yl group which is N-substituted with R¹⁶ and optionally further substituted with one or more R³, and optionally containing one or two oxo groups; a 1,3-thiaza-C₅₋₇-cycloalk-2-yl group which is N-substituted with R¹⁶ and optionally further substituted with one or more R³ and optionally containing one or two oxo groups; an 1,3-oxaza-C₅₋₇-cycloalk-2-yl group which is N-substituted with R¹⁶ and optionally further substituted with one or more R³, and optionally containing one or two oxo groups, wherein in all three instances two R³'s on the same carbon atom may together form a spiro group;
R¹⁶ is selected from hydrogen, —C(O)R⁷, —C(O)C(O)R⁷, —C(O)C(O)OR⁷, and —C(O)C(O)NR⁶R⁷;
when Q is —CH(OR¹⁷)₂, each R¹⁷ independently is R³, or wherein two R¹⁷ substituents together with the intervening —O—CH(−)—O— may form a heterocyclyl optionally substituted with one or more R³ and containing up to two oxo groups;
R¹⁹ is selected from the group consisting of C₁₋₆ alkyl, C₁₋₆ alkoxy, C₁₋₄ fluoroalkyl, C₁₋₄ hydroxyalkyl, C₂₋₆ alkenyl, C₂₋₆ alkynyl, C₃₋₁₀ cycloalkyl, —Z-heterocyclyl, —Z-aryl, and —Z-heteroaryl;
Z is selected from a single bond, C₁₋₄ alkylene, heterocyclylene, and C₃₋₆ cycloalkylene;
R⁵⁰ and R⁵¹ are each independently selected from the group consisting of C₁₋₄ alkyl, C₁₋₄ alkoxy, C₁₋₄ fluoroalkyl, and C₁₋₄ hydroxyalkyl;
p is 0, 1, 2, 3, or 4; and
q is 0, 1, 2, or 3.

An eighth aspect of the present disclosure relates to a compound of the Formula (Ih)

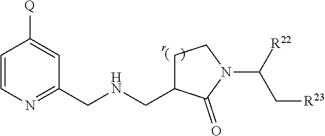

wherein
Q is selected from CO₂H, —CH═NR¹², —W, —CHR²⁰NR²¹R¹³, —CH═O and —CH(OR¹⁷)₂;
each R³ is independently selected from C₁₋₆ alkyl, C₁₋₄ fluoroalkyl, C₁₋₄ hydroxyalkyl, C₂₋₆ alkenyl, C₂₋₆ alkynyl, C₃₋₁₀ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—NR⁶—C(═O)—OR⁷, —Z—C(═O)—R⁷, —Z—OR⁷, halogen, —Z—SR⁷, —Z—SOR⁷, —Z—SO₂R⁷, —Z—SO₂NR⁶R⁷, and —Z—COOR⁷, wherein any heterocyclyl may be substituted with one or more R⁴, and wherein any heteroaryl and any aryl may be substituted with one or more R⁵;
each R⁴ is independently selected from C₁₋₆ alkyl, C₁₋₄ fluoroalkyl, C₁₋₄ hydroxyalkyl, C₁₋₄ alkoxy, C₃₋₁₀ cycloalkyl, NR⁶R⁷, C(═O)—NR⁶R⁷, NR⁶—C(═O)—R⁷, Z—C(═O)—R⁷, —Z—C(═O)—H, OR⁷, halogen, SR⁷, SOR⁷, SO₂R⁷, SO₂NR⁶R⁷ and COOR⁷ and —OH;
each R⁵ is independently selected from C₁₋₆ alkyl, C₁₋₄ fluoroalkyl, C₁₋₄ hydroxyalkyl, C₁₋₄ alkoxy, C₃₋₆ cycloalkyl, —Z—C(═O)—NR⁶R⁷, —Z—NR⁶—C(═O)—R⁷, —Z—C(═O)—R⁷, —Z—C(═O)OR⁷, —Z—NR⁶C(═O)OR⁷, OR⁷, —CN and halogen;
each of R⁶ and R⁷ is independently selected from hydrogen, optionally not both being hydrogen, C₁₋₈ alkyl, C₁₋₄ fluoroalkyl, C₁₋₄ perfluoroalkyl, C₁₋₄ hydroxyalkyl, C₂₋₈ alkenyl, C₂₋₈ alkynyl, C₃₋₁₀ cycloalkyl, —Z-heterocyclyl, —Z-heteroaryl and —Z-aryl, which alkyl, alkenyl, alkynyl, cycloalkyl, heterocyclyl, heteroaryl and aryl may optionally be substituted with one or more independently selected R⁸; or, alternatively, R⁶ and R⁷ may together with the N-atom to which they are attached form an N-heterocyclic ring optionally substituted with one or more independently selected R⁸;
each R⁸ is independently selected from C₁₋₆ alkyl, C₁₋₄ fluoroalkyl, C₁₋₄ hydroxyalkyl, C₂₋₆ alkenyl, C₂₋₆ alkynyl, C₃₋₁₀ cycloalkyl, —Z-heterocyclyl, —Z-heteroaryl, —Z-aryl, —Z—NR¹⁰R¹¹, —Z—C(═O)—NR¹⁰R¹¹, —Z—OR⁹, halogen, —CN, —Z—SR⁹, —Z—SOR⁹, —Z—SO₂R⁹ and —Z—COOR⁹, which alkyl, alkenyl, alkynyl, cycloalkyl, heterocyclics, heteroaryl and aryl may optionally be substituted with one or more selected from C₁₋₄ alkyl, C₁₋₄ fluoroalkyl, C₁₋₄ hydroxyalkyl, C₃₋₆ cycloalkyl, —Z-heterocyclyl, —Z-heteroaryl, —Z-aryl, —Z—NR¹⁰R¹¹, —Z—C(═O)—NR¹⁰R¹¹, —Z—OR⁹, halogen, —CN, —Z—SR⁹, —Z—SOR⁹, —Z—SO₂R⁹ and —Z—COOR⁹; wherein any heterocyclyl may be further substituted with one or more R⁴ as defined above, and wherein any heteroaryl and any aryl may be further substituted with one or more R⁵ as defined above;
each R⁹ is independently selected from —H, C₁₋₈ alkyl, C₁₋₄ fluoroalkyl, C₁₋₄ hydroxyalkyl, C₂₋₈ alkenyl, C₂₋₈ alkynyl, C₃₋₁₀ cycloalkyl, —Z-heterocyclyl, —Z-aryl, and —Z-heteroaryl, wherein any heterocyclyl may be substituted with one or more R⁴ as defined above, and wherein any heteroaryl and any aryl may be substituted with one or more R⁵ as defined above;
each of R¹⁰ and R¹¹ is independently selected from —H, C₁₋₆ alkyl, C₁₋₄ fluoroalkyl, C₁₋₄ hydroxyalkyl, C₂₋₈ alkenyl, C₂₋₈ alkynyl, C₃₋₁₀ cycloalkyl, heterocyclyl, heteroaryl, and aryl, wherein any heterocyclyl may be substituted with one or more R⁴ as defined above, and wherein any heteroaryl and any aryl may be substituted with one or more R⁵ as defined above, or, alternatively, R¹⁰ and R¹¹ may together with the N-atom to which they are attached form an optionally 5 to 7 membered, N-heterocyclic ring optionally substituted with one or more R⁴ as defined above;
when Q is —CH═NR¹², R¹² is selected from C₁₋₁₀ alkyl, C₂₋₁₀ alkenyl, C₂₋₁₀ alkynyl, C₃₋₁₀ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—C(═O)—R⁷, —Z—OR⁷, halogen, —Z—SR⁷, —Z—SOR⁷, —Z—SO₂R⁷ and —Z—COOR⁷, which alkyl, alkenyl, alkynyl, cycloalkyl, heterocyclyl, heteroaryl and aryl may optionally be substituted with one or more R³;
when Q is —CHR²⁰NR²¹R¹³, R¹³ is selected from hydrogen, —C(O)R⁷, —C(O)C(O)R⁷, —C(O)C(O)OR⁷, C₁₋₈ alkyl, C₁₋₄ fluoroalkyl, C₁₋₄ perfluoroalkyl, C₁₋₄ hydroxyalkyl, C₂₋₈ alkenyl, C₂₋₈ alkynyl, C₃₋₁₀ cycloalkyl, —Z-heterocyclyl, —Z-aryl and —Z-heteroaryl, which alkyl, alkenyl, alkynyl, cycloalkyl, heterocyclyl, aryl and heteroaryl may optionally be substituted with one or more independently selected R⁸, or is —CR¹⁴R¹⁵—NR⁶R⁷, —CR¹⁴R¹⁵CN, or —CR¹⁴R¹⁵OR⁷, wherein each of R¹⁴ and R¹⁵ is independently selected from —H, C₁₋₈ alkyl, C₂₋₈ alkenyl, C₂₋₈ alkynyl, C₃₋₁₀ cycloalkyl, heterocyclyl, heteroaryl and aryl, and wherein R¹⁴ and R¹⁵ together with the intervening carbon atom may designate a C₃₋₁₀ cycloalkyl or C₅₋₁₀-cycloalkenyl ring, which alkyl, alkenyl, alkynyl, cycloalkyl (ring), cycloalkenyl ring, heterocyclyl, heteroaryl and aryl may optionally be substituted with one or more R³;
R²⁰ and R²¹ are hydrogen, or together form a 1,3-diaza-C₅₋₇-cycloalk-2-yl group which is N-substituted with R¹⁶ and optionally further substituted with one or more R³, and optionally containing one or two oxo groups; a 1,3-thiaza-C₅₋₇-cycloalk-2-yl group which is N-substituted with R¹⁶ and optionally further substituted with one or more R³ and optionally containing one or two oxo groups; an 1,3-oxaza-C₅₋₇-cycloalk-2-yl group which is N-substituted with R¹⁶ and optionally further substituted with one or more R³, and optionally containing one or two oxo groups, wherein in all three instances two R³'s on the same carbon atom may together form a spiro group;
when Q is W, W is selected from an 1,3-diaza-C₅₋₇-cycloalk-2-yl group which is N-substituted with R¹⁶ and optionally further substituted with one or more R³, and optionally containing one or two oxo groups; a 1,3-thiaza-C₅₋₇-cycloalk-2-yl group which is N-substituted with R¹⁶ and optionally further substituted with one or more R³ and optionally containing one or two oxo groups; an 1,3-oxaza-C₅₋₇-cycloalk-2-yl group which is N-substituted with R¹⁶ and optionally further substituted with one or more R³, and optionally containing one or two oxo groups, wherein in all three instances two R³'s on the same carbon atom may together form a spiro group;
R¹⁶ is selected from hydrogen, —C(O)R⁷, —C(O)C(O)R⁷, —C(O)C(O)OR⁷, and —C(O)C(O)NR⁶R⁷;
when Q is —CH(OR¹⁷)₂, each R¹⁷ independently is R³, or wherein two R¹⁷ substituents together with the intervening —O—CH(−)—O— may form a heterocyclyl optionally substituted with one or more R³ and containing up to two oxo groups;
R²² and R²³ are each independently selected from the group consisting of hydrogen, C₁₋₆ alkyl, and aryl, wherein C₁₋₆ alkyl and aryl are optionally substituted with halogen, hydroxy, or C₁₋₆ alkoxy; and
r is 0, 1, 2, 3, or 4.

A ninth aspect of the present disclosure relates to a compound of the Formula (Ii)

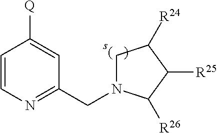

wherein
Q is selected from CO₂H, —CH═NR¹², —W, —CHR²⁰NR²¹R¹³, —CH═O and —CH(OR¹⁷)₂;
each R³ is independently selected from C₁₋₆ alkyl, C₁₋₄ fluoroalkyl, C₁₋₄ hydroxyalkyl, C₂₋₆ alkenyl, C₂₋₆ alkynyl, C₃₋₁₀ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—NR⁶—C(═O)—OR⁷, —Z—C(═O)—R⁷, —Z—OR⁷, halogen, —Z—SR⁷, —Z—SOR⁷, —Z—SO₂R⁷, —Z—SO₂NR⁶R⁷, and —Z—COOR⁷, wherein any heterocyclyl may be substituted with one or more R⁴, and wherein any heteroaryl and any aryl may be substituted with one or more R⁵;
each R⁴ is independently selected from C₁₋₆ alkyl, C₁₋₄ fluoroalkyl, C₁₋₄ hydroxyalkyl, C₁₋₄ alkoxy, C₃₋₁₀ cycloalkyl, NR⁶R⁷, C(═O)—NR⁶R⁷, NR⁶—C(═O)—R⁷, Z—C(═O)—R⁷, —Z—C(═O)—H, OR⁷, halogen, SR⁷, SOR⁷, SO₂R⁷, SO₂NR⁶R⁷ and COOR⁷ and —OH;
each R⁵ is independently selected from C₁₋₆ alkyl, C₁₋₄ fluoroalkyl, C₁₋₄ hydroxyalkyl, C₁₋₄ alkoxy, C₃₋₆ cycloalkyl, —Z—C(═O)—NR⁶R⁷, —Z—NR⁶—C(═O)—R⁷, —Z—C(═O)—R⁷, —Z—C(═O)OR⁷, —Z—NR⁶C(═O)OR⁷, OR⁷, —CN and halogen;
each of R⁶ and R⁷ is independently selected from hydrogen, optionally not both being hydrogen, C₁₋₈ alkyl, C₁₋₄ fluoroalkyl, C₁₋₄ perfluoroalkyl, C₁₋₄ hydroxyalkyl, C₂₋₈ alkenyl, C₂₋₈ alkynyl, C₃₋₁₀ cycloalkyl, —Z-heterocyclyl, —Z-heteroaryl and —Z-aryl, which alkyl, alkenyl, alkynyl, cycloalkyl, heterocyclyl, heteroaryl and aryl may optionally be substituted with one or more independently selected R⁸; or, alternatively, R⁶ and R⁷ may together with the N-atom to which they are attached form an N-heterocyclic ring optionally substituted with one or more independently selected R⁸;
each R⁸ is independently selected from C₁₋₆ alkyl, C₁₋₄ fluoroalkyl, C₁₋₄ hydroxyalkyl, C₂₋₆ alkenyl, C₂₋₆ alkynyl, C₃₋₁₀ cycloalkyl, —Z-heterocyclyl, —Z-heteroaryl, —Z-aryl, —Z—NR¹⁰R¹¹, —Z—C(═O)—NR¹⁰R¹¹, —Z—OR⁹, halogen, —CN, —Z—SR⁹, —Z—SOR⁹, —Z—SO₂R⁹ and —Z—COOR⁹, which alkyl, alkenyl, alkynyl, cycloalkyl, heterocyclics, heteroaryl and aryl may optionally be substituted with one or more selected from C₁₋₄ alkyl, C₁₋₄ fluoroalkyl, C₁₋₄ hydroxyalkyl, C₃₋₆ cycloalkyl, —Z-heterocyclyl, —Z-heteroaryl, —Z-aryl, —Z—NR¹⁰R¹¹, —Z—C(═O)—NR¹⁰R¹¹, —Z—OR⁹, halogen, —CN, —Z—SR⁹, —Z—SOR⁹, —Z—SO₂R⁹ and —Z—COOR⁹; wherein any heterocyclyl may be further substituted with one or more R⁴ as defined above, and wherein any heteroaryl and any aryl may be further substituted with one or more R⁵ as defined above;
each R⁹ is independently selected from —H, C₁₋₈ alkyl, C₁₋₄ fluoroalkyl, C₁₋₄ hydroxyalkyl, C₂₋₈ alkenyl, C₂₋₈ alkynyl, C₃₋₁₀ cycloalkyl, —Z-heterocyclyl, —Z-aryl, and —Z-heteroaryl, wherein any heterocyclyl may be substituted with one or more R⁴ as defined above, and wherein any heteroaryl and any aryl may be substituted with one or more R⁵ as defined above;
each of R¹⁰ and R¹¹ is independently selected from —H, C₁₋₆ alkyl, C₁₋₄ fluoroalkyl, C₁₋₄ hydroxyalkyl, C₂₋₈ alkenyl, C₂₋₈ alkynyl, C₃₋₁₀ cycloalkyl, heterocyclyl, heteroaryl, and aryl, wherein any heterocyclyl may be substituted with one or more R⁴ as defined above, and wherein any heteroaryl and any aryl may be substituted with one or more R⁵ as defined above, or, alternatively, R¹⁰ and R¹¹ may together with the N-atom to which they are attached form an optionally 5 to 7 membered, N-heterocyclic ring optionally substituted with one or more R⁴ as defined above;
when Q is —CH═NR¹², R¹² is selected from C₁₋₁₀ alkyl, C₂₋₁₀ alkenyl, C₂₋₁₀ alkynyl, C₃₋₁₀ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—C(═O)—R⁷, —Z—OR⁷, halogen, —Z—SR⁷, —Z—SOR⁷, —Z—SO₂R⁷ and —Z—COOR⁷, which alkyl, alkenyl, alkynyl, cycloalkyl, heterocyclyl, heteroaryl and aryl may optionally be substituted with one or more R³;
when Q is —CHR²⁰NR²¹R¹³, R¹³ is selected from hydrogen, —C(O)R⁷, —C(O)C(O)R⁷, —C(O)C(O)OR⁷, C₁₋₈ alkyl, C₁₋₄ fluoroalkyl, C₁₋₄ perfluoroalkyl, C₁₋₄ hydroxyalkyl, C₂₋₈ alkenyl, C₂₋₈ alkynyl, C₃₋₁₀ cycloalkyl, —Z-heterocyclyl, —Z-aryl and —Z-heteroaryl, which alkyl, alkenyl, alkynyl, cycloalkyl, heterocyclyl, aryl and heteroaryl may optionally be substituted with one or more independently selected R⁸, or is —CR¹⁴R¹⁵—NR⁶R⁷, —CR¹⁴R¹⁵CN, or —CR¹⁴R¹⁵OR⁷, wherein each of R¹⁴ and R¹⁵ is independently selected from —H, C₁₋₈ alkyl, C₂₋₈ alkenyl, C₂₋₈ alkynyl, C₃₋₁₀ cycloalkyl, heterocyclyl, heteroaryl and aryl, and wherein R¹⁴ and R¹⁵ together with the intervening carbon atom may designate a C₃₋₁₀ cycloalkyl or C₅₋₁₀-cycloalkenyl ring, which alkyl, alkenyl, alkynyl, cycloalkyl (ring), cycloalkenyl ring, heterocyclyl, heteroaryl and aryl may optionally be substituted with one or more R³;
R²⁰ and R²¹ are hydrogen, or together form a 1,3-diaza-C₅₋₇-cycloalk-2-yl group which is N-substituted with R¹⁶ and optionally further substituted with one or more R³, and optionally containing one or two oxo groups; a 1,3-thiaza-C₅₋₇-cycloalk-2-yl group which is N-substituted with R¹⁶ and optionally further substituted with one or more R³ and optionally containing one or two oxo groups; an 1,3-oxaza-C₅₋₇-cycloalk-2-yl group which is N-substituted with R¹⁶ and optionally further substituted with one or more R³, and optionally containing one or two oxo groups, wherein in all three instances two R³'s on the same carbon atom may together form a spiro group;
when Q is W, W is selected from an 1,3-diaza-C₅₋₇-cycloalk-2-yl group which is N-substituted with R¹⁶ and optionally further substituted with one or more R³, and optionally containing one or two oxo groups; a 1,3-thiaza-C₅₋₇-cycloalk-2-yl group which is N-substituted with R¹⁶ and optionally further substituted with one or more R³ and optionally containing one or two oxo groups; an 1,3-oxaza-C₅₋₇-cycloalk-2-yl group which is N-substituted with R¹⁶ and optionally further substituted with one or more R³, and optionally containing one or two oxo groups, wherein in all three instances two R³'s on the same carbon atom may together form a spiro group;
R¹⁶ is selected from hydrogen, —C(O)R⁷, —C(O)C(O)R⁷, —C(O)C(O)OR⁷, and —C(O)C(O)NR⁶R⁷;
when Q is —CH(OR¹⁷)₂, each R¹⁷ independently is R³, or wherein two R¹⁷ substituents together with the intervening —O—CH(−)—O— may form a heterocyclyl optionally substituted with one or more R³ and containing up to two oxo groups;
R²⁴, R²⁵, and R²⁶ are each independently selected from the group consisting of hydrogen, C₁₋₆ alkyl, C₁₋₆ alkoxy, C₃₋₁₀ cycloalkyl, aryl, halogen, hydroxymethyl, and C(═O)—R²⁷;
R²⁷ is unsubstituted amine, substituted amine, or heterocycle; and
s is 0, 1, 2, 3, or 4;
with the proviso that at least one of R²⁴, R²⁵, and R²⁶ is not hydrogen.

A tenth aspect of the present disclosure relates to a compound of the Formula (Ij)

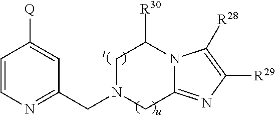

wherein
Q is selected from CO₂H, —CH═NR¹², —W, —CHR²⁰NR²¹R¹³, —CH═O and —CH(OR¹⁷)₂;
each R³ is independently selected from C₁₋₆ alkyl, C₁₋₄ fluoroalkyl, C₁₋₄ hydroxyalkyl, C₂₋₆ alkenyl, C₂₋₆ alkynyl, C₃₋₁₀ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—NR⁶—C(═O)—OR⁷, —Z—C(═O)—R⁷, —Z—OR⁷, halogen, —Z—SR⁷, —Z—SOR⁷, —Z—SO₂R⁷, —Z—SO₂NR⁶R⁷, and —Z—COOR⁷, wherein any heterocyclyl may be substituted with one or more R⁴, and wherein any heteroaryl and any aryl may be substituted with one or more R⁵;
each R⁴ is independently selected from C₁₋₆ alkyl, C₁₋₄ fluoroalkyl, C₁₋₄ hydroxyalkyl, C₁₋₄ alkoxy, C₃₋₁₀ cycloalkyl, NR⁶R⁷, C(═O)—NR⁶R⁷, NR⁶—C(═O)—R⁷, Z—C(═O)—R⁷, —Z—C(═O)—H, OR⁷, halogen, SR⁷, SOR⁷, SO₂R⁷, SO₂NR⁶R⁷ and COOR⁷ and —OH;
each R⁵ is independently selected from C₁₋₆ alkyl, C₁₋₄ fluoroalkyl, C₁₋₄ hydroxyalkyl, C₁₋₄ alkoxy, C₃₋₆ cycloalkyl, —Z—C(═O)—NR⁶R⁷, —Z—NR⁶—C(═O)—R⁷, —Z—C(═O)—R⁷, —Z—C(═O)OR⁷, —Z—NR⁶C(═O)OR⁷, OR⁷, —CN and halogen;
each of R⁶ and R⁷ is independently selected from hydrogen, optionally not both being hydrogen, C₁₋₈ alkyl, C₁₋₄ fluoroalkyl, C₁₋₄ perfluoroalkyl, C₁₋₄ hydroxyalkyl, C₂₋₈ alkenyl, C₂₋₈ alkynyl, C₃₋₁₀ cycloalkyl, —Z-heterocyclyl, —Z-heteroaryl and —Z-aryl, which alkyl, alkenyl, alkynyl, cycloalkyl, heterocyclyl, heteroaryl and aryl may optionally be substituted with one or more independently selected R⁸; or, alternatively, R⁶ and R⁷ may together with the N-atom to which they are attached form an N-heterocyclic ring optionally substituted with one or more independently selected R⁸;
each R⁸ is independently selected from C₁₋₆ alkyl, C₁₋₄ fluoroalkyl, C₁₋₄ hydroxyalkyl, C₂₋₆ alkenyl, C₂₋₆ alkynyl, C₃₋₁₀ cycloalkyl, —Z-heterocyclyl, —Z-heteroaryl, —Z-aryl, —Z—NR¹⁰R¹¹, —Z—C(═O)—NR¹⁰R¹¹, —Z—OR⁹, halogen, —CN, —Z—SR⁹, —Z—SOR⁹, —Z—SO₂R⁹ and —Z—COOR⁹, which alkyl, alkenyl, alkynyl, cycloalkyl, heterocyclics, heteroaryl and aryl may optionally be substituted with one or more selected from C₁₋₄ alkyl, C₁₋₄ fluoroalkyl, C₁₋₄ hydroxyalkyl, C₃₋₆ cycloalkyl, —Z-heterocyclyl, —Z-heteroaryl, —Z-aryl, —Z—NR¹⁰R¹¹, —Z—C(═O)—NR¹⁰R¹¹, —Z—OR⁹, halogen, —CN, —Z—SR⁹, —Z—SOR⁹, —Z—SO₂R⁹ and —Z—COOR⁹; wherein any heterocyclyl may be further substituted with one or more R⁴ as defined above, and wherein any heteroaryl and any aryl may be further substituted with one or more R⁵ as defined above;
each R⁹ is independently selected from —H, C₁₋₈ alkyl, C₁₋₄ fluoroalkyl, C₁₋₄ hydroxyalkyl, C₂₋₈ alkenyl, C₂₋₈ alkynyl, C₃₋₁₀ cycloalkyl, —Z-heterocyclyl, —Z-aryl, and —Z-heteroaryl, wherein any heterocyclyl may be substituted with one or more R⁴ as defined above, and wherein any heteroaryl and any aryl may be substituted with one or more R⁵ as defined above;
each of R¹⁰ and R¹¹ is independently selected from —H, C₁₋₆ alkyl, C₁₋₄ fluoroalkyl, C₁₋₄ hydroxyalkyl, C₂₋₈ alkenyl, C₂₋₈ alkynyl, C₃₋₁₀ cycloalkyl, heterocyclyl, heteroaryl, and aryl, wherein any heterocyclyl may be substituted with one or more R⁴ as defined above, and wherein any heteroaryl and any aryl may be substituted with one or more R⁵ as defined above, or, alternatively, R¹⁰ and R¹¹ may together with the N-atom to which they are attached form an optionally 5 to 7 membered, N-heterocyclic ring optionally substituted with one or more R⁴ as defined above;
when Q is —CH═NR¹², R¹² is selected from C₁₋₁₀ alkyl, C₂₋₁₀ alkenyl, C₂₋₁₀ alkynyl, C₃₋₁₀ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—C(═O)—R⁷, —Z—OR⁷, halogen, —Z—SR⁷, —Z—SOR⁷, —Z—SO₂R⁷ and —Z—COOR⁷, which alkyl, alkenyl, alkynyl, cycloalkyl, heterocyclyl, heteroaryl and aryl may optionally be substituted with one or more R³;
when Q is —CHR²⁰NR²¹R¹³, R¹³ is selected from hydrogen, —C(O)R⁷, —C(O)C(O)R⁷, —C(O)C(O)OR⁷, C₁₋₈ alkyl, C₁₋₄ fluoroalkyl, C₁₋₄ perfluoroalkyl, C₁₋₄ hydroxyalkyl, C₂₋₈ alkenyl, C₂₋₈ alkynyl, C₃₋₁₀ cycloalkyl, —Z-heterocyclyl, —Z-aryl and —Z-heteroaryl, which alkyl, alkenyl, alkynyl, cycloalkyl, heterocyclyl, aryl and heteroaryl may optionally be substituted with one or more independently selected R⁸, or is —CR¹⁴R¹⁵—NR⁶R⁷, —CR¹⁴R¹⁵CN, or —CR¹⁴R¹⁵OR⁷, wherein each of R¹⁴ and R¹⁵ is independently selected from —H, C₁₋₈ alkyl, C₂₋₈ alkenyl, C₂₋₈ alkynyl, C₃₋₁₀ cycloalkyl, heterocyclyl, heteroaryl and aryl, and wherein R¹⁴ and R¹⁵ together with the intervening carbon atom may designate a C₃₋₁₀ cycloalkyl or C₅₋₁₀-cycloalkenyl ring, which alkyl, alkenyl, alkynyl, cycloalkyl (ring), cycloalkenyl ring, heterocyclyl, heteroaryl and aryl may optionally be substituted with one or more R³;
R²⁰ and R²¹ are hydrogen, or together form a 1,3-diaza-C₅₋₇-cycloalk-2-yl group which is N-substituted with R¹⁶ and optionally further substituted with one or more R³, and optionally containing one or two oxo groups; a 1,3-thiaza-C₅₋₇-cycloalk-2-yl group which is N-substituted with R¹⁶ and optionally further substituted with one or more R³ and optionally containing one or two oxo groups; an 1,3-oxaza-C₅₋₇-cycloalk-2-yl group which is N-substituted with R¹⁶ and optionally further substituted with one or more R³, and optionally containing one or two oxo groups, wherein in all three instances two R³'s on the same carbon atom may together form a spiro group;
when Q is W, W is selected from an 1,3-diaza-C₅₋₇-cycloalk-2-yl group which is N-substituted with R¹⁶ and optionally further substituted with one or more R³, and optionally containing one or two oxo groups; a 1,3-thiaza-C₅₋₇-cycloalk-2-yl group which is N-substituted with R¹⁶ and optionally further substituted with one or more R³ and optionally containing one or two oxo groups; an 1,3-oxaza-C₅₋₇-cycloalk-2-yl group which is N-substituted with R¹⁶ and optionally further substituted with one or more R³, and optionally containing one or two oxo groups, wherein in all three instances two R³'s on the same carbon atom may together form a spiro group;
R¹⁶ is selected from hydrogen, —C(O)R⁷, —C(O)C(O)R⁷, —C(O)C(O)OR⁷, and —C(O)C(O)NR⁶R⁷;
when Q is —CH(OR¹⁷)₂, each R¹⁷ independently is R³, or wherein two R¹⁷ substituents together with the intervening —O—CH(−)—O— may form a heterocyclyl optionally substituted with one or more R³ and containing up to two oxo groups;
R³⁰ is selected from the group consisting of hydrogen, halogen, C₁₋₆ alkyl, and aryl, wherein C₁₋₆ alkyl and aryl groups may optionally be further substituted by halogen, hydroxy, C₁₋₆ alkyl, C₁₋₆ alkoxy, unsubstituted amine, or substituted amine;
R²⁸ and R²⁹ are independently selected from the group consisting of hydrogen, halogen, and C₁₋₆ alkyl;
t is 1, 2, or 3; and
u is 1, 2, or 3.

In some of the above aspects, A is a group containing a double bond. It will be appreciated that in compliance with the general formula, A is not bonded to the adjacent nitrogen by such a double bond.

The term ‘Formula (I)’ is used herein to encompass all of Formulae (Ia) to (If) above.

A in any of the compounds defined by general formula herein may be —CHR²C(O)—.

A in any of the compounds defined by general formula herein may be —CH₂—C(O)—.

Y in any of the compounds defined by general formula herein may be

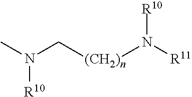

wherein n is from 1 to 3 and each of R¹⁰ and R¹¹ independently is as defined above.

Y in any of the compounds defined by general formula herein may be

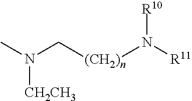

for instance

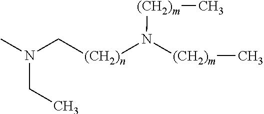

wherein n is from 1 to 3 and each m independently is from 0 to 2.

Y in any of the compounds defined by general formula herein may be selected from heterocyclyl, heteroaryl and aryl, which may be optionally substituted with one or more R³.

R¹³ may be H in any of the compounds defined by general formula herein.

Q may be of the formula:

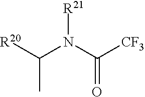

wherein R²⁰ and R²¹ are hydrogen, or together form a 1,3-diaza-C₅₋₇-cycloalk-2-yl group which is N-substituted with R¹⁶ and optionally further substituted with one or more R³, and optionally containing one or two oxo groups; a 1,3-thiaza-C₅₋₇-cycloalk-2-yl group which is N-substituted with R¹⁰ and optionally further substituted with one or more R³ and optionally containing one or two oxo groups; an 1,3-oxaza-C₅₋₇-cycloalk-2-yl group which is N-substituted with R¹⁶ and optionally further substituted with one or more R³, and optionally containing one or two oxo groups, wherein in all three instances two R³'s on the same carbon atom may together form a spiro group.

In some preferred instances, the compound may be one wherein the moiety -A-Y includes 1-3 cyclic moieties selected from monocyclic cycloalkyl, monocyclic heterocyclyl, monocyclic heteroaryl, dicyclic heteroaryl and monocyclic aryl.

In preferred aspects of the disclosure, the compound may be as shown in Table 1 in the Examples section below.

A compound according to the disclosure may have a molecular weight of 130-1,000 g/mol, such as 180-800 g/mol, e.g. 225-600 g/mol or 250-500 g/mol.

The disclosure includes a pharmaceutical composition comprising at least one compound of Formula (I) as defined in any paragraph herein containing such a definition and optionally one or more pharmaceutically acceptable excipients, diluents or carriers.

The disclosure includes such a pharmaceutical composition, which comprises one or more further active substances.

The disclosure includes a compound for use as a medicament which is a compound of the Formula (I).

The disclosure includes a compound for use in the treatment of a HDME dependent disease which is of the Formula (I).

The disclosure includes the use of a compound for the preparation of a pharmaceutical composition for the treatment of a HDME dependent disease, which compound is of the Formula (I). The HDME may be a member of at least one of the KDM7, KDM6, KDM5, KDM4, KDM3 or KDM2 families. In some aspects of the disclosure, the HDME is at least one of PHF8, KDM6A, KDM5A, KDM5B, KDM4A, KDM4C, KDM3A, KDM2A, or KDM2B.

The disclosure includes a method of treating a HDME dependent disease in a subject, said method comprises administering to said subject a therapeutically effective amount of at least one compound of Formula (I) as defined in any one of the above paragraphs.

Conditions treatable using compounds or formulations or compositions according to the disclosure include cancer in the broadest sense, including solid and non-solid tumours. Further details of treatable conditions appear below.

### DETAILED DISCLOSURE OF THE DISCLOSURE

The above definitions of the compounds of Formula (I) are referred to herein by the expressions “compounds of Formula (I)” as defined herein, “compound of Formula (I) as defined herein”, or simply “compounds of Formula (I)”, etc. It should be understood, that such references are intended to encompass not only the above general formula in its stated aspects, but also each and every of the embodiments, etc. discussed above or in the following. It should also be understood, that unless stated to the opposite, such references also encompass isomers, mixtures of isomers, isotopic variants, pharmaceutically acceptable salts, solvates and prodrugs of the compounds of Formula (I).

Without being bound by any particular theory, it is believed that the substituent combination -A-Y plays a role in establishing affinity for said histone demethylases. Furthermore, it is believed that the aromatic ring nitrogen and the side chain nitrogen atom of Formula (I) also play a role in the binding of a particular cavity of the histone demethylases where the iron atom lies. It is also believed that the A-Y chain itself, and through its substituents, interacts with the area of the demethylase known to accommodate the lysine chain of the substrate in many cases.

In several aspect of the disclosure, A is typically selected from —CHR²C(O)—, C₁₋₈ alkylene, C₂₋₈ alkenylene, C₂₋₈ alkynylene, C₃₋₁₀ cycloalkylene, heterocyclylene, heteroarylene and arylene. The alkylene, alkenylene, alkynylene, cycloalkylene, heterocyclylene, heteroarylene and arylene as A may optionally be substituted with one or more R³ (see further below). A may be selected from —CHR²C(O)—, C₁₋₈ alkylene, C₃₋₁₀ cycloalkylene, heterocyclylene, heteroarylene and arylene, in particular from —CHR²C(O)—, C₁₋₈ alkylene and heterocyclylene, such as —CHR²C(O)—, or C₁₋₈ alkylene, or heterocyclylene.

Y is typically selected from —H, —NR⁶R⁷, —OR⁷, C₁₋₈ alkyl, C₂₋₈ alkenyl, C₂₋₈ alkynyl, C₃₋₁₀ cycloalkyl, heterocyclyl, heteroaryl and aryl. R⁶ and R⁷ are exemplified further below.

The alkyl, alkenyl, alkynyl, cycloalkyl, heterocyclyl, heteroaryl and aryl as Y may optionally be substituted with one or more R³ (see further below);

In some embodiments, Y is —NR⁶R⁷. In one variant type, A is —CHR²C(O)— and Y is —NR⁶R⁷. In another variant type, A is C₁₋₈ alkyl and Y is —NR⁶R⁷. In one scenario within these embodiments and these variants, —NR⁶R⁷ represents an N-heterocyclic ring optionally substituted with one or more independently selected R⁸, preferably substituted with one to two independently selected R⁸. In another scenario within these embodiments and these variants wherein Y is —NR⁶R⁷, one of R⁶ and R⁷ represents —H or C₁₋₆ alkyl. In still another scenario within these embodiment types and these variants wherein Y is —NR⁶R⁷, R⁶ and R⁷ are independently selected from C₁₋₈ alkyl, C₁₋₄ fluoroalkyl, C₁₋₄ hydroxyalkyl, C₂₋₈ alkenyl, and C₂₋₈ alkynyl, e.g. such that R⁶ and R⁷ are the same. In still another scenario within these embodiment types and these variants wherein Y is —NR⁶R⁷, one of R⁶ and R⁷ is selected from heterocyclyl, heteroaryl and aryl.

Y may be —H. In such compounds and in others, A may be selected from C₁₋₈ alkylene, C₂₋₈ alkenylene, C₂₋₈ alkynylene, and C₃₋₁₀ cycloalkylene. In such compounds and in others, A may also be selected from heterocyclyl.

Y may be selected from heterocyclyl, heteroaryl and aryl. In such compounds and others, A may be selected from C₁₋₈ alkylene, C₂₋₈ alkenylene, C₂₋₈ alkynylene, in particular from C₁₋₈ alkylene, such as from C₁₋₆ alkylene, in particular from C₁₋₄ alkylene.

In several aspects of the disclosure, R¹ is typically selected from —H and C₁₋₄ alkyl (such as methyl, ethyl, propyl and butyl), in particular from —H and methyl.

In several aspects of the disclosure, R² is typically selected from —H, C₁₋₈ alkyl, C₂₋₈ alkenyl, C₂₋₈ alkynyl, C₃₋₁₀ cycloalkyl, which alkyl, alkenyl, alkynyl and cycloalkyl may be optionally substituted with one or more selected from —OH, aryl, C₁₋₆ alkoxy, heteroaryl, aryloxy, heteroaryloxy, F, and C₃₋₆ cycloalkyl. In some embodiments, R² is selected from —H, C₁₋₄ alkyl (such as methyl, ethyl, propyl and butyl) and C₁₋₄ hydroxyalkyl (such as hydroxymethyl, hydroxyethyl, hydroxypropyl and hydroxybutyl), in particular from —H, methyl and hydroxymethyl. The same is true of R^{2a}.

The R³ (possible substituents to some of the meanings of A and Y) is typically independently selected from C₁₋₆ alkyl, C₁₋₄ fluoroalkyl, C₁₋₄ hydroxyalkyl, C₂₋₆ alkenyl, C₂₋₆ alkynyl, C₃₋₁₀ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—C(═O)—R⁷, —Z—OR⁷, halogen, —Z—SR⁷, —Z—SOR⁷, —Z—SO₂R⁷, —Z—SO₂NR⁶R⁷ and —Z—COOR⁷, wherein any heterocyclyl may be substituted with one or more R⁴, and wherein any heteroaryl and any aryl may be substituted with one or more R⁵.

Z is typically selected from C₁₋₄ alkylene, heterocyclylene and C₃₋₆ cycloalkylene. In one embodiment, Z is selected from C₁₋₄ alkylene. In another embodiment, Z is selected from a single bond. It should be understood that the group Z may appear several times in Formula (I) and that such Z's are independently selected. The same is true of Z′. Z is sometimes a single bond.

Each R⁴ (possible substituents of heterocyclyl) may be independently selected from C₁₋₆ alkyl, C₁₋₄ fluoroalkyl, C₁₋₄ hydroxyalkyl, C₁₋₄ alkoxy, C₃₋₁₀ cycloalkyl, —N(R¹)₂, carbamoyl, and —OH.

Each R⁵ (possible substituents of heteroaryl and aryl) may be independently selected from C₁₋₆ alkyl, C₁₋₄ fluoroalkyl, C₁₋₄ hydroxyalkyl, C₁₋₄ alkoxy, C₃₋₆ cycloalkyl, —CN, —F, —Cl, —Br, carbamoyl and —OH.

Generally, each of R⁶ and R⁷ (e.g. of the moiety —NR⁶R⁷) may be independently selected from —H (in certain aspects), C₁₋₈ alkyl, C₁₋₄ fluoroalkyl, C₁₋₄ perfluoroalkyl, C₁₋₄ hydroxyalkyl, C₂₋₈ alkenyl, C₂₋₈ alkynyl, C₃₋₁₀ cycloalkyl, —Z-heterocyclyl, —Z-heteroaryl and —Z-aryl, which alkyl, alkenyl, alkynyl, cycloalkyl, heterocyclyl, heteroaryl and aryl may optionally be substituted with one or more independently selected R⁸; or, alternatively, R⁶ and R⁷ may together with the N-atom to which they are attached form an N-heterocyclic ring optionally substituted with one or more independently selected R⁸.

Each R⁸ may be independently selected from C₁₋₆ alkyl, C₁₋₄ fluoroalkyl, C₁₋₄ hydroxyalkyl, C₂₋₆ alkenyl, C₂₋₆ alkynyl, C₃₋₁₀ cycloalkyl, —Z-heterocyclyl, —Z-heteroaryl, —Z-aryl, —Z—NR¹⁰R¹¹, —Z—C(═O)—NR¹⁰R¹¹, —Z—OR⁹, halogen, —CN, —Z—SR⁹, —Z—SOR⁹, —Z—SO₂R⁹ and —Z—COOR⁹, which alkyl, alkenyl, alkynyl, cycloalkyl, heterocyclics, heteroaryl and aryl may optionally be substituted with one or more selected from C₁₋₄ alkyl, C₁₋₄ fluoroalkyl, C₁₋₄ hydroxyalkyl, C₃₋₆ cycloalkyl, —Z-heterocyclyl, —Z-heteroaryl, —Z-aryl, —Z—NR¹⁰R¹¹, —Z—C(═O)—NR¹⁰R¹¹, —Z—OR⁹, halogen, —CN, —Z—SR⁹, —Z—SOR⁹, —Z—SO₂R⁹ and —Z—COOR⁹; wherein any heterocyclyl may be further substituted with one or more R⁴ as defined above, and wherein any heteroaryl and any aryl may be further substituted with one or more R⁵ as defined above.

Each R⁹ may be independently selected from —H, C₁₋₈ alkyl, C₁₋₄ fluoroalkyl, C₁₋₄ hydroxyalkyl, C₂₋₈ alkenyl, C₂₋₈ alkynyl, C₃₋₁₀ cycloalkyl, —Z-heterocyclyl, —Z-aryl, and —Z-heteroaryl, wherein any heterocyclyl may be substituted with one or more R⁴ as defined above, and wherein any heteroaryl and any aryl may be substituted with one or more R⁵ as defined above.

Each of R¹⁰ and R¹¹ (of the moiety —NR¹⁰R¹¹) may be independently selected from —H, C₁₋₆ alkyl, C₁₋₄ fluoroalkyl, C₁₋₄ hydroxyalkyl, C₂₋₈ alkenyl, C₂₋₈ alkynyl, C₃₋₁₀ cycloalkyl, heterocyclyl, heteroaryl, and aryl, wherein any heterocyclyl may be substituted with one or more R⁴ as defined above, and wherein any heteroaryl and any aryl may be substituted with one or more R⁵ as defined above, or, alternatively, R¹⁰ and R¹¹ may together with the N-atom to which they are attached form an N-heterocyclic ring optionally substituted with one or more R⁴ as defined above.

In some embodiments, Q is —CH═N—R¹². If so, R¹² may be selected from C₁₋₁₀ alkyl, C₂₋₁₀ alkenyl, C₂₋₁₀ alkynyl, C₃₋₁₀ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—C(═O)—R⁷, —Z—OR⁷, halogen, —Z—SR⁷, —Z—SOR⁷, —Z—SO₂R⁷ and —Z—COOR⁷, which alkyl, alkenyl, alkynyl, cycloalkyl, heterocyclyl, heteroaryl and aryl may optionally be substituted with one or more R³. In some embodiments hereof, R¹² is C₁₋₈ alkyl, C₁₋₄ fluoroalkyl, C₁₋₄ perfluoroalkyl, C₂₋₈ alkenyl, C₂₋₈ alkynyl, C₃₋₈ cycloalkyl, —Z-heterocyclyl, —Z-aryl, —Z-heteroaryl, —Z—NR⁶R⁷, and —Z—OR⁷, wherein —Z— is a single bond or C₁₋₄ alkylene, which alkyl, alkenyl, alkynyl, cycloalkyl, heterocyclyl, heteroaryl and aryl may optionally be substituted with one or more R³.

In other embodiments, Q is —W, wherein —W may be an 1,3-azo-C₅₋₇-cycloalk-2-yl group which is N-substituted with R¹⁶ and optionally further substituted with one or more R³. W may be 1,3-diazacyclopent-2-yl (imidazolidin-2-yl), 1,3-diazacyclohex-2-yl (hexahydropyrimidin-2-yl), or 1,3-diazacyclohept-2-yl, for example. The N-substituent may be selected among those defined for R¹⁶ (see above). W may be further substituted with one or more R³, wherein two R³'s on the same carbon atom may together form a spiro group.

In yet other embodiments, Q is —W, wherein —W may be an 1,3-oxaza-C₅₋₇-cycloalk-2-yl group which is N-substituted with R¹⁶ and optionally further substituted with one or more R³. W may be 1,3-oxazacyclopent-2-yl, 1,3-oxazacyclohex-2-yl, 1,3-oxazacyclohept-2-yl, or 7-oxa-9-azaspiro[4,5]decan-8-yl, for example. The N-substituent may be selected among those defined for R¹⁶ (see above). W may be further substituted with one or more R³, wherein two R³'s on the same carbon atom may together form a spiro group.

In some embodiments of the above, W may be further substituted with one or more R³, but is typically not further substituted.

R¹⁶ may be selected from hydrogen, —C(O)R⁷, —C(O)C(O)R⁷, —C(O)C(O)OR⁷, and —C(O)C(O)NR⁶R⁷, in particular from hydrogen and —C(O)R⁷, wherein R⁷ is C₁₋₄ fluoroalkyl or C₁₋₄ perfluoroalkyl. In one embodiment, R⁷ is trifluoromethyl.

In some embodiments Q is —CH₂NHR¹³, and R¹³ may be selected from hydrogen, —C(O)R⁷, —C(O)C(O)R⁷, —R⁷ (in some aspects), —CR¹⁴R¹⁵—NR⁶R⁷, —CR¹⁴R¹⁵CN, —CR¹⁴R¹⁵OR⁷, wherein each of R¹⁴ and R¹⁵ is independently selected from —H, C₁₋₈ alkyl, C₂₋₈ alkenyl, C₂₋₈ alkynyl, C₃₋₁₀ cycloalkyl, heterocyclyl, heteroaryl and aryl, and wherein R¹⁴ and R¹⁵ together with the intervening carbon atom may designate a C₃₋₁₀ cycloalkyl or C₅₋₁₀-cycloalkenyl ring, which alkyl, alkenyl, alkynyl, cycloalkyl (ring), cycloalkenyl ring, heterocyclyl, heteroaryl and aryl may optionally be substituted with one or more R³. In some aspects, rather than —R⁷, R¹³ may be C₁₋₈ alkyl, C₁₋₄ fluoroalkyl, C₁₋₄ perfluoroalkyl, C₁₋₄ hydroxyalkyl, C₂₋₈ alkenyl, C₂₋₈ alkynyl, C₃₋₁₀ cycloalkyl, —Z-heterocyclyl, and —Z-monocyclic-heteroaryl, which alkyl, alkenyl, alkynyl, cycloalkyl, heterocyclyl, and heteroaryl may optionally be substituted with one or more independently selected R⁸.

In some embodiments Q is —CH(OR¹⁷)₂ and each R¹⁷ independently may be R³, or the two R¹⁷ substituents together with the intervening —O—CH(−)—O— may form a heterocyclyl optionally substituted with one or more R³.

It is to be understood that it is generally preferred that in the Formula (I), Y is not H when A is —CH₂—. Generally speaking, it is believed to be advantageous if the moiety -A-Y has a certain “size” with respect to the number of atom (disregarding hydrogen atoms) and/or the molecular weight. Also a limited flexibility of the moiety -A-Y appears to play a certain role.

Hence, it is believed that the moiety -A-Y should preferably consist of at the most 40 heavy atoms, such as at the most 30 heavy atoms, or at the most 25 heavy atoms, or at the most 20 heavy atoms. Preferably, the moiety -A-Y will consist of at least 3, or at least 4, or at least 8 or at least 10 heavy atoms. In some embodiments, the moiety -A-Y preferably consists of 3-40 heavy atoms, such as 4-30 heavy atoms, or 4-25 heavy atoms, or 4-20, or 8-30, or 8-20, or 8-15 heavy atoms. By the term “heavy atom” is meant all atoms in the moiety except the hydrogen atom(s).

Moreover, it is believed that the compounds of Formula (I) should preferably have a molecular weight of at least 130, or at least 150, or at least 180, or at least 250, but not more than 1,000, or not more than 800, or not more than 500, or not more than 400 and may be within any range constructable from these preferred upper and lower limits, such as 130-1,000 g/mol, or 150-1,000 g/mol, such as 180-800 g/mol, e.g. 225-600 g/mol or 250-500 g/mol, or 250 to 400.

In some embodiments, and in order to introduce a limited flexibility of the moiety -A-Y, the moiety includes 1-4 rings, i.e. rings derived from cycloalkyl, cycloalkenyl, heterocyclyl, heteroaryl and/or aryl. In some variant, the moiety -A-Y includes 1-3 cyclic moieties selected from monocyclic cycloalkyl, monocyclic heterocyclyl, monocyclic heteroaryl, dicyclic heteroaryl and monocyclic aryl. Small substituents such as alkyls groups or hydroxyl on alkyl chains also reduce flexibility and favor certain conformations.

It may be preferable that if -A-Y does not include a ring, it includes at least one, for instance from 1 to 3, branches, each of which independently may be of from one heavy atom to six heavy atoms, for instance from one to three heavy atoms, or from one to two heavy atoms. It is preferred that -A-Y should contain at least one hetero-atom, preferably at least one nitrogen atom or at least one oxygen.

### Definitions

The term “alkyl” as used herein refers to a saturated, straight or branched hydrocarbon chain. The hydrocarbon chain preferably contains from one to 8 carbon atoms (C₁₋₈-alkyl), more preferred from one to six carbon atoms (C₁₋₆-alkyl), in particular from one to four carbon atoms (C₁₋₄-alkyl), including methyl, ethyl, propyl, isopropyl, butyl, isobutyl, secondary butyl, tertiary butyl, pentyl, isopentyl, neopentyl, tertiary pentyl, hexyl, isohexyl, heptyl and octyl. In a preferred embodiment “alkyl” represents a C₁₋₄-alkyl group, which may in particular include methyl, ethyl, propyl, isopropyl, butyl, isobutyl, secondary butyl, and tertiary butyl. Correspondingly, the term “alkylene” means the corresponding biradical (-alkyl-).

The term “cycloalkyl” as used herein refers to a cyclic alkyl group, preferably containing from three to ten carbon atoms (C₃₋₁₀-cycloalkyl), such as from three to eight carbon atoms (C₃₋₈-cycloalkyl), preferably from three to six carbon atoms (C₃₋₆-cycloalkyl), including cyclopropyl, cyclobutyl, cyclopentyl, cyclohexyl, cycloheptyl and cyclooctyl. Furthermore, the term “cycloalkyl” as used herein may also include polycyclic groups such as for example bicyclo[2.2.2]octyl, bicyclo[2.2.1]heptanyl, decalinyl and adamantyl. Correspondingly, the term “cycloalkylene” means the corresponding biradical (-cycloalkyl-).

The term “alkenyl” as used herein refers to a straight or branched hydrocarbon chain or cyclic hydrocarbons containing one or more double bonds, including di-enes, tri-enes and poly-enes. Typically, the alkenyl group comprises from two to eight carbon atoms (C₂₋₈-alkenyl), such as from two to six carbon atoms (C₂₋₆-alkenyl), in particular from two to four carbon atoms (C₂₋₄-alkenyl), including at least one double bond. Examples of alkenyl groups include ethenyl; 1- or 2-propenyl; 1-, 2- or 3-butenyl, or 1,3-but-dienyl; 1-, 2-, 3-, 4- or 5-hexenyl, or 1,3-hex-dienyl, or 1,3,5-hex-trienyl; 1-, 2-, 3-, 4-, 5-, 6-, or 7-octenyl, or 1,3-octadienyl, or 1,3,5-octatrienyl, or 1,3,5,7-octatetraenyl, or cyclohexenyl. Correspondingly, the term “alkenylene” means the corresponding biradical (-alkenyl-).

The term “alkynyl” as used herein refers to a straight or branched hydrocarbon chain containing one or more triple bonds, including di-ynes, tri-ynes and poly-ynes. Typically, the alkynyl group comprises of from two to eight carbon atoms (C₂₋₈-alkynyl), such as from two to six carbon atoms (C₂₋₆-alkynyl), in particular from two to four carbon atoms (C₂₋₄-alkynyl), including at least one triple bond. Examples of preferred alkynyl groups include ethynyl; 1- or 2-propynyl; 1-, 2- or 3-butynyl, or 1,3-but-diynyl; 1-, 2-, 3-, 4- or 5-hexynyl, or 1,3-hex-diynyl, or 1,3,5-hex-triynyl; 1-, 2-, 3-, 4-, 5-, 6-, or 7-octynyl, or 1,3-oct-diynyl, or 1,3,5-oct-triynyl, or 1,3,5,7-oct-tetraynyl. Correspondingly, the term “alkynylene” means the corresponding biradical (-alkynyl-).

The terms “halo” and “halogen” as used herein refer to fluoro, chloro, bromo or iodo. Thus a trihalomethyl group represents e.g. a trifluoromethyl group, or a trichloromethyl group. Preferably, the terms “halo” and “halogen” designate fluoro or chloro.

The term “fluoroalkyl” as used herein refers to an alkyl group as defined herein which is substituted one or more times with one or more fluoro, preferably perfluorated. The term “perfluoroalkyl” as used herein refers to an alkyl group as defined herein wherein all hydrogen atoms are replaced by fluoro atoms. Preferred fluoroalkyl groups include trifluoromethyl, pentafluoroethyl, etc.

The term “alkoxy” as used herein refers to an “alkyl-O—” group, wherein alkyl is as defined above.

The term “oxyalkyl” as used herein refers to an alkoxy (alkyl-O—) group or an alkoxyalkyl (alkyl-O-alkylene-) group.

The term “hydroxyalkyl” as used herein refers to an alkyl group (as defined hereinabove), which alkyl group is substituted one or more times with hydroxy. Examples of hydroxyalkyl groups include HO—CH₂—, HO—CH₂—CH₂— and CH₃—CH(OH)—.

The term “oxy” as used herein refers to an “—O—” group.

The term “oxo” as used herein refers to an “═O” group.

The term “amine” as used herein refers to primary (R—NH₂, R≠H), secondary (R₂—NH, R₂≠H) and tertiary (R₃—N, R≠H) amines. A substituted amine is intended to mean an amine where at least one of the hydrogen atoms has been replaced by the substituent.

The term “carbamoyl” as used herein refers to a “H₂N(C═O)—” group.

The term “aryl”, as used herein, unless otherwise indicated, includes carbocyclic aromatic ring systems derived from an aromatic hydrocarbon by removal of a hydrogen atom. Aryl furthermore includes bi-, tri- and polycyclic ring systems. Examples of preferred aryl moieties include phenyl, naphthyl, indenyl, indanyl, fluorenyl, biphenyl, indenyl, naphthyl, anthracenyl, phenanthrenyl, pentalenyl, azulenyl, and biphenylenyl. Preferred “aryl” is phenyl, naphthyl or indanyl, in particular phenyl, unless otherwise stated. Any aryl used may be optionally substituted. Correspondingly, the term “arylene” means the corresponding biradical (-aryl-).

The term “heteroaryl”, as used herein, refers to aromatic groups containing one or more heteroatoms selected from O, S, and N, preferably from one to four heteroatoms, and more preferably from one to three heteroatoms. Heteroaryl furthermore includes bi-, tri- and polycyclic groups, wherein at least one ring of the group is aromatic, and at least one of the rings contains a heteroatom selected from O, S, and N. Heteroaryl also include ring systems substituted with one or more oxo moieties. Examples of preferred heteroaryl moieties include N-hydroxytetrazolyl, N-hydroxytriazolyl, N-hydroxyimidazolyl, furanyl, triazolyl, pyranyl, thiadiazinyl, benzothiophenyl, dihydro-benzo[b]thiophenyl, xanthenyl, isoindanyl, acridinyl, benzisoxazolyl, quinolinyl, isoquinolinyl, phteridinyl, azepinyl, diazepinyl, imidazolyl, thiazolyl, carbazolyl, pyridinyl, pyridazinyl, pyrimidinyl, pyrazolyl, pyrazinyl, tetrazolyl, furyl, thienyl, isoxazolyl, oxazolyl, isothiazolyl, pyrrolyl, indolyl, benzimidazolyl, benzofuranyl, cinnolinyl, indazolyl, indolizinyl, phthalazinyl, triazinyl, isoindolyl, purinyl, oxadiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, azaindolyl, pyrazolinyl, and pyrazolidinyl. Non-limiting examples of partially hydrogenated derivatives are 1,2,3,4-tetrahydronaphthyl, 1,4-dihydronaphthyl, and 1-octalin. Correspondingly, the term “heteroarylene” means the corresponding biradical (-heteroaryl-).

The term “heterocyclyl” as used herein, refers to cyclic non-aromatic groups containing one or more heteroatoms selected from O, S, and N, preferably from one to four heteroatoms, and more preferably from one to three heteroatoms. Heterocyclyl furthermore includes bi-, tri- and polycyclic non-aromatic groups, and at least one of the rings contains a heteroatom selected from O, S, and N. Heterocyclyl also include ring systems substituted with one or more oxo moieties. Examples of heterocyclic groups are oxetane, tetrahydrofuryl, azetidinyl, azacycloheptanyl, azacyclooctanyl, pyrrolidinyl, pyrrolyl, 3H-pyrrolyl, oxolanyl, furanyl, thiolanyl, S,S-dioxo-thiolanyl, thiophenyl, pyrazolyl, pyrazolidinyl, imidazolyl, imidazolidinyl, 3H-pyrazolyl, 1,2-oxazolyl, 1,3-oxazolyl, 1,2-thiazolyl, 1,3-thiazolyl, 1,2,5-oxadiazolyl, piperidinyl, pyridinyl, oxanyl, 2-H-pyranyl, 4-H-pyranyl, thianyl, 2H-thiopyranyl, pyridazinyl, 1,2-diazinanyl, pyrimidinyl, 1,3-diazinanyl, pyrazinyl, piperazinyl, 1,4-dioxinyl, 1,4-dioxanyl, 1,3-diazinanyl, 1,4-oxazinyl, morpholinyl, thiomorpholinyl, 1,4-oxathianyl, benzofuranyl, isobenzofuranyl, indazolyl, benzimidazolyl, quinolinyl, isoquinolinyl, chromanyl, isochromanyl, 4H-chromenyl, 1H-isochromenyl, cinnolinyl, quinazolinyl, quinoxalinyl, phthalazinyl, purinyl, naphthyridinyl, pteridinyl, indolizinyl, 1H-pyrrolizinyl, 4H-quinolizinyl, beta-lactam, gamma-lactam, delta-lactam, epsilon-lactam, zeta-lactam, and aza-8-bicyclo[3.2.1]octane. Correspondingly, the term “heterocyclylene” means the corresponding biradical (-heterocyclyl-).

The term “N-heterocyclic ring” as used herein, refers to a heterocyclyl or a heteroaryl as defined hereinabove having at least one nitrogen atom, and being bound via a nitrogen atom. Examples of such N-heterocyclic rings are pyrrolidinyl, pyrrolyl, 3H-pyrrolyl, pyrazolyl, pyrazolidinyl, imidazolyl, imidazolidinyl, 3H-pyrazolyl, 1,2-oxazolyl, 1,2-thiazolyl, 1,3-thiazolyl, piperidinyl, pyridinyl, pyridazinyl, pyrazinyl, piperazinyl, morpholinyl, pyridinyl, pyridazinyl, pyrimidinyl, pyrazolyl, pyrazinyl, tetrazolyl, etc.

### Isomers

The compounds of Formula (I) may exist as geometric isomers (i.e. cis-trans isomers), optical isomers or stereoisomers, such as diastereomers, as well as tautomers. Accordingly, it should be understood that the definition of compounds of Formula (I) includes each and every individual isomers corresponding to the structural formula: Formula (I), including cis-trans isomers, stereoisomers and tautomers, as well as racemic mixtures of these and pharmaceutically acceptable salts thereof. Hence, the definition of compounds of Formula (I) is also intended to encompass all R- and S-isomers of a chemical structure in any ratio, e.g. with enrichment (i.e. enantiomeric excess or diastereomeric excess) of one of the possible isomers and corresponding smaller ratios of other isomers.

Diastereoisomers, i.e. non-superimposable stereochemical isomers, can be separated by conventional means such as chromatography, distillation, crystallization or sublimation. The optical isomers can be obtained by resolution of the racemic mixtures according to conventional processes, for example by formation of diastereoisomeric salts by treatment with an optically active acid or base. Examples of appropriate acids include, without limitation, tartaric, diacetyltartaric, dibenzoyltartaric, ditoluoyltartaric and camphorsulfonic acid. The mixture of diastereomers can be separated by crystallization followed by liberation of the optically active bases from these salts. An alternative process for separation of optical isomers includes the use of a chiral chromatography column optimally chosen to maximize the separation of the enantiomers. Still another available method involves synthesis of covalent diastereoisomeric molecules by reacting compounds of Formula (I) with an optically pure acid in an activated form or an optically pure isocyanate. The synthesized diastereoisomers can be separated by conventional means such as chromatography, distillation, crystallization or sublimation, and then hydrolyzed to obtain the enantiomerically pure compound. The optically active compounds of Formula (I) can likewise be obtained by utilizing optically active starting materials and/or by utilizing a chiral catalyst. These isomers may be in the form of a free acid, a free base, an ester or a salt. Examples of chiral separation techniques are given in Chiral Separation Techniques, A Practical Approach, 2^{nd} ed. by G. Subramanian, Wiley-VCH, 2001.

### General Synthetic Procedures

The compounds of this disclosure are prepared according to the following synthetic plans. In all these plans, protecting groups were used as required on peripheral functional groups.

For Q=COOH, these acids can be obtained from hydrolysis of a corresponding alkyl ester. These alkyl esters were in turn obtained by a reductive amination of an aldehyde-amine or ketone-amine pair.

For Q=CHO, these aldehydes were obtained by oxidation of the corresponding primary alcohol or by reduction of the corresponding alkyl esters (vide supra). These primary alcohols were obtained either by reduction of the corresponding alkyl esters (vide supra) or by deprotection of the protected alcohol. The primary alcohols were in turn obtained by reductive amination of an aldehyde-amine or ketone-amine pair bearing the alcohol.

For Q=CH═NR¹², these imines were obtained by reacting the corresponding aldehyde with the appropriate primary amine H₂NR¹².

For Q=CHR²⁰NR²¹R¹³, these amines by reductive amination of an aldehyde-amine or ketone-amine pair bearing the amine or were obtained by reductive amination of the aldehydes (vide supra) or ketones with the appropriate amine HNR²¹R¹³. The ketones were obtained by by reductive amination of an aldehyde-amine or ketone-amine pair bearing the ketone or by reaction of the corresponding aldehyde (vide supra) with a Grignard reagent of R₂₀, followed by oxidation of the resulting secondary alcohol. If R¹³ is an acyl group, it may have to be introduced after the reductive amination step.

For Q=CH(OR¹⁷)₂, these acetals were obtained by treatment of the aldehydes (vide supra) with the alcohol HOR¹⁷.

For Q=W, these capped heterocycles were obtained by reacting the corresponding heterocycles with an electrophilic form of R¹⁶, such as an acid chloride, if R¹⁶ is not H. These heterocycles were in turn obtained by reacting the aldehydes (vide supra), with the appropriate diamine, aminothiol or aminoalcohol under dehydrating conditions.

### Biological Assays

Histone lysine demethylase AlphaLISA assays are performed to determine IC₅₀ values. This example demonstrates the ability of compounds of the disclosure to inhibit the activity in vitro of tested enzymes. Assays are performed analogously to the protocol described by PerkinElmer (Roy et al. PerkinElmer Technical Note: AlphaLISA #12, April 2011).

Histone lysine demethylase immunofluorescence assays are performed to determine the IC₅₀ value for endogenous protein, which may be used to demonstrate the ability of compounds of the disclosure to inhibit demethylation of histone 3 lysine 4 in a human cell line, such as U2OS. Generally, the cells are incubated with compounds, washed and incubated with a methylation specific antibody before imaging. IC₅₀ values are determined by measurement of the H3K4me3 staining.

Additional histone lysine demethylase immunofluorescence assays are performed to demonstrate the ability of the compounds of the disclosure to inhibit the activity of a specific histone lysine demethylases overexpressed in a cell line. Cells ectopically expressing the relevant histone lysine demethylase are incubated with compound, washed and incubated with a methylation specific antibody before imaging. The IC₅₀ values are determined by changes in the specific methylation state of specific histone lysine residues in the cells overexpressing the relevant histone lysine demethylase. Cell proliferation assays are performed to determine EC₅₀ values, which may be used to demonstrate the ability of the compounds of the disclosure to inhibit the proliferation of a human cancer or other cell line. Generally, cells, such as MCF7 cells, are incubated with compounds for a certain time, such as 5 days. EC₅₀ values are determined by life cell imaging or by tox assays, such the ATPlite 1 Step assay.

### Pharmaceutically Acceptable Salts

The compound of Formula (I) may be provided in any form suitable for the intended administration, in particular including pharmaceutically acceptable salts, solvates and prodrugs of the compound of Formula (I).

Pharmaceutically acceptable salts refer to salts of the compounds of Formula (I), which are considered to be acceptable for clinical and/or veterinary use. Typical pharmaceutically acceptable salts include those salts prepared by reaction of the compounds of Formula (I) a mineral or organic acid or an organic or inorganic base. Such salts are known as acid addition salts and base addition salts, respectively. It will be recognized that the particular counter-ion or multiple counter-ions forming a part of any salt is not of a critical nature, so long as the salt as a whole is pharmaceutically acceptable and as long as the counter-ion does not contribute undesired qualities to the salt as a whole. These salts may be prepared by methods known to the skilled person. Pharmaceutically acceptable salts are, e.g., those described and discussed in Remington's Pharmaceutical Sciences, 17. Ed. Alfonso R. Gennaro (Ed.), Mack Publishing Company, Easton, Pa., U.S.A., 1985 and more recent editions and in Encyclopedia of Pharmaceutical Technology.

Examples of pharmaceutically acceptable addition salts include acid addition salts formed with inorganic acids e.g. hydrochloric, hydrobromic, sulfuric, nitric, hydroiodic, metaphosphoric, or phosphoric acid; and organic acids e.g. succinic, maleic, acetic, fumaric, citric, tartaric, benzoic, trifluoroacetic, malic, lactic, formic, propionic, glycolic, gluconic, camphorsulfuric, isothionic, mucic, gentisic, isonicotinic, saccharic, glucuronic, furoic, glutamic, ascorbic, anthranilic, salicylic, phenylacetic, mandelic, embonic (pamoic), ethanesulfonic, pantothenic, stearic, sulfinilic, alginic and galacturonic acid; and arylsulfonic, for example benzenesulfonic, p-toluenesulfonic, oxalic, methanesulfonic or naphthalenesulfonic acid; and base addition salts formed with alkali metals and alkaline earth metals and organic bases such as N,N-dibenzylethylenediamine, chloroprocaine, choline, diethanolamine, ethylenediamine, meglumine (N-methylglucamine), lysine and procaine; and internally formed salts.

### Solvates

The compound of Formula (I) may be provided in dissoluble or indissoluble forms together with a pharmaceutically acceptable solvent such as water, ethanol, and the like. Dissoluble forms may also include hydrated forms such as the mono-hydrate, the dihydrate, the hemihydrate, the trihydrate, the tetrahydrate, and the like.

### Isotopic Variations

Elemental symbols and element names are used herein to include isotopes of the named elements. In particular one, some, or all hydrogens may be deuterium. Radioactive isotopes may be used, for instance to facilitate tracing the fate of the compounds or their metabolic products after administration.

### Prodrugs

The compound of Formula (I) may be provided as a prodrug. The term “prodrug” used herein is intended to mean a compound which—upon exposure to certain physiological conditions—will liberate the compound of Formula (I) which then will be able to exhibit the desired biological action. A typical example is a labile carbamate of an amine and a further example would be a trialkylsilyl ether of an alcohol or a trialkylsilyl ester of an acid, each optionally being trimethylsilyl.

### Inhibitory Effect

The inventors have surprisingly found that compounds of Formula (I) as defined herein have an inhibitory effect on the activity of one or more HDMEs. In this respect said one or more HDMEs may be any HDME, however preferably the one or more HDMEs are selected from the JmjC (Jumonji) family, more preferably said one or more HDME(s) are HDME of the human JmjC family and even more preferably are HDME belonging to the KDM7, KDM6, KDM5, KDM4, KDM3 or KDM2 families, and most preferably said one or more HDME(s) are PHF8, KDM6A, KDM5A, KDM5B, KDM4A, KDM4C, KDM3A, KDM2A, and/or KDM2B. The present disclosure also relates to a compound of Formula (I) as defined herein in a method for inhibiting HDMEs. The method includes contacting a cell with a compound of Formula (I). In a related embodiment, the method further provides that the compound is present in an amount effective to produce a concentration sufficient to inhibit the demethylation of a histone in the cell.

Thus, preferably in an assay for demethylation of a histone substrate by said HDME, then preferred compounds of Formula (I) are compounds capable of reducing or preferably inhibiting said demethylation by said HDME. Said histone substrate may be any histone, but preferably is histone H3 or a fragment thereof, even more preferred: a fragment comprising K4, K9, K27, or K36 of H3. Preferably, said inhibition is determined as the IC₅₀ of said compound of Formula (I) in respect of the said demethylation assay.

Preferred compounds of Formula (I) which have an IC₅₀ at or below 1 μM, more preferably less than 300 nM, for example less than 100 nM, such as less than 50 nM in respect of demethylation of any of said histone substrates by any of said HDME. Thus very preferred compounds of Formula (I) which have an IC₅₀ at or below 1 μM, more preferably less than 500 nM, for example less than 100 nM, such as less than 50 nM in respect of demethylation of histone H3 methylated at least on one lysine.

In a preferred embodiment IC₅₀ is determined as described in Example 2 herein below. Thus, particularly preferred are compounds of Formula (I) which have an IC₅₀ at or below 1 μM, more preferably less than 500 nM, for example less than 100 nM, such as less than 50 nM when said IC₅₀ is determined as described in and one of the Examples herein below.

Particularly preferred compounds of Formula (I) are compounds that lead to a decreased tumour size and/or decreased number of metastases when tested in a xenograft model (Morton and Houghton, Nature Protocols, 2 (2) 247-250, 2007).

### Pharmaceutical Compositions

In one aspect of this disclosure, there is provided a pharmaceutical composition comprising at, as an active ingredient, at least one compound of Formula (I) as defined herein and optionally one or more pharmaceutically acceptable excipients, diluents and/or carriers. The compounds of Formula (I) may be administered alone or in combination with pharmaceutically acceptable carriers, diluents or excipients, in either single or multiple doses. Suitable pharmaceutically acceptable carriers, diluents and excipients include inert solid diluents or fillers, sterile aqueous solutions and various organic solvents.

The pharmaceutical compositions may be formulated with pharmaceutically acceptable carriers or diluents as well as any other known adjuvants and excipients in accordance with conventional techniques such as those disclosed in Remington: The Science and Practice of Pharmacy, 21st Edition, 2000, Lippincott Williams & Wilkins.

The pharmaceutical compositions formed by combining a compound of Formula (I) as defined herein with pharmaceutically acceptable carriers, diluents or excipients can be readily administered in a variety of dosage forms such as tablets, powders, lozenges, syrups, suppositories, injectable solutions and the like. In powders, the carrier is a finely divided solid such as talc or starch which is in a mixture with the finely divided active component. In tablets, the active component is mixed with the carrier having the necessary binding properties in suitable proportions and compacted in the shape and size desired.

The pharmaceutical compositions may be specifically prepared for administration by any suitable route such as the oral and parenteral (including subcutaneous, intramuscular, intrathecal, intravenous and intradermal) route. It will be appreciated that the preferred route will depend on the general condition and age of the subject to be treated, the nature of the condition to be treated and the active ingredient chosen.

Pharmaceutical compositions for oral administration include solid dosage forms such as capsules, tablets, dragees, pills, lozenges, powders and granules. Where appropriate, they can be prepared with coatings such as enteric coatings or they can be prepared so as to provide controlled release of the active ingredient such as sustained or prolonged release according to methods well known in the art.

For oral administration in the form of a tablet or capsule, a compound of Formula (I) as defined herein may suitably be combined with an oral, non-toxic, pharmaceutically acceptable carrier such as ethanol, glycerol, water or the like. Furthermore, suitable binders, lubricants, disintegrating agents, flavouring agents and colourants may be added to the mixture, as appropriate. Suitable binders include, e.g., lactose, glucose, starch, gelatin, acacia gum, tragacanth gum, sodium alginate, carboxymethylcellulose, polyethylene glycol, waxes or the like. Lubricants include, e.g., sodium oleate, sodium stearate, magnesium stearate, sodium benzoate, sodium acetate, sodium chloride or the like. Disintegrating agents include, e.g., starch, methyl cellulose, agar, bentonite, xanthan gum, sodium starch glycolate, crospovidone, croscarmellose sodium or the like. Additional excipients for capsules include macrogols or lipids.

For the preparation of solid compositions such as tablets, the active compound of Formula (I) is mixed with one or more excipients, such as the ones described above, and other pharmaceutical diluents such as water to make a solid pre-formulation composition containing a homogenous mixture of a compound of Formula (I). The term “homogenous” is understood to mean that the compound of Formula (I) is dispersed evenly throughout the composition so that the composition may readily be subdivided into equally effective unit dosage forms such as tablets or capsules.

Liquid compositions for either oral or parenteral administration of the compound of Formula (I) include, e.g., aqueous solutions, syrups, elixirs, aqueous or oil suspensions and emulsion with edible oils such as cottonseed oil, sesame oil, coconut oil or peanut oil. Suitable dispersing or suspending agents for aqueous suspensions include synthetic or natural gums such as tragacanth, alginate, acacia, dextran, sodium carboxymethylcellulose, gelatin, methylcellulose or polyvinylpyrrolidone.

Pharmaceutical compositions for parenteral administration include sterile aqueous and non-aqueous injectable solutions, dispersions, suspensions or emulsions as well as sterile powders to be reconstituted in sterile injectable solutions or dispersions prior to use. For parenteral administration, solutions containing a compound of Formula (I) in sesame or peanut oil, aqueous propylene glycol, or in sterile aqueous solution may be employed. Such aqueous solutions should be suitably buffered if necessary and the liquid diluent first rendered isotonic with sufficient saline or glucose. These particular aqueous solutions are especially suitable for intravenous, intramuscular, subcutaneous and intraperitoneal administration. The oily solutions are suitable for intra-articular, intra-muscular and subcutaneous injection purposes.

The preparation of all these solutions under sterile conditions is readily accomplished by standard pharmaceutical techniques well known to those skilled in the art.

Depot injectable compositions are also contemplated as being within the scope of the present disclosure.

In addition to the aforementioned ingredients, the compositions of a compound of Formula (I) may include one or more additional ingredients such as diluents, buffers, flavouring agents, colourant, surface active agents, thickeners, preservatives, e.g. methyl hydroxybenzoate (including anti-oxidants), emulsifying agents and the like.

A suitable dosage of the compound of Formula (I) will depend on the age and condition of the patient, the severity of the disease to be treated and other factors well known to the practicing physician. The compound may be administered for example either orally, parenterally or topically according to different dosing schedules, e.g. daily or with intervals, such as weekly intervals. In general a single dose will be in the range from 0.01 to 100 mg/kg body weight, preferably from about 0.05 to 75 mg/kg body weight, more preferably between 0.1 to 50 mg/kg body weight, and most preferably between 0.1 to 25 mg/kg body weight. The compound may be administered as a bolus (i.e. the entire daily dose is administered at once) or in divided doses two or more times a day. Variations based on the aforementioned dosage ranges may be made by a physician of ordinary skill taking into account known considerations such as weight, age, and condition of the person being treated, the severity of the affliction, and the particular route of administration.

The compounds of Formula (I) may also be prepared in a pharmaceutical composition comprising one or more further active substances alone, or in combination with pharmaceutically acceptable carriers, diluents, or excipients in either single or multiple doses. The suitable pharmaceutically acceptable carriers, diluents and excipients are as described herein above, and the one or more further active substances may be any active substances, or preferably an active substance as described in the section “combination treatment” herein below.

### Clinical Conditions and Other Uses of Compounds

The compounds according to Formula (I) as defined herein are useful for treatment of a HDME dependent disease, disorder or condition. The treatment may include administering to a mammal, preferably a human, more preferably a human suffering from a HDME dependent disease, a therapeutically effective amount of a compound according to Formula (I) as defined herein.

Said HDME may be any HDME, however preferably the HDME of the present method is selected from the JmjC (Jumonji) family, as described in Cloos et. al., Genes & Development 22, 1115-1140, 2008, which is incorporated herein by reference in its entirety. More preferably said HDME is a HDME of the human JmjC family. Even more preferably said HDME belongs to one or more of the KDM7, KDM6, KDM5, KDM4, KDM3 or KDM2 families. Most preferably said HDME is chosen from PHF8, KDM6A, KDM5A, KDM5B, KDM4A, KDM4C, KDM3A, KDM2A, or KDM2B.

The present disclosure also relates to a compound of Formula (I) as defined herein for use in the treatment of a HDME dependent disease, such as for the treatment of cancer.

By the term “HDME dependent disease” is meant any disease characterized by elevated HDME expression and/or activity in at least in some instances of the disease, or a disease which is ameliorated by lowering the activity of HDMEs. Thus, the disease to be treated with the inhibitors of HDME, i.e. compounds of Formula (I), may be a proliferative or hyperproliferative disease, which includes benign or malignant tumors, for example a proliferative or hyperproliferative disease selected from the group consisting of a carcinoma of the brain, kidney, liver, adrenal gland, bladder, breast, stomach (for example gastric tumors), ovaries, esophagus, colon, rectum, prostate, pancreas, lung, vagina, thyroid, sarcoma, glioblastomas, multiple myeloma or gastrointestinal cancer, for example, colon carcinoma or colorectal adenoma, or a tumor of the neck and head, an epidermal hyperproliferation, for example, psoriasis, prostate hyperplasia, a neoplasia, including a neoplasia of epithelial character, including mammary carcinoma, and a leukemia.

In one embodiment, compounds of Formula (I) as defined herein are useful in the treatment of one or more cancers. The term “cancer” refers to any cancer caused by the proliferation of neoplastic cells, such as solid tumors, neoplasms, carcinomas, sarcomas, leukemias, lymphomas and the like. In particular, cancers that may be treated by the compounds, compositions and methods of the disclosure include, but are not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma, (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor, nephroblastoma, lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord (neurofibroma, meningioma, glioma, sarcoma); Gynecological: uterus (endometrial carcinoma), cervix (cervical carcinoma, pre-tumor cervical dysplasia), ovaries (ovarian carcinoma, serous cystadenocarcinoma, mucinous cystadenocarcinoma, unclassified carcinoma, granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (acute myeloid leukemia, chronic myeloid leukemia, acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma.

In one embodiment, the compounds of Formula (I) as defined herein are useful in the treatment of one or more cancers selected from the group consisting of: leukemias including acute leukemias and chronic leukemias such as acute lymphocytic leukemia (ALL), Acute myeloid leukemia (AML), chronic lymphocytic leukemia (CLL), chronic myelogenous leukemia (CML) and Hairy Cell Leukemia; lymphomas such as cutaneous T-cell lymphomas (CTCL), noncutaneous peripheral T-cell lymphomas, lymphomas associated with human T-cell lymphotrophic virus (HTLV) such as adult T-cell leukemia/Iymphoma (ATLL), Hodgkin's disease and non-Hodgkin's lymphomas, large-cell lymphomas, diffuse large B-cell lymphoma (DLBCL); Burkitt's lymphoma; mesothelioma, primary central nervous system (CNS) lymphoma; multiple myeloma; childhood solid tumors such as brain tumors, neuroblastoma, retinoblastoma, Wilm's tumor, bone tumors, and soft-tissue sarcomas, common solid tumors of adults such as head and neck cancers (e.g., oral, laryngeal and esophageal), genito urinary cancers (e.g., prostate, bladder, renal, uterine, ovarian, testicular, rectal and colon), lung cancer, breast cancer, pancreatic cancer, melanoma and other skin cancers, stomach cancer, brain tumors, liver cancer and thyroid cancer.

In another very preferred embodiment, the compound of Formula (I) as defined herein are useful for the treatment of squamous cell carcinomas. Preferably said squamous cell carcinomas are cancers of the carcinoma type of squamous epithelium that may occur in many different organs, including the skin, lips, mouth, esophagus, urinary bladder, prostate, lungs, vagina, and cervix; brain cancer, that is neuroblastoma, glioblastoma and other malignant and benign brain tumors; breast cancer, pancreatic cancer, and multiple myeloma.

In yet another embodiment, the compounds of Formula (I) as defined herein are useful for treatment of brain cancer, tumors of adults such as head and neck cancers (e.g., oral, laryngeal and esophageal), genito urinary cancers (e.g., prostate, bladder, renal, uterine, ovarian, testicular, rectal and colon), and breast cancer.

Other cancer forms for which the compounds of Formula (I) are useful as treatment can be found in Stedman's Medical Dictionary (Lippincott Williams & Wilkins, 28^{th} Ed., 2005), which is incorporated herein by reference in its entirety.

In still another related embodiment, the disease to be treated by compounds of Formula (I) as defined herein is selected from persistent proliferative or hyperproliferative conditions such as angiogenesis, such as psoriasis; Kaposi's sarcoma; restenosis, e.g., stent-induced restenosis; endometriosis; Hodgkin's disease; leukemia; hemangioma; angiofibroma; eye diseases, such as neovascular glaucoma; renal diseases, such as glomerulonephritis; malignant nephrosclerosis; thrombotic microangiopathic syndromes; transplant rejections and glomerulopathy; fibrotic diseases, such as cirrhosis of the liver; mesangial cell-proliferative diseases; injuries of the nerve tissue; and inhibiting the re-occlusion of vessels after balloon catheter treatment, for use in vascular prosthetics or after inserting mechanical devices for holding vessels open, such as, e.g., stents, as immune-suppressants, as an aid in scar-free wound healing, and treating age spots and contact dermatitis.

The compounds of Formula (I) are suitable as active agents in pharmaceutical compositions that are efficacious particularly for treating cellular proliferative or hyperproliferative ailments and/or ailments associated with dysregulated gene expression. Such pharmaceutical compositions have a therapeutically effective amount of the compound of Formula (I) along with other pharmaceutically acceptable excipients, carriers, and diluents and. The phrase, “therapeutically effective amount” as used herein indicates an amount necessary to administer to a host, or to a cell, tissue, or organ of a host, to achieve a therapeutic effect, such as an ameliorating or alternatively a curative effect, for example an anti-tumor effect, e.g. reduction of or preferably inhibition of proliferation of malignant cancer cells, benign tumor cells or other proliferative cells, or of any other HDME dependent disease.

Another aspect of the disclosure is a pharmaceutical composition comprising a therapeutically effective amount of at least one compound of Formula (I) as defined herein, or a pharmaceutically acceptable salt, solvate or prodrug thereof, in combination with at least one further anti-neoplastic compound, and a pharmaceutically acceptable excipient, carrier or diluent.

### Method of Treatment

In a further aspect the present disclosure relates to a method of treating a diseases in a subject, said method comprises administering to said subject a therapeutically effective amount of at least one compound of Formula (I) as defined herein. The disease may be any disease or disorder as mentioned herein, such as for example mentioned in the section “HDME dependent diseases”, and the compound may be administered alone or in a pharmaceutical composition, such as for example mentioned in the section “Pharmaceutical compositions”.

Hence, the disclosure also relates to a compound of Formula (I) as defined herein for use as a medicament.

The term “treating” and “treatment”, as used herein, unless otherwise indicated, refers to reversing, alleviating, inhibiting the process of, or preventing the disease, disorder or condition to which such term applies, or one or more symptoms of such disease, disorder or condition and includes the administration of a compound of Formula (I) to prevent the onset of the symptoms or the complications, or alleviating the symptoms or the complications, or eliminating the disease, condition, or disorder. Preferably treatment is curative or ameliorating.

In a preferred embodiment of this aspect of the disclosure the method is a method of treating a HDME dependent disease in a subject, said method comprises administering to said subject a therapeutically effective amount of a compound of Formula (I) as defined herein to a subject in need of such treatment. The HDME dependent disease may be any HDME dependent disease as described herein above. Preferably the HDME dependent disease is squamous cell carcinomas or any other of the cancer conditions mentioned above.

Hence, the disclosure also relates to a compound of Formula (I) as defined herein for use in the treatment of a HDME dependent disease, such as for the treatment of cancer.

Further, the disclosure relates to the use of a compound of Formula (I) as defined herein for the preparation of a pharmaceutical composition for the treatment of a HDME dependent disease.

In one embodiment of the method of treatment of a HDME dependent disease, the compound of Formula (I) as defined herein is administered in combination with one or more further active substances. The active substances may be any active substances, and preferably an active substance as described herein above in the section “combination treatment”. More preferably the one or more additional active substances are selected from the group consisting of anti-proliferative or anti-neoplastic agents.

### Combination Treatment

A compound of Formula (I) may also be used to advantage in combination with one or more other anti-proliferative or anti-neoplastic agents. Such anti-proliferative agents include, but are not limited to other HDME inhibitors, proteasome inhibitors, including bortezomib (Velcade) and Carfilzomib, aromatase inhibitors; antiestrogens; topoisomerase I inhibitors; topoisomerase II inhibitors; microtubule active agents; alkylating agents; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein tyrosine or serine or threonine kinase activity; compounds targeting/decreasing a lipid kinase activity; compounds targeting/decreasing a carbohydrate kinase activity and further anti-angiogenic compounds; compounds which target, decrease or inhibit the activity of a protein or lipid phosphatase; gonadorelin agonists; anti-androgens; angiostatic steroids; methionine aminopeptidase inhibitors; bisphosphonates; biological response modifiers; antiproliferative antibodies; DNA methyl transferase inhibitors; histone methyltransferase inhibitors; heparanase inhibitors; inhibitors of Ras oncogenic isoforms; telomerase inhibitors; proteasome inhibitors; agents used in the treatment of hematologic malignancies; compounds which target, decrease or inhibit the activity of Flt-3; Hsp90 inhibitors; temozolomide (TEMOD AL(R)); leucovorin; immunomodulators, such as thalidomide, pomalidomide, lenalidomide, and their derivatives; immune stimulating agents, such as BCG, IL-2 or IFN-α, antibodies such as anti-CTLA-4 monoclonal antibody ipilimumab (Yervoy), rituximab or herceptin and cancer vaccines; inhibitors/modulators of mitochondrial activity such as metformin.

A compound of Formula (I) as defined herein may also be used to advantage in combination with known therapeutic processes, e.g., the administration of hormones or tumor cell damaging approaches, especially ionizing radiation.

A compound of Formula (I) as defined herein may also be used as a radiosensitizer, including, for example, the treatment of tumors which exhibit poor sensitivity to radiotherapy.

By the term “combination”, is meant either a fixed combination in one dosage unit form, or a kit of parts for the combined administration where a compound of Formula (I) and a combination partner may be administered independently at the same time or separately within time intervals that especially allow that the combination partners show a cooperative, e.g., synergistic, effect, or any combination thereof.

The phrase, “aromatase inhibitor” as used herein relates to a compound which inhibits the estrogen production, i.e., the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane can be administered, e.g., in the form as it is marketed, e.g., under the trademark AROMASIN. Formestane can be administered, e.g., in the form as it is marketed, e.g., under the trademark LENTARON. Fadrozole can be administered, e.g., in the form as it is marketed, e.g., under the trademark AFEMA. Anastrozole can be administered, e.g., in the form as it is marketed, e.g., under the trademark ARIMIDEX. Letrozole can be administered, e.g., in the form as it is marketed, e.g., under the trademark FEMARA or FEMAR.

Aminoglutethimide can be administered, e.g., in the form as it is marketed, e.g., under the trademark ORIMETEN. A combination of the disclosure comprising a chemotherapeutic agent which is an aromatase inhibitor is particularly useful for the treatment of hormone receptor positive tumors, e.g., breast tumors.

The term “antiestrogen” as used herein relates to a compound that antagonizes the effect of estrogens at the estrogen receptor level. The term includes, but is not limited to tamoxifen, fulvestrant, raloxifene and raloxifene hydrochloride. Tamoxifen can be administered, e.g., in the form as it is marketed, e.g., under the trademark NOLVADEX. Raloxifene hydrochloride can be administered, e.g., in the form as it is marketed, e.g., under the trademark EVISTA. Fulvestrant can be formulated as disclosed in U.S. Pat. No. 4,659,516 or it can be administered, e.g., in the form as it is marketed, e.g., under the trademark FASLODEX. A combination of the disclosure comprising a chemotherapeutic agent which is an antiestrogen is particularly useful for the treatment of estrogen receptor positive tumors, e.g., breast tumors.

The term “anti-androgen” as used herein relates to any substance which is capable of inhibiting the biological effects of androgenic hormones and includes, but is not limited to, bicalutamide (CASODEX), which can be formulated, e.g., as disclosed in U.S. Pat. No. 4,636,505.

The phrase, “gonadorelin agonist” as used herein includes, but is not limited to abarelix, goserelin and goserelin acetate. Goserelin is disclosed in U.S. Pat. No. 4,100,274 and can be administered, e.g., in the form as it is marketed, e.g., under the trademark ZOLADEX. Abarelix can be formulated, e.g., as disclosed in U.S. Pat. No. 5,843,901.

The phrase, “topoisomerase I inhibitor” as used herein includes, but is not limited to topotecan, gimatecan, irinotecan, camptothecan and its analogues, 9-nitrocamptothecin and the macromolecular camptothecin conjugate PNU-166148 (compound A1 in WO99/17804). Irinotecan can be administered, e.g., in the form as it is marketed, e.g., under the trademark CAMPTOSAR. Topotecan can be administered, e.g., in the form as it is marketed, e.g., under the trademark HYCAMTIN.

The phrase, “topoisomerase II inhibitor” as used herein includes, but is not limited to the anthracyclines such as doxorubicin (including liposomal formulation, e.g., CAELYX), daunorubicin, epirubicin, idarubicin and nemorubicin, the anthraquinones mitoxantrone and losoxantrone, and the podophyllotoxins etoposide and teniposide. Etoposide can be administered, e.g., in the form as it is marketed, e.g., under the trademark ETOPOPHOS. Teniposide can be administered, e.g., in the form as it is marketed, e.g., under the trademark VM 26-BRISTOL. Doxorubicin can be administered, e.g., in the form as it is marketed, e.g., under the trademark ADRIBLASTIN or ADRIAMYCIN. Epirubicin can be administered, e.g., in the form as it is marketed, e.g., under the trademark FARMORUBICIN. Idarubicin can be administered, e.g., in the form as it is marketed, e.g., under the trademark ZAVEDOS. Mitoxantrone can be administered, e.g., in the form as it is marketed, e.g., under the trademark NOVANTRON.

The phrase, “microtubule active agent” relates to microtubule stabilizing, microtubule destabilizing agents and microtublin polymerization inhibitors including, but not limited to taxanes, e.g., paclitaxel and docetaxel, vinca alkaloids, e.g., vinblastine, including vinblastine sulfate, vincristine including vincristine sulfate, and vinorelbine, discodermolides, cochicine and epothilones and derivatives thereof, e.g., epothilone B or D or derivatives thereof. Paclitaxel may be administered e.g., in the form as it is marketed, e.g., TAXOL. Docetaxel can be administered, e.g., in the form as it is marketed, e.g., under the trademark TAXOTERE. Vinblastine sulfate can be administered, e.g., in the form as it is marketed, e.g., under the trademark VINBLASTIN R.P. Vincristine sulfate can be administered, e.g., in the form as it is marketed, e.g., under the trademark FARMISTIN. Discodermolide can be obtained, e.g., as disclosed in U.S. Pat. No. 5,010,099. Also included are Epothilone derivatives which are disclosed in WO 98/10121, U.S. Pat. No. 6,194,181, WO 98/25929, WO 98/08849, WO 99/43653, WO 98/22461 and WO 00/31247. Included are Epothilone A and/or B.

The phrase, “alkylating agent” as used herein includes, but is not limited to, cyclophosphamide, ifosfamide, melphalan or nitrosourea (BCNU or Gliadel).

Cyclophosphamide can be administered, e.g., in the form as it is marketed, e.g., under the trademark CYCLOSTIN. Ifosfamide can be administered, e.g., in the form as it is marketed, e.g., under the trademark HOLOXAN.

The phrase, “histone deacetylase inhibitors” or “HDAC inhibitors” relates to compounds which inhibit at least one example of the class of enzymes known as a histone deacetylase, and which compounds generally possess antiproliferative activity. Previously disclosed HDAC inhibitors include compounds disclosed in, e.g., WO 02/22577, including N-hydroxy-3-[4-{[(2-hydroxyethyl)[2-(1H-indol-3-yl)ethyl]-amino]methyl]phenyl]-2E-2-propenamide, N-hydroxy-3-[4-[[[2-(2-methyl-1H-indol-3-yl)-ethyl]-amino]methyl]phenyl]-2E-2-propenamide and pharmaceutically acceptable salts thereof. It further includes Suberoylanilide hydroxamic acid (SAHA). Other publicly disclosed HDAC inhibitors include butyric acid and its derivatives, including sodium phenylbutyrate, thalidomide, trichostatin A and trapoxin.

The term “antineoplastic antimetabolite” includes, but is not limited to, 5-Fluorouracil or 5-FU, capecitabine, gemcitabine, DNA demethylating agents, such as 5-azacytidine and decitabine, methotrexate and edatrexate, and folic acid antagonists such as pemetrexed. Capecitabine can be administered, e.g., in the form as it is marketed, e.g., under the trademark XELODA. Gemcitabine can be administered, e.g., in the form as it is marketed, e.g., under the trademark GEMZAR. Also included is the monoclonal antibody trastuzumab which can be administered, e.g., in the form as it is marketed, e.g., under the trademark HERCEPTIN.

The phrase, “platin compound” as used herein includes, but is not limited to, carboplatin, cis-platin, cisplatinum and oxaliplatin. Carboplatin can be administered, e.g., in the form as it is marketed, e.g., under the trademark CARBOPLAT. Oxaliplatin can be administered, e.g., in the form as it is marketed, e.g., under the trademark ELOXATIN.

The phrase, “compounds targeting/decreasing a protein tyrosine or serine or threonine kinase activity” as used herein includes, but is not limited to, gefinitib, erlotinib, lapatinib, foretinib, cabozantinib, vemurafenib or selumetinib (AZD6244). Gefinitib can be administered, e.g., in the form as it is marketed, e.g., under the trademark IRESSA. Erlotinib can be administered, e.g., in the form as it is marketed, e.g., under the trademark TARCEVA. Lapatinib can be administered, e.g., in the form as it is marketed, e.g., under the trademarks TYKERB and TYVERB. Cabozantinib can be administered, e.g., in the form as it is marketed, e.g., under the trademark COMETRIQ. Vemurafenib can be administered, e.g., in the form as it is marketed, e.g., under the trademark CELBORAF. Foretinib can be formulated, e.g., as disclosed in US 20,120,282,179. Selumetinib (AZD6244) can be formulated, e.g., as disclosed in US 20,080,177,082 and US 20,090,246,274. Other suitable protein kinase inhibitors include without limitation Afatanib (Gilotrif, Boeringer Ingelheim), Axitinib (Inlyta, Pfizer), Bosutinib (Bosulif, Wyeth), Crizotinib (Xalkori, Pfizer), Dabrafenib (Tafinlar, GSK), Dasatinib (Sprycel, Bristol-Myers Squib), Elotinib (Tarceva, OSI), Everolimus (Afinitor, Novartis), Gefitinib (Iressa, Astrazeneca), Ibrutinib (Imbruvica, Pharmacyclics and J&J), Imatanib (Gleevec, Novartis), Nilotinib (Tasigna, Novartis), Pazopanib (Votrient, GlaxoSmithKline), Ponatinib (Iclusig, Ariad), Regorafenib (Stivarga, Bayer), Ruxolitinib (Jakafi, Incyte), Sirolimus (Rapamune, Wyeth), Sorafenib (Nexavar, Bayer), Sunitinib (Sutent, Pfizer), Tofacitinib (Xeljanz, Pfizer), Temsirolimus (Torisel, Wyeth), Trametinib (Mekinist, GSK), Vandetanib (Caprelsa, IPR Pharms) as well as other proposed protein kinase inhibitors that can be found in the literature.

Tumor cell damaging approaches refer to approaches such as ionizing radiation. The phrase, “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See, e.g., Hellman, Principles of Radiation Therapy, Cancer, in Principles and Practice of Oncology, Devita et al., Eds., 4th Edition, Vol. 1, pp. 248-275 (1993).

The phrase, “angiostatic steroids” as used herein refers to agents which block or inhibit angiogenesis, such as, e.g., anecortave, triamcinolone, hydrocortisone, 11-[alpha]-epihydrocotisol, cortexolone, 17[alpha]-hydroxyprogesterone, corticosterone, desoxycorticosterone, testosterone, estrone and dexamethasone.

Other chemotherapeutic agents include, but are not limited to, plant alkaloids, hormonal agents and antagonists; biological response modifiers, preferably lymphokines or interferons; antisense oligonucleotides or oligonucleotide derivatives; or miscellaneous agents or agents with other or unknown mechanism of action.

The structure of the active agents identified by code numbers, generic or trade names may be taken from the actual edition of the standard compendium “The Merck Index” or from databases, e.g., Patents International (e.g., IMS World Publications).

The above-mentioned compounds, which can be used in combination with a compound of Formula (I), can be prepared and administered as described in the art such as in the documents cited above.

Furthermore, the compounds of the disclosure may be used in a method of profiling the functional and structural similarity of histone demethylases comprising taking a panel of at least two histone demethylases and a panel of at least two compounds of formula 1 and determining the extent to which each said compound of formula 1 inhibits the activity of each of said histone demethylases, and generating a similarity index reflecting the degree of similarity between the histone demethylases in respect of their inhibition by said compounds.

### EXAMPLES

### Example 1—Preparation of Compounds of the Disclosure

### General Methods and Materials

All chemicals were purchased from Sigma-Aldrich, Alfa Aesar, Matrix, Combiblock, Oakwood, and Chembridge. Anhydrous solvents were Aldrich Sure/Seal™ brand. All reactions were carried out under a dry nitrogen atmosphere using dry solvents. Reactions were monitored by thin-layer chromatography carried out on Sigma-Aldrich 0.25 mm silica gel plates (60 Å, fluorescent indicator). Spots were visualized under UV light (254 nm). Flash column chromatography was performed on Biotage SNAP Flash System, or silica gel 60 (particle size 0.032-0.063 mm) obtained from Silicycle, Inc. Low-resolution ES (electrospray) mass spectra were obtained using a Micromass Quattro Ultima mass spectrometer in the electrospray positive (ES+) or negative (ES−) ion mode. 1H-NMR spectra were recorded on a Bruker AM-300 spectrometer and were calibrated using residual nondeuterated solvent as internal reference. Spectra were processed using Spinworks version 2.5 (developed by Dr. Kirk Marat, Department of Chemistry, University of Manitoba). Preparative HPLC was performed on Waters 2996 with Photodiode Array Detector, Waters 600 Controller, Waters 100 pump, and Waters 717 auto sampler, with UV detection at 254 and 280 nm. Flow rate: 15 mL/minute, run time 30 minutes. Solvents: 0-100% (H₂O-MeOH), with and without added TFA (0.1%). Column used was Supelco C18, 25 cm×21.2 mm, particle size 10 micrometer.

Ethyl 2-formylpyridine-4-carboxylate was prepared analogously to Queguiner, G. and Pastour, P. (Comptes Rendus des Séances de l'Académie des Sciences, Série C: Sciences Chimiques (1969), 268(2), 182-185).

### Examples of Compounds of Formula I

| | | | May be | |
|---|---|---|---|---|
| | | | prepared | |
| | | | analogously | |
| | | | to Synthetic | |
| Structure | # | Name | Route | NMR |
| 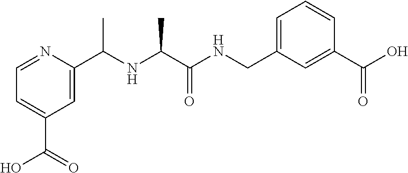 [SMILES: CC(N[C@@H](C)C(=O)NCc1cccc(C(=O)O)c1)c1cc(C(=O)O)ccn1] | 1 | 2-(1-{[(1S)-1-{[(3- carboxyphenyl)methyl] carbamoyl}ethyl]amino} ethyl)pyridine-4- carboxylic acid | A | ¹H NMR (300 MHz, CD₃OD), δ ppm: 8.39 (d), 4.21 (s), 3.90 (q), 3.17 (q) |
| 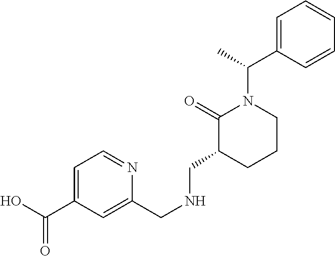 [SMILES: C[C@H](c1ccccc1)N1CCC[C@H](CNCc2cc(C(=O)O)ccn2)C1=O] | 2 | 2-[({[(3R)-2-oxo-1- [(1R)-1- phenylethyl]piperidin- 3- yl]methyl}amino) methyl]pyridine-4- carboxylic acid | A | ¹H-NMR (300 MHz, CD₃OD), δ ppm: 8.57(d, 1H), 3.98 (s, 2H), 2.93-2.77(m, 2H), 1.53(d, 3H) |
| 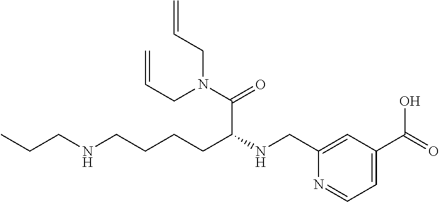 [SMILES: C=CCN(CC=C)C(=O)[C@@H](CCCCNCCC)NCc1cc(C(=O)O)ccn1] | 3 | 2-({[(1R)-1-[bis(prop- 2-en-1-yl)carbamoyl]- 5- (propylamino)pentyl] amino}methyl)pyridine- 4-carboxylic acid | A | ¹H-NMR (300 MHz, CD₃OD): δ 8.83 (d, 1H), 8.04 (s, 1H), 7.96 (d, 1H), 1.03 (t, 3H). |
| 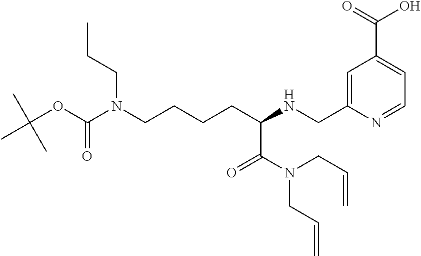 [SMILES: C=CCN(CC=C)C(=O)[C@@H](CCCCN(CCC)C(=O)OC(C)(C)C)NCc1cc(C(=O)O)ccn1] | 4 | 2-({[(1R)-1-[bis(prop- 2-en-1-yl)carbamoyl]- 5-{[(tert- butoxy)carbonyl](propyl) amino}pentyl]amino} methyl)pyridine-4- carboxylic acid | A | ¹H-NMR (300 MHz, CD₃OD): δ 8.56 (d, 1H), 7.89 (s, 1H), 7.74 (d, 1H), 1.44 (s, 9H). |
| 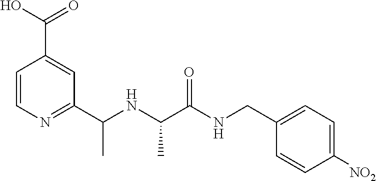 [SMILES: CC(N[C@@H](C)C(=O)NCc1ccc([N+](=O)[O-])cc1)c1cc(C(=O)O)ccn1] | 5 | 2-(1-{[(1S)-1-{[(4- nitrophenyl)methyl] carbamoyl}ethyl]amino} ethyl)pyridine-4- carboxylic acid | A | ¹H NMR (300 MHz, CD₃OD), δ ppm: 8.48 (d), 4.47 (d), 3.98 (q), 3.25 (q) |
| 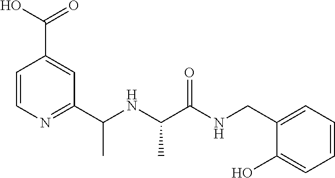 [SMILES: CC(N[C@@H](C)C(=O)NCc1ccccc1O)c1cc(C(=O)O)ccn1] | 6 | 2-(1-{[(1S)-1-{[(2- hydroxyphenyl)methyl] carbamoyl}ethyl] amino}ethyl)pyridine-4- carboxylic acid | A | ¹H NMR (300 MHz, CD₃OD), δ ppm: 8.42 (d), 4.24 (d), 3.91 (q), 3.22 (q) |
| 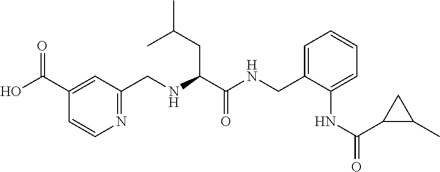 [SMILES: CC(C)C[C@H](NCc1cc(C(=O)O)ccn1)C(=O)NCc1ccccc1NC(=O)C1CC1C] | 7 | 2-({[(1S)-3-methyl-1- ({[2-(2- methylcyclopropane- amido)phenyl]methyl} carbamoyl)butyl]amino} methyl)pyridine-4- carboxylic acid | A | ¹H-NMR (300 MHz, CD₃OD): δ 8.45 (d, 1H), 7.30 (s, 1H), 7.10 (m, 3H), 4.40 (s, 2H), 3.80 (m, 2H), 0.80 (2 d, 6H). |
| 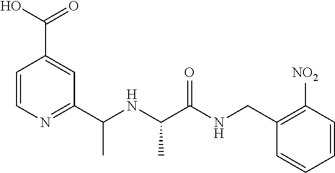 [SMILES: CC(N[C@@H](C)C(=O)NCc1ccccc1[N+](=O)[O-])c1cc(C(=O)O)ccn1] | 8 | 2-(1-{[(1S)-1-{[(2- nitrophenyl)methyl] carbamoyl}ethyl]amino} ethyl)pyridine-4- carboxylic acid | A | ¹H NMR (300 MHz, CD₃OD), δ ppm: 8.47 (d), 4.57 (d), 3.97 (q), 3.26 (q) |
| 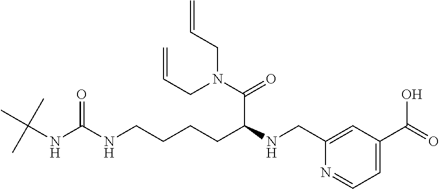 [SMILES: C=CCN(CC=C)C(=O)[C@H](CCCCNC(=O)NC(C)(C)C)NCc1cc(C(=O)O)ccn1] | 9 | 2-({[(1S)-1-[bis(prop- 2-en-1-yl)carbamoyl]- 5-[(tert- butylcarbamoyl)amino] pentyl]amino}methyl) pyridine-4-carboxylic acid | A | ¹H-NMR (300 MHz, CD₃OD): δ 8.48 (d, 1H), 7.97 (s, 1H), 7.69 (d, 1H), 1.28 (s, 9H). |
| 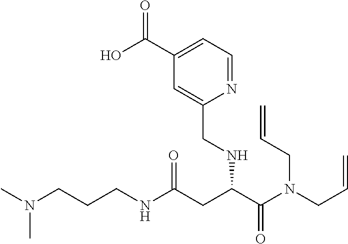 [SMILES: C=CCN(CC=C)C(=O)[C@H](CC(=O)NCCCN(C)C)NCc1cc(C(=O)O)ccn1] | 10 | 2-({[(1S)-1-[bis(prop- 2-en-1-yl)carbamoyl]- 2-{[3- (dimethylamino)propyl] carbamoyl}ethyl] amino}methyl)pyridine- 4-carboxylic acid | A | ¹H-NMR (300 MHz, CD₃OD): δ 8.49 (d, 1H), 7.88 (s, 1H), 7.69 (d, 1H), 2.19 (s, 6H). |
| 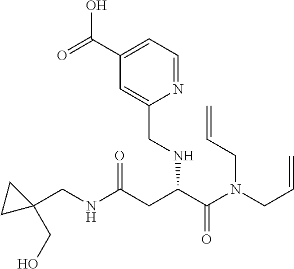 [SMILES: C=CCN(CC=C)C(=O)[C@H](CC(=O)NCC1(CO)CC1)NCc1cc(C(=O)O)ccn1] | 11 | 2-({[(1S)-1-[bis(prop- 2-en-1-yl)carbamoyl]- 2-({[1- (hydroxymethyl)cyclo- propyl]methyl}carba- moyl)ethyl]amino} methyl)pyridine-4- carboxylic acid | A | ¹H-NMR (300 MHz, CD₃OD): δ 8.49 (d, 1H), 7.90 (s, 1H), 7.69 (d, 1H), 0.44 (m, 4H). |
| 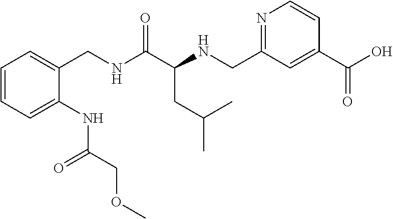 [SMILES: COCC(=O)Nc1ccccc1CNC(=O)[C@H](CC(C)C)NCc1cc(C(=O)O)ccn1] | 12 | 2-({[(1S)-1-({[2-(2- methoxyacetamido) phenyl]methyl} carbamoyl)-3- methylbutyl]amino} methyl)pyridine-4- carboxylic acid | A | ¹H-NMR (300 MHz, CD₃OD): δ 8.30 (d, 1H), 7.50 (s, 1H), 7.00 (m, 3H), 4.20 (s, 2H), 3.30 (s, 3H), 0.70 (2 d, 6H) |
| 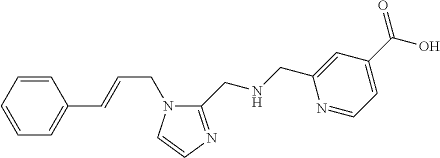 [SMILES: O=C(O)c1ccnc(CNCc2nccn2C/C=C/c2ccccc2)c1] | 13 | 2-{[({1-[(2E)-3- phenylprop-2-en-1- yl]-1H-imidazol-2- yl}methyl)amino] methyl}pyridine-4- carboxylic acid | A | ¹H NMR (300 MHz, CD₃OD), δ ppm: 8.47 (d, 1H), 7.88 (s, 1H), 7.08 (s, 1H), 6.46-6.24 (m, 2H), 3.93 (d, 4H). |
| 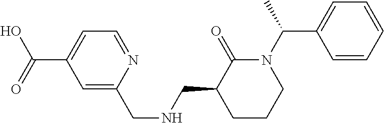 [SMILES: C[C@H](c1ccccc1)N1CCC[C@@H](CNCc2cc(C(=O)O)ccn2)C1=O] | 14 | 2-[({[(3S)-2-oxo-1- [(1R)-1- phenylethyl]piperidin- 3- yl]methyl}amino) methyl]pyridine-4- carboxylic acid | A | ¹H-NMR (300 MHz, CD₃OD), δ ppm: 8.60(d, 1H), 4.1(s, 2H), 2.82-2.64 (m, 2H), 1.52(d, 3H) |
| 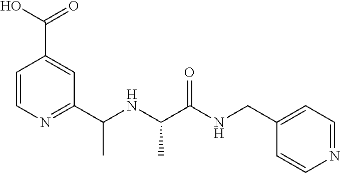 [SMILES: CC(N[C@@H](C)C(=O)NCc1ccncc1)c1cc(C(=O)O)ccn1] | 15 | 2-(1-{[(1S)-1- [(pyridin-4- ylmethyl)carbamoyl] ethyl]amino}ethyl)pyri- dine-4-carboxylic acid | A | ¹H NMR (300 MHz, CD₃OD), δ ppm: 8.47 (d), 4.30 (s), 3.99 (q), 3.25 (q) |
| 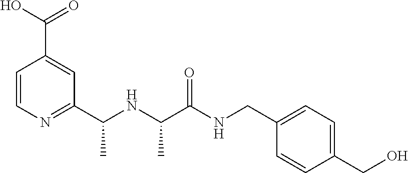 [SMILES: C[C@H](N[C@H](C)c1cc(C(=O)O)ccn1)C(=O)NCc1ccc(CO)cc1] | 16 | 2-[(1R)-1-{[(1S)-1- ({[4- (hydroxymethyl)phenyl] methyl}carbamoyl) ethyl]amino}ethyl] pyridine-4-carboxylic acid | A | ¹H NMR (300 MHz, CD₃OD), δ ppm: 8.46 (d), 4.25 (d), 3.95 (q), 3.26 (q) |
| 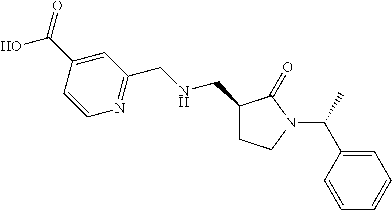 [SMILES: C[C@H](c1ccccc1)N1CC[C@@H](CNCc2cc(C(=O)O)ccn2)C1=O] | 17 | 2-[({[(3S)-2-oxo-1- [(1R)-1- phenylethyl]pyrrolidin- 3- yl]methyl}amino) methyl]pyridine-4- carboxylic acid | A | ¹H-NMR (300 MHz, CD₃OD), δ ppm 8.65 (d, 1H), 5.40 (q, 1H), 3.32 (s, 2H), 3.49 (q, 1H), 2.39-2.28 (m, 1H), 1.58 (d, 3H) |
| 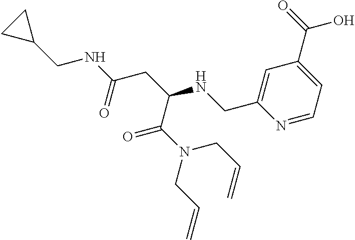 [SMILES: C=CCN(CC=C)C(=O)[C@@H](CC(=O)NCC1CC1)NCc1cc(C(=O)O)ccn1] | 18 | 2-({[(1R)-1-[bis(prop- 2-en-1-yl)carbamoyl]- 2- [(cyclopropylmethyl) carbamoyl]ethyl]amino} methyl)pyridine-4- carboxylic acid | A | ¹H NMR (300 MHz, CD₃OD), δ ppm: 8.48 (d, 1H), 7.87 (s, 1H), 5.93-5.62 (m, 2H), 3.03 (d, 2H), 1.02-0.84 (m, 1H), 0.16 (q, 2H). |
| 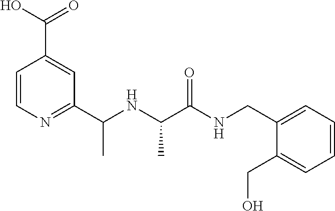 [SMILES: CC(N[C@@H](C)C(=O)NCc1ccccc1CO)c1cc(C(=O)O)ccn1] | 19 | 2-(1-{[(1S)-1-({[2- (hydroxymethyl)phenyl] methyl}carbamoyl) ethyl]amino}ethyl) pyridine-4-carboxylic acid | A | ¹H NMR (300 MHz, CD₃OD), δ ppm: 8.41 (d), 4.34 (s), 3.93 (q), 3.21 (q) |
| 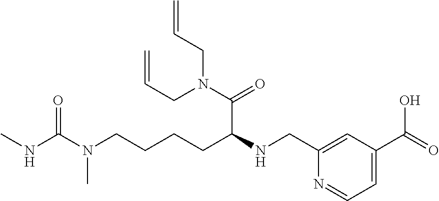 [SMILES: C=CCN(CC=C)C(=O)[C@H](CCCCN(C)C(=O)NC)NCc1cc(C(=O)O)ccn1] | 20 | 2-({[(1S)-1-[bis(prop- 2-en-1-yl)carbamoyl]- 5- [methyl(methylcarba- moyl)amino]pentyl] amino}methyl)pyridine- 4-carboxylic acid | A | ¹H-NMR (300 MHz, CD₃OD): δ 8.49 (d, 1H), 7.93 (s, 1H), 7.69 (d, 1H), 2.84 (s, 3H), 2.70 (s, 3H). |
| 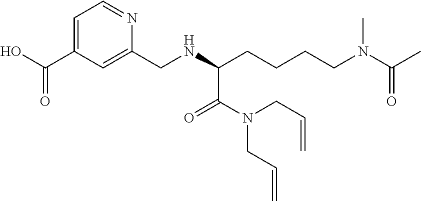 [SMILES: C=CCN(CC=C)C(=O)[C@H](CCCCN(C)C(C)=O)NCc1cc(C(=O)O)ccn1] | 21 | 2-({[(1S)-1-[bis(prop- 2-en-1-yl)carbamoyl]- 5-(N- methylacetamido)pentyl] amino}methyl)pyri- dine-4-carboxylic acid | A | ¹H-NMR (300 MHz, CD₃OD): δ 8.48 (d, 1H), 7.93 (s, 1H), 7.69 (d, 1H), 2.94 (d, 3H), 2.07 (s, 3H). |
| 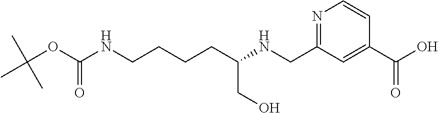 [SMILES: CC(C)(C)OC(=O)NCCCC[C@@H](CO)NCc1cc(C(=O)O)ccn1] | 22 | 2-({[(2S)-6-{[(tert- butoxy)carbonyl]amino}- 1-hydroxyhexan-2- yl]amino}methyl)pyri- dine-4-carboxylic acid | A | ¹H NMR (300 MHz, CD₃OD), δ ppm: 8.53 (d, 1H), 3.96 (m, 2H), 2.61 (m, 1H), 1.43 (s, 9H). |
| 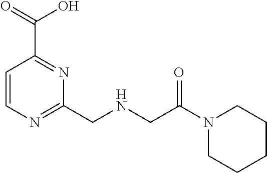 [SMILES: O=C(O)c1ccnc(CNCC(=O)N2CCCCC2)n1] | 23 | 2-({[2-oxo-2- (piperidin-1- yl)ethyl]amino}methyl) pyrimidine-4- carboxylic acid | A | ¹H NMR (300 MHz, CD₃OD), δ ppm: 8.82 (d, 1H), 4.06 (s, 2H), 3.39 (m, 2H), 1.70- 1.48 (m, 6H). |
| 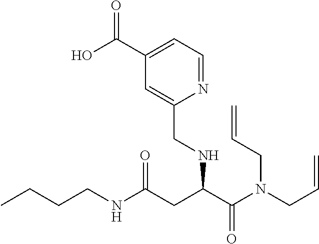 [SMILES: C=CCN(CC=C)C(=O)[C@@H](CC(=O)NCCCC)NCc1cc(C(=O)O)ccn1] | 24 | 2-({[(1R)-1-[bis(prop- 2-en-1-yl)carbamoyl]- 2- (butylcarbamoyl)ethyl] amino}methyl)pyridine- 4-carboxylic acid | A | ¹H NMR (300 MHz, CD₃OD), δ ppm: 8.49 (d, 1H), 7.86 (s, 1H), 5.90-5.65 (m, 2H), 3.16 (t, 2H), 0.89 (t, 3H). |
| 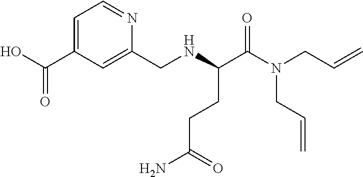 [SMILES: C=CCN(CC=C)C(=O)[C@@H](CCC(N)=O)NCc1cc(C(=O)O)ccn1] | 25 | 2-({[(1R)-1-[bis(prop- 2-en-1-yl)carbamoyl]- 3- carbamoylpropyl]amino} methyl)pyridine-4- carboxylic acid | A | ¹H-NMR (300 MHz, CD₃OD): δ 8.47 (d, 1H), 8.02 (s, 1H), 7.69 (d, 1H), 2.52 (m, 2H), 1.84 (m, 2H). |
| 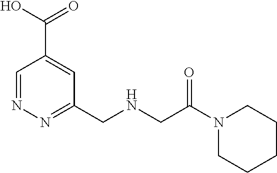 [SMILES: O=C(O)c1cnnc(CNCC(=O)N2CCCCC2)c1] | 26 | 6-({[2-oxo-2- (piperidin-1- yl)ethyl]amino}methyl) pyridazine-4- carboxylic acid | A | ¹H-NMR (300 MHz, CD₃OD): δ 9.62 (d, 1H), 4.72 (s, 2H), 4.27 (s, 2H), 1.72-1.54 (m, 6H) ppm. |
| 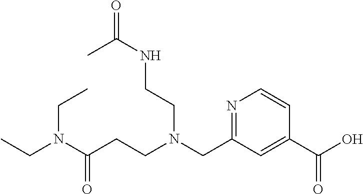 [SMILES: CCN(CC)C(=O)CCN(CCNC(C)=O)Cc1cc(C(=O)O)ccn1] | 27 | 2-({[2- (diethylcarbamoyl) ethyl](2- acetamidoethyl)amino} methyl)pyridine-4- carboxylic acid | A | ¹H-NMR (300 MHz, CD₃OD): δ 8.76 (d, 1H), 4.65 (s, 2H), 1.94 (s, 3H), 1.17-1.04 (m, 6H) ppm. |
| 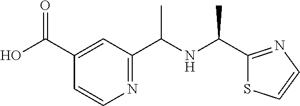 [SMILES: CC(N[C@@H](C)c1nccs1)c1cc(C(=O)O)ccn1] | 28 | 2-(1-{[(1S)-1-(1,3- thiazol-2- yl)ethyl]amino}ethyl) pyridine-4-carboxylic acid | A | ¹H-NMR (300 MHz, CD₃OD): δ 8.50 (d, 1H), 7.80 (s, 1H), 7.70 (d, 1H), 7.45 (d, 1H), 3.80 (m, 2H), 0.40 (2 d, 6H). |
| 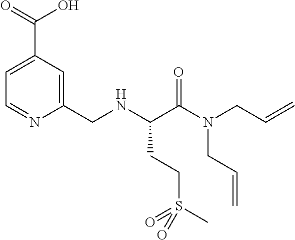 [SMILES: C=CCN(CC=C)C(=O)[C@H](CCS(C)(=O)=O)NCc1cc(C(=O)O)ccn1] | 29 | 2-({[(1S)-1-[bis(prop- 2-en-1-yl)carbamoyl]- 3- methanesulfonylpropyl] amino}methyl)pyridine- 4-carboxylic acid | A | ¹H-NMR (300 MHz, CD₃OD): δ 8.48 (d, 1H), 7.96 (s, 1H), 7.69 (d, 1H), 2.99 (s, 3H). |
| 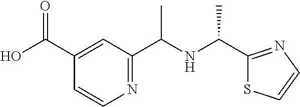 [SMILES: CC(N[C@H](C)c1nccs1)c1cc(C(=O)O)ccn1] | 30 | 2-(1-{[(1R)-1-(1,3- thiazol-2- yl)ethyl]amino}ethyl) pyridine-4-carboxylic acid | A | ¹H-NMR (300 MHz, CD₃OD): δ 8.45 (d, 1H), 7.80 (s, 1H), 7.70 (d, 1H), 7.45 (d, 1H), 3.80 (m, 2H), 0.50 (2 d, 6H) |
| 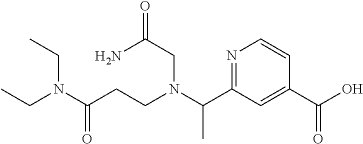 [SMILES: CCN(CC)C(=O)CCN(CC(N)=O)C(C)c1cc(C(=O)O)ccn1] | 31 | 2-{1- [(carbamoylmethyl)[2- (diethylcarbamoyl)ethyl] amino]ethyl}pyridine- 4-carboxylic acid | A | ¹H-NMR (300 MHz, CD₃OD): δ 8.5 (d, 1H), 4.07 (q, 1H), 1.42 (d, 3H), 1.09-1.03 (m, 6H) ppm. |
| 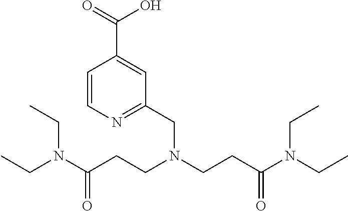 [SMILES: CCN(CC)C(=O)CCN(CCC(=O)N(CC)CC)Cc1cc(C(=O)O)ccn1] | 32 | 2-({bis[2- (diethylcarbamoyl)ethyl] amino}methyl)pyri- dine-4-carboxylic acid | A | ¹H-NMR (300 MHz, CD₃OD): δ 8.50 (d, 1H), 3.81 (s, 2H), 2.59 (t, 2H), 1.19-1.07 (m, 12H) ppm. |
| 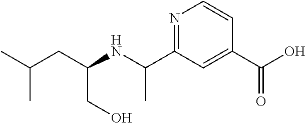 [SMILES: CC(C)C[C@H](CO)NC(C)c1cc(C(=O)O)ccn1] | 33 | 2-(1-{[(2R)-1- hydroxy-4- methylpentan-2- yl]amino}ethyl)pyridine- 4-carboxylic acid | A | ¹H NMR (300 MHz, CD₃OD), δ ppm: 8.53 (d), 4.12 (q), 2.48 (m), |
| 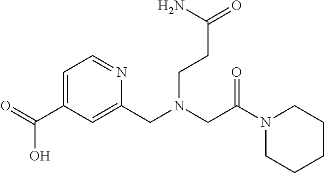 [SMILES: NC(=O)CCN(CC(=O)N1CCCCC1)Cc1cc(C(=O)O)ccn1] | 34 | 2-{[(2- carbamoylethyl)[2- oxo-2-(piperidin-1- yl)ethyl]amino]methyl} pyridine-4- carboxylic acid | A | ¹H-NMR (300 MHz, CD₃OD): δ 8.50 (d, 1H), 7.95 (s, 1H), 7.71 (d, 1H), 2.94 (t, 2H), 2.46 (t, 2H). |
| 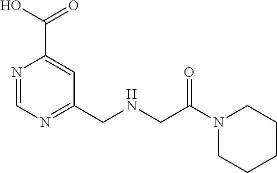 [SMILES: O=C(O)c1cc(CNCC(=O)N2CCCCC2)ncn1] | 35 | 6-({[2-oxo-2- (piperidin-1- yl)ethyl]amino}methyl) pyrimidine-4- carboxylic acid | A | ¹H NMR (300 MHz, CD₃OD), δ ppm: 9.10 (d, 1H), 3.97 (s, 2H), 3.39 (m, 2H), 1.70- 1.50 (m, 6H). |
| 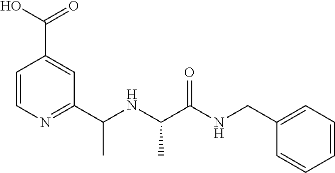 [SMILES: CC(N[C@@H](C)C(=O)NCc1ccccc1)c1cc(C(=O)O)ccn1] | 36 | 2-(1-{[(1S)-1- (benzylcarbamoyl)ethyl] amino}ethyl)pyridine- 4-carboxylic acid | A | ¹H NMR (300 MHz, CD₃OD), δ ppm: 8.47 (d), 4.27 (s), 3.97 (q), 3.26 (q) |
| 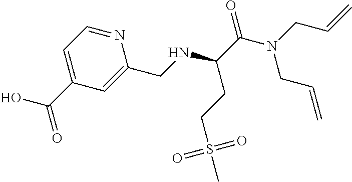 [SMILES: C=CCN(CC=C)C(=O)[C@@H](CCS(C)(=O)=O)NCc1cc(C(=O)O)ccn1] | 37 | 2-({[(1R)-1-[bis(prop- 2-en-1-yl)carbamoyl]- 3- methanesulfonylpropyl] amino}methyl)pyridine- 4-carboxylic acid | A | ¹H-NMR (300 MHz, CD₃OD): δ 8.49 (d, 1H), 7.97 (s, 1H), 7.71 (d, 1H), 3.00 (s, 3H). |
| 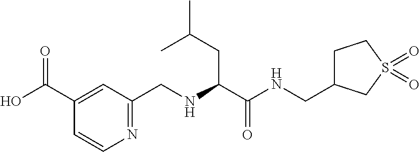 [SMILES: CC(C)C[C@H](NCc1cc(C(=O)O)ccn1)C(=O)NCC1CCS(=O)(=O)C1] | 38 | 2-({[(1S)-1-{[(1,1- dioxo-1-thiolan-3- yl)methyl]carbamoyl}- 3- methylbutyl]amino} methyl)pyridine-4- carboxylic acid | A | ¹H NMR (300 MHz, CD₃OD), δ ppm: 8.53 (d, 1H), 7.88 (s, 1H), 7.71 (d, 1H), 2.39-2.22 (m, 2H), 3.24-3.17 (m, 3H), 0.94 (d, 3H), 0.86 (d, 3H). |
| 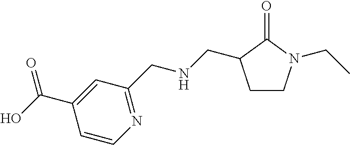 [SMILES: CCN1CCC(CNCc2cc(C(=O)O)ccn2)C1=O] | 39 | 2-({[(1-ethyl-2- oxopyrrolidin-3- yl)methyl]amino} methyl)pyridine-4- carboxylic acid | A | ¹H-NMR (300 MHz, CD₃OD ): δ 8.83 (d, 1H), 4.54 (AB, 2H), 2.37 (m, 1H), 1.16 (t, 3H) ppm. |
| 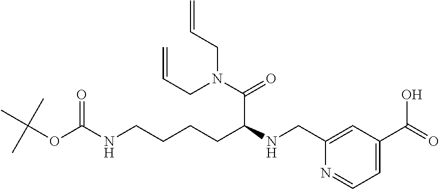 [SMILES: C=CCN(CC=C)C(=O)[C@H](CCCCNC(=O)OC(C)(C)C)NCc1cc(C(=O)O)ccn1] | 40 | 2-({[(1S)-1-[bis(prop- 2-en-1-yl)carbamoyl]- 5-{[(tert- butoxy)carbonyl]amino} pentyl]amino}methyl) pyridine-4- carboxylic acid | A | ¹H-NMR (300 MHz, CD₃OD): δ 8.50 (d, 1H), 7.93 (s, 1H), 7.70 (d, 1H), 1.43 (s, 9H). |
| 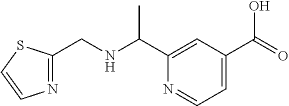 [SMILES: CC(NCc1nccs1)c1cc(C(=O)O)ccn1] | 41 | 2-{1-[(1,3-thiazol-2- ylmethyl)amino]ethyl} pyridine-4- carboxylic acid | A | ¹H-NMR (300 MHz, CD₃OD): δ 8.40 (d, 1H), 7.80 (s, 1H), 7.40 (d, 1H), 3.95 (m, 1H), 1.40 (d, 6H) |
| 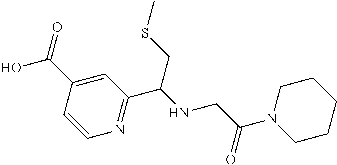 [SMILES: CSCC(NCC(=O)N1CCCCC1)c1cc(C(=O)O)ccn1] | 42 | 2-[2-(methylsulfanyl)- 1-{[2-oxo-2- (piperidin-1- yl)ethyl]amino}ethyl] pyridine-4-carboxylic acid | A | ¹H NMR (300 MHz, CD₃OD), δ ppm: 8.53 (m, 1H), 3.99 (m, 1H), 2.85 (m, 2H), 2.07 (s, 3H). |
| 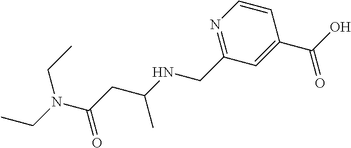 [SMILES: CCN(CC)C(=O)CC(C)NCc1cc(C(=O)O)ccn1] | 43 | 2-({[1- (diethylcarbamoyl) propan-2- yl]amino}methyl)pyri- dine-4-carboxylic acid | A | ¹H-NMR (300 MHz, CD₃OD): δ 8.55 (d, 1H), 3.96 (AB, 2H), 3.19 (m, 1H), 1.19 (d, 3H), 1.12 (t, 6H) ppm. |
| 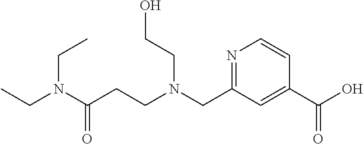 [SMILES: CCN(CC)C(=O)CCN(CCO)Cc1cc(C(=O)O)ccn1] | 44 | 2-({[2- (diethylcarbamoyl) ethyl](2- hydroxyethyl)amino} methyl)pyridine-4- carboxylic acid | A | ¹H-NMR (300 MHz, CD₃OD): δ 8.52 (d, 1H), 3.86 (s, 2H), 3.66 (t, 2H), 1.15-1.05 (m, 6H) ppm. |
| 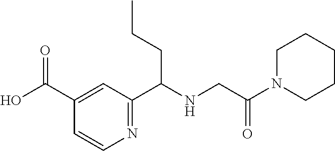 [SMILES: CCCC(NCC(=O)N1CCCCC1)c1cc(C(=O)O)ccn1] | 45 | 2-(1-{[2-oxo-2- (piperidin-1- yl)ethyl]amino}butyl) pyridine-4-carboxylic acid | A | ¹H NMR (300 MHz, CD₃OD), δ ppm: 8.52 (m, 1H), 3.79 (t, 1H), 1.79 (m, 2H), 0.88 (t, 3H). |
| 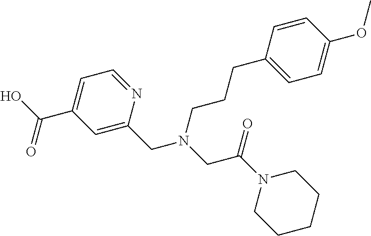 [SMILES: COc1ccc(CCCN(CC(=O)N2CCCCC2)Cc2cc(C(=O)O)ccn2)cc1] | 46 | 2-({[3-(4- methoxyphenyl)propyl] [2-oxo-2-(piperidin- 1- yl)ethyl]amino}methyl) pyridine-4- carboxylic acid | A | ¹H-NMR (300 MHz, CD₃OD): δ 8.48 (d, 1H), 7.98 (s, 1H), 7.72 (d, 1H), 7.04 (d, 2H), 6.78 (d, 2H), 3.75 (s, 3H). |
| 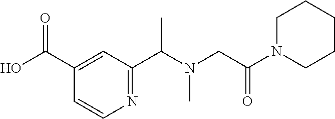 [SMILES: CC(c1cc(C(=O)O)ccn1)N(C)CC(=O)N1CCCCC1] | 47 | 2-(1-{methyl[2-oxo-2- (piperidin-1- yl)ethyl]amino}ethyl) pyridine-4-carboxylic acid | A | ¹H NMR (300 MHz, CD₃OD), δ ppm: 8.51 (m, 1H), 3.89 (q, 1H), 2.25 (s, 3H), 1.59 (m, 6H). |
| 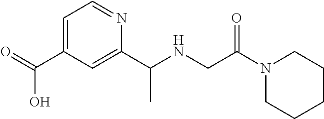 [SMILES: CC(NCC(=O)N1CCCCC1)c1cc(C(=O)O)ccn1] | 48 | 2-(1-{[2-oxo-2- (piperidin-1- yl)ethyl]amino}ethyl) pyridine-4-carboxylic acid | A | ¹H-NMR (300 MHz, CD₃OD): δ 8.52 (d, 1H), 7.86 (s, 1H), 7.69 (d, 1H), 3.94 (q, 1H), 1.42 (d, 3H). |
| 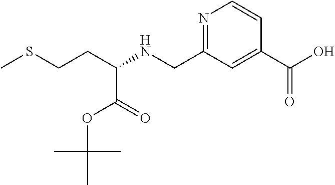 [SMILES: CSCC[C@H](NCc1cc(C(=O)O)ccn1)C(=O)OC(C)(C)C] | 49 | 2-({[(2S)-1-(tert- butoxy)-4- (methylsulfanyl)-1- oxobutan-2- yl]amino}methyl)pyri- dine-4-carboxylic acid | A | ¹H NMR (300 MHz, CD₃OD), δ ppm: 8.49 (d, 1H), 3.82 (m, 3H), 2.15 (s, 3H), 1.85(m, 2H). |
| 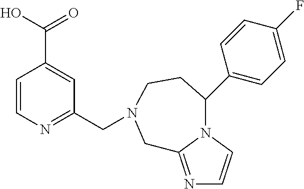 [SMILES: O=C(O)c1ccnc(CN2CCC(c3ccc(F)cc3)n3ccnc3C2)c1] | 50 | 2-{[5-(4- fluorophenyl)- 5H,6H,7H,8H,9H- imidazo[1,2- a][1,4]diazepin-8- yl]methyl}pyridine-4- carboxylic acid | A | ¹H-NMR (300 MHz, CD₃OD): δ 8.5 (d, 1H), 7.6 (d, 1H), 6.9 (m, 5H), 4.5 (m, 1H), 3.8 (s, 4H), |
| 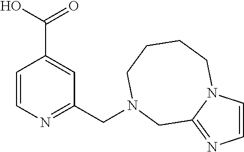 [SMILES: O=C(O)c1ccnc(CN2CCCCn3ccnc3C2)c1] | 51 | 2- {5H,6H,7H,8H,9H,10H- imidazo[1,2- a][1,4]diazocin-9- ylmethyl}pyridine-4- carboxylic acid | B | ¹H NMR (300 MHz, CD₃OD), δ 8.50 (d, 1H), 4.42 (t, 2H), 3.86 (s, 2H), 1.47 (m, 2H) |
| 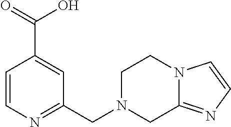 [SMILES: O=C(O)c1ccnc(CN2CCn3ccnc3C2)c1] | 52 | 2-{5H,6H,7H,8H- imidazo[1,2- a]pyrazin-7- ylmethyl}pyridine-4- carboxylic acid | B | ¹H NMR (300 MHz, CD₃OD), δ 8.00 (s, 1H), 7.00 (s, 1H), 4.07 (t, 2H), 2.98 (t, 2H) ppm. |
| 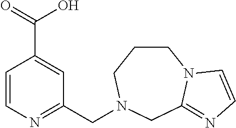 [SMILES: O=C(O)c1ccnc(CN2CCCn3ccnc3C2)c1] | 53 | 2-{5H,6H,7H,8H,9H- imidazo[1,2- a][1,4]diazepin-8- ylmethyl}pyridine-4- carboxylic acid | B | ¹H NMR (300 MHz, CD₃OD), δ 7.97 (s, 1H), 4.17-4.14 (m, 2H), 3.13 (m, 2H), 1.92-1.89 (m, 2H) ppm. |
| 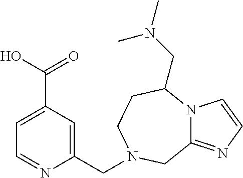 [SMILES: CN(C)CC1CCN(Cc2cc(C(=O)O)ccn2)Cc2nccn21] | 54 | 2-({5- [(dimethylamino)methyl]- 5H,6H,7H,8H,9H- imidazo[1,2- a][1,4]diazepin-8- yl}methyl)pyridine-4- carboxylic acid | A | ¹H-NMR (300 MHz, CD₃OD): δ 8.3 (d, 1H), 4.3 (m, 1H), 3.9 (s, 2H), 2.1 (s, 6H), |
| 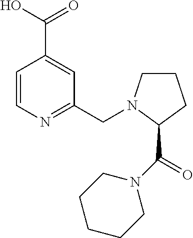 [SMILES: O=C(O)c1ccnc(CN2CCC[C@H]2C(=O)N2CCCCC2)c1] | 55 | 2-{[(2S)-2- (piperidine-1- carbonyl)pyrrolidin-1- yl]methyl}pyridine-4- carboxylic acid | A | ¹H NMR (300 MHz, CD₃OD), δ 8.51 (d, 1H), 7.88 (s, 1H), 3.97 (d, 1H), 2.41 (t, 1H) ppm. |
| 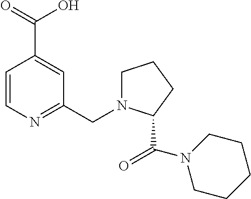 [SMILES: O=C(O)c1ccnc(CN2CCC[C@@H]2C(=O)N2CCCCC2)c1] | 56 | 2-{[(2R)-2- (piperidine-1- carbonyl)pyrrolidin-1- yl]methyl}pyridine-4- carboxylic acid | A | ¹H NMR (300 MHz, CD₃OD), δ 8.49 (d, 1H), 3.98 (d, 1H), 2.46 (q, 1H), 2.18 (m, 1H). |
| 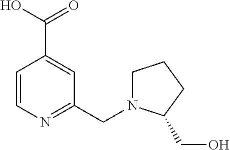 [SMILES: O=C(O)c1ccnc(CN2CCC[C@@H]2CO)c1] | 57 | 2-{[(2R)-2- (hydroxymethyl) pyrrolidin-1- yl]methyl}pyridine-4- carboxylic acid | A | ¹H NMR (300 MHz, CD₃OD), δ 8.50 (d, 1H), 4.20 (d, 1H), 2.90 (m, 1H), 1.76-1.68 (m, 3H) ppm. |
| 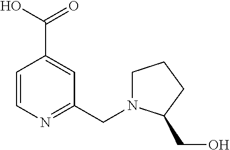 [SMILES: O=C(O)c1ccnc(CN2CCC[C@H]2CO)c1] | 58 | 2-{[(2S)-2- (hydroxymethyl) pyrrolidin-1- yl]methyl}pyridine-4- carboxylic acid | A | ¹H NMR (300 MHz, CD₃OD), δ 8.50 (d, 1H), 2.92 (m, 1H), 2.33 (m, 1H), 1.77-1.66 (m, 3H) ppm. |
| 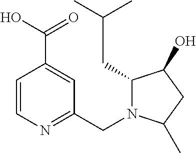 [SMILES: CC(C)C[C@@H]1[C@@H](O)CC(C)N1Cc1cc(C(=O)O)ccn1] | 59 | 2-{[(2R,3S)-3- hydroxy-5-methyl-2- (2- methylpropyl)pyrrolidin- 1- yl]methyl}pyridine-4- carboxylic acid | A | ¹H-NMR (300 MHz, CD₃OD): δ 8.50 (d, 1H), 7.71 (d, 1H), 4.04- 3.85 (m, 3H), 0.76 (d, 3H) |
| 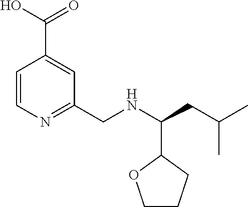 [SMILES: CC(C)C[C@H](NCc1cc(C(=O)O)ccn1)C1CCCO1] | 60 | 2-({[(1S)-3-methyl-1- (oxolan-2- yl)butyl]amino}methyl) pyridine-4- carboxylic acid | A | ¹H-NMR (300 MHz, CD₃OD): δ 8.53 (d, 1H), 7.71 (d, 1H), 1.44- 1.23 (m, 2H), 0.85- 0.78 (m, 3H) |
| 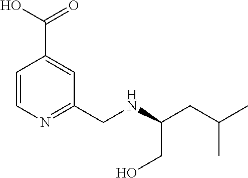 [SMILES: CC(C)C[C@@H](CO)NCc1cc(C(=O)O)ccn1] | 61 | (S)-2-{[(1-hydroxy-4- methylpentan-2- yl)amino]methyl}pyri- dine-4-carboxylic acid | A | ¹H-NMR (300 MHz, CD₃OD): δ 8.55 (d, 1H), 4.00 (AB, 2H), 2.72 (m, 1H), 1.70 (m, 1H) ppm. |
| 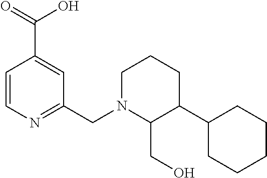 [SMILES: O=C(O)c1ccnc(CN2CCCC(C3CCCCC3)C2CO)c1] | 62 | 2-{[3-cyclohexyl-2- (hydroxymethyl) piperidin-1- yl]methyl}pyridine-4- carboxylic acid | A | ¹H-NMR (300 MHz, CD₃OD): δ 8.30 (d, 1H), 7.75 (s, 1H), 4.00 (d, 1H), 3.80 (m, 2H). |
| 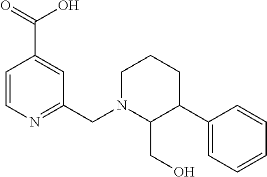 [SMILES: O=C(O)c1ccnc(CN2CCCC(c3ccccc3)C2CO)c1] | 63 | 2-{[2- (hydroxymethyl)-3- phenylpiperidin-1- yl]methyl}pyridine-4- carboxylic acid | A | ¹H-NMR (300 MHz, CD₃OD): δ 8.45 (d, 1H), 7.90 (s, 1H), 4.00- (dd, 2H), 3.80 (t, 1H). |
| 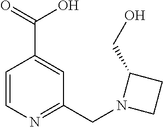 [SMILES: O=C(O)c1ccnc(CN2CC[C@H]2CO)c1] | 64 | 2-{[(2S)-2- (hydroxymethyl) azetidin-1- yl]methyl}pyridine-4- carboxylic acid | A | ¹H-NMR (300 MHz, CD₃OD): δ 8.30 (d, 1H), 7.60 (s, 1H), 3.70 (dd, 2H), 3.30 (s, 2H). |
| 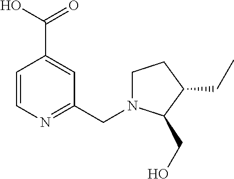 [SMILES: CC[C@H]1CCN(Cc2cc(C(=O)O)ccn2)[C@@H]1CO] | 65 | 2-{[(2S,3S)-3-ethyl-2- (hydroxymethyl) pyrrolidin-1- yl]methyl}pyridine-4- carboxylic acid | A | ¹H NMR (300 MHz, CD₃OD), δ 8.50 (d, 1H), 3.67 (m, 1H), 1.89 (m, 2H), 0.93 (t, 3H) ppm. |
| 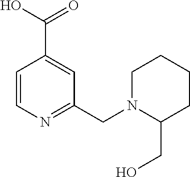 [SMILES: O=C(O)c1ccnc(CN2CCCCC2CO)c1] | 66 | 2-{[2- (hydroxymethyl)piperidin- 1-yl]methyl} pyridine-4-carboxylic acid | A | ¹H-NMR (300 MHz, CD₃OD): δ 8.53 (d, 1H), 3.74 (s, 2H), 2.86 (m, 1H), 1.56 (m, 3H) ppm. |
| 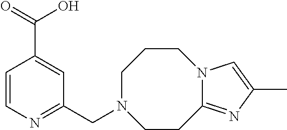 [SMILES: Cc1cn2c(n1)CCN(Cc1cc(C(=O)O)ccn1)CCC2] | 67 | 2-({2-methyl- 5H,6H,7H,8H,9H,10H- imidazo[1,2- a][1,5]diazocin-8- yl}methyl)pyridine-4- carboxylic acid | A | ¹H-NMR (300 MHz, CD₃OD): δ 7.78 (s, 1H), 4.10 (m, 2H), 3.82(s, 2H), 1.78 (m, 2H) ppm. |
| 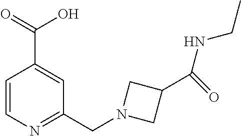 [SMILES: CCNC(=O)C1CN(Cc2cc(C(=O)O)ccn2)C1] | 68 | 2-{[3- (ethylcarbamoyl)azetidin- 1- yl]methyl}pyridine-4- carboxylic acid | A | ¹H NMR (300 MHz, CD₃OD), δ 8.48 (d, 1H), 7.71 (s, 1H), 3.60 (m, 2H), 1.1 (t, 3H) ppm. |
| 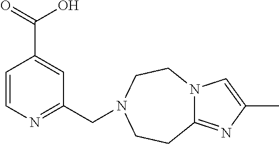 [SMILES: Cc1cn2c(n1)CCN(Cc1cc(C(=O)O)ccn1)CC2] | 69 | 2-({2-methyl- 5H,6H,7H,8H,9H- imidazo[1,2- d][1,4]diazepin-7- yl}methyl)pyridine-4- carboxylic acid | A | ¹H NMR (300 MHz, CD₃OD), δ 8.49 (d, 1H), 8.02 (s, 1H), 6.60 (s, 1H) 2.76 (m, 4H), 2.08 (s, 3H) ppm. |
| 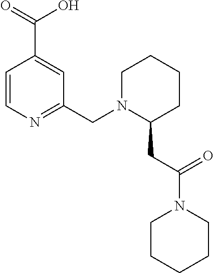 [SMILES: O=C(O)c1ccnc(CN2CCCC[C@H]2CC(=O)N2CCCCC2)c1] | 70 | 2-{[(2S)-2-[2-oxo-2- (piperidin-1- yl)ethyl]piperidin-1- yl]methyl}pyridine-4- carboxylic acid | A | ¹H NMR (300 MHz, CD₃OD), δ 8.48 (d, 1H), 7.68 (d, 1H), 2.80-2.73 (m, 1H), 2.51-2.32 (m, 2H) ppm. |
| 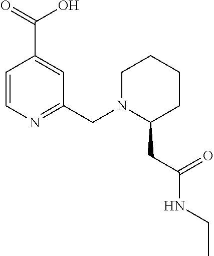 [SMILES: CCNC(=O)C[C@@H]1CCCCN1Cc1cc(C(=O)O)ccn1] | 71 | 2-{[(2S)-2- [(ethylcarbamoyl) methyl]piperidin-1- yl]methyl}pyridine-4- carboxylic acid | A | ¹H NMR (300 MHz, CD₃OD), δ 8.48 (d, 1H), 3.20 (m, 2H), 2.74 (m, 1H), .10 (t, 3H) ppm. |
| 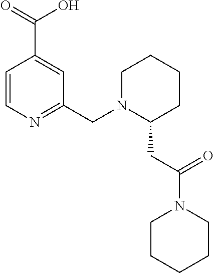 [SMILES: O=C(O)c1ccnc(CN2CCCC[C@@H]2CC(=O)N2CCCCC2)c1] | 72 | 2-{[(2R)-2-[2-oxo-2- (piperidin-1- yl)ethyl]piperidin-1- yl]methyl}pyridine-4- carboxylic acid | A | ¹H NMR (300 MHz, CD₃OD), δ 8.48 (d, 1H), 7.68 (d, 1H), 2.80-2.73 (m, 1H), 2.51-2.32 (m, 2H) ppm. |
| 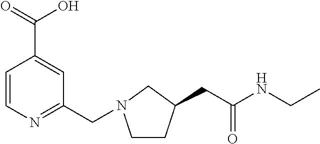 [SMILES: CCNC(=O)C[C@H]1CCN(Cc2cc(C(=O)O)ccn2)C1] | 73 | 2-{[(3R)-3- [(ethylcarbamoyl) methyl]pyrrolidin-1- yl]methyl}pyridine-4- carboxylic acid | A | ¹H-NMR (300 MHz, CD₃OD): δ 8.45 (d, 1H), 3.80 (d, 2H), 3.18 (q, 2H), 1.50 (m, 1H) ppm. |
| 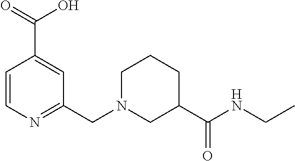 [SMILES: CCNC(=O)C1CCCN(Cc2cc(C(=O)O)ccn2)C1] | 74 | 2-{[3- (ethylcarbamoyl)piper- idin-1- yl]methyl}pyridine-4- carboxylic acid | A | ¹H-NMR (300 MHz, CD₃OD): δ 8.49 (d, 1H), 7.75 (s, 1H), 3.68 (s, 2H), 3.15 (q, 2H), 1.68 (m, 4H). |
| 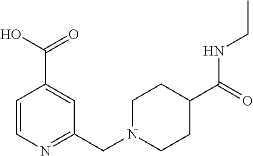 [SMILES: CCNC(=O)C1CCN(Cc2cc(C(=O)O)ccn2)CC1] | 75 | 2-{[4- (ethylcarbamoyl)piper- idin-1-yl]methyl} pyridine-4-carboxylic acid | A | ¹H-NMR (300 MHz, CD₃OD): δ 7.90 (s, 1H), 3.70 (s, 2H), 2.95(m, 2H), 1.15 (t, 3H) ppm. |
| 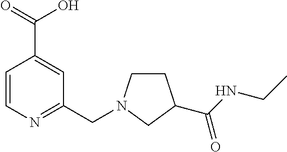 [SMILES: CCNC(=O)C1CCN(Cc2cc(C(=O)O)ccn2)C1] | 76 | 2-{[3- (ethylcarbamoyl) pyrrolidin-1- yl]methyl}pyridine-4- carboxylic acid | A | ¹H-NMR (300 MHz, CD₃OD): δ 8.51 (d, 1H), 7.86 (s, 1H), 3.2 (q, 2H), 2.05 (m, 2H). |
| 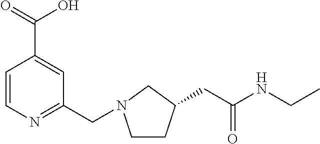 [SMILES: CCNC(=O)C[C@@H]1CCN(Cc2cc(C(=O)O)ccn2)C1] | 77 | 2-{[(3S)-3- [(ethylcarbamoyl) methyl]pyrrolidin-1- yl]methyl}pyridine-4- carboxylic acid | A | ¹H-NMR (300 MHz, CD₃OD): δ 8.42 (d, 1H), 3.79 (d, 2H), 3.15(q, 2H), 2.05 (m, 1H) ppm. |
| 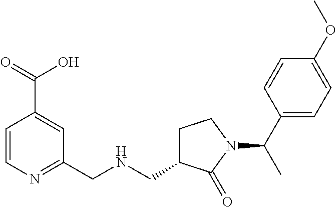 [SMILES: COc1ccc([C@@H](C)N2CC[C@@H](CNCc3cc(C(=O)O)ccn3)C2=O)cc1] | 78 | 2-[({[(3S)-1-[(1R)-1- (4- methoxyphenyl)ethyl]- 2-oxopyrrolidin-3- yl]methyl}amino) methyl]pyridine-4- carboxylic acid | A | ¹H-NMR (300 MHz, CD₃OD): δ 7.99 (s, 1H), 7.26 (s, 1H), 3.56 (s, 2H), 1.27 (d, 3H) |
| 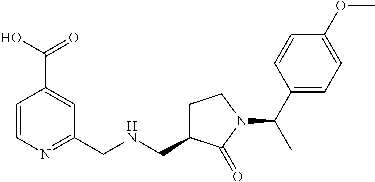 [SMILES: COc1ccc([C@@H](C)N2CC[C@H](CNCc3cc(C(=O)O)ccn3)C2=O)cc1] | 79 | 2-[({[(3R)-1-[(1R)-1- (4- methoxyphenyl)ethyl]- 2-oxopyrrolidin-3- yl]methyl}amino) methyl]pyridine-4- carboxylic acid | A | ¹H-NMR (300 MHz, CD₃OD): δ 8.55 (d, 1H), 7.91 (s, 1H), 3.97 (s, 2H), 1.52 (d, 3H) |
| 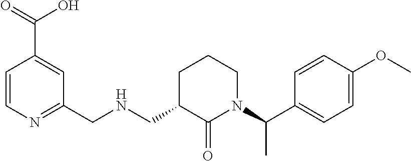 [SMILES: COc1ccc([C@@H](C)N2CCC[C@@H](CNCc3cc(C(=O)O)ccn3)C2=O)cc1] | 80 | 2-[({[(3S)-1-[(1R)-1- (4- methoxyphenyl)ethyl]- 2-oxopiperidin-3- yl]methyl}amino) methyl]pyridine-4- carboxylic acid | A | ¹H-NMR (300 MHz, CD₃OD): δ 8.55 (d, 1H), ), 7.73 (s, 1H), 1.72-1.58 (m, 2H), 1.46 (d, 3H) |
| 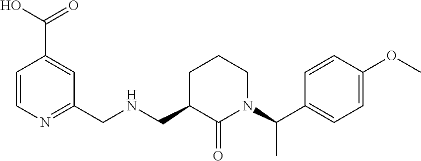 [SMILES: COc1ccc([C@@H](C)N2CCC[C@H](CNCc3cc(C(=O)O)ccn3)C2=O)cc1] | 81 | 2-[({[(3R)-1-[(1R)-1- (4- methoxyphenyl)ethyl]- 2-oxopiperidin-3- yl]methyl}amino) methyl]pyridine-4- carboxylic acid | A | ¹H-NMR (300 MHz, CD₃OD): δ 8.58 (d, 1H), 7.74 (s, 1H), 3.99 (s, 2H), 3.22-3.13 (m, 1H), 1.50 (d, 3H |
| 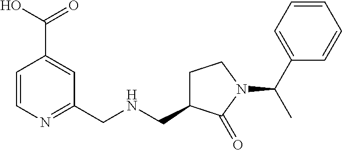 [SMILES: C[C@H](c1ccccc1)N1CC[C@H](CNCc2cc(C(=O)O)ccn2)C1=O] | 82 | 2-[({[(3R)-2-oxo-1- [(1R)-1- phenylethyl]pyrrolidin- 3- yl]methyl}amino) methyl]pyridine-4- carboxylic acid | A | ¹H-NMR (300 MHz, CD₃OD): δ 8.63 (d, 1H), 7.41-7.31 (m, 5H), 5.40 (q, 1H), 1.58 (d, 3H) |
| 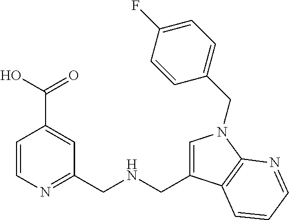 [SMILES: O=C(O)c1ccnc(CNCc2cn(Cc3ccc(F)cc3)c3ncccc23)c1] | 83 | 2-[({[1-(4- fluorobenzyl)-1H- pyrrolo[2,3-b]pyridin- 3- yl]methyl}amino) methyl]pyridine-4- carboxylic acid | A | ¹H-NMR (300 MHz, CD₃OD): δ 8.49 (d, 1H), 8.21 (d, 1H), 5.42 (s, 2H), 3.93 (s, 4H) ppm |
| 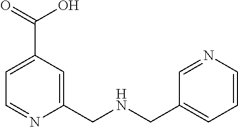 [SMILES: O=C(O)c1ccnc(CNCc2cccnc2)c1] | 84 | 2-{[(pyridin-3- ylmethyl)amino] methyl}pyridine-4- carboxylic acid | A | ¹H-NMR (300 MHz, CD₃OD): δ 8.55 (s, 1H), 7.43 (dd, 1H), 3.96 (s, 2H), 3.86 (s, 2H) ppm |
| 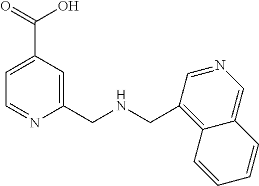 [SMILES: O=C(O)c1ccnc(CNCc2cncc3ccccc23)c1] | 85 | 2-{[(isoquinolin-4- ylmethyl)amino] methyl}pyridine-4- carboxylic acid | A | ¹H-NMR (300 MHz, CD₃OD): δ 9.11 (s, 1H), 8.51 (d, 1H), 4.20 (s, 2H), 4.02 (s, 2H) ppm |
| 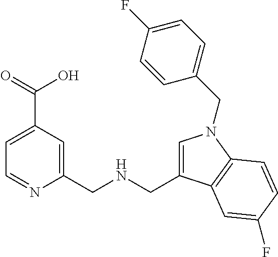 [SMILES: O=C(O)c1ccnc(CNCc2cn(Cc3ccc(F)cc3)c3ccc(F)cc23)c1] | 86 | 2-{[({5-fluoro-1-[(4- fluorophenyl)methyl]- 1H-indol-3- yl}methyl)amino] methyl}pyridine-4- carboxylic acid | A | ¹H-NMR (300 MHz, CD₃OD): δ 8.53 (d, 1H), 7.84 (s, 1H), 5.32 (s, 2H), 4.01 (d, 4H) ppm |
| 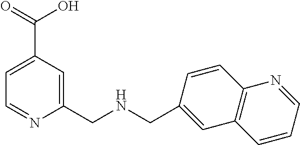 [SMILES: O=C(O)c1ccnc(CNCc2ccc3ncccc3c2)c1] | 87 | 2-{[(quinolin-6- ylmethyl)amino] methyl}pyridine-4- carboxylic acid | A | ¹H-NMR (300 MHz, CD₃OD): δ 8.78 (d, 1H), 8.51 (d, 1H), 3.98 (s, 2H), 3.95 (d, 2H) ppm |
| 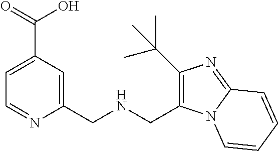 [SMILES: CC(C)(C)c1nc2ccccn2c1CNCc1cc(C(=O)O)ccn1] | 88 | 2-{[({2-tert- butylimidazo[1,2- a]pyridin-3- yl}methyl)amino] methyl}pyridine-4- carboxylic acid | A | ¹H-NMR (300 MHz, CD₃OD): δ 8.50 (d, 1H), 8.33 (d, 1H), 4.16 (s, 2H), 1.35 (s, 9H) ppm |
| 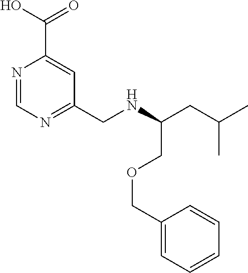 [SMILES: CC(C)C[C@@H](COCc1ccccc1)NCc1cc(C(=O)O)ncn1] | 89 | 6-({[(2S)-1- (benzyloxy)-4- methylpentan-2- yl]amino}methyl) pyrimidine-4-carboxylic acid | A | ¹H NMR (300 MHz, CD₃OD), δ 9.10 (s, 1H), 7.27 (m, 5H), 4.51 (s, 2H), 1.67 (m, 1H) ppm. |
| 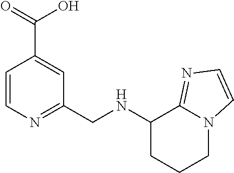 [SMILES: O=C(O)c1ccnc(CNC2CCCn3ccnc32)c1] | 90 | 2-[({5H,6H,7H,8H- imidazo[1,2-a]pyridin- 8- yl}amino)methyl]pyri- dine-4-carboxylic acid | A | ¹H-NMR (300 MHz, CD₃OD): δ 8.54 (d, 1H), 6.99 (s, 1H), 6.95 (s, 1H), 4.01 (m, 3H), ppm. |
| 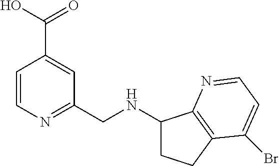 [SMILES: O=C(O)c1ccnc(CNC2CCc3c(Br)ccnc32)c1] | 91 | 2-[({4-bromo- 5H,6H,7H- cyclopenta[b]pyridin- 7- yl}amino)methyl]pyri- dine-4-carboxylic acid | A | ¹H-NMR (300 MHz, CD₃OD): δ 8.56 (d, 1H), 8.22 (d, 1H), 7.56 (d, 1H, 2.07-1.95 (m, 1H) |
| 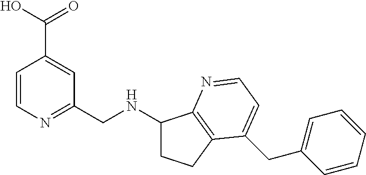 [SMILES: O=C(O)c1ccnc(CNC2CCc3c(Cc4ccccc4)ccnc32)c1] | 92 | 2-[({4-benzyl- 5H,6H,7H- cyclopenta[b]pyridin- 7- yl}amino)methyl]pyri- dine-4-carboxylic acid | A | ¹H-NMR (300 MHz, CD₃OD): δ 8.20 (d, 1H), 6.96 (d, 1H), 4.15 (t, 1H), 1.96-1.81 (m, 1H) ppm |
| 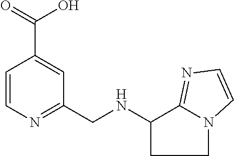 [SMILES: O=C(O)c1ccnc(CNC2CCn3ccnc32)c1] | 93 | 2-[({5H,6H,7H- pyrrolo[1,2- a]imidazol-7- yl}amino)methyl]pyri- dine-4-carboxylic acid | A | ¹H-NMR (300 MHz, CD₃OD): δ 8.51 (d, 1H), 7.03 (s, 1H), 6.99 (s, 1H), 3.96 (m, 1H) ppm. |
| 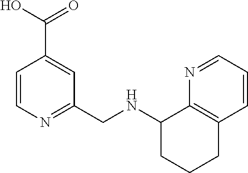 [SMILES: O=C(O)c1ccnc(CNC2CCCc3cccnc32)c1] | 94 | 2-{[(5,6,7,8- tetrahydroquinolin-8- yl)amino]methyl} pyridine-4-carboxylic acid | A | ¹H-NMR (300 MHz, CD₃OD): δ 8.47 (d, 1H), 7.07 (dd, 1H), 3.90 (t, 1H), 2.61 (m, 2H) ppm. |
| 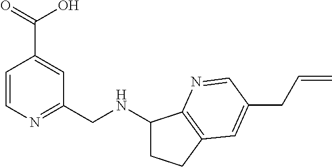 [SMILES: C=CCc1cnc2c(c1)CCC2NCc1cc(C(=O)O)ccn1] | 95 | 2-({[3-(prop-2-en-1- yl)-5H,6H,7H- cyclopenta[b]pyridin- 7- yl]amino}methyl)pyri- dine-4-carboxylic acid | A | ¹H-NMR (300 MHz, CD₃OD): δ 8.81 (d, 1H), 7.63 (s, 1H), 3.45 (d, 2H), 2.27 (m, 1H) ppm. |
| 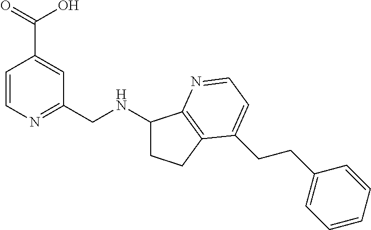 [SMILES: O=C(O)c1ccnc(CNC2CCc3c(CCc4ccccc4)ccnc32)c1] | 96 | 2-({[4-(2- phenylethyl)- 5H,6H,7H- cyclopenta[b]pyridin- 7- yl]amino}methyl)pyri- dine-4-carboxylic acid | A | ¹H-NMR (300 MHz, CD₃OD): δ 8.34 (d, 1H), 7.99 (s, 1H), 2.94 (s, 4H), 2.23-2.07 (m, 1H) ppm |
| 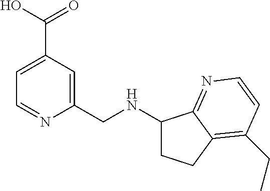 [SMILES: CCc1ccnc2c1CCC2NCc1cc(C(=O)O)ccn1] | 97 | 2-[({4-ethyl- 5H,6H,7H- cyclopenta[b]pyridin- 7- yl}amino)methyl]pyri- dine-4-carboxylic acid | A | ¹H-NMR (300 MHz, CD₃OD): δ 8.03 (s, 1H), 7.27 (d, 1H), 1.27 (t, 3H) ppm |
| 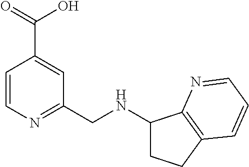 [SMILES: O=C(O)c1ccnc(CNC2CCc3cccnc32)c1] | 98 | 2-[({5H,6H,7H- cyclopenta[b]pyridin- 7- yl}amino)methyl]pyri- dine-4-carboxylic acid | A | ¹H-NMR (300 MHz, CD₃OD): δ 8.40 (d, 1H), 8.20 (d, 1H), 4.05 (t, 1H), 3.90 (s, 2H). |
| 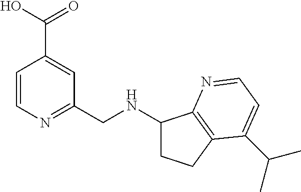 [SMILES: CC(C)c1ccnc2c1CCC2NCc1cc(C(=O)O)ccn1] | 99 | 2-({[4-(propan-2-yl)- 5H,6H,7H- cyclopenta[b]pyridin- 7- yl]amino}methyl)pyri- dine-4-carboxylic acid | A | ¹H-NMR (300 MHz, CD₃OD): δ 7.98 (s, 1H), 7.27 (d, 1H), 1.24 (m, 6H) ppm |
| 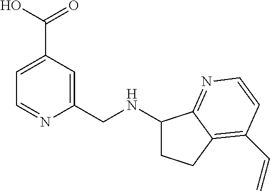 [SMILES: C=Cc1ccnc2c1CCC2NCc1cc(C(=O)O)ccn1] | 100 | 2-[({4-ethenyl- 5H,6H,7H- cyclopenta[b]pyridin- 7- yl}amino)methyl]pyri- dine-4-carboxylic acid | A | ¹H-NMR (300 MHz, CD₃OD): δ 8.05 (s, 1H), 7.52 (d, 1H), 6.87 (dd, 1H), 2.38-2.22 (m, 1H) ppm |
| 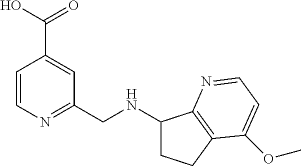 [SMILES: COc1ccnc2c1CCC2NCc1cc(C(=O)O)ccn1] | 101 | 2-[({4-methoxy- 5H,6H,7H- cyclopenta[b]pyridin- 7- yl}amino)methyl]pyri- dine-4-carboxylic acid | A | ¹H-NMR (300 MHz, CD₃OD): δ 7.87 (s, 1H), 6.84 (d, 1H), 4.21 (t, 1H), 3.88 (s, 3H) ppm |
| 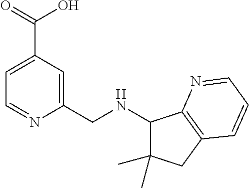 [SMILES: CC1(C)Cc2cccnc2C1NCc1cc(C(=O)O)ccn1] | 102 | 2-[({6,6-dimethyl- 5H,6H,7H- cyclopenta[b]pyridin- 7- yl}amino)methyl]pyri- dine-4-carboxylic acid | A | ¹H-NMR (300 MHz, CD₃OD): δ 7.79 (s, 1H), 7.04 (dd, 1H), 4.07 (d, 2H), 1.03 (s, 3H) ppm |
| 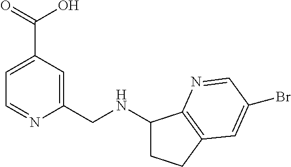 [SMILES: O=C(O)c1ccnc(CNC2CCc3cc(Br)cnc32)c1] | 103 | 2-[({3-bromo- 5H,6H,7H- cyclopenta[b]pyridin- 7- yl}amino)methyl]pyri- dine-4-carboxylic acid | A | ¹H-NMR (300 MHz, CD₃OD): δ 8.54 (d, 1H), 7.71 (d, 1H), 4.10 (s, 2H), 1.98 (m, 1H) ppm. |
| 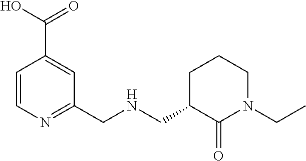 [SMILES: CCN1CCC[C@@H](CNCc2cc(C(=O)O)ccn2)C1=O] | 104 | 2-[({[(3S)-1-ethyl-2- oxopiperidin-3- yl]methyl}amino) methyl]pyridine-4- carboxylic acid | A | ¹H-NMR (300 MHz, CD₃OD): δ 8.55 (d, 1H), 7.88 (s, 1H), 7.72 (d, 1H), 2.94 (dd, 1H), 1.11 (t, 3H) |
| 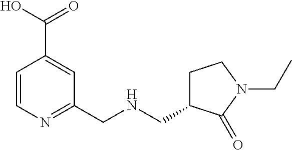 [SMILES: CCN1CC[C@@H](CNCc2cc(C(=O)O)ccn2)C1=O] | 105 | 2-[({[(3S)-1-ethyl-2- oxopyrrolidin-3- yl]methyl}amino) methyl]pyridine-4- carboxylic acid | A | ¹H-NMR (300 MHz, CD₃OD): δ8.54 (d, 1H), 7.89 (s, 1H), 7.73 (d, 1H), 3.95 (s, 2H), 1 1.12 (t, 3H) |
| 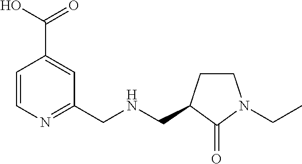 [SMILES: CCN1CC[C@H](CNCc2cc(C(=O)O)ccn2)C1=O] | 106 | 2-[({[(3R)-1-ethyl-2- oxopyrrolidin-3- yl]methyl}amino) methyl]pyridine-4- carboxylic acid | A | ¹H-NMR (300 MHz, CD₃OD): δ 8.55 (d, 1H), 7.91 (s, 1H), 2.94 (q, 1H), 1.13 (t, 3H) |
| 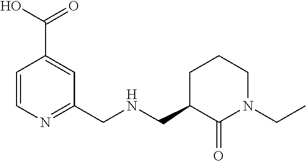 [SMILES: CCN1CCC[C@H](CNCc2cc(C(=O)O)ccn2)C1=O] | 107 | 2-[({[(3R)-1-ethyl-2- oxopiperidin-3- yl]methyl}amino) methyl]pyridine-4- carboxylic acid | A | ¹H-NMR (300 MHz, CD₃OD): δ 8.55 (d, 1H), 7.93 (d, 1H) 3.96 (dd, 1H), 1.74-1.64 (m, 1H), 1.12 (t, 3H) |
| 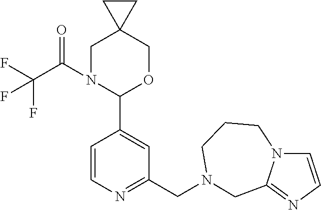 [SMILES: O=C(N1CC2(CC2)COC1c1ccnc(CN2CCCn3ccnc3C2)c1)C(F)(F)F] | 108 | 2,2,2-trifluoro-1-[6- (2-{5H,6H,7H,8H,9H- imidazo[1,2- a][1,4]diazepin-8- ylmethyl}pyridin-4- yl)-5-oxa-7- azaspiro[2.5]octan-7- yl]ethan-1-one | C | ¹H-NMR (300 MHz, CDCl₃): δ 8.64 (m, 1H), 7.14 (m, 1H), 6.81 (m, 2H), 3.86-3.73 (m, 3H), 2.03 (m, 3H), 0.68 (m, 2H), 0.44 (m, 2H) ppm. |

### General Procedures

### General Procedure A (Reductive Amination)

A solution of aldehyde and amine with optionally protected functional groups (1.3 equiv.) in a solvent such as 1,2-dichloroethane was stirred for 1-24 h at room temperature, before NaBH(AcO)₃ (2 equiv.) was added. The mixture was stirred at room temperature. The product was optionally deprotected and purified by chromatography if needed.

### General Procedure B (Ester Hydrolysis)

The ester was dissolved in a solvent such as MeOH-THF-H₂O (1:1:1) and an alkali hydroxide such as LiOH, NaOH or KOH (1.0 equiv.) was added. The reaction mixture was stirred at room temperature. Solvents were removed in vacuo to give the alkali salt of the product.

General Procedure C (Acids from Tert-Butyl Esters or Amines from Tert-Butyl Carbamates)

Trifluoroacetic acid (100 equiv.) was added to a solution of the tert-butyl carbamate or tert-butyl ester in a solvent such as DCM at 0° C. The mixture was stirred at room temperature. The product was purified by chromatography if needed.

### General Procedure D (Reduction of Ester to Aldehyde)

DIBAL-H (1.5 equiv., 1.0 M in a solvent such as toluene) was added to a solution of the ester in a solvent such as toluene at −78° C. Stirring at the same temperature before saturated NH₄Cl(aq) was added. The product was purified by chromatography if needed.

### General Procedure E: (Stereo Directed Alpha-Alkylation of Lactam)

A solution of the N—((R or S)-1-phenylethyl) lactam (obtained analogously to the procedures outlined in JOC, 2008, 73, 8627-830) in a solvent such as THF was treated with lithium diisopropyl amine (1.2 equiv.) and alkyl halide (1.5 equiv.) at −78° C. The product was isolated by aqueous workup and column chromatography if needed.

### General Procedure F: (Cleavage of Benzyl Ethers)

A slurry of a benzyl ether and Pd/C in a solvent such as MeOH was stirred in the presence of H₂. The product was isolated by filtration and chromatography if needed.

### General Procedure G (Formation of Sulfonate Ester)

An alcohol dissolved in a solvent such as dichloromethane was treated with sulfonyl chloride (2 equiv.) and triethylamine (2 equiv.). The product was isolated by aqueous workup and chromatography if needed.

### General Procedure H (Nucleophilic Substitution of Sulfonate)

A nucleophile, such as an azide (2.0 equiv.), was added to a solution of a sulfonate ester in a solvent such as dimethylformamide and the product was isolated by concentration of the reaction mixture, trituration with a solvent such as dichloromethane and purification by chromatography if needed.

### General Procedure I: (Reduction of Azides or Unsaturated C—C Bonds)

A slurry of a azide and Pd/C in a solvent such as MeOH was stirred in the presence of H₂. The product was isolated by filtration and purified chromatography if needed.

### General Procedure J (Swern Oxidation)

Oxalyl chloride (2 equiv.) was added slowly to a solution of DMSO (4 equiv.) in anhydrous DCM at −78° C. Stirred for 30 to 60 min at approximately −78° C. A solution of an alcohol (1.0 equiv.) in DCM was added slowly keeping the same temperature. Stirring continued. Triethylamine (5.0 equiv.) was added and stirring was continued at the same temperature. The product was isolated by aqueous workup and chromatography if needed.

### General Procedure K (N-Alkylation)

K₂CO₃ was added to a solution of alkyl halide and amine in a solvent such as acetonitrile. Heated. The product was isolated by aqueous workup and chromatography if needed.

### General Procedure L (Formation of Trifluoroacetamide)

Trifluoroacetic anhydride (1.2 equiv.) was added dropwise to a solution of the amine and DIPEA (2.5 equiv.) in an anhydrous solvent such as DCM or DCE at approximately 0° C. The mixture was allowed to warm to room temperature and stirred. The product was isolated by aqueous workup and chromatography if needed.

### General Procedure M (Formation of Imines, Acetals, Thioacetals, Hemiaminals, and Aminals)

The nucleophile such as an amine (1.01 equiv.) was added to a stirred solution of aldehyde in a solvent such as DCE, optionally mixed with H₂O and optionally Na₂CO₃ (2 equiv.) at room temperature and stirred. Evaporated to dryness. Suspended in a solvent such as DCM, filtered and evaporated to give the product.

### General Procedure N (Reduction of Ester to Alcohol)

NaBH₄ (2.0 equiv.) was added at room temperature to a solution of ester in a solvent such as EtOH. Stirred at reflux. The product was isolated by aqueous workup and chromatography if needed.

### General Procedure O (Boc Protection of Amine)

The amine was dissolved in a solvent mixture such as THF/H2O. Di-tert-butyl dicarbonate (1.2 equiv.) was added, followed by NaHCO₃ (4.0 eq). The reaction mixture was stirred at room temperature. The product was isolated by aqueous workup and chromatography if needed.

### General Procedure P (Grignard Displacement of Amide)

The grignard reagent, such as a alkylmagnesium bromide, was added to a solution of amide in a solvent such as THF approximately −78° C. The product was isolated by aqueous workup and column chromatography if needed.

### General Procedure Q (Reduction of Aromatic Rings)

A slurry of the aromatic compound and PtO₂ in a solvent such as MeOH was stirred in the presence of H₂. The product was isolated and purified chromatography if needed.

### General Procedure R (Reduction of Amide or Nitrile to Amine)

Amide or nitrile was added to a suspension of LiAlH₄ (3.0 equiv.) in a solvent such as THF was added at approximately 0° C. Stirred at reflux. Cooled to 0° C. and 4M NaOH solution was added. The product was isolated and purified chromatography if needed.

General Procedure S (Amines from Tert Butyl Carbamates)

HCl in in solvent such as dioxane was added to a solution of tert butyl carbamate in a solvent such as DCM. The mixture was stirred at room temperature. The product was isolated and purified chromatography if needed.

### General Procedure T (Dess-Martin Oxidation)

1,1,1-Triacetoxy-1,1-dihydro-1,2-benziodoxol-3(1H)-one (1.1 equiv.) was added at to a solution of alcohol in a solvent such as DCM and stirred at room temperature. The product was isolated by aqueous workup (Na₂S₂O₃ and NaHCO₃) and column chromatography if needed.

### General Procedure U (N-Oxide to Benzylic Alcohol Rearrangement)

A solution of the N-oxide in acetic anhydride is heated at 100° C. then concentrated in vacuo. The acetate ester was purified by chromatography if needed then dissolved in a solvent such as MeOH and K₂CO₃ (3 equiv.) was added. The reaction mixture was stirred at room temperature. Solvents were removed in vacuo and the product was purified by chromatography if needed.

General Procedure V (mCPBA N-Oxidation of Pyridine)

3-chlorobenzene-1-carboperoxoic acid (1.5 equiv.) was added to a solution of the pyridine in a solvent such as DCM at 0° C. and then stirred at room temperature. The product was isolated by aqueous workup (Na₂S₂O₃ and NaHCO₃) and column chromatography if needed.

### General Procedure X (Suzuki Coupling)

A slurry of the halogenated compound, the boronic acid (2 equiv.), Pd(dba)₂ (0.05 equiv.), SPhos (0.05 equiv.) and K₂CO₃ (3 equiv.) in a mixture solvents such as toluene/water was heated at 100° C. The product was isolated by aqueous workup and column chromatography if needed.

### Synthetic Routes

### Synthetic Route A

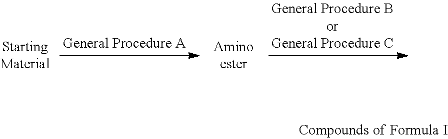

### Synthetic Route B

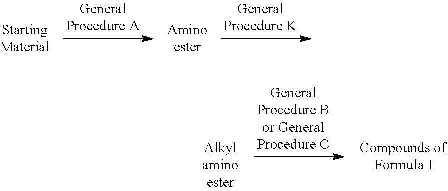

### Synthetic Route C

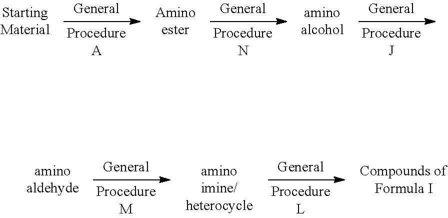

### 6-({[2-Oxo-2-(piperidin-1-yl)ethyl]amino}methyl)pyrimidine-4-carboxylic Acid (Compound 35)

### (By Synthetic Route A)

By General Procedure B from methyl 6-({[2-oxo-2-(piperidin-1-yl)ethyl]amino}methyl)pyrimidine-4-carboxylate. Evaporation to dryness gave the title compound as orange solid as a potassium salt.

¹H-NMR (300 MHz, CD₃OD): δ 9.10 (d, 1H), 7.95 (d, 1H), 3.97 (s, 2H), 3.54 (m, 4H), 3.39 (m, 2H), 1.70-1.50 (m, 6H) ppm.

### Methyl 6-({[2-oxo-2-(piperidin-1-yl)ethyl]amino}methyl)pyrimidine-4-carboxylate

By General Procedure A from methyl 6-formylpyrimidine-4-carboxylate and 2-amino-1-(piperidin-1-yl)ethan-1-one. Purification by preparative TLC gave the title compound as brown oil.

¹H-NMR (300 MHz, CDCl₃): δ 9.30 (s, 1H), 8.17 (s, 1H), 4.06 (s, 2H), 4.04 (s, 3H), 3.58 (m, 2H), 3.50 (m, 2H), 3.29 (m, 2H), 1.70-1.50 (m, 6H) ppm.

### 2-{5H,6H,7H,8H,9H,10H-imidazo[1,2-a][1,4]diazocin-9-ylmethyl}pyridine-4-carboxylic Acid (Compound 51)

### (By Synthetic Route B)

By General Procedure B from methyl 2-{5H,6H,7H,8H,9H,10H-imidazo[1,2-a][1,4]diazocin-9-ylmethyl}pyridine-4-carboxylate. Evaporation to dryness gave the title compound as white solid as a potassium salt.

¹H-NMR (300 MHz, CD₃OD): δ 8.50 (d, 1H), 7.97 (s, 1H), 7.71 (d, 1H), 6.96 (s, 1H), 6.88 (s, 1H), 4.42 (t, 2H), 3.97 (s, 2H), 3.86 (s, 2H), 2.62 (t, 2H), 1.81 (m, 2H), 1.47 (m, 2H) ppm.

### Methyl 2-{5H,6H,7H,8H,9H,10H-imidazo[1,2-a][1,4]diazocin-9-ylmethyl}pyridine-4-carboxylate

By General Procedure K from Methyl 2-{[(1H-imidazol-2-ylmethyl)amino]methyl}pyridine-4-carboxylate and 1,4-dibromobutane. Purification by chromatography gave the title compound as colorless oil.

¹H-NMR (300 MHz, CDCl₃): δ 8.74 (d, 1H), 7.99 (s, 1H), 7.73 (d, 1H), 7.00 (s, 1H), 6.78 (s, 1H), 4.38 (m, 2H), 4.01 (s, 2H), 3.96 (s, 3H), 3.93 (s, 2H), 2.68 (t, 2H), 1.83 (m, 2H), 1.50 (m, 2H) ppm.

### Methyl 2-{[(1H-imidazol-2-ylmethyl)amino]methyl}pyridine-4-carboxylate

By General Procedure A from methyl 2-(aminomethyl)pyridine-4-carboxylate and 1H-imidazole-2-carbaldehyde. Purification by preparative TLC gave the title compound as colorless oil.

¹H-NMR (300 MHz, CDCl₃): δ 8.73 (d, 1H), 7.83 (s, 1H), 7.76 (d, 1H), 7.00 (s, 2H), 4.01 (s, 2H), 3.99 (s, 2H), 3.97 (s, 3H) ppm.

### 2,2,2-trifluoro-1-[6-(2-{5H,6H,7H,8H,9H-imidazo[1,2-a][1,4]diazepin-8-ylmethyl}pyridin-4-yl)-5-oxa-7-azaspiro[2.5]octan-7-yl]ethan-1-one (Compound 108)

### (By Synthetic Route C)

General Procedure L from 6-(2-{5H,6H,7H,8H,9H-imidazo[1,2-a][1,4]diazepin-8-ylmethyl}pyridin-4-yl)-5-oxa-7-azaspiro[2.5]octane gave the title compound colorless glue.

¹H-NMR (300 MHz, CDCl₃): δ 8.64 (m, 1H), 7.38 (m, 1H), 7.14 (m, 1H), 6.81 (m, 2H), 4.28-3.92 (m, 5H), 3.86-3.73 (m, 3H), 3.45-3.06 (m, 4H), 2.03 (m, 3H), 0.68 (m, 2H), 0.44 (m, 2H) ppm.

### 6-(2-{5H,6H,7H,8H,9H-imidazo[1,2-a][1,4]diazepin-8-ylmethyl}pyridin-4-yl)-5-oxa-7-azaspiro[2.5]octane

General Procedure M from 2-{5H,6H,7H,8H,9H-imidazo[1,2-a][1,4]diazepin-8-ylmethyl}pyridine-4-carbaldehyde and [1-(aminomethyl)cyclopropyl]methanol gave the title compound as yellow oil.

¹H-NMR (300 MHz, CDCl₃): δ 853 (m, 1H), 7.66-7.32 (m, 2H), 6.82 (m, 2H), 5.24 (s, 1H), 4.04 (m, 2H), 3.73 (m, 2H), 3.67-3.32 (m, 4H), 3.09 (m, 2H), 2.62 (m, 2H), 1.90 (m, 2H), 0.66-0.35 (m, 4H) ppm.

### 2-{5H,6H,7H,8H,9H-imidazo[1,2-a][1,4]diazepin-8-ylmethyl}pyridine-4-carbaldehyde

General Procedure J from (2-{5H,6H,7H,8H,9H-imidazo[1,2-a][1,4]diazepin-8-ylmethyl}pyridin-4-yl)methanol gave the title product.

¹H-NMR (300 MHz, CDCl₃): δ 10.02 (s, 1H), 8.74 (m, 1H), 7.78 (s, 1H), 7.55 (m, 1H), 6.81 (m, 2H), 4.03 (m, 2H), 3.97 (s, 2H), 3.77 (s, 2H), 3.12 (m, 2H), 1.91 (m, 2H) ppm.

### (2-{5H,6H,7H,8H,9H-imidazo[1,2-a][1,4]diazepin-8-ylmethyl}pyridin-4-yl)methanol

General Procedure N from ethyl 2-{5H,6H,7H,8H,9H-imidazo[1,2-a][1,4]diazepin-8-ylmethyl}pyridine-4-carboxylate gave the title compound as colorless gum.

¹H-NMR (300 MHz, CDCl₃): δ 8.48 (d, 1H), 7.39 (s, 1H), 7.12 (d, 1H), 6.83 (s, 2H), 4.65 (s, 2H), 4.06 (m, 2H), 3.88 (s, 2H), 3.65 (s, 2H), 3.20 (m, 2H), 1.94 (m, 2H) ppm.

### Ethyl 2-{5H,6H,7H,8H,9H-imidazo[1,2-a][1,4]diazepin-8-ylmethyl}pyridine-4-carboxylate

General Procedure A from ethyl 2-formylpyridine-4-carboxylate and 5H,6H,7H,8H,9H-imidazo[1,2-a][1,4]diazepine ethyl pyridine-4-carboxylate gave the title compound as yellow solid.

¹H-NMR (300 MHz, CDCl₃): δ 8.70 (m, 1H), 7.95 (t, 1H), 7.74 (m, 2H), 6.87 (t, 2H), 4.35 (q, 2H), 4.18 (m, 2H), 4.10 (s, 2H), 3.99 (s, 2H), 3.08 (m, 2H), 2.01 (m, 2H), 1.42 (t, 3H) ppm.

### Reagents

### Methyl 6-formylpyrimidine-4-carboxylate

By General Procedure D from diethyl pyrimidine-2,4-dicarboxylate. Purification by column chromatography gave the title compound as yellow solid.

¹H-NMR (300 MHz, CDCl₃): δ 10.13 (s, 1H), 9.37 (d, 1H), 8.16 (d, 1H), 4.04 (s, 3H) ppm.

### Methyl 6-cyanopyridazine-4-carboxylate

A mixture of methyl 6-chloropyridazine-4-carboxylate, Zn(CN)₂, and Pd(PPh₃)₄ in anhydrous DMF was heated to 100° C. for 3 hours under nitrogen. Aqueous work up and purification by column chromatography gave the title product as white solid.

¹H-NMR (300 MHz, CDCl₃): δ 9.83 (d, J=1.9 Hz, 1H), 8.34 (d, J=1.9 Hz, 1H), 4.08 (s, 3H) ppm.

### 3, 5-Dimethyl pyridazine-3, 5-dicarboxylate

Concentrated HCl was added to a solution of methyl 6-cyanopyridazine-4-carboxylate in MeOH. Refluxed overnight. Purification by column chromatography gave the title product as white solid.

¹H-NMR (300 MHz, CDCl₃): δ 9.81 (d, J=2.0 Hz, 1H), 8.68 (d, J=2.0 Hz, 1H), 4.14 (s, 3H), 4.06 (s, 3H) ppm.

### Methyl 3-formylpyridazine-5-carboxylate

By General Procedure D from dimethyl pyridazine-3, 5-dicarboxylate. Aqueous work up gave the title compound as brown oil, which was used without further purification.

¹H-NMR (300 MHz, CDCl₃): δ 10.42 (s, 1H), 9.74 (d, 1H), 8.62 (d, 1H), 4.07 (s, 3H) ppm.

### Ethyl 2-formylpyrimidine-4-carboxylate

By General Procedure D from diethyl pyrimidine-2,4-dicarboxylate. Aqueous work up gave the title compound as brown oil, which was used without further purification.

¹H-NMR (300 MHz, CDCl₃): δ 10.24 (s, 1H), 9.24 (d, 1H), 8.17 (d, 1H), 4.55 (q, 2H), 1.48 (t, 3H) ppm.

### Methyl 2-(2-bromoacetyl)pyridine-4-carboxylate

Bromine (1 equiv.) was added to a solution of methyl 2-acetylpyridine-4-carboxylate and HBr (2 equiv.) in a solvent such as methanol and heated at 60° C. Purification by chromatography gave the title product as white solid.

¹H-NMR (300 MHz, CDCl₃): δ 8.84 (d, 1H), 8.58 (s, 1H), 8.06 (m, 1H), 4.83 (s, 2H), 3.99 (s, 3H) ppm.

### Methyl 2-[2-(methylsulfanyl)acetyl]pyridine-4-carboxylate

Sodium methanethiolate (1 equiv.) was added to a solution of methyl 2-(2-bromoacetyl)pyridine-4-carboxylate in a solvent such as MeOH. Stirred at room temperature. Aqueous work up gave the title compound as orange oil, which was used without further purification.

¹H-NMR (300 MHz, CDCl₃): δ 8.83 (d, 1H), 8.62 (s, 1H), 8.03 (d, 1H), 4.01 (s, 2H), 3.99 (s, 3H), 2.15 (s, 3H) ppm.

### Ethyl 2-(1-hydroxybutyl)pyridine-4-carboxylate

Propylmagnesium bromide (27% in THF) (1 equiv.) was added to solution of ethyl 2-formylpyridine-4-carboxylate in a solvent such as THF at 0° C. Stirred at room temperature. Aqueous work up and purification by chromatography gave the title product as brown oil.

¹H-NMR (300 MHz, CDCl₃): δ 8.70 (d, 1H), 7.82 (s, 1H), 7.76 (d, 1H), 4.85 (m, 1H), 4.32 (q, 2H), 3.90 (br s, 1H), 1.90-1.70 (m, 2H), 1.45 (m, 5H), 1.0 (t, 3H) ppm.

### Ethyl 2-butanoylpyridine-4-carboxylate

By General Procedure J from ethyl 2-(1-hydroxybutyl)pyridine-4-carboxylate. Aqueous work up gave the title compound as yellow oil, which was used without further purification.

¹H-NMR (300 MHz, CDCl₃): δ 8.80 (d, 1H), 8.50 (s, 1H), 8.00 (d, 1H), 4.45 (q, 2H), 3.20 (t, 2H), 1.80 (m, 2H), 1.40 (t, 3H), 1.0 (t, 3H) ppm.

### 1-Ethyl-2-oxopyrrolidine-3-carbaldehyde

1-Ethyl pyrrolidin-2-one in THF was treated by lithium diisopropyl amine (1.2 equiv.) and DMF (1.5) at −78° C. The product was isolated by aqueous workup to give the title compound as brown oil.

¹H-NMR (300 MHz, CDCl₃): δ 9.98 (s, 1H), 3.42-3.30 (m, 4H), 3.26-3.21 (m, 1H) 1.08 (t, 3H) ppm.

### (R) or (S)-3-(benzyloxymethyl)-1-((R)-1-phenylethyl)pyrrolidin-2-one

By General Procedure E from (R)-1-(1-phenylethyl)pyrrolidin-2-one and BOMCl. The diasteromers were separated by chromatography.

(R, R)-isomer: ¹H-NMR (300 MHz, CDCl₃), δ ppm: 7.38-7.28 (m, 10H), 5.53 (q, 1H), 4.56 (dd, 2H), 3.78 (s, 2H), 3.38-3.30 (m, 1H), 2.96-2.87 (m, 1H), 2.76-2.67 (m, 1H), 2.18-1.92 (m, 2H), 1.54 (d, 3H). (S, R)-isomer: ¹H-NMR (300 MHz, CDCl₃), δ ppm: 7.36-7.23 (m, 10H), 5.52 (q, 1H), 4.56 (dd, 2H), 3.84-3.75 (m, 2H), 3.32-3.22 (m, 1H), 3.07-2.98 (m, 1H), 2.83-2.72 (m, 1H), 2.25-2.12 (m, 1H), 2.02-1.91 (m, 1H), 1.55 (d, 3H)

### (S)-3-(hydroxymethyl)-1-((R)-1-phenylethyl)pyrrolidin-2-one

By General Procedure F using (S)-3-(benzyloxymethyl)-1-((R)-1-phenylethyl)pyrrolidin-2-one.

¹H-NMR (300 MHz, CDCl₃), δ ppm: 7.37-7.26 (m, 5H), 5.47 (q, 1H), 3.86 (dd, 1H), 3.73 (dd, 1H), 3.30 (dd, 1H), 3.00 (td, 1H), 2.79-2.69 (m, 1H) 2.15-2.05 (m, 1H), 1.75-1.61 (m, 1H), 1.53 (d, 3H)

### (S)-2-oxo-1-((R)-1-phenylethyl)pyrrolidin-3-yl)methyl 4-methylbenzenesulfonate

By General Procedure G from (S)-3-(hydroxymethyl)-1-((R)-1-phenylethyl)pyrrolidin-2-one. The product was purified by chromatography.

¹H-NMR (300 MHz, CDCl₃), δ ppm: 7.75 (d, 2H), 7.37-7.23 (m, 7H), 5.44 (q, 1H), 4.33 (dd, 1H), 4.19 (dd, 1H), 3.26 (q, 1H), 3.04-2.96 (m, 1H), 2.87-2.78 (m, 1H), 2.45 (s, 3H), 2.28-2.18 (m, 1H), 1.92-1.79 (m, 1H), 1.53 (d, 3H).

### (S)-3-(azidomethyl)-1-((R)-1-phenylethyl)pyrrolidin-2-one

By General Procedure H from (S)-2-oxo-1-((R)-1-phenylethyl)pyrrolidin-3-yl)methyl 4-methylbenzenesulfonate

¹H-NMR (300 MHz, CDCl₃), δ ppm: 7.37-7.24 (m, 5H), 5.49 (q, 1H), 3.64 (d, 2H), 3.26 (q, 1H), 2.98 (td, 1H), 2.77-2.67 (m, 1H), 2.23-2.12 (m, 1H), 1.87-1.74 (m, 1H), 1.53 (d, 3H)

### (S)-3-(aminomethyl)-1-((R)-1-phenylethyl)pyrrolidin-2-one

By general procedure I from (S)-3-(azidomethyl)-1-((R)-1-phenylethyl)pyrrolidin-2-one. The product was purified by chromatography.

¹H-NMR (300 MHz, CDCl₃), δ ppm: 7.31-7.23 (m, 5H), 5.47 (q, 1H), 3.25 (q, 1H), 2.99-2.87 (m, 3H), 2.61-2.52 (m, 1H), 2.15-2.06 (m, 1H), 1.75-1.62 (m, 1H), 1.52 (d, 5H).

### (R) or (S)-3-(benzyloxymethyl)-1-((R)-1-phenylethyl)piperidin-2-one

By General Procedure E from (R)-1-(1-phenylethyl)piperidin-2-one and BOMCl. The diasteromers were separated by chromatography.

(R, R)-Isomer: ¹H-NMR (300 MHz, CDCl₃), δ ppm: 7.38-7.28 (m, 10H), 6.16 (q, 1H), 4.59 (d, 2H), 3.88 (d, 2H), 3.18-3.10 (m, 1H), 2.90-2.82 (m, 1H), 2.74-2.66 (m, 1H), 2.06-1.98 (m, 1H), 1.89-1.75 (m, 2H), 1.64-1.53 (m, 1H), 1.51 (d, 3H). (S, R)-Isomer: ¹H-NMR (300 MHz, CDCl₃), δ ppm: 7.35-7.26 (m, 10H), 6.15 (q, 1H), 4.58 (s, 2H), 3.96 (dd, 1H), 3.84 (dd, 1H), 3.12-3.04 (m, 1H), 2.80-2.68 (m, 2H), 2.02-1.94 (m, 1H), 1.90-1.76 (m, 2H), 1.71-1.61 (m, 1H), 1.52 (d, 3H)

### (3R)-3-(aminomethyl)-1-[(1R)-1-phenylethyl]piperidin-2-one

Prepared analogously to (S)-3-(aminomethyl)-1-((R)-1-phenylethyl)pyrrolidin-2-one from (R)-3-(benzyloxymethyl)-1-((R)-1-phenylethyl)piperidin-2-one.

¹H-NMR (300 MHz, CDCl₃), δ ppm: 7.53-7.24 (m, 5H), 6.11 (q, 1H), 3.15-2.96 (m, 3H), 2.89-2.82 (m, 1H), 2.51-2.42 (m, 1H), 2.23 (s, 2H), 1.95-1.87 (m, 1H), 1.85-1.76 (m, 1H), 1.67-1.57 (m, 2H), 1.50 (d, 3H)

### (3S)-3-(aminomethyl)-1-[(1R)-1-phenylethyl]piperidin-2-one

Prepared analogously to (S)-3-(aminomethyl)-1-((R)-1-phenylethyl)pyrrolidin-2-one from (S)-3-(benzyloxymethyl)-1-((R)-1-phenylethyl)piperidin-2-one.

¹H-NMR (300 MHz, CDCl₃), δ ppm: 7.36-7.26 (m, 5H), 6.09 (q, 1H), 3.14-3.05 (m, 1H), 3.02 (d, 2H), 2.78-2.69 (m, 1H), 2.50-2.43 (m, 1H), 2.37 (s, 2H), 1.94-1.83 (m, 1H), 1.73-1.66 (m, 2H), 1.63-1.53 (m, 1H), 1.49 (d, 3H).

### (3R)-3-(aminomethyl)-1-[(1R)-1-phenylethyl]pyrrolidin-2-one

Prepared analogously to (S)-3-(aminomethyl)-1-((R)-1-phenylethyl)pyrrolidin-2-one.

¹H-NMR (300 MHz, CDCl₃): δ 7.36-7.24 (m, 5H), 5.55 (q, 1H), 3.28 (dt, 1H), 3.02-2.85 (m, 3H), 2.57-2.48 (m, 1H), 2.12-2.12 (m, 1H), 1.86-1.73 (m, 1H), 1.55-1.49 (d, 5H).

### (3S)-3-(aminomethyl)-1-[(1R)-1-(4-methoxyphenyl)ethyl]pyrrolidin-2-one

Prepared analogously to (S)-3-(aminomethyl)-1-((R)-1-phenylethyl)pyrrolidin-2-one.

¹H-NMR (300 MHz, CDCl₃): δ 7.21 (d, 2H), 6.85 (d, 2H), 5.42 (q, 1H) 3.80 (s, 3H), 3.24 (q, 1H), 3.01-2.89 (m, 3H), 2.65-2.55 (m, 1H), 2.17-2.06 (m, 3H), 1.73-1.63 (m, 1H), 1.49 (d, 3H).

### (3S)-3-(aminomethyl)-1-[(1R)-1-(4-methoxyphenyl)ethyl]piperidin-2-one

Prepared analogously to (S)-3-(aminomethyl)-1-((R)-1-phenylethyl)pyrrolidin-2-one

¹H-NMR (300 MHz, CDCl₃): δ 7.20 (d, 2H), 6.86 (d, 2H), 6.06 (q, 1H), 3.81 (s, 3H), 3.1-3.03 (m, 1H), 3.0 (d, 2H), 2.77-2.69 (m, 1H), 2.46-2.37 (m, 1H), 1.91-1.83 (m, 1H), 1.71 (s, 2H), 1.69-1.54 (m, 3H), 1.47 (d, 3H).

### (3R)-3-(aminomethyl)-1-[(1R)-1-(4-methoxyphenyl)ethyl]piperidin-2-one

Prepared analogously to (S)-3-(aminomethyl)-1-((R)-1-phenylethyl)pyrrolidin-2-one.

¹H-NMR (300 MHz, CDCl₃): δ 7.21 (d, 2H), 6.85 (d, 2H), 5.44 (q, 1H), 3.78 (s, 3H), 3.25 (td, 1H), 2.98-2.91 (m, 2H), 2.87 (q, 1H), 2.54-2.44 (m, 1H), 2.10-2.00 (m, 1H), 1.83-1.70 (m, 1H1.48 (d, 3H).

### (3R)-3-(aminomethyl)-1-[(1R)-1-(4-methoxyphenyl)ethyl]pyrrolidin-2-one

Prepared analogously to (S)-3-(aminomethyl)-1-((R)-1-phenylethyl)pyrrolidin-2-one.

¹H-NMR (300 MHz, CDCl₃): δ 7.22 (d, 2H), 6.86 (d, 2H), 6.08 (q, 1H), 3.81 (s, 3H), 3.14-2.98 (m, 3H), 2.90-2.86 (m, 1H), 2.47-2.37 (m, 1H), 1.95-1.86 (m, 1H), 1.84-1.78 (m, 1H), 1.74 (s, 2H), 1.65-1.57 (m, 2H), 1.47 (d, 3H).

### (3R)-3-(aminomethyl)-1-ethylpiperidin-2-one

Prepared from (R)-3-(benzyloxymethyl)-1-ethylpiperidin-2-one analogously to (S)-3-(aminomethyl)-1-((R)-1-phenylethyl)pyrrolidin-2-one from (R) or (S)-3-(benzyloxymethyl)-1-((R)-1-phenylethyl)pyrrolidin-2-one.

¹H-NMR (300 MHz, CDCl₃): δ 3.48-3.32 (m, 2H), 3.29-3.25 (m, 2H), 3.94 (d, 2H), 2.36-2.27 (m, 1H), 1.94-1.84 (m, 2H), 1.81-1.58 (m, 4H), 1.11 (t, 3H)

### (R)-3-(benzyloxymethyl)-1-ethylpiperidin-2-one

(R)-3-(benzyloxymethyl)piperidin-2-one was dissolved dimethylformamide at 0° C. Sodium hydride was added and the mixture was stirred. Ethyl iodide was added and the mixture was warmed to room temperature. The product was isolated by aqueous workup and column chromatography.

¹H-NMR (300 MHz, CDCl₃): δ 7.37-7.29 (m, 5H), 4.52 (s, 2H), 3.87-3.73 (m, 2H), 3.40 (q, 2H), 3.33-3.25 (m, 2H), 2.62-2.54 (m, 1H), 2.06-1.99 (m, 1H), 1.95-1.86 (m, 1H), 1.83-1.72 (m, 2H), 1.12 (t, 3H).

### (R)-3-(benzyloxymethyl)piperidin-2-one

(R)-3-(benzyloxymethyl)-1-((R)-1-(4-methoxyphenyl)ethyl)piperidin-2-one was dissolved in acetonitrile at 0° C. An aqueous solution of Ceric ammonium nitrate (2.2 eq). The product was isolated by aqueous workup and column chromatography.

¹H-NMR (300 MHz, CDCl₃): δ 7.35-7.31 (m, 5H), 7.03 (bs, 1H), 4.51 (s, 2H), 3.83 (dd, 2H), 3.37 (dd, 2H), 2.71-2.62 (m, 1H), 2.13-2.04 (m, 1H), 1.98-1.92 (m, 2H), 1.84-1.73 (m, 1H).

### (3S)-3-(aminomethyl)-1-ethylpyrrolidin-2-one

Prepared analogously to (3R)-3-(aminomethyl)-1-ethylpiperidin-2-one.

¹H-NMR (300 MHz, CDCl₃), δ 3.36-3.26 (m, 4H), 2.91 (bs, 2H), 2.56-2.45 (m, 1H), 2.21-2.10 (m, 1H), 1.87-1.75 (m, 1H), 1.50 (bs, 2H), 1.10 (t, 3H).

### (3S)-3-(aminomethyl)-1-ethylpiperidin-2-one

Prepared analogously to (3R)-3-(aminomethyl)-1-ethylpiperidin-2-one.

¹H-NMR (300 MHz, CDCl₃): δ 3.48-3.32 (m, 2H), 3.29-3.25 (m, 2H), 3.94 (d, 2H), 2.36-2.27 (m, 1H), 1.94-1.84 (m, 2H), 1.81-1.58 (m, 4H), 1.11 (t, 3H).

### (3R)-3-(aminomethyl)-1-ethylpyrrolidin-2-one

Prepared analogously to (3R)-3-(aminomethyl)-1-ethylpiperidin-2-one.

¹H-NMR (300 MHz, CDCl₃), δ 3.36-3.26 (m, 4H), 2.91 (bs, 2H), 2.56-2.45 (m, 1H), 2.21-2.10 (m, 1H), 1.87-1.75 (m, 1H), 1.50 (bs, 2H), 1.10 (t, 3H).

### 5H,6H,7H,8H,9H-imidazo[1,2-a][1,4]diazepine

tert-butyl N-[3-(2-formyl-1H-imidazol-1-yl)propyl]carbamate and 4M HCl in dioxane stirred at rt, after basic work up and concentration, the crude compound was dissolved in MeOH, NaBH₄ (1.2 equiv.) was added. Stirred at rt. Basic work gave the title compound which was used for next step without further purification.

¹H-NMR (300 MHz, CDCl₃): δ 6.79 (d, 2H), 4.18 (m, 2H), 4.02 (s, 2H), 2.60 (m, 2H), 1.98 (m, 2H) ppm.

### (S)-3-methyl-1-(tetrahydrofuran-2-yl)butan-1-one

General Procedure P from (S)—N-methoxy-N-methyltetrahydrofuran-2-carboxamide gave the title product.

¹H-NMR (300 MHz, CDCl₃): δ 4.26 (dd, 1H), 3.97-3.85 (m, 2H), 2.49-2.30 (m, 2H), 2.20-2.11 (m, 2H), 1.92-1.84 (m, 3H), 0.91 (dd, 6H).

### (3-cyclohexylpiperidin-2-yl)methanol

General Procedure Q from (3-phenylpyridin-2-yl)methanol gave the title compound as colorless oil.

¹H-NMR (300 MHz, CDCl₃): δ 3.60 (m, 1H), 3.40 (m, 1H), 3.00 (m, 1H), 2.50 (m, 3H), 1.60 (m, 13H).

### (R)-tert-butyl 1-(methoxy(methyl)amino)-4-methyl-1-oxopentan-2-ylcarbamate

General Procedure P from tert-butyl N-{1-[methoxy(methyl)carbamoyl]-3-methylbutyl}carbamate and allylmagnesium bromide. The reaction mixture was diluted with methanol and NaBH₄ was added to the mixture. The title product was isolated by column chromatography.

¹H-NMR (300 MHz, CDCl₃): δ 5.94-5.80 (m, 1H), 5.15 (dd, 2H), 4.57 (bs, 1H), 3.73-3.65 (m, 1H), 2.45 (bs, 1H), 2.30-2.13 (m, 2H), 1.72-1.62 (m, 1H), 1.64 (s, 1H), 1.45 (s, 9H), 1.34-1.31 (m, 2H), 0.94 (dd, 6H)

### Tert-butyl (2R,3S)-3-hydroxy-5-methyl-2-(2-methylpropyl)pyrrolidine-1-carboxylate

From tert-butyl N-(5-hydroxy-2-methyloct-7-en-4-yl)carbamate following the procedure outlined in Eur. J. Org. Chem., 2004, 1973-1982 and Tetrahedron Lett. 2002, 43, 6771-6773. After aqueous workup the crude was subject to Pd/C according to General Procedure I. Column chromatography gave the title product.

¹H-NMR (300 MHz, CDCl₃): δ 4.13 (s, 1H), 3.98-3.72 (m, 2H), 2.33-2.24 (m, 1H), 1.79-1.59 (m, 4H), 1.48 (s, 10H), 1.42-1.39 (m, 3H), 1.01-0.96 (d, 3H), 0.92 (d, 3H).

### (2R,3S)-5-methyl-2-(2-methylpropyl)pyrrolidin-3-ol

A compound such as tert-butyl (2R,3S)-3-hydroxy-5-methyl-2-(2-methylpropyl)pyrrolidine-1-carboxylate was treated with a reagent such as conc. HCl. The product was obtained concentration and neutralization of the HCl salt with a reagent such as KOH.

¹H-NMR (300 MHz, CD₃OD): δ 4.17 (q, 1H), 3.84-3.76 (m, 1H), 3.72-3.60 (m, 1H), 3.46 (q, 1H), 2.56-2.57 (m, 1H), 1.83-1.65 (m, 2H), 1.63-1.54 (m, 2H), 1.49 (d, 3H), 1.03 (dd, 6H).

### {5H,6H,7H,8H,9H-imidazo[1,2-a][1,4]diazepin-5-ylmethyl}dimethylamine

General Procedure R from N,N-dimethyl-5H,6H,7H,8H,9H-imidazo[1,2-a][1,4]diazepine-5-carboxamide gave the title product as yellow gum.

ES-MS: 195 [M+1].

### N,N-dimethyl-5H,6H,7H,8H,9H-imidazo[1,2-a][1,4]diazepine-5-carboxamide

General Procedure S from tert-butyl 5-(dimethylcarbamoyl)-5H,6H,7H,8H,9H-imidazo[1,2-a][1,4]diazepine-8-carboxylate gave the title compound.

¹H-NMR (300 MHz, CD₃OD): δ 7.7 (s, 1H),), 7.6 (s, 1H), 6.2 (m, 1H), 3.6 (m, 2H), 3.2 (s, 3H), 2.8 (s, 3H), 2.5 (m, 2H).

### Tert-butyl 5-(dimethylcarbamoyl)-5H,6H,7H,8H,9H-imidazo[1,2-a][1,4]diazepine-8-carboxylate

1,1′-Carbonyldiimidazole was added to a solution of tert-butyl N-[4-(dimethylamino)-3-hydroxybutyl]-N-(1H-imidazol-2-ylmethyl) carbamate in CH₃CN. The mixture was heated in Microwave at 150° C. The title compound was isolated as colorless gum.

¹H-NMR (300 MHz, CD₃OD): δ 6.9 (s, 1H), 6.7 (s, 1H), 5.2 (m, 1H), 4.7 (m, 2H), 3.2 (s, 3H), 2.9 (s, 3H), 2.2 (m, 2H), (1.4 s, 9H).

### Tert-butyl N-[4-(dimethylamino)-3-hydroxybutyl]-N-(1H-imidazol-2-ylmethyl)carbamate

General Procedure O from [4-(dimethylamino)-3-hydroxybutyl](1H-imidazol-2-ylmethyl)amine gave the title product as light yellow gum.

¹H-NMR (300 MHz, CDCl₃): δ 6.9 (brs, 2H), 4.5 (m, 3H), 3.5 (m, 2H), 2.9 (s, 3H), 2.8 (s, 3H), 2.5 (m, 1H), 2.2 (m, 1H), 1.4 (s, 9H).

### [4-(dimethylamino)-3-hydroxybutyl](1H-imidazol-2-ylmethyl) Amine

General Procedure A (1H-imidazole-2-carbaldehyde) and 4-amino-2-hydroxy-N,N-dimethylbutanamide gave the title product.

¹H-NMR (300 MHz, CDCl₃): δ 6.9 (s, 1H), 6.8 (s, 1H), 4.4 (m, 1H), 3.9 (s, 2H), 3.2 (m, 2H), 2.8 (s, 3H), 2.7 (s, 3H).

### 4-amino-2-hydroxy-N,N-dimethylbutanamide

General Procedure S from tert-butyl N-[4-(dimethylamino)-3-hydroxybutyl]carbamate gave the title compound.

¹H-NMR (300 MHz, CD₃OD): δ 4.6 (m, 1H), 3.1 (brs, 5H), 2.8 (s, 3H), 1.9 (m, 2H)

### Tert-butyl N-[4-(dimethylamino)-3-hydroxybutyl]carbamate

The title compound was obtained analogously to the procedures outlined in J. Org. Chem. (2006) 71(9) 3364-3374.

¹H NMR (300 MHz, CDCl₃), δ ppm: 5.0 (brs, 1H), 4.5 (m, 1H), 3.8 (m, 1H), 3.4 (m, 2H), 3.1 (s, 3H), 3.2 (s, 1H), 2.0 (m, 1H), 1.6 (m, 1H), 1.4 (s, 9H).

### 5-(4-fluorophenyl)-5H,6H,7H,8H,9H-imidazo[1,2-a][1,4]diazepine

General Procedure S from tert-butyl 5-(4-fluorophenyl)-5H,6H,7H,8H,9H-imidazo[1,2-a][1,4]diazepine-8-carboxylate gave the title compound.

¹H-NMR (300 MHz, CDCl₃): 7.4 (m, 2H), 7.0 (m, 4H), 4.5 (m, 1H), 3.0 (m, 2H), 2.2 (m, 2H) ES-MS: 232 [M+1].

### Tert-butyl 5-(4-fluorophenyl)-5H,6H,7H,8H,9H-imidazo[1,2-a][1,4]diazepine-8-carboxylate

1,1′-Carbonyldiimidazole was added to a solution of tert-butyl N-[4-(dimethylamino)-3-hydroxybutyl]-N-(1H-imidazol-2-ylmethyl) carbamate in CH₃CN. The mixture was heated in Microwave at 150° C. The title compound was isolated as colorless gum.

¹H-NMR (300 MHz, CDCl₃): δ 8.1 (brs, 1H), 7.4 (m, 2H), 7.0 (m, 2H), 6.9 (brs, 1H), 6.0 (m, 1H), 4.7 (m, 2H), 3.5 (m, 2H), 2.3 (m, 2H), 1.4 (s, 9H).

### Tert-butyl N-[3-(4-fluorophenyl)-3-hydroxypropyl]-N-(1H-imidazol-2-ylmethyl)carbamate

General Procedure O from 1-(4-fluorophenyl)-3-[(1H-imidazol-2-ylmethyl)amino]propan-1-ol) gave the title product as light yellow gum.

¹H-NMR (300 MHz, CDCl₃): δ 7.4 (brs, 1H), 7.3 (brs, 1H), 7.0 (m, 4H), 4.7 (m, 1H), 4.1 (s, 2H), 3.1 (m, 2H), 1.9 (m, 2H), 1.4 (s, 9H).

### 1-(4-fluorophenyl)-3-[(1H-imidazol-2-ylmethyl)amino]propan-1-ol

General Procedure A from 1H-imidazole-2-carbaldehyde and 3-amino-1-(4-fluorophenyl)propan-1-ol gave the title compound.

¹H-NMR (300 MHz, CDCl₃): δ 7.3 (m, 2H), 7.0 (m, 4H), 4.9 (m, 1H), 3.9 (s, 2H), 2.9 (m, 2H), 1.8 (m, 2H).

### 3-amino-1-(4-fluorophenyl)propan-1-ol

General Procedure R gave the title product as yellow gum.

¹H-NMR (300 MHz, CDCl₃): δ 7.3 (m, 2H), 6.9 (m, 2H), 4.4 (m, 1H), 2.9 (m, 2H), 1.7 (m, 2H)

### 5H,6H,7H-pyrrolo[1,2-a]imidazol-7-amine

By General Procedure I from 7-azido-5H,6H,7H-pyrrolo[1,2-a]imidazole.

¹H-NMR (300 MHz, CD₃OD): δ 7.02 (s, 1H), 6.93 (s, 1H), 4.56 (m, 1H), 4.07 (m, 1H), 3.92 (m, 1H), 2.97 (m, 1H), 2.49 (m, 1H) ppm.

### 7-azido-5H,6H,7H-pyrrolo[1,2-a]imidazole

Made from 5H,6H,7H-pyrrolo[1,2-a]imidazol-7-ol by treating with MsCl, NaN₃ and DMAP (cat.) in mixture of THF and DMSO. Purification by chromatography gave the title compound as colorless oil.

¹H-NMR (300 MHz, CD₃OD): δ 7.67 (s, 1H), 6.84 (s, 1H), 5.19 (m, 1H), 4.18 (m, 1H), 3.90 (m, 1H), 2.92 (m, 1H), 2.57 (m, 1H) ppm.

### 4-Benzyl-5H,6H,7H-cyclopenta[b]pyridin-7-one

General Procedure T from 4-benzyl-5H,6H,7H-cyclopenta[b]pyridin-7-ol gave the title compound.

¹H-NMR (300 MHz, CDCl₃): δ 8.69 (d, 1H), 7.38-7.24 (m, 3H), 7.22 (d, 1H), 7.17 (d, 2H), 4.09 (s, 2H), 3.01 (dd, 2H), 2.73 (dd, 2H) ppm.

### 4-Benzyl-5H,6H,7H-cyclopenta[b]pyridin-7-ol

General Procedure U from 4-benzyl-5H,6H,7H-cyclopenta[b]pyridin-1-ium-1-olate gave the title compound.

¹H-NMR (300 MHz, CDCl₃): δ 8.34 (d, 1H), 7.37-7.22 (m, 3H), 7.15 (d, 2H), 6.94 (d, 1H), 5.22 (t, 1H), 3.96 (s, 2H), 3.04-2.91 (m, 1H), 2.77-2.47 (m, 2H), 2.12-1.96 (m, 1H) ppm.

### 4-Benzyl-5H,6H,7H-cyclopenta[b]pyridin-1-ium-1-olate

General Procedure V from 4-benzyl-5H,6H,7H-cyclopenta[b]pyridine gave the title compound.

¹H-NMR (300 MHz, CDCl₃): δ 8.00 (d, 1H), 7.36-7.20 (m, 3H), 7.12 (d, 2H), 6.84 (d, 1H), 3.88 (s, 2H), 3.18 (t, 2H), 2.90 (t, 2H), 2.16 (m, 2H) ppm.

### 4-Benzyl-5H,6H,7H-cyclopenta[b]pyridine

General Procedure X from 4-chloro-5H,6H,7H-cyclopenta[b]pyridine and 2-benzyl-4,4,5,5-tetramethyl-1,3,2-dioxaborolane gave the title compound.

¹H-NMR (300 MHz, CDCl₃): δ 8.27 (d, 1H), 7.36-7.19 (m, 3H), 7.15 (d, 2H), 6.82 (d, 1H), 3.93 (s, 2H), 3.03 (t, 2H), 2.85 (t, 2H), 2.11 (m, 2H) ppm.

### 4-ethyl-5H,6H,7H-cyclopenta[b]pyridin-7-one

Prepared analogously to 4-Benzyl-5H,6H,7H-cyclopenta[b]pyridin-7-one.

¹H-NMR (300 MHz, CDCl₃): δ 8.68 (d, 1H), 7.29 (d, 1H), 3.09 (dd, 2H), 2.81-2.70 (m, 4H), 1.32 (t, 3H) ppm.

### 4-(propan-2-yl)-5H,6H,7H-cyclopenta[b]pyridin-7-one

Prepared analogously to 4-Benzyl-5H,6H,7H-cyclopenta[b]pyridin-7-one.

¹H-NMR (300 MHz, CDCl₃): δ 8.70 (d, 1H), 7.32 (d, 1H), 3.20-3.05 (m, 3H), 2.79-2.72 (m, 2H), 1.31 (d, 6H) ppm.

### 4-ethenyl-5H,6H,7H-cyclopenta[b]pyridin-7-one

Prepared analogously to 4-Benzyl-5H,6H,7H-cyclopenta[b]pyridin-7-one.

¹H-NMR (300 MHz, CDCl₃): δ 8.71 (d, 1H), 7.47 (d, 1H), 6.82 (dd, 1H), 6.05 (br s, 1H), 5.68 (d, 1H), 3.17 (dd, 2H), 2.76 (dd, 2H) ppm.

### 4-(2-phenylethyl)-5H,6H,7H-cyclopenta[b]pyridin-7-one

Prepared analogously to 4-Benzyl-5H,6H,7H-cyclopenta[b]pyridin-7-one.

¹H-NMR (300 MHz, CDCl₃): δ 8.67 (d, 1H), 7.33-7.19 (m, 4H), 7.11 (d, 2H), 3.06-2.96 (m, 4H), 2.88 (dd, 2H), 2.69 (dd, 2H) ppm.

### 3-(prop-2-en-1-yl)-5H,6H,7H-cyclopenta[b]pyridin-7-one

Prepared analogously to 4-Benzyl-5H,6H,7H-cyclopenta[b]pyridin-7-one.

¹H-NMR (300 MHz, CDCl₃): δ 8.69 (s, 1H), 7.85 (s, 1H), 5.97 (m, 1H), 5.29 (m, 2H), 3.46 (m, 1H), 3.04 (m, 2H), 2.29 (m, 2H) ppm.

### 4-Methoxy-5H,6H,7H-cyclopenta[b]pyridin-7-amine

By General Procedure I from 7-azido-4-methoxy-5H,6H,7H-cyclopenta[b]pyridine. The crude material was used in the next step without any further purification.

### 7-Azido-4-methoxy-5H,6H,7H-cyclopenta[b]pyridine

General Procedure H from 4-methoxy-5H,6H,7H-cyclopenta[b]pyridin-7-yl methanesulfonate and sodium azide gave the title compound.

¹H-NMR (300 MHz, CDCl₃): δ 8.40 (d, 1H), 6.68 (d, 1H), 4.88 (dd, 1H), 3.87 (s, 3H), 3.03-2.90 (m, 1H), 2.85-2.72 (m, 1H), 2.53-2.39 (m, 1H), 2.12-1.99 (m, 1H) ppm.

### 4-Methoxy-5H,6H,7H-cyclopenta[b]pyridin-7-yl methanesulfonate

By General Procedure G from 4-methoxy-5H,6H,7H-cyclopenta[b]pyridin-7-ol. The crude material was used in the next step without any further purification.

¹H-NMR (300 MHz, CDCl₃): δ 8.44 (d, 1H), 6.76 (d, 1H), 6.01 (dd, 1H), 3.92 (s, 3H), 3.24 (s, 3H), 3.21-3.10 (m, 1H), 2.96-2.83 (m, 1H), 2.70-2.52 (m, 1H), 2.48-2.36 (m, 1H) ppm.

### 4-Methoxy-5H,6H,7H-cyclopenta[b]pyridin-7-ol

General Procedure U from 4-methoxy-5H,6H,7H-cyclopenta[b]pyridin-1-ium-1-olate gave the title compound.

¹H-NMR (300 MHz, CDCl₃): δ 8.34 (d, 1H), 6.65 (d, 1H), 6.44 (br s, 1H), 5.26 (t, 1H), 3.87 (s, 3H), 3.06-2.94 (m, 1H), 2.78-2.65 (m, 1H), 2.59-2.45 (m, 1H), 2.12-1.98 (m, 1H) ppm.

### 4-bromo-5H,6H,7H-cyclopenta[b]pyridin-7-one

Prepared from 4-Bromo-5H,6H,7H-cyclopenta[b]pyridin-1-ium-1-olate analogously to 4-Benzyl-5H,6H,7H-cyclopenta[b]pyridin-7-one from 4-Benzyl-5H,6H,7H-cyclopenta[b]pyridin-1-ium-1-olate.

¹H-NMR (300 MHz, CDCl₃): δ 8.56 (d, 1H), 7.65 (d, 1H), 3.19 (dd, 2H), 2.79 (dd, 2H).

### Example 2: Histone Lysine Demethylase AlphaLISA Assays for IC₅₀ Value Determination

This example demonstrates the ability of compounds of the disclosure to inhibit the activity in vitro of tested enzymes.

Assays are performed analogously to the protocol described by PerkinElmer (Roy et al. PerkinElmer Technical Note: AlphaLISA #12, April 2011).

### General Method

Enzymes (final assay concentration 0.1-2.5 nM) are dissolved in enzyme buffer and incubated for 10 min before 5 μL is added to 5 μL 3% DMSO solutions of compounds in enzyme buffer, incubated for another 10 minutes, before 5 μL substrate solution is added and the reaction mixture is incubated at room temperature. 10 μL acceptor beads, suspended Epigenetic Buffer (Perkin Elmer AL008) from stock, are added and the suspension is incubated in the dark at room temperature, before 10 μL suspension of streptavidin donor beads (Perkin Elmer 6760002) in Epigenetic Buffer is added. After incubation at room temperature in the dark the plates are read.

### Enzymes:

| | | | Expression |
|---|---|---|---|
| Protein name | Vendor/source | Sequence | organism |
| KDM2B | BPS, | 1-650 | Bac |
| (FBXL10) | Bioscience, | | |
| | US | | |
| KDM3B | BRIC | 842-1761 | Bac |
| (JMJD1B) | | | |
| KDM4A | BPS, | 1-350 | E. coli |
| (JMJD2A) | Bioscience, | | |
| | US | | |
| KDM4B | BPS | 2-500 | Bac |
| (JMJD2B) | | | |
| KDM4C | BRIC, | 1-349 | E. coli |
| (JMJD2C) | Denmark | | |
| KDM5C | BPS | 2-1560 | Bac |
| (JARID1C) | | | |
| KDM5B | BRIC | 1-809 | E. coli |
| (PLU-1) | | | |
| KDM6A | BRIC | 919-1401 | E. coli |
| (UTX) | | | |
| KDM6B | BPS | 1043-end | Bac |
| (JMJD3) | | | |
| KDM7 | BRIC | 1-1322 | Bac |
| (PHF8) | | | |
| KDM3A | BPS, | 2-end | Bac |
| (JMJD1A) | Bioscience, | | |
| | US | | |

### Substrates:

| | |
|---|---|
| BK9M2: | Biotin-ARTKQTAR(KMe₂)STGGKAPRKQ-NH₂ (AnaSpec 64359) |
| BK9M1: | Biotin-ARTKQTAR(KMe₁)STGGKAPRKQ-NH₂ (AnaSpec 64358) |
| H3K4M3B: | H-ART(Kme3)QTARKSTGGKAPRKQLA-NH-Biotin (Caslo, Denmark) |
| BK27M3: | Biotin-ATKAAR(Kme3)SAPATGGVKKPHRY-NH2 (Caslo, Denmark) |
| BH3K36M2: | RKAAPATGGVK(Me2)KPHRYRPGTVK-(BIOTIN) (Anaspec) |

Substrate solution: Substrate (final assay concentration 50-200 nM), 50 mM Hepes (pH 7.4-8.0), 0.003% Tween-20, 0.1% BS, 25 μM L-Asc, 10 μM α-KG.
Enzyme Buffer: 50 mM Hepes (pH 7.4-8.0), 0.003% Tween-20, 0.1% BSA; 5 μM (NH₄)₂Fe(SO₄)₂

### HDME Inhibition

| Compound Name | Compound # | KDM4C | KDM2B | PHF8 | KDM6A | KDM5B |
|---|---|---|---|---|---|---|
| 2-(1-{[(1S)-1-{[(3- | 1 | +(a) | | | + | |
| carboxyphenyl)methyl]carbamoyl}eth- | | | | | | |
| yl]amino}ethyl)pyridine-4-carboxylic | | | | | | |
| acid | | | | | | |
| 2-[({[(3R)-2-oxo-1-[(1R)- | 2 | +++ | +++ | ++ | + | +++ |
| 1-phenylethyl]piperidin-3- | | | | | | |
| yl]methyl}amino)methyl] | | | | | | |
| pyridine-4-carboxylic acid | | | | | | |
| 2-({[(1R)-1-[bis(prop-2- | 3 | | | ++ | | + |
| en-1-yl)carbamoyl]-5- | | | | | | |
| (propylamino)pentyl]ami- | | | | | | |
| no}methyl)pyridine-4- | | | | | | |
| carboxylic acid | | | | | | |
| 2-({[(1R)-1-[bis(prop-2- | 4 | + | ++ | | | + |
| en-1-yl)carbamoyl]-5- | | | | | | |
| {[(tert- | | | | | | |
| butoxy)carbonyl](propyl)ami- | | | | | | |
| no}pentyl]amino}meth- | | | | | | |
| yl)pyridine-4-carboxylic acid | | | | | | |
| 2-(1-{[(1S)-1-{[(4- | 5 | + | | + | | + |
| nitrophenyl)meth- | | | | | | |
| yl]carbamoyl}ethyl]ami- | | | | | | |
| no}ethyl)pyridine-4-carboxylic | | | | | | |
| acid | | | | | | |
| 2-(1-{[(1S)-1-{[(2- | 6 | | ++ | ++ | + | |
| hydroxyphenyl)meth- | | | | | | |
| yl]carbamoyl}ethyl]ami- | | | | | | |
| no}ethyl)pyridine-4-carboxylic | | | | | | |
| acid | | | | | | |
| 2-({[(1S)-3-methyl-1-({[2- | 7 | | ++ | ++ | + | |
| (2- | | | | | | |
| methylcyclopropaneamido)phenyl]meth- | | | | | | |
| yl}carbamoyl)butyl]amino}meth- | | | | | | |
| yl)pyridine-4-carboxylic acid | | | | | | |
| 2-(1-{[(1S)-1-{[(2- | 8 | | | + | + | + |
| nitrophenyl)methyl]carbamoyl}eth- | | | | | | |
| yl]amino}ethyl)pyridine-4-carboxylic | | | | | | |
| acid | | | | | | |
| 2-({[(1S)-1-[bis(prop-2- | 9 | | | + | + | ++ |
| en-1-yl)carbamoyl]-5- | | | | | | |
| [(tert- | | | | | | |
| butylcarbamoyl)amino]pentyl]ami- | | | | | | |
| no}methyl)pyridine-4-carboxylic acid | | | | | | |
| 2-({[(1S)-1-[bis(prop-2- | 10 | + | + | | | +++ |
| en-1-yl)carbamoyl]-2-{[3- | | | | | | |
| (dimethylamino)propyl]carbamoyl}eth- | | | | | | |
| yl]amino}methyl)pyridine-4-carboxylic | | | | | | |
| acid | | | | | | |
| 2-({[(1S)-1-[bis(prop-2- | 11 | | ++ | | | ++ |
| en-1-yl)carbamoyl]-2- | | | | | | |
| ({[1- | | | | | | |
| (hydroxymethyl)cyclopropyl]meth- | | | | | | |
| yl}carbamoyl)ethyl]amino}meth- | | | | | | |
| yl)pyridine-4-carboxylic acid | | | | | | |
| 2-({[(1S)-1-({[2-(2- | 12 | + | ++ | ++ | | |
| methoxyacetamido)phenyl]meth- | | | | | | |
| yl}carbamoyl)-3-methylbutyl]ami- | | | | | | |
| no}methyl)pyridine-4-carboxylic acid | | | | | | |
| 2-{[({1-[(2E)-3- | 13 | + | | | + | +++ |
| phenylprop-2-en-1-yl]- | | | | | | |
| 1H-imidazol-2- | | | | | | |
| yl}methyl)amino]methyl} | | | | | | |
| pyridine-4-carboxylic acid | | | | | | |
| 2-[({[(3S)-2-oxo-1-[(1R)- | 14 | ++ | ++ | ++ | + | +++ |
| 1-phenylethyl]piperidin-3- | | | | | | |
| yl]methyl}amino)methyl] | | | | | | |
| pyridine-4-carboxylic acid | | | | | | |
| 2-(1-{[(1S)-1-[(pyridin-4- | 15 | + | | | + | + |
| ylmethyl)carbamoyl]ethyl] | | | | | | |
| amino}ethyl)pyridine-4- | | | | | | |
| carboxylic acid | | | | | | |
| 2-[(1R)-1-{[(1S)-1-({[4- | 16 | + | ++ | | + | |
| (hydroxymethyl)phenyl]meth- | | | | | | |
| yl}carbamoyl)ethyl]ami- | | | | | | |
| no}ethyl]pyridine- | | | | | | |
| 4-carboxylic acid | | | | | | |
| 2-[({[(3S)-2-oxo-1-[(1R)- | 17 | ++ | +++ | ++ | + | +++ |
| 1-phenylethyl]pyrrolidin- | | | | | | |
| 3- | | | | | | |
| yl]methyl}amino)methyl] | | | | | | |
| pyridine-4-carboxylic acid | | | | | | |
| 2-({[(1R)-1-[bis(prop-2- | 18 | | ++ | | + | ++ |
| en-1-yl)carbamoyl]-2- | | | | | | |
| [(cyclopropylmethyl)carbamoyl]eth- | | | | | | |
| yl]amino}methyl)pyridine-4-carboxylic | | | | | | |
| acid | | | | | | |
| 2-(1-{[(1S)-1-({[2- | 19 | | ++ | ++ | + | |
| (hydroxymethyl)phenyl]meth- | | | | | | |
| yl}carbamoyl)ethyl]ami- | | | | | | |
| no}ethyl)pyridine-4- | | | | | | |
| carboxylic acid | | | | | | |
| 2-({[(1S)-1-[bis(prop-2- | 20 | | | + | + | ++ |
| en-1-yl)carbamoyl]-5- | | | | | | |
| [methyl(methylcarbamoyl) | | | | | | |
| amino]pentyl]amino}meth- | | | | | | |
| yl)pyridine-4-carboxylic | | | | | | |
| acid | | | | | | |
| 2-({[(1S)-1-[bis(prop-2- | 21 | + | | | + | ++ |
| en-1-yl)carbamoyl]-5-(N- | | | | | | |
| methylacetamido)pentyl]ami- | | | | | | |
| no}methyl)pyridine-4-carboxylic | | | | | | |
| acid | | | | | | |
| 2-({[(2S)-6-{[(tert- | 22 | ++ | +++ | | + | +++ |
| butoxy)carbonyl]amino}- | | | | | | |
| 1-hydroxyhexan-2- | | | | | | |
| yl]amino}methyl)pyridine- | | | | | | |
| 4-carboxylic acid | | | | | | |
| 2-({[2-oxo-2-(piperidin-1- | 23 | | ++ | | | + |
| yl)ethyl]amino}meth- | | | | | | |
| yl)pyrimidine-4-carboxylic acid | | | | | | |
| 2-({[(1R)-1-[bis(prop-2- | 24 | | ++ | | + | + |
| en-1-yl)carbamoyl]-2- | | | | | | |
| (butylcarbamoyl)ethyl]ami- | | | | | | |
| no}methyl)pyridine-4- | | | | | | |
| carboxylic acid | | | | | | |
| 2-({[(1R)-1-[bis(prop-2- | 25 | | ++ | ++ | | ++ |
| en-1-yl)carbamoyl]-3- | | | | | | |
| carbamoylpropyl]amino} | | | | | | |
| methyl)pyridine-4-carboxylic | | | | | | |
| acid | | | | | | |
| 6-({[2-oxo-2-(piperidin-1- | 26 | +++ | | | + | +++ |
| yl)ethyl]amino}meth- | | | | | | |
| yl)pyridazine-4-carboxylic acid | | | | | | |
| 2-({[2- | 27 | | ++ | ++ | + | ++ |
| (diethylcarbamoyl)ethyl] | | | | | | |
| (2- | | | | | | |
| acetamidoethyl)amino}meth- | | | | | | |
| yl)pyridine-4-carboxylic acid | | | | | | |
| 2-(1-{[(1S)-1-(1,3-thiazol- | 28 | | ++ | ++ | + | |
| 2- | | | | | | |
| yl)ethyl]amino}eth- | | | | | | |
| yl)pyridine-4-carboxylic acid | | | | | | |
| 2-({[(1S)-1-[bis(prop-2- | 29 | | | + | | + |
| en-1-yl)carbamoyl]-3- | | | | | | |
| methanesulfonylpropyl]ami- | | | | | | |
| no}methyl)pyridine-4- | | | | | | |
| carboxylic acid | | | | | | |
| 2-(1-{[(1R)-1-(1,3-thiazol- | 30 | | | ++ | + | + |
| 2- | | | | | | |
| yl)ethyl]amino}eth- | | | | | | |
| yl)pyridine-4-carboxylic acid | | | | | | |
| 2-{1- | 31 | + | + | + | | |
| [(carbamoylmethyl)[2- | | | | | | |
| (diethylcarbamoyl)ethyl]ami- | | | | | | |
| no]ethyl}pyridine-4- | | | | | | |
| carboxylic acid | | | | | | |
| 2-({bis[2- | 32 | + | ++ | + | | |
| (diethylcarbamoyl)ethyl]ami- | | | | | | |
| no}methyl)pyridine-4- | | | | | | |
| carboxylic acid | | | | | | |
| 2-(1-{[(2R)-1-hydroxy-4- | 33 | + | | | + | ++ |
| methylpentan-2- | | | | | | |
| yl]amino}ethyl)pyridine- | | | | | | |
| 4-carboxylic acid | | | | | | |
| 2-{[(2-carbamoylethyl)[2- | 34 | ++ | ++ | ++ | + | +++ |
| oxo-2-(piperidin-1- | | | | | | |
| yl)ethyl]amino]meth- | | | | | | |
| yl}pyridine-4-carboxylic acid | | | | | | |
| 6-({[2-oxo-2-(piperidin-1- | 35 | ++ | ++ | ++ | + | |
| yl)ethy]amino}meth- | | | | | | |
| yl)pyrimidine-4-carboxylic acid | | | | | | |
| 2-(1-{[(1S)-1- | 36 | | ++ | ++ | + | |
| (benzylcarbamoyl)ethyl]ami- | | | | | | |
| no}ethyl)pyridine-4- | | | | | | |
| carboxylic acid | | | | | | |
| 2-({[(1R)-1-[bis(prop-2- | 37 | + | +++ | +++ | + | ++ |
| en-1-yl)carbamoyl]-3- | | | | | | |
| methanesulfonylpropyl]ami- | | | | | | |
| no}methyl)pyridine-4- | | | | | | |
| carboxylic acid | | | | | | |
| 2-({[(1S)-1-{[(1,1-dioxo- | 38 | | ++ | ++ | + | |
| 1-thiolan-3- | | | | | | |
| yl)methyl]carbamoyl}-3- | | | | | | |
| methylbutyl]amino}meth- | | | | | | |
| yl)pyridine-4-carboxylic | | | | | | |
| acid | | | | | | |
| 2-({[(1-ethyl-2- | 39 | ++ | +++ | ++ | + | +++ |
| oxopyrrolidin-3- | | | | | | |
| yl)methyl]amino}methyl) | | | | | | |
| pyridine-4-carboxylic acid | | | | | | |
| 2-({[(1S)-1-[bis(prop-2- | 40 | | ++ | | | ++ |
| en-1-yl)carbamoyl]-5- | | | | | | |
| {[(tert- | | | | | | |
| butoxy)carbonyl]ami- | | | | | | |
| no}pentyl]amino}meth- | | | | | | |
| yl)pyridine-4-carboxylic acid | | | | | | |
| 2-{1-[(1,3-thiazol-2- | 41 | ++ | ++ | | | ++ |
| ylmethyl)amino]eth- | | | | | | |
| yl}pyridine-4-carboxylic | | | | | | |
| acid | | | | | | |
| 2-[2-(methylsulfanyl)-1- | 42 | + | | | + | + |
| {[2-oxo-2-(piperidin-1- | | | | | | |
| yl)ethyl]amino}eth- | | | | | | |
| yl]pyridine-4-carboxylic | | | | | | |
| acid | | | | | | |
| 2-({[1- | 43 | + | ++ | ++ | | |
| (diethylcarbamoyl)propan- | | | | | | |
| 2- | | | | | | |
| yl]amino}meth- | | | | | | |
| yl)pyridine-4-carboxylic | | | | | | |
| acid | | | | | | |
| 2-({[2- | 44 | | ++ | | | +++ |
| (diethylcarbamoyl)eth- | | | | | | |
| yl](2- | | | | | | |
| hydroxyethyl)amino}meth- | | | | | | |
| yl)pyridine-4-carboxylic | | | | | | |
| acid | | | | | | |
| 2-(1-{[2-oxo-2-(piperidin- | 45 | | ++ | ++ | | |
| 1- | | | | | | |
| yl)ethyl]amino}butyl)pyri- | | | | | | |
| dine-4-carboxylic acid | | | | | | |
| 2-({[3-(4- | 46 | | ++ | ++ | | |
| methoxyphenyl)propyl][2- | | | | | | |
| oxo-2-(piperidin-1- | | | | | | |
| yl)ethyl]amino}meth- | | | | | | |
| yl)pyridine-4-carboxylic acid | | | | | | |
| 2-(1-{methyl[2-oxo-2- | 47 | | ++ | | | + |
| (piperidin-1- | | | | | | |
| yl)ethyl]amino}eth- | | | | | | |
| yl)pyridine-4-carboxylic acid | | | | | | |
| 2-(1-{[2-oxo-2-(piperidin- | 48 | ++ | ++ | | | ++ |
| 1- | | | | | | |
| yl)ethyl]amino}eth- | | | | | | |
| yl)pyridine-4-carboxylic acid | | | | | | |
| 2-({[(2S)-1-(tert-butoxy)- | 49 | | ++ | ++ | + | |
| 4-(methylsulfanyl)-1- | | | | | | |
| oxobutan-2- | | | | | | |
| yl]amino}methyl)pyridine- | | | | | | |
| 4-carboxylic acid | | | | | | |
| 2-{[5-(4-fluorophenyl)- | 50 | + | | + | | ++ |
| 5H,6H,7H,8H,9H- | | | | | | |
| imidazo[1,2- | | | | | | |
| a][1,4]diazepin-8- | | | | | | |
| yl]methyl}pyridine-4- | | | | | | |
| carboxylic acid | | | | | | |
| 2-{5H,6H,7H,8H,9H,10H- | 51 | +++ | | ++ | + | |
| imidazo[1,2- | | | | | | |
| a][1,4]diazocin-9- | | | | | | |
| ylmethyl}pyridine-4- | | | | | | |
| carboxylic acid | | | | | | |
| 2-{5H,6H,7H,8H- | 52 | | + | | + | ++ |
| imidazo[1,2-a]pyrazin-7- | | | | | | |
| ylmethyl}pyridine-4- | | | | | | |
| carboxylic acid | | | | | | |
| 2-{5H,6H,7H,8H,9H- | 53 | ++ | | + | + | |
| imidazo[1,2- | | | | | | |
| a][1,4]diazepin-8- | | | | | | |
| ylmethyl}pyridine-4- | | | | | | |
| carboxylic acid | | | | | | |
| 2-({5- | 54 | | + | | | ++ |
| [(dimethylamino)methyl]- | | | | | | |
| 5H,6H,7H,8H,9H- | | | | | | |
| imidazo[1,2- | | | | | | |
| a][1,4]diazepin-8- | | | | | | |
| yl}methyl)pyridine-4- | | | | | | |
| carboxylic acid | | | | | | |
| 2-{[(2S)-2-(piperidine-1- | 55 | | | + | | + |
| carbonyl)pyrrolidin-1- | | | | | | |
| yl]methyl}pyridine-4- | | | | | | |
| carboxylic acid | | | | | | |
| 2-{[(2R)-2-(piperidine-1- | 56 | | | ++ | | + |
| carbonyl)pyrrolidin-1- | | | | | | |
| yl]methyl}pyridine-4- | | | | | | |
| carboxylic acid | | | | | | |
| 2-{[(2R)-2- | 57 | | | +++ | | +++ |
| (hydroxymethyl)pyrrolidin- | | | | | | |
| 1-yl]methyl}pyridine-4- | | | | | | |
| carboxylic acid | | | | | | |
| 2-{[(2S)-2- | 58 | | | ++ | | +++ |
| (hydroxymethyl)pyrrolidin- | | | | | | |
| 1-yl]methyl}pyridine-4- | | | | | | |
| carboxylic acid | | | | | | |
| 2-{[(2R,3S)-3-hydroxy-5- | 59 | | ++ | ++ | | ++ |
| methyl-2-(2- | | | | | | |
| methylpropyl)pyrrolidin- | | | | | | |
| 1-yl]methyl}pyridine-4- | | | | | | |
| carboxylic acid | | | | | | |
| 2-({[(1S)-3-methyl-1- | 60 | | + | | + | +++ |
| (oxolan-2- | | | | | | |
| yl)butyl]amino}meth- | | | | | | |
| yl)pyridine-4-carboxylic acid | | | | | | |
| (S)-2-{[(1-hydroxy-4- | 61 | +++ | | | | +++ |
| methylpentan-2- | | | | | | |
| yl)amino]methyl}pyridine- | | | | | | |
| 4-carboxylic acid | | | | | | |
| 2-{[3-cyclohexyl-2- | 62 | + | | | | ++ |
| (hydroxymethyl)piperidin- | | | | | | |
| 1-yl]methyl}pyridine-4- | | | | | | |
| carboxylic acid | | | | | | |
| 2-{[2-(hydroxymethyl)-3- | 63 | + | | | | + |
| phenylpiperidin-1- | | | | | | |
| yl]methyl}pyridine-4- | | | | | | |
| carboxylic acid | | | | | | |
| 2-{[(2S)-2- | 64 | | | + | + | ++ |
| (hydroxymethyl)azetidin- | | | | | | |
| 1-yl]methyl}pyridine-4- | | | | | | |
| carboxylic acid | | | | | | |
| 2-{[(2S,3S)-3-ethyl-2- | 65 | | | + | + | + |
| (hydroxymethyl)pyrrolidin- | | | | | | |
| 1-yl]methyl}pyridine-4- | | | | | | |
| carboxylic acid | | | | | | |
| 2-{[2- | 66 | | ++ | + | | + |
| (hydroxymethyl)piperidin- | | | | | | |
| 1-yl]methyl} pyridine-4- | | | | | | |
| carboxylic acid | | | | | | |
| 2-({2-methyl- | 67 | + | | | + | + |
| 5H,6H,7H,8H,9H,10H- | | | | | | |
| imidazo[1,2- | | | | | | |
| a][1,5]diazocin-8- | | | | | | |
| yl}methyl)pyridine-4- | | | | | | |
| carboxylic acid | | | | | | |
| 2-{[3- | 68 | ++ | | +++ | | ++ |
| (ethylcarbamoyl)azetidin- | | | | | | |
| 1-yl]methyl}pyridine-4- | | | | | | |
| carboxylic acid | | | | | | |
| 2-({2-methyl- | 69 | ++ | + | ++ | + | +++ |
| 5H,6H,7H,8H,9H- | | | | | | |
| imidazo[1,2- | | | | | | |
| d][1,4]diazepin-7- | | | | | | |
| yl}methyl)pyridine-4- | | | | | | |
| carboxylic acid | | | | | | |
| 2-{[(2S)-2-[2-oxo-2- | 70 | + | | + | | |
| (piperidin-1- | | | | | | |
| yl)ethyl]piperidin-1- | | | | | | |
| yl]methyl}pyridine-4- | | | | | | |
| carboxylic acid | | | | | | |
| 2-{[(2S)-2- | 71 | + | | | + | + |
| [(ethylcarbamoyl)methyl] | | | | | | |
| piperidin-1- | | | | | | |
| yl]methyl}pyridine-4- | | | | | | |
| carboxylic acid | | | | | | |
| 2-{[(2R)-2-[2-oxo-2- | 72 | + | | | + | + |
| (piperidin-1- | | | | | | |
| yl)ethyl]piperidin-1- | | | | | | |
| yl]methyl}pyridine-4- | | | | | | |
| carboxylic acid | | | | | | |
| 2-{[(3R)-3- | 73 | + | ++ | ++ | | |
| [(ethylcarbamoyl)methyl] | | | | | | |
| pyrrolidin-1- | | | | | | |
| yl]methyl}pyridine-4- | | | | | | |
| carboxylic acid | | | | | | |
| 2-{[3- | 74 | | | + | + | ++ |
| (ethylcarbamoyl)piperidin- | | | | | | |
| 1-yl]methyl}pyridine-4- | | | | | | |
| carboxylic acid | | | | | | |
| 2-{[4- | 75 | | ++ | ++ | + | + |
| (ethylcarbamoyl)piperidin- | | | | | | |
| 1-yl]methyl} pyridine-4- | | | | | | |
| carboxylic acid | | | | | | |
| 2-{[3- | 76 | | | ++ | + | ++ |
| (ethylcarbamoyl)pyrrolidin- | | | | | | |
| 1-yl]methyl}pyridine-4- | | | | | | |
| carboxylic acid | | | | | | |
| 2-{[(3S)-3- | 77 | | | ++ | + | ++ |
| [(ethylcarbamoyl)methyl] | | | | | | |
| pyrrolidin-1- | | | | | | |
| yl]methyl}pyridine- | | | | | | |
| 4-carboxylic acid | | | | | | |
| 2-[({[(3S)-1-[(1R)-1-(4- | 78 | | +++ | +++ | + | |
| methoxyphenyl)ethyl]-2- | | | | | | |
| oxopyrrolidin-3- | | | | | | |
| yl]methyl}amino)methyl] | | | | | | |
| pyridine-4-carboxylic acid | | | | | | |
| 2-[({[(3R)-1-[(1R)-1-(4- | 79 | +++ | | | ++ | +++ |
| methoxyphenyl)ethyl]-2- | | | | | | |
| oxopyrrolidin-3- | | | | | | |
| yl]methyl}amino)methyl] | | | | | | |
| pyridine-4-carboxylic acid | | | | | | |
| 2-[({[(3S)-1-[(1R)-1-(4- | 80 | ++ | +++ | ++ | + | +++ |
| methoxyphenyl)ethyl]-2- | | | | | | |
| oxopiperidin-3- | | | | | | |
| yl]methyl}amino)methyl] | | | | | | |
| pyridine-4-carboxylic acid | | | | | | |
| 2-[({[(3R)-1-[(1R)-1-(4- | 81 | ++ | +++ | ++ | + | +++ |
| methoxyphenyl)ethyl]-2- | | | | | | |
| oxopiperidin-3- | | | | | | |
| yl]methyl}amino)methyl] | | | | | | |
| pyridine-4-carboxylic acid | | | | | | |
| 2-[({[(3R)-2-oxo-1-[(1R)- | 82 | +++ | | | ++ | +++ |
| 1-phenylethyl]pyrrolidin- | | | | | | |
| 3- | | | | | | |
| yl]methyl}amino)methyl] | | | | | | |
| pyridine-4-carboxylic acid | | | | | | |
| 2-[({[1-(4-fluorobenzyl)- | 83 | | | +++ | + | ++ |
| 1H-pyrrolo[2,3-b]pyridin- | | | | | | |
| 3- | | | | | | |
| yl]methyl}amino)methyl] | | | | | | |
| pyridine-4-carboxylic acid | | | | | | |
| 2-{[(pyridin-3- | 84 | | | +++ | + | +++ |
| ylmethyl)amino]meth- | | | | | | |
| yl}pyridine-4-carboxylic acid | | | | | | |
| 2-{[(isoquinolin-4- | 85 | | +++ | | + | |
| ylmethyl)amino]meth- | | | | | | |
| yl}pyridine-4-carboxylic acid | | | | | | |
| 2-{[({5-fluoro-1-[(4- | 86 | + | ++ | | + | |
| fluorophenyl)methyl]-1H- | | | | | | |
| indol-3- | | | | | | |
| yl}methyl)amino]methyl} | | | | | | |
| pyridine-4-carboxylic acid | | | | | | |
| 2-{[(quinolin-6- | 87 | | ++ | +++ | | +++ |
| ylmethyl)amino]meth- | | | | | | |
| yl}pyridine-4-carboxylic acid | | | | | | |
| 2-{[({2-tert- | 88 | | ++ | +++ | + | +++ |
| butylimidazo[1,2- | | | | | | |
| a]pyridin-3- | | | | | | |
| y1}methyl)amino]methyl} | | | | | | |
| pyridine-4-carboxylic acid | | | | | | |
| 6-({[(2S)-1-(benzyloxy)- | 89 | | | ++ | + | |
| 4-methylpentan-2- | | | | | | |
| yl]amino}methyl)pyrimidine- | | | | | | |
| 4-carboxylic acid | | | | | | |
| 2-[({5H,6H,7H,8H- | 90 | ++ | | +++ | | +++ |
| imidazo[1,2-a]pyridin-8- | | | | | | |
| yl}amino)methyl]pyridine- | | | | | | |
| 4-carboxylic acid | | | | | | |
| 2-[({4-bromo-5H,6H,7H- | 91 | ++ | | | | +++ |
| cyclopenta[b]pyridin-7- | | | | | | |
| yl}amino)methyl]pyridine- | | | | | | |
| 4-carboxylic acid | | | | | | |
| 2-[({4-benzyl-5H,6H,7H- | 92 | | ++ | ++ | + | +++ |
| cyclopenta[b]pyridin-7- | | | | | | |
| yl}amino)methyl]pyridine- | | | | | | |
| 4-carboxylic acid | | | | | | |
| 2-[({5H,6H,7H- | 93 | ++ | | | + | +++ |
| pyrrolo[1,2-a]imidazol-7- | | | | | | |
| yl}amino)methyl]pyridine- | | | | | | |
| 4-carboxylic acid | | | | | | |
| 2-{[(5,6,7,8- | 94 | + | | | + | + |
| tetrahydroquinolin-8- | | | | | | |
| yl)amino]methyl} | | | | | | |
| pyridine-4-carboxylic acid | | | | | | |
| 2-({[3-(prop-2-en-1-yl)- | 95 | ++ | +++ | +++ | | |
| 5H,6H,7H- | | | | | | |
| cyclopenta[b]pyridin-7- | | | | | | |
| yl]amino}methyl)pyridine- | | | | | | |
| 4-carboxylic acid | | | | | | |
| 2-({[4-(2-phenylethyl)- | 96 | | +++ | +++ | | |
| 5H,6H,7H- | | | | | | |
| cyclopenta[b]pyridin-7- | | | | | | |
| yl]amino}methyl)pyridine- | | | | | | |
| 4-carboxylic acid | | | | | | |
| 2-[({4-ethyl-5H,6H,7H- | 97 | | +++ | +++ | | +++ |
| cyclopenta[b]pyridin-7- | | | | | | |
| yl}amino)methyl]pyridine- | | | | | | |
| 4-carboxylic acid | | | | | | |
| 2-[({5H,6H,7H- | 98 | ++ | ++ | | | +++ |
| cyclopenta[b]pyridin-7- | | | | | | |
| yl}amino)methyl]pyridine- | | | | | | |
| 4-carboxylic acid | | | | | | |
| 2-({[4-(propan-2-yl)- | 99 | ++ | +++ | | + | |
| 5H,6H,7H- | | | | | | |
| cyclopenta[b]pyridin-7- | | | | | | |
| yl]amino}methyl)pyridine- | | | | | | |
| 4-carboxylic acid | | | | | | |
| 2-[({4-ethenyl-5H,6H,7H- | 100 | ++ | | +++ | | +++ |
| cyclopenta[b]pyridin-7- | | | | | | |
| yl}amino)methyl]pyridine- | | | | | | |
| 4-carboxylic acid | | | | | | |
| 2-[({4-methoxy- | 101 | ++ | +++ | | | +++ |
| 5H,6H,7H- | | | | | | |
| cyclopenta[b]pyridin-7- | | | | | | |
| yl}amino)methyl]pyridine- | | | | | | |
| 4-carboxylic acid | | | | | | |
| 2-[({6,6-dimethyl- | 102 | | ++ | ++ | | ++ |
| 5H,6H,7H- | | | | | | |
| cyclopenta[b]pyridin-7- | | | | | | |
| yl}amino)methyl]pyridine- | | | | | | |
| 4-carboxylic acid | | | | | | |
| 2-[({3-bromo-5H,6H,7H- | 103 | ++ | +++ | +++ | + | |
| cyclopenta[b]pyridin-7- | | | | | | |
| yl}amino)methyl]pyridine- | | | | | | |
| 4-carboxylic acid | | | | | | |
| 2-[({[(3S)-1-ethyl-2- | 104 | ++ | | ++ | + | +++ |
| oxopiperidin-3- | | | | | | |
| yl]methyl}amino)methyl] | | | | | | |
| pyridine-4-carboxylic acid | | | | | | |
| 2-[({[(3S)-1-ethyl-2- | 105 | | +++ | +++ | + | |
| oxopyrrolidin-3- | | | | | | |
| yl]methyl}amino)methyl] | | | | | | |
| pyridine-4-carboxylic acid | | | | | | |
| 2-[({[(3R)-1-ethyl-2- | 106 | +++ | +++ | | + | |
| oxopyrrolidin-3- | | | | | | |
| yl]methyl}amino)methyl] | | | | | | |
| pyridine-4-carboxylic acid | | | | | | |
| 2-[({[(3R)-1-ethyl-2- | 107 | | +++ | | + | +++ |
| oxopiperidin-3- | | | | | | |
| yl]methyl}amino)methyl] | | | | | | |
| pyridine-4-carboxylic acid | | | | | | |
| 2,2,2-trifluoro-1-[6-(2- | 108 | ++ | ++ | | | +++ |
| (5H,6H,7H,8H,9H- | | | | | | |
| imidazo[1,2- | | | | | | |
| a][1,4]diazepin-8- | | | | | | |
| ylmethyl}pyridin-4-yl)-5- | | | | | | |
| oxa-7-azaspiro[2.5]octan- | | | | | | |
| 7-yl]ethan-1-one | | | | | | |

(a)+++: IC₅₀ < 250 nM;
++: 250 nM ≦ IC₅₀ ≦ 2500 nM;
+: IC₅₀ > 2500 nM

### Example 3: Cell Assays for IC₅₀ Value Determination

Histone Lysine Demethylase Immunofluorescence Assays for IC₅₀ value Determination, non-transfected cells

This procedure may be used to demonstrate the ability of compounds of the disclosure to inhibit demethylation of a specific histone lysine mark in a human osteosarcoma cancer cell line.

### General Method

U2OS cells are harvested and seeded into multi well plates into media containing compound. The media is DMEM containing 5% FBS and pen/strep. 20 hours after incubation of cells with compounds, the cells are washed once in PBS, harvested by fixation with formaldehyde 4% aqueous solution, and washed in PBS. Subsequently, the cells are permeabilized in PBS with 0.2% Triton X-100. Blocking is performed in PBS with 0.2% Triton X-100 and 5% FBS. The cells are incubated with αH3K4me3 primary antibody (Cell Signaling, #9751S) in blocking solution over night at 4° C. After incubation with primary antibody, the cells are washed with PBS, incubated with secondary antibody (Alexa fluor 594 goat anti rabbit IgG, Invitrogen, A11012) and Hoechst, (Sigma, 33342) in blocking solution, and washed again with PBS. Finally, PBS is added and high throughput imaging and analysis are performed by an IN Cell Analyzer 1000 (GE Healthcare). IC₅₀ values are determined based on an average measure of the staining of the H3K4me3 mark in cells.

Compound 108, 2,2,2-trifluoro-1-[6-(2-{5H,6H,7H,8H,9H-imidazo[1,2-a][1,4]diazepin-8-ylmethyl}pyridin-4-yl)-5-oxa-7-azaspiro[2.5]octan-7-yl]ethan-1-one, has an IC₅₀ value <2.5 μM as determined by the method of this example.

### Example 4: Histone Lysine Demethylase Immunofluorescence Assays for IC₅₀ Value Determination

This procedure may be used to demonstrate the ability of the compounds of the disclosure to inhibit specific histone lysine demethylases expressed in a human osteosarcoma cell line.

### General Method

U2OS cells are seeded 24 hours before transfection. Transfection is performed with Fugene HD transfection reagent as recommended by the manufacturer. 6 hours after transfection, the cells are harvested and seeded into multi well plates into media containing compound. The media used is DMEM containing 10% FBS and pen/strep. 20 hours after incubation of cells with compounds, the cells are washed in PBS, harvested by fixation with formaldehyde 4% aqueous solution, and washed in PBS. Subsequently, the cells are permeabilized in PBS with 0.2% Triton X-100 for. Blocking is performed in PBS with 0.2% Triton X-100 and 5% FBS. The cells are incubated with primary antibodies in blocking solution over night at 4° C. The primary antibodies used in the assays are HA.11 (Covance, MMS-101P) and an antibody detecting the relevant mark. After incubation with primary antibodies, the cells are washed with PBS, incubated with secondary antibodies (Alexa fluor 594 goat anti rabbit IgG, Invitrogen, A11012; Alexa flour 488 donkey anti mouse IgG, Invitrogen, A21202) and Hoechst, (Sigma, 33342) in blocking solution, and washed again with PBS. Finally, PBS is added and high throughput imaging and analysis are performed by an IN Cell Analyzer 1000 (GE Healthcare). The robot software analyzed individual cells and divided these into HA⁺ (transfected cells) and HA⁻ (non-transfected cells). The IC₅₀ values are based on an average measure of the staining of the mark specified in the table below in the transfected cells.

### Example 5: Cell Proliferation Assays for EC₅₀ Value Determination

This procedure may be used to demonstrate the ability of the compounds of the disclosure to inhibit the proliferation of a human breast cancer or other cancer cell line.

### General Method

MCF7 cells or other cancer cell line cells are seeded in multi well plates at a density optimized to give approximately 90% confluent cells at the time of harvest. Cells are incubated for 24 hours before addition of compound. Compounds are diluted in complete medium and added to the plates in duplicates. The final concentration of DMSO is maximum 0.5%. Complete medium used is DMEM with GlutaMAX containing 10% FBS and pen/strep.

120 hours after addition of compounds, the plates are harvested and analyzed by ATPlite 1 Step (Perkin Elmer, cat no 6016739) according to the manufacturers' recommendation.

In this specification, unless expressly otherwise indicated, the word ‘or’ is used in the sense of an operator that returns a true value when either or both of the stated conditions is met, as opposed to the operator ‘exclusive or’ which requires that only one of the conditions is met. The word ‘comprising’ is used in the sense of ‘including’ rather than in to mean ‘consisting of’. All prior teachings acknowledged above are hereby incorporated by reference. No acknowledgement of any prior published document herein should be taken to be an admission or representation that the teaching thereof was common general knowledge in Australia or elsewhere at the date hereof.

1. A compound of the Formula (Ia) 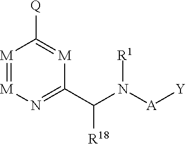
wherein
Q is selected from CO₂H, —CH═NR¹², —W, —CHR²⁰NR²¹R¹³, —CH═O and —CH(OR¹⁷)₂;
A is selected from —C(R^{2a})₂C(O)—, —C(R²)₂C(R²)₂C(O)—, —Z′—C₃₋₁₀ cycloalkylene, —Z′-heterocyclylene, —Z′-heteroarylene and —Z′-arylene, which —Z′-cycloalkylene, —Z′-heterocyclylene, —Z′-heteroarylene and —Z′-arylene may optionally be substituted with one or more R³ and may form a cyclic or heterocyclic structure with Y, wherein said cyclic or heterocyclic structure formed with Y is optionally fused to an optionally substituted aryl or heteroaryl group;
Z′ is selected from C₁₋₄ alkylene, C₂₋₅ alkenene, C₂₋₅ alkynene, heterocyclylene and C₃₋₆ cycloalkylene;
each M is independently selected from CH or N;
Y is selected from —H, —NR⁶R⁷, —OR⁷, C₁₋₈ alkyl, C₂₋₈ alkenyl, C₂₋₈ alkynyl, C₃₋₁₀ cycloalkyl, heterocyclyl, heteroaryl and aryl, which alkyl, alkenyl, alkynyl, cycloalkyl, heterocyclyl, heteroaryl and aryl may optionally be substituted with one or more R³;
R¹ is selected from —H, C₁₋₈ alkyl, C₂₋₈ alkenyl, C₂₋₈ alkynyl, C₃₋₁₀ cycloalkyl, which alkyl, alkenyl, alkynyl and cycloalkyl may be optionally substituted with one or more selected from —OH, aryl, C₁₋₆ alkoxy, heteroaryl, aryloxy, heteroaryloxy, —NR⁶R⁷, F, and C₃₋₆ cycloalkyl; or with -A-Y forms a nitrogen containing optionally substituted heterocyclic group where the optional substitution may be C₁₋₈ alkyl, C₂₋₈ alkenyl, C₂₋₈ alkynyl, C₃₋₁₀ cycloalkyl, C₁₋₆ alkoxy, C₁₋₆ hydroxyalkyl, aryl, —C(═O)NR⁶R⁷, —NR⁶R⁷, —OH, and halogen, which alkyl, alkenyl, alkynyl, cycloalkyl and aryl may be optionally substituted with one or more selected from —OH, aryl, C₁₋₆ alkoxy, heteroaryl, aryloxy, heteroaryloxy, —NR⁶R⁷, F, and C₃₋₆ cycloalkyl; or said nitrogen containing optionally substituted heterocyclic group formed with -A-Y is optionally fused to an optionally substituted aryl or heteroaryl group; or with R¹⁸ forms a nitrogen containing optionally substituted heterocyclic group where the optional substitution may be C₁₋₈ alkyl, C₂₋₈ alkenyl, C₂₋₈ alkynyl, or C₃₋₁₀ cycloalkyl, which alkyl, alkenyl, alkynyl and cycloalkyl may be optionally substituted with one or more selected from —OH, aryl, C₁₋₆ alkoxy, heteroaryl, aryloxy, heteroaryloxy, —NR⁶R⁷, F, and C₃₋₆ cycloalkyl;
R² is selected from —H, C₁₋₈ alkyl, C₂₋₈ alkenyl, C₂₋₈ alkynyl, and C₃₋₁₀ cycloalkyl, which alkyl, alkenyl, alkynyl and cycloalkyl may be optionally substituted with one or more selected from —OH, aryl, C₁₋₆ alkoxy, heteroaryl, aryloxy, heteroaryloxy, F, and C₃₋₆ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—C(═O)—R⁷, —Z—OR⁷, halogen, —Z—SR⁷, —Z—SOR⁷, —Z—SO₂R⁷, —Z—SO₂NR⁶R⁷ and —Z—COOR⁷; alternatively, R² may form a cyclic or heterocyclic structure with another R², R¹ R¹⁸ or Y;
R^{2a} is selected from —H, C₁₋₈ alkyl, C₂₋₈ alkenyl, C₂₋₈ alkynyl, and C₃₋₁₀ cycloalkyl, which alkyl, alkenyl, alkynyl and cycloalkyl may be optionally substituted with one or more selected from —OH, aryl, C₁₋₆ alkoxy, heteroaryl, aryloxy, heteroaryloxy, F, and C₃₋₆ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—NR⁶—C(═O)—OR⁷, —Z—C(═O)—OR⁷, —Z—OR⁷, halogen, —Z—SR⁷, —Z—SOR⁷, —Z—SO₂R⁷, —Z—SO₂NR⁶R⁷ and —Z—COOR⁷; with the proviso that the two R^{2a} groups are either both non-hydrogen, or that one of the R^{2a} forms a ring with R¹ or R¹⁸;
each R³ is independently selected from C₁₋₆ alkyl, C₁₋₄ fluoroalkyl, C₁₋₄ hydroxyalkyl, C₂₋₆ alkenyl, C₂₋₆ alkynyl, C₃₋₁₀ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—NR⁶—C(═O)—OR⁷, —Z—C(═O)—R⁷, —Z—OR⁷, halogen, —Z—SR⁷, —Z—SOR⁷, —Z—SO₂R⁷, —Z—SO₂NR⁶R⁷, and —Z—COOR⁷, wherein any heterocyclyl may be substituted with one or more R⁴, and wherein any heteroaryl and any aryl may be substituted with one or more R⁵;
Z is selected from a single bond, C₁₋₄ alkylene, heterocyclylene and C₃₋₆ cycloalkylene;
each R⁴ is independently selected from C₁₋₆ alkyl, C₁₋₄ fluoroalkyl, C₁₋₄ hydroxyalkyl, C₁₋₄ alkoxy, C₃₋₁₀ cycloalkyl, NR⁶R⁷, C(═O)—NR⁶R⁷, NR⁶—C(═O)—R⁷, Z—C(═O)—R⁷, —Z—C(═O)—H, OR⁷, halogen, SR⁷, SOR⁷, SO₂R⁷, SO₂NR⁶R⁷ and COOR⁷ and —OH;
each R⁵ is independently selected from C₁₋₆ alkyl, C₁₋₄ fluoroalkyl, C₁₋₄ hydroxyalkyl, C₁₋₄ alkoxy, C₃₋₆ cycloalkyl, —Z—C(═O)—NR⁶R⁷, —Z—NR⁶—C(═O)—R⁷, —Z—C(═O)—R⁷, —Z—C(═O)OR⁷, —Z—NR⁶C(═O)OR⁷, OR⁷, —CN and halogen;
each of R⁶ and R⁷ is independently selected from hydrogen, C₁₋₈ alkyl, C₁₋₄ fluoroalkyl, C₁₋₄ perfluoroalkyl, C₁₋₄ hydroxyalkyl, C₂₋₈ alkenyl, C₂₋₈ alkynyl, C₃₋₁₀ cycloalkyl, —Z-heterocyclyl, —Z-heteroaryl and —Z-aryl, which alkyl, alkenyl, alkynyl, cycloalkyl, heterocyclyl, heteroaryl and aryl may optionally be substituted with one or more independently selected R⁸; or, alternatively, R⁶ and R⁷ may together with the N-atom to which they are attached form an N-heterocyclic ring optionally substituted with one or more independently selected R⁸;
each R⁸ is independently selected from C₁₋₆ alkyl, C₁₋₄ fluoroalkyl, C₁₋₄ hydroxyalkyl, C₂₋₆ alkenyl, C₂₋₆ alkynyl, C₃₋₁₀ cycloalkyl, —Z-heterocyclyl, —Z-heteroaryl, —Z-aryl, —Z—NR¹⁰R¹¹, —Z—C(═O)—NR¹⁰R¹¹, —Z—OR⁹, halogen, —CN, —Z—SR⁹, —Z—SOR⁹, —Z—SO₂R⁹ and —Z—COOR⁹, which alkyl, alkenyl, alkynyl, cycloalkyl, heterocyclics, heteroaryl and aryl may optionally be substituted with one or more selected from C₁₋₄ alkyl, C₁₋₄ fluoroalkyl, C₁₋₄ hydroxyalkyl, C₃₋₆ cycloalkyl, —Z-heterocyclyl, —Z-heteroaryl, —Z-aryl, —Z—NR¹⁰R¹¹, —Z—C(═O)—NR¹⁰R¹¹, —Z—OR⁹, halogen, —CN, —Z—SR⁹, —Z—SOR⁹, —Z—SO₂R⁹ and —Z—COOR⁹; wherein any heterocyclyl may be further substituted with one or more R⁴ as defined above, and wherein any heteroaryl and any aryl may be further substituted with one or more R⁵ as defined above;
each R⁹ is independently selected from —H, C₁₋₈ alkyl, C₁₋₄ fluoroalkyl, C₁₋₄ hydroxyalkyl, C₂₋₈ alkenyl, C₂₋₈ alkynyl, C₃₋₁₀ cycloalkyl, —Z-heterocyclyl, —Z-aryl, and —Z-heteroaryl, wherein any heterocyclyl may be substituted with one or more R⁴ as defined above, and wherein any heteroaryl and any aryl may be substituted with one or more R⁵ as defined above;
each of R¹⁰ and R¹¹ independently selected from —H, C₁₋₆ alkyl, C₁₋₄ fluoroalkyl, C₁₋₄ hydroxyalkyl, C₂₋₈ alkenyl, C₂₋₈ alkynyl, C₃₋₁₀ cycloalkyl, heterocyclyl, heteroaryl, and aryl, wherein any heterocyclyl may be substituted with one or more R⁴ as defined above, and wherein any heteroaryl and any aryl may be substituted with one or more R⁵ as defined above, or, alternatively, R¹⁰ and R¹¹ may together with the N-atom to which they are attached form an optionally 5 to 7 membered, N-heterocyclic ring optionally substituted with one or more R⁴ as defined above;
when Q is —CH═NR¹², R¹² is selected from C₁₋₁₀ alkyl, C₂₋₁₀ alkenyl, C₂₋₁₀ alkynyl, C₃₋₁₀ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—C(═O)—R⁷, —Z—OR⁷, halogen, —Z—SR⁷, —Z—SOR⁷, —Z—SO₂R⁷ and —Z—COOR⁷, which alkyl, alkenyl, alkynyl, cycloalkyl, heterocyclyl, heteroaryl and aryl may optionally be substituted with one or more R³;
when Q is —CHR²⁰NR²¹R¹³, R¹³ is selected from hydrogen, —C(O)R⁷, —C(O)C(O)R⁷, —C(O)C(O)OR⁷, C₁₋₈ alkyl, C₁₋₄ fluoroalkyl, C₁₋₄ perfluoroalkyl, C₁₋₄ hydroxyalkyl, C₂₋₈ alkenyl, C₂₋₈ alkynyl, C₃₋₁₀ cycloalkyl, —Z-heterocyclyl, —Z-aryl and —Z-heteroaryl, which alkyl, alkenyl, alkynyl, cycloalkyl, heterocyclyl, aryl and heteroaryl may optionally be substituted with one or more independently selected R⁸, or is —CR¹⁴R¹⁵—NR⁶R⁷, —CR¹⁴R¹⁵CN, or —CR¹⁴R¹⁵OR⁷, wherein each of R¹⁴ and R¹⁵ is independently selected from —H, C₁₋₈ alkyl, C₂₋₈ alkenyl, C₂₋₈ alkynyl, C₃₋₁₀ cycloalkyl, heterocyclyl, heteroaryl and aryl, and wherein R¹⁴ and R¹⁵ together with the intervening carbon atom may designate a C₃₋₁₀ cycloalkyl or C₅₋₁₀-cycloalkenyl ring, which alkyl, alkenyl, alkynyl, cycloalkyl ring, cycloalkenyl ring, heterocyclyl, heteroaryl and aryl may optionally be substituted with one or more R³;
R²⁰ and R²¹ are hydrogen, or together form a 1,3-diaza-C₅₋₇-cycloalk-2-yl group which is N-substituted with R¹⁶ and optionally further substituted with one or more R³, and optionally containing one or two oxo groups; a 1,3-thiaza-C₅₋₇-cycloalk-2-yl group which is N-substituted with R¹⁶ and optionally further substituted with one or more R³ and optionally containing one or two oxo groups; an 1,3-oxaza-C₅₋₇-cycloalk-2-yl group which is N-substituted with R¹⁶ and optionally further substituted with one or more R³, and optionally containing one or two oxo groups, wherein in all three instances two R³'s on the same carbon atom may together form a spiro group;
when Q is W, W is selected from an 1,3-diaza-C₅₋₇-cycloalk-2-yl group which is N-substituted with R¹⁶ and optionally further substituted with one or more R³, and optionally containing one or two oxo groups; a 1,3-thiaza-C₅₋₇-cycloalk-2-yl group which is N-substituted with R¹⁶ and optionally further substituted with one or more R³ and optionally containing one or two oxo groups; an 1,3-oxaza-C₅₋₇-cycloalk-2-yl group which is N-substituted with R¹⁶ and optionally further substituted with one or more R³, and optionally containing one or two oxo groups, wherein in all three instances two R³'s on the same carbon atom may together form a spiro group;
R¹⁶ is selected from hydrogen, —C(O)R⁷, —C(O)C(O)R⁷, —C(O)C(O)OR⁷, and —C(O)C(O)NR⁶R⁷;
when Q is —CH(OR¹⁷)₂, each R¹⁷ independently is R³, or wherein two R¹⁷ substituents together with the intervening —O—CH(−)—O— may form a heterocyclyl optionally substituted with one or more R³ and containing up to two oxo groups;
R¹⁸ is selected from hydrogen, C₁₋₆ alkyl, C₁₋₆ fluoroalkyl, C₁₋₆ hydroxyalkyl, C₂₋₇ alkenyl, C₂₋₇ alkynyl, C₃₋₇ cycloalkyl, C₃₋₇ oxyalkyl and may form a cyclic or heterocyclic structure with A, Y or R¹;
or an isomer or a mixture of isomers thereof, or a pharmaceutically acceptable salt, or solvate or prodrug thereof.

2-5. (canceled)

6. A compound of the Formula (If) 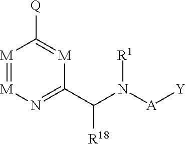
wherein
Q is selected from CO₂H, —CH═NR¹², —W, —CHR²⁰NR²¹R¹³, —CH═O and —CH(OR¹⁷)₂;
A is selected from —C(R^{2a})₂C(O)—, —C(R²)₂C(R²)₂C(O)—, C₁₋₈ alkylene, C₂₋₈ alkenylene, C₂₋₈ alkynylene, —Z′—C₃₋₁₀ cycloalkylene, —Z′-heterocyclylene, —Z′-heteroarylene and —Z′-arylene, which alkylene, alkenylene, alkynylene, —Z′-cycloalkylene, —Z′-heterocyclylene, —Z′-heteroarylene and —Z′-arylene may optionally be substituted with one or more R³ and may form a cyclic or heterocyclic structure with Y; with the proviso that when Q is —CH═O, A is not alkynylene;
Z′ is selected from C₁₋₄ alkylene, C₂₋₅ alkenene, C₂₋₅ alkynene, heterocyclylene and C₃₋₆ cycloalkylene;
each M is independently selected from CH or N;
Y is selected from —H, —NR⁶R⁷, —OR⁷, C₁₋₈ alkyl, C₂₋₈ alkenyl, C₂₋₈ alkynyl, C₃₋₁₀ cycloalkyl, heterocyclyl, heteroaryl and aryl, which alkyl, alkenyl, alkynyl, cycloalkyl, heterocyclyl, heteroaryl and aryl may optionally be substituted with one or more R³;
R¹ is selected from —H, C₁₋₈ alkyl, C₂₋₈ alkenyl, C₂₋₈ alkynyl, C₃₋₁₀ cycloalkyl, which alkyl, alkenyl, alkynyl and cycloalkyl may be optionally substituted with one or more selected from —OH, aryl, C₁₋₆ alkoxy, heteroaryl, aryloxy, heteroaryloxy, —NR⁶R⁷, F, and C₃₋₆ cycloalkyl; or with -A-Y forms a nitrogen containing optionally substituted heterocyclic group where the optional substitution may be C₁₋₈ alkyl, C₂₋₈ alkenyl, C₂₋₈ alkynyl, or C₃₋₁₀ cycloalkyl, which alkyl, alkenyl, alkynyl and cycloalkyl may be optionally substituted with one or more selected from —OH, aryl, C₁₋₆ alkoxy, heteroaryl, aryloxy, heteroaryloxy, —NR⁶R⁷, F, and C₃₋₆ cycloalkyl; or with R¹⁸ forms a nitrogen containing optionally substituted heterocyclic group where the optional substitution may be C₁₋₈ alkyl, C₂₋₈ alkenyl, C₂₋₈ alkynyl, or C₃₋₁₀ cycloalkyl, which alkyl, alkenyl, alkynyl and cycloalkyl may be optionally substituted with one or more selected from —OH, aryl, C₁₋₆ alkoxy, heteroaryl, aryloxy, heteroaryloxy, —NR⁶R⁷, F, and C₃₋₆ cycloalkyl;
R² is selected from —H, C₁₋₈ alkyl, C₂₋₈ alkenyl, C₂₋₈ alkynyl, and C₃₋₁₀ cycloalkyl, which alkyl, alkenyl, alkynyl and cycloalkyl may be optionally substituted with one or more selected from —OH, aryl, C₁₋₆ alkoxy, heteroaryl, aryloxy, heteroaryloxy, F, and C₃₋₆ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—C(═O)—R⁷, —Z—OR⁷, halogen, —Z—SR⁷, —Z—SOR⁷, —Z—SO₂R⁷, —Z—SO₂NR⁶R⁷ and —Z—COOR⁷; alternatively, R² may form a cyclic or heterocyclic structure with another R², R¹ R¹⁸ or Y;
R^{2a} is selected from —H, C₁₋₈ alkyl, C₂₋₈ alkenyl, C₂₋₈ alkynyl, and C₃₋₁₀ cycloalkyl, which alkyl, alkenyl, alkynyl and cycloalkyl may be optionally substituted with one or more selected from —OH, aryl, C₁₋₆ alkoxy, heteroaryl, aryloxy, heteroaryloxy, F, and C₃₋₆ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—NR⁶—C(═O)—OR⁷, —Z—C(═O)—OR⁷, —Z—OR⁷, halogen, —Z—SR⁷, —Z—SOR⁷, —Z—SO₂R⁷, —Z—SO₂NR⁶R⁷ and —Z—COOR⁷; with the proviso that the two R^{2a} groups are either both non-hydrogen, or that one of the R^{2a} forms a ring with R¹ or R¹⁸;
each R³ is independently selected from C₁₋₆ alkyl, C₁₋₄ fluoroalkyl, C₁₋₄ hydroxyalkyl, C₂₋₆ alkenyl, C₂₋₆ alkynyl, C₃₋₁₀ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—C(═O)—R⁷, —Z—OR⁷, halogen, —Z—SR⁷, —Z—SOR⁷, —Z—SO₂R⁷, —Z—SO₂NR⁶R⁷ and —Z—COOR⁷, wherein any heterocyclyl may be substituted with one or more R⁴, and wherein any heteroaryl and any aryl may be substituted with one or more R⁵;
Z is selected from a single bond, C₁₋₄ alkylene, heterocyclylene and C₃₋₆ cycloalkylene;
each R⁴ is independently selected from C₁₋₆ alkyl, C₁₋₄ fluoroalkyl, C₁₋₄ hydroxyalkyl, C₁₋₄ alkoxy, C₃₋₁₀ cycloalkyl, NR⁶R⁷, C(═O)—NR⁶R⁷, NR⁶—C(═O)—R⁷, Z—C(═O)—R⁷, —Z—C(═O)—H, OR⁷, halogen, SR⁷, SOR⁷, SO₂R⁷, SO₂NR⁶R⁷ and COOR⁷ and —OH;
each R⁵ is independently selected from C₁₋₆ alkyl, C₁₋₄ fluoroalkyl, C₁₋₄ hydroxyalkyl, C₁₋₄ alkoxy, C₃₋₆ cycloalkyl, —Z—C(═O)—NR⁶R⁷, —Z—NR⁶—C(═O)—R⁷, —Z—C(═O)—R⁷, —Z—NR⁶C(═O)OR⁷, —Z—C(═O)OR⁷, OR⁷, —CN and halogen;
each of R⁶ and R⁷ is independently selected from hydrogen, C₁₋₈ alkyl, C₁₋₄ fluoroalkyl, C₁₋₄ perfluoroalkyl, C₁₋₄ hydroxyalkyl, C₂₋₈ alkenyl, C₂₋₈ alkynyl, C₃₋₁₀ cycloalkyl, —Z-heterocyclyl, —Z-heteroaryl and —Z-aryl, which alkyl, alkenyl, alkynyl, cycloalkyl, heterocyclyl, heteroaryl and aryl may optionally be substituted with one or more independently selected R⁸; or, alternatively, R⁶ and R⁷ may together with the N-atom to which they are attached form an N-heterocyclic ring optionally substituted with one or more independently selected R⁸;
each R⁸ is independently selected from C₁₋₆ alkyl, C₁₋₄ fluoroalkyl, C₁₋₄ hydroxyalkyl, C₂₋₆ alkenyl, C₂₋₆ alkynyl, C₃₋₁₀ cycloalkyl, —Z-heterocyclyl, —Z-heteroaryl, —Z-aryl, —Z—NR¹⁰R¹¹, —Z—C(═O)—NR¹⁰R¹¹, —Z—OR⁹, halogen, —CN, —Z—SR⁹, —Z—SOR⁹, —Z—SO₂R⁹ and —Z—COOR⁹, which alkyl, alkenyl, alkynyl, cycloalkyl, heterocyclics, heteroaryl and aryl may optionally be substituted with one or more selected from C₁₋₄ alkyl, C₁₋₄ fluoroalkyl, C₁₋₄ hydroxyalkyl, C₃₋₆ cycloalkyl, —Z-heterocyclyl, —Z-heteroaryl, —Z-aryl, —Z—NR¹⁰R¹¹, —Z—C(═O)—NR¹⁰R¹¹, —Z—OR⁹, halogen, —CN, —Z—SR⁹, —Z—SOR⁹, —Z—SO₂R⁹ and —Z—COOR⁹; wherein any heterocyclyl may be further substituted with one or more R⁴ as defined above, and wherein any heteroaryl and any aryl may be further substituted with one or more R⁵ as defined above;
each R⁹ is independently selected from —H, C₁₋₈ alkyl, C₁₋₄ fluoroalkyl, C₁₋₄ hydroxyalkyl, C₂₋₈ alkenyl, C₂₋₈ alkynyl, C₃₋₁₀ cycloalkyl, —Z-heterocyclyl, —Z-aryl, and —Z-heteroaryl, wherein any heterocyclyl may be substituted with one or more R⁴ as defined above, and wherein any heteroaryl and any aryl may be substituted with one or more R⁵ as defined above;
each of R¹⁰ and R¹¹ is independently selected from —H, C₁₋₆ alkyl, C₁₋₄ fluoroalkyl, C₁₋₄ hydroxyalkyl, C₂₋₈ alkenyl, C₂₋₈ alkynyl, C₃₋₁₀ cycloalkyl, heterocyclyl, heteroaryl, and aryl, wherein any heterocyclyl may be substituted with one or more R⁴ as defined above, and wherein any heteroaryl and any aryl may be substituted with one or more R⁵ as defined above, or, alternatively, R¹⁰ and R¹¹ may together with the N-atom to which they are attached form an optionally 5 to 7 membered, N-heterocyclic ring optionally substituted with one or more R⁴ as defined above;
when Q is —CH═NR¹², R¹² is selected from C₁₋₁₀ alkyl, C₂₋₁₀ alkenyl, C₂₋₁₀ alkynyl, C₃₋₁₀ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—C(═O)—R⁷, —Z—OR⁷, halogen, —Z—SR⁷, —Z—SOR⁷, —Z—SO₂R⁷ and —Z—COOR⁷, which alkyl, alkenyl, alkynyl, cycloalkyl, heterocyclyl, heteroaryl and aryl may optionally be substituted with one or more R³;
when Q is —CHR²⁰NR²¹R¹³, R¹³ is selected from hydrogen, —C(O)R⁷, —C(O)C(O)R⁷, C₁₋₈ alkyl, C₁₋₄ fluoroalkyl, C₁₋₄ perfluoroalkyl, C₁₋₄ hydroxyalkyl, C₂₋₈ alkenyl, C₂₋₈ alkynyl, C₃₋₁₀ cycloalkyl, —Z-heterocyclyl, —Z-heteroaryl and —Z-aryl, which alkyl, alkenyl, alkynyl, cycloalkyl, heterocyclyl, heteroaryl and aryl may optionally be substituted with one or more independently selected R⁸, or is —CR¹⁴R¹⁵—NR⁶R⁷, —CR¹⁴R¹⁵CN, or —CR¹⁴R¹⁵OR⁷, wherein each of R¹⁴ and R¹⁵ is independently selected from —H, C₁₋₈ alkyl, C₂₋₈ alkenyl, C₂₋₈ alkynyl, C₃₋₁₀ cycloalkyl, heterocyclyl, heteroaryl and aryl, and wherein R¹⁴ and R¹⁵ together with the intervening carbon atom may designate a C₃₋₁₀ cycloalkyl or C₅₋₁₀-cycloalkenyl ring, which alkyl, alkenyl, alkynyl, cycloalkyl ring, cycloalkenyl ring, heterocyclyl, heteroaryl and aryl may optionally be substituted with one or more R³;
R²⁰ and R²¹ are hydrogen, or together form a 1,3-diaza-C₅₋₇-cycloalk-2-yl group which is N-substituted with R¹⁶ and optionally further substituted with one or more R³, and optionally containing one or two oxo groups; a 1,3-thiaza-C₅₋₇-cycloalk-2-yl group which is N-substituted with R¹⁶ and optionally further substituted with one or more R³ and optionally containing one or two oxo groups; an 1,3-oxaza-C₅₋₇-cycloalk-2-yl group which is N-substituted with R¹⁶ and optionally further substituted with one or more R³, and optionally containing one or two oxo groups, wherein in all three instances two R³'s on the same carbon atom may together form a spiro group;
when Q is W, W is selected from an 1,3-diaza-C₅₋₇-cycloalk-2-yl group which is N-substituted with R¹⁶ and optionally further substituted with one or more R³, and optionally containing one or two oxo groups; a 1,3-thiaza-C₅₋₇-cycloalk-2-yl group which is N-substituted with R¹⁶ and optionally further substituted with one or more R³ and optionally containing one or two oxo groups; an 1,3-oxaza-C₅₋₇-cycloalk-2-yl group which is N-substituted with R¹⁶ and optionally further substituted with one or more R³, and optionally containing one or two oxo groups, wherein in all three instances two R³'s on the same carbon atom may together form a spiro group;
R¹⁶ is selected from hydrogen, —C(O)R⁷, —C(O)C(O)R⁷, —C(O)C(O)OR⁷, and —C(O)C(O)NR⁶R⁷;
when Q is —CH(OR¹⁷)₂, each R¹⁷ independently is R³, or wherein two R¹⁷ substituents together with the intervening —O—CH(−)—O— may form a heterocyclyl optionally substituted with one or more R³ and containing up to two oxo groups;
R¹⁸ is selected from C₁₋₆ alkyl, C₁₋₆ fluoroalkyl, C₁₋₆ hydroxyalkyl, C₂₋₇ alkenyl, C₂₋₇ alkynyl, C₃₋₇ cycloalkyl, C₃₋₇ oxyalkyl and may form a cyclic or heterocyclic structure with A, Y or R¹;
or an isomer or a mixture of isomers thereof, or a pharmaceutically acceptable salt, or solvate or prodrug thereof.

7. The compound of claim 6, wherein A is selected from —CHR²C(O)—, or C₁₋₈ alkylene, or heterocyclylene.

8. The compound of claim 1, wherein Y is —NR⁶R⁷.

9. The compound of claim 8, wherein A is —CHR²C(O)—.

10. (canceled)

11. The compound of claim 1, wherein Y is 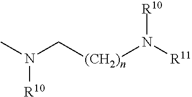
wherein n is from 1 to 3.

12. The compound of claim 11, wherein Y is 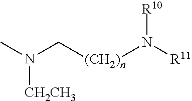
wherein n is from 1 to 3.

13. The compound of claim 11, wherein Y is 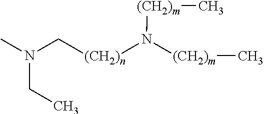
wherein n is from 1 to 3 and each m independently is from 0 to 2.

14. The compound of claim 1, wherein Y is selected from heterocyclyl, heteroaryl and aryl, which may be optionally substituted with one or more R³.

15-16. (canceled)

17. The compound of claim 1, wherein the moiety -A-Y includes 1-3 cyclic moieties selected from monocyclic cycloalkyl, monocyclic heterocyclyl, monocyclic heteroaryl, dicyclic heteroaryl and monocyclic aryl.

18. The compound of claim 1, wherein said compound has the Formula (Ig) 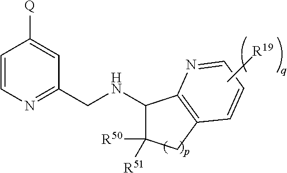
wherein
Q is selected from CO₂H, —CH═NR¹², —W, —CHR²⁰NR²¹R¹³, —CH═O and —CH(OR¹⁷)₂;
each R³ is independently selected from C₁₋₆ alkyl, C₁₋₄ fluoroalkyl, C₁₋₄ hydroxyalkyl, C₂₋₆ alkenyl, C₂₋₆ alkynyl, C₃₋₁₀ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—NR⁶—C(═O)—OR⁷, —Z—C(═O)—R⁷, —Z—OR⁷, halogen, —Z—SR⁷, —Z—SOR⁷, —Z—SO₂R⁷, —Z—SO₂NR⁶R⁷, and —Z—COOR⁷, wherein any heterocyclyl may be substituted with one or more R⁴, and wherein any heteroaryl and any aryl may be substituted with one or more R⁵;
each R⁴ is independently selected from C₁₋₆ alkyl, C₁₋₄ fluoroalkyl, C₁₋₄ hydroxyalkyl, C₁₋₄ alkoxy, C₃₋₁₀ cycloalkyl, NR⁶R⁷, C(═O)—NR⁶R⁷, NR⁶—C(═O)—R⁷, Z—C(═O)—R⁷, —Z—C(═O)—H, OR⁷, halogen, SR⁷, SOR⁷, SO₂R⁷, SO₂NR⁶R⁷ and COOR⁷ and —OH;
each R⁵ is independently selected from C₁₋₆ alkyl, C₁₋₄ fluoroalkyl, C₁₋₄ hydroxyalkyl, C₁₋₄ alkoxy, C₃₋₆ cycloalkyl, —Z—C(═O)—NR⁶R⁷, —Z—NR⁶—C(═O)—R⁷, —Z—C(═O)—R⁷, —Z—C(═O)OR⁷, —Z—NR⁶C(═O)OR⁷, OR⁷, —CN and halogen;
each of R⁶ and R⁷ is independently selected from hydrogen, C₁₋₈ alkyl, C₁₋₄ fluoroalkyl, C₁₋₄ perfluoroalkyl, C₁₋₄ hydroxyalkyl, C₂₋₈ alkenyl, C₂₋₈ alkynyl, C₃₋₁₀ cycloalkyl, —Z-heterocyclyl, —Z-heteroaryl and —Z-aryl, which alkyl, alkenyl, alkynyl, cycloalkyl, heterocyclyl, heteroaryl and aryl may optionally be substituted with one or more independently selected R⁸; or, alternatively, R⁶ and R⁷ may together with the N-atom to which they are attached form an N-heterocyclic ring optionally substituted with one or more independently selected R⁸;
each R⁸ is independently selected from C₁₋₆ alkyl, C₁₋₄ fluoroalkyl, C₁₋₄ hydroxyalkyl, C₂₋₆ alkenyl, C₂₋₆ alkynyl, C₃₋₁₀ cycloalkyl, —Z-heterocyclyl, —Z-heteroaryl, —Z-aryl, —Z—NR¹⁰R¹¹, —Z—C(═O)—NR¹⁰R¹¹, —Z—OR⁹, halogen, —CN, —Z—SR⁹, —Z—SOR⁹, —Z—SO₂R⁹ and —Z—COOR⁹, which alkyl, alkenyl, alkynyl, cycloalkyl, heterocyclics, heteroaryl and aryl may optionally be substituted with one or more selected from C₁₋₄ alkyl, C₁₋₄ fluoroalkyl, C₁₋₄ hydroxyalkyl, C₃₋₆ cycloalkyl, —Z-heterocyclyl, —Z-heteroaryl, —Z-aryl, —Z—NR¹⁰R¹¹, —Z—C(═O)—NR¹⁰R¹¹, —Z—OR⁹, halogen, —CN, —Z—SR⁹, —Z—SOR⁹, —Z—SO₂R⁹ and —Z—COOR⁹; wherein any heterocyclyl may be further substituted with one or more R⁴ as defined above, and wherein any heteroaryl and any aryl may be further substituted with one or more R⁵ as defined above;
each R⁹ is independently selected from —H, C₁₋₈ alkyl, C₁₋₄ fluoroalkyl, C₁₋₄ hydroxyalkyl, C₂₋₈ alkenyl, C₂₋₈ alkynyl, C₃₋₁₀ cycloalkyl, —Z-heterocyclyl, —Z-aryl, and —Z-heteroaryl, wherein any heterocyclyl may be substituted with one or more R⁴ as defined above, and wherein any heteroaryl and any aryl may be substituted with one or more R⁵ as defined above;
each of R¹⁰ and R¹¹ is independently selected from —H, C₁₋₆ alkyl, C₁₋₄ fluoroalkyl, C₁₋₄ hydroxyalkyl, C₂₋₈ alkenyl, C₂₋₈ alkynyl, C₃₋₁₀ cycloalkyl, heterocyclyl, heteroaryl, and aryl, wherein any heterocyclyl may be substituted with one or more R⁴ as defined above, and wherein any heteroaryl and any aryl may be substituted with one or more R⁵ as defined above, or, alternatively, R¹⁰ and R¹¹ may together with the N-atom to which they are attached form an optionally 5 to 7 membered, N-heterocyclic ring optionally substituted with one or more R⁴ as defined above;
when Q is —CH═NR¹², R¹² is selected from C₁₋₁₀ alkyl, C₂₋₁₀ alkenyl, C₂₋₁₀ alkynyl, C₃₋₁₀ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—C(═O)—R⁷, —Z—OR⁷, halogen, —Z—SR⁷, —Z—SOR⁷, —Z—SO₂R⁷ and —Z—COOR⁷, which alkyl, alkenyl, alkynyl, cycloalkyl, heterocyclyl, heteroaryl and aryl may optionally be substituted with one or more R³;
when Q is —CHR²⁰NR²¹R¹³, R¹³ is selected from hydrogen, —C(O)R⁷, —C(O)C(O)R⁷, —C(O)C(O)OR⁷, C₁₋₈ alkyl, C₁₋₄ fluoroalkyl, C₁₋₄ perfluoroalkyl, C₁₋₄ hydroxyalkyl, C₂₋₈ alkenyl, C₂₋₈ alkynyl, C₃₋₁₀ cycloalkyl, —Z-heterocyclyl, —Z-aryl and —Z-heteroaryl, which alkyl, alkenyl, alkynyl, cycloalkyl, heterocyclyl, aryl and heteroaryl may optionally be substituted with one or more independently selected R⁸, or is —CR¹⁴R¹⁵—NR⁶R⁷, —CR¹⁴R¹⁵CN, or —CR¹⁴R¹⁵OR⁷, wherein each of R¹⁴ and R¹⁵ is independently selected from —H, C₁₋₈ alkyl, C₂₋₈ alkenyl, C₂₋₈ alkynyl, C₃₋₁₀ cycloalkyl, heterocyclyl, heteroaryl and aryl, and wherein R¹⁴ and R¹⁵ together with the intervening carbon atom may designate a C₃₋₁₀ cycloalkyl or C₅₋₁₀-cycloalkenyl ring, which alkyl, alkenyl, alkynyl, cycloalkyl ring, cycloalkenyl ring, heterocyclyl, heteroaryl and aryl may optionally be substituted with one or more R³;
R²⁰ and R²¹ are hydrogen, or together form a 1,3-diaza-C₅₋₇-cycloalk-2-yl group which is N-substituted with R¹⁶ and optionally further substituted with one or more R³, and optionally containing one or two oxo groups; a 1,3-thiaza-C₅₋₇-cycloalk-2-yl group which is N-substituted with R¹⁶ and optionally further substituted with one or more R³ and optionally containing one or two oxo groups; an 1,3-oxaza-C₅₋₇-cycloalk-2-yl group which is N-substituted with R¹⁶ and optionally further substituted with one or more R³, and optionally containing one or two oxo groups, wherein in all three instances two R³'s on the same carbon atom may together form a spiro group;
when Q is W, W is selected from an 1,3-diaza-C₅₋₇-cycloalk-2-yl group which is N-substituted with R¹⁶ and optionally further substituted with one or more R³, and optionally containing one or two oxo groups; a 1,3-thiaza-C₅₋₇-cycloalk-2-yl group which is N-substituted with R¹⁶ and optionally further substituted with one or more R³ and optionally containing one or two oxo groups; an 1,3-oxaza-C₅₋₇-cycloalk-2-yl group which is N-substituted with R¹⁶ and optionally further substituted with one or more R³, and optionally containing one or two oxo groups, wherein in all three instances two R³'s on the same carbon atom may together form a spiro group;
R¹⁶ is selected from hydrogen, —C(O)R⁷, —C(O)C(O)R⁷, —C(O)C(O)OR⁷, and —C(O)C(O)NR⁶R⁷;
when Q is —CH(OR¹⁷)₂, each R¹⁷ independently is R³, or wherein two R¹⁷ substituents together with the intervening —O—CH(−)—O— may form a heterocyclyl optionally substituted with one or more R³ and containing up to two oxo groups;
R¹⁹ is selected from the group consisting of C₁₋₆ alkyl, C₁₋₆ alkoxy, C₁₋₄ fluoroalkyl, C₁₋₄ hydroxyalkyl, C₂₋₆ alkenyl, C₂₋₆ alkynyl, C₃₋₁₀ cycloalkyl, —Z-heterocyclyl, —Z-aryl, and —Z-heteroaryl;
Z is selected from the group consisting of a single bond, C₁₋₄ alkylene, heterocyclylene, and C₃₋₆ cycloalkylene;
R⁵⁰ and R⁵¹ are each independently selected from the group consisting of C₁₋₄ alkyl, C₁₋₄ alkoxy, C₁₋₄ fluoroalkyl, and C₁₋₄ hydroxyalkyl;
p is 0, 1, 2, 3, or 4;
q is 0, 1, 2, or 3;
or an isomer or a mixture of isomers thereof, or a pharmaceutically acceptable salt, or solvate or prodrug thereof.

19. The compound of claim 1, wherein said compound has the Formula (Ih) 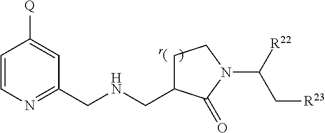
wherein
Q is selected from CO₂H, —CH═NR¹², —W, —CHR²⁰NR²¹R¹³, —CH═O and —CH(OR¹⁷)₂;
each R³ is independently selected from C₁₋₆ alkyl, C₁₋₄ fluoroalkyl, C₁₋₄ hydroxyalkyl, C₂₋₆ alkenyl, C₂₋₆ alkynyl, C₃₋₁₀ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—NR⁶—C(═O)—OR⁷, —Z—C(═O)—R⁷, —Z—OR⁷, halogen, —Z—SR⁷, —Z—SOR⁷, —Z—SO₂R⁷, —Z—SO₂NR⁶R⁷, and —Z—COOR⁷, wherein any heterocyclyl may be substituted with one or more R⁴, and wherein any heteroaryl and any aryl may be substituted with one or more R⁵;
each R⁴ is independently selected from C₁₋₆ alkyl, C₁₋₄ fluoroalkyl, C₁₋₄ hydroxyalkyl, C₁₋₄ alkoxy, C₃₋₁₀ cycloalkyl, NR⁶R⁷, C(═O)—NR⁶R⁷, NR⁶—C(═O)—R⁷, Z—C(═O)—R⁷, —Z—C(═O)—H, OR⁷, halogen, SR⁷, SOR⁷, SO₂R⁷, SO₂NR⁶R⁷ and COOR⁷ and —OH;
each R⁵ is independently selected from C₁₋₆ alkyl, C₁₋₄ fluoroalkyl, C₁₋₄ hydroxyalkyl, C₁₋₄ alkoxy, C₃₋₆ cycloalkyl, —Z—C(═O)—NR⁶R⁷, —Z—NR⁶—C(═O)—R⁷, —Z—C(═O)—R⁷, —Z—C(═O)OR⁷, —Z—NR⁶C(═O)OR⁷, OR⁷, —CN and halogen;
each of R⁶ and R⁷ is independently selected from hydrogen, C₁₋₈ alkyl, C₁₋₄ fluoroalkyl, C₁₋₄ perfluoroalkyl, C₁₋₄ hydroxyalkyl, C₂₋₈ alkenyl, C₂₋₈ alkynyl, C₃₋₁₀ cycloalkyl, —Z-heterocyclyl, —Z-heteroaryl and —Z-aryl, which alkyl, alkenyl, alkynyl, cycloalkyl, heterocyclyl, heteroaryl and aryl may optionally be substituted with one or more independently selected R⁸; or, alternatively, R⁶ and R⁷ may together with the N-atom to which they are attached form an N-heterocyclic ring optionally substituted with one or more independently selected R⁸;
each R⁸ is independently selected from C₁₋₆ alkyl, C₁₋₄ fluoroalkyl, C₁₋₄ hydroxyalkyl, C₂₋₆ alkenyl, C₂₋₆ alkynyl, C₃₋₁₀ cycloalkyl, —Z-heterocyclyl, —Z-heteroaryl, —Z-aryl, —Z—NR¹⁰R¹¹, —Z—C(═O)—NR¹⁰R¹¹, —Z—OR⁹, halogen, —CN, —Z—SR⁹, —Z—SOR⁹, —Z—SO₂R⁹ and —Z—COOR⁹, which alkyl, alkenyl, alkynyl, cycloalkyl, heterocyclics, heteroaryl and aryl may optionally be substituted with one or more selected from C₁₋₄ alkyl, C₁₋₄ fluoroalkyl, C₁₋₄ hydroxyalkyl, C₃₋₆ cycloalkyl, —Z-heterocyclyl, —Z-heteroaryl, —Z-aryl, —Z—NR¹⁰R¹¹, —Z—C(═O)—NR¹⁰R¹¹, —Z—OR⁹, halogen, —CN, —Z—SR⁹, —Z—SOR⁹, —Z—SO₂R⁹ and —Z—COOR⁹; wherein any heterocyclyl may be further substituted with one or more R⁴ as defined above, and wherein any heteroaryl and any aryl may be further substituted with one or more R⁵ as defined above;
each R⁹ is independently selected from —H, C₁₋₈ alkyl, C₁₋₄ fluoroalkyl, C₁₋₄ hydroxyalkyl, C₂₋₈ alkenyl, C₂₋₈ alkynyl, C₃₋₁₀ cycloalkyl, —Z-heterocyclyl, —Z-aryl, and —Z-heteroaryl, wherein any heterocyclyl may be substituted with one or more R⁴ as defined above, and wherein any heteroaryl and any aryl may be substituted with one or more R⁵ as defined above;
each of R¹⁰ and R¹¹ is independently selected from —H, C₁₋₆ alkyl, C₁₋₄ fluoroalkyl, C₁₋₄ hydroxyalkyl, C₂₋₈ alkenyl, C₂₋₈ alkynyl, C₃₋₁₀ cycloalkyl, heterocyclyl, heteroaryl, and aryl, wherein any heterocyclyl may be substituted with one or more R⁴ as defined above, and wherein any heteroaryl and any aryl may be substituted with one or more R⁵ as defined above, or, alternatively, R¹⁰ and R¹¹ may together with the N-atom to which they are attached form an optionally 5 to 7 membered, N-heterocyclic ring optionally substituted with one or more R⁴ as defined above;
when Q is —CH═NR¹², R¹² is selected from C₁₋₁₀ alkyl, C₂₋₁₀ alkenyl, C₂₋₁₀ alkynyl, C₃₋₁₀ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—C(═O)—R⁷, —Z—OR⁷, halogen, —Z—SR⁷, —Z—SOR⁷, —Z—SO₂R⁷ and —Z—COOR⁷, which alkyl, alkenyl, alkynyl, cycloalkyl, heterocyclyl, heteroaryl and aryl may optionally be substituted with one or more R³;
when Q is —CHR²⁰NR²¹R¹³, R¹³ is selected from hydrogen, —C(O)R⁷, —C(O)C(O)R⁷, —C(O)C(O)OR⁷, C₁₋₈ alkyl, C₁₋₄ fluoroalkyl, C₁₋₄ perfluoroalkyl, C₁₋₄ hydroxyalkyl, C₂₋₈ alkenyl, C₂₋₈ alkynyl, C₃₋₁₀ cycloalkyl, —Z-heterocyclyl, —Z-aryl and —Z-heteroaryl, which alkyl, alkenyl, alkynyl, cycloalkyl, heterocyclyl, aryl and heteroaryl may optionally be substituted with one or more independently selected R⁸, or is —CR¹⁴R¹⁵—NR⁶R⁷, —CR¹⁴R¹⁵CN, or —CR¹⁴R¹⁵OR⁷, wherein each of R¹⁴ and R¹⁵ is independently selected from —H, C₁₋₈ alkyl, C₂₋₈ alkenyl, C₂₋₈ alkynyl, C₃₋₁₀ cycloalkyl, heterocyclyl, heteroaryl and aryl, and wherein R¹⁴ and R¹⁵ together with the intervening carbon atom may designate a C₃₋₁₀ cycloalkyl or C₅₋₁₀-cycloalkenyl ring, which alkyl, alkenyl, alkynyl, cycloalkyl ring, cycloalkenyl ring, heterocyclyl, heteroaryl and aryl may optionally be substituted with one or more R³;
R²⁰ and R²¹ are hydrogen, or together form a 1,3-diaza-C₅₋₇-cycloalk-2-yl group which is N-substituted with R¹⁶ and optionally further substituted with one or more R³, and optionally containing one or two oxo groups; a 1,3-thiaza-C₅₋₇-cycloalk-2-yl group which is N-substituted with R¹⁶ and optionally further substituted with one or more R³ and optionally containing one or two oxo groups; an 1,3-oxaza-C₅₋₇-cycloalk-2-yl group which is N-substituted with R¹⁶ and optionally further substituted with one or more R³, and optionally containing one or two oxo groups, wherein in all three instances two R³'s on the same carbon atom may together form a spiro group;
when Q is W, W is selected from an 1,3-diaza-C₅₋₇-cycloalk-2-yl group which is N-substituted with R¹⁶ and optionally further substituted with one or more R³, and optionally containing one or two oxo groups; a 1,3-thiaza-C₅₋₇-cycloalk-2-yl group which is N-substituted with R¹⁶ and optionally further substituted with one or more R³ and optionally containing one or two oxo groups; an 1,3-oxaza-C₅₋₇-cycloalk-2-yl group which is N-substituted with R¹⁶ and optionally further substituted with one or more R³, and optionally containing one or two oxo groups, wherein in all three instances two R³'s on the same carbon atom may together form a spiro group;
R¹⁶ is selected from hydrogen, —C(O)R⁷, —C(O)C(O)R⁷, —C(O)C(O)OR⁷, and —C(O)C(O)NR⁶R⁷;
when Q is —CH(OR¹⁷)₂, each R¹⁷ independently is R³, or wherein two R¹⁷ substituents together with the intervening —O—CH(−)—O— may form a heterocyclyl optionally substituted with one or more R³ and containing up to two oxo groups;
R²² and R²³ are each independently selected from the group consisting of hydrogen, C₁₋₆ alkyl, and aryl, wherein C₁₋₆ alkyl and aryl are optionally substituted with halogen, hydroxy, or C₁₋₆ alkoxy;
r is 0, 1, 2, 3, or 4;
or an isomer or a mixture of isomers thereof, or a pharmaceutically acceptable salt, or solvate or prodrug thereof.

20. The compound of claim 1, wherein said compound has the Formula (Ii) 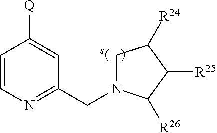
wherein
Q is selected from CO₂H, —CH═NR¹², —W, —CHR²⁰NR²¹R¹³, —CH═O and —CH(OR¹⁷)₂;
each R³ is independently selected from C₁₋₆ alkyl, C₁₋₄ fluoroalkyl, C₁₋₄ hydroxyalkyl, C₂₋₆ alkenyl, C₂₋₆ alkynyl, C₃₋₁₀ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—NR⁶—C(═O)—OR⁷, —Z—C(═O)—R⁷, —Z—OR⁷, halogen, —Z—SR⁷, —Z—SOR⁷, —Z—SO₂R⁷, —Z—SO₂NR⁶R⁷, and —Z—COOR⁷, wherein any heterocyclyl may be substituted with one or more R⁴, and wherein any heteroaryl and any aryl may be substituted with one or more R⁵;
each R⁴ is independently selected from C₁₋₆ alkyl, C₁₋₄ fluoroalkyl, C₁₋₄ hydroxyalkyl, C₁₋₄ alkoxy, C₃₋₁₀ cycloalkyl, NR⁶R⁷, C(═O)—NR⁶R⁷, NR⁶—C(═O)—R⁷, Z—C(═O)—R⁷, —Z—C(═O)—H, OR⁷, halogen, SR⁷, SOR⁷, SO₂R⁷, SO₂NR⁶R⁷ and COOR⁷ and —OH;
each R⁵ is independently selected from C₁₋₆ alkyl, C₁₋₄ fluoroalkyl, C₁₋₄ hydroxyalkyl, C₁₋₄ alkoxy, C₃₋₆ cycloalkyl, —Z—C(═O)—NR⁶R⁷, —Z—NR⁶—C(═O)—R⁷, —Z—C(═O)—R⁷, —Z—C(═O)OR⁷, —Z—NR⁶C(═O)OR⁷, OR⁷, —CN and halogen;
each of R⁶ and R⁷ is independently selected from hydrogen, C₁₋₈ alkyl, C₁₋₄ fluoroalkyl, C₁₋₄ perfluoroalkyl, C₁₋₄ hydroxyalkyl, C₂₋₈ alkenyl, C₂₋₈ alkynyl, C₃₋₁₀ cycloalkyl, —Z-heterocyclyl, —Z-heteroaryl and —Z-aryl, which alkyl, alkenyl, alkynyl, cycloalkyl, heterocyclyl, heteroaryl and aryl may optionally be substituted with one or more independently selected R⁸; or, alternatively, R⁶ and R⁷ may together with the N-atom to which they are attached form an N-heterocyclic ring optionally substituted with one or more independently selected R⁸;
each R⁸ is independently selected from C₁₋₆ alkyl, C₁₋₄ fluoroalkyl, C₁₋₄ hydroxyalkyl, C₂₋₆ alkenyl, C₂₋₆ alkynyl, C₃₋₁₀ cycloalkyl, —Z-heterocyclyl, —Z-heteroaryl, —Z-aryl, —Z—NR¹⁰R¹¹, —Z—C(═O)—NR¹⁰R¹¹, —Z—OR⁹, halogen, —CN, —Z—SR⁹, —Z—SOR⁹, —Z—SO₂R⁹ and —Z—COOR⁹, which alkyl, alkenyl, alkynyl, cycloalkyl, heterocyclics, heteroaryl and aryl may optionally be substituted with one or more selected from C₁₋₄ alkyl, C₁₋₄ fluoroalkyl, C₁₋₄ hydroxyalkyl, C₃₋₆ cycloalkyl, —Z-heterocyclyl, —Z-heteroaryl, —Z-aryl, —Z—NR¹⁰R¹¹, —Z—C(═O)—NR¹⁰R¹¹, —Z—OR⁹, halogen, —CN, —Z—SR⁹, —Z—SOR⁹, —Z—SO₂R⁹ and —Z—COOR⁹; wherein any heterocyclyl may be further substituted with one or more R⁴ as defined above, and wherein any heteroaryl and any aryl may be further substituted with one or more R⁵ as defined above;
each R⁹ is independently selected from —H, C₁₋₈ alkyl, C₁₋₄ fluoroalkyl, C₁₋₄ hydroxyalkyl, C₂₋₈ alkenyl, C₂₋₈ alkynyl, C₃₋₁₀ cycloalkyl, —Z-heterocyclyl, —Z-aryl, and —Z-heteroaryl, wherein any heterocyclyl may be substituted with one or more R⁴ as defined above, and wherein any heteroaryl and any aryl may be substituted with one or more R⁵ as defined above;
each of R¹⁰ and R¹¹ is independently selected from —H, C₁₋₆ alkyl, C₁₋₄ fluoroalkyl, C₁₋₄ hydroxyalkyl, C₂₋₈ alkenyl, C₂₋₈ alkynyl, C₃₋₁₀ cycloalkyl, heterocyclyl, heteroaryl, and aryl, wherein any heterocyclyl may be substituted with one or more R⁴ as defined above, and wherein any heteroaryl and any aryl may be substituted with one or more R⁵ as defined above, or, alternatively, R¹⁰ and R¹¹ may together with the N-atom to which they are attached form an optionally 5 to 7 membered, N-heterocyclic ring optionally substituted with one or more R⁴ as defined above;
when Q is —CH═NR¹², R¹² is selected from C₁₋₁₀ alkyl, C₂₋₁₀ alkenyl, C₂₋₁₀ alkynyl, C₃₋₁₀ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—C(═O)—R⁷, —Z—OR⁷, halogen, —Z—SR⁷, —Z—SOR⁷, —Z—SO₂R⁷ and —Z—COOR⁷, which alkyl, alkenyl, alkynyl, cycloalkyl, heterocyclyl, heteroaryl and aryl may optionally be substituted with one or more R³;
when Q is —CHR²⁰NR²¹R¹³, R¹³ is selected from hydrogen, —C(O)R⁷, —C(O)C(O)R⁷, —C(O)C(O)OR⁷, C₁₋₈ alkyl, C₁₋₄ fluoroalkyl, C₁₋₄ perfluoroalkyl, C₁₋₄ hydroxyalkyl, C₂₋₈ alkenyl, C₂₋₈ alkynyl, C₃₋₁₀ cycloalkyl, —Z-heterocyclyl, —Z-aryl and —Z-heteroaryl, which alkyl, alkenyl, alkynyl, cycloalkyl, heterocyclyl, aryl and heteroaryl may optionally be substituted with one or more independently selected R⁸, or is —CR¹⁴R¹⁵—NR⁶R⁷, —CR¹⁴R¹⁵CN, or —CR¹⁴R¹⁵OR⁷, wherein each of R¹⁴ and R¹⁵ is independently selected from —H, C₁₋₈ alkyl, C₂₋₈ alkenyl, C₂₋₈ alkynyl, C₃₋₁₀ cycloalkyl, heterocyclyl, heteroaryl and aryl, and wherein R¹⁴ and R¹⁵ together with the intervening carbon atom may designate a C₃₋₁₀ cycloalkyl or C₅₋₁₀-cycloalkenyl ring, which alkyl, alkenyl, alkynyl, cycloalkyl ring, cycloalkenyl ring, heterocyclyl, heteroaryl and aryl may optionally be substituted with one or more R³;
R²⁰ and R²¹ are hydrogen, or together form a 1,3-diaza-C₅₋₇-cycloalk-2-yl group which is N-substituted with R¹⁶ and optionally further substituted with one or more R³, and optionally containing one or two oxo groups; a 1,3-thiaza-C₅₋₇-cycloalk-2-yl group which is N-substituted with R¹⁶ and optionally further substituted with one or more R³ and optionally containing one or two oxo groups; an 1,3-oxaza-C₅₋₇-cycloalk-2-yl group which is N-substituted with R¹⁶ and optionally further substituted with one or more R³, and optionally containing one or two oxo groups, wherein in all three instances two R³'s on the same carbon atom may together form a spiro group;
when Q is W, W is selected from an 1,3-diaza-C₅₋₇-cycloalk-2-yl group which is N-substituted with R¹⁶ and optionally further substituted with one or more R³, and optionally containing one or two oxo groups; a 1,3-thiaza-C₅₋₇-cycloalk-2-yl group which is N-substituted with R¹⁶ and optionally further substituted with one or more R³ and optionally containing one or two oxo groups; an 1,3-oxaza-C₅₋₇-cycloalk-2-yl group which is N-substituted with R¹⁶ and optionally further substituted with one or more R³, and optionally containing one or two oxo groups, wherein in all three instances two R³'s on the same carbon atom may together form a spiro group;
R¹⁶ is selected from hydrogen, —C(O)R⁷, —C(O)C(O)R⁷, —C(O)C(O)OR⁷, and —C(O)C(O)NR⁶R⁷;
when Q is —CH(OR¹⁷)₂, each R¹⁷ independently is R³, or wherein two R¹⁷ substituents together with the intervening —O—CH(−)—O— may form a heterocyclyl optionally substituted with one or more R³ and containing up to two oxo groups;
R²⁴, R²⁵, and R²⁶ are each independently selected from the group consisting of hydrogen, C₁₋₆ alkyl, C₁₋₆ alkoxy, C₃₋₁₀ cycloalkyl, aryl, halogen, hydroxymethyl, and C(═O)—R²⁷;
R²⁷ is unsubstituted amine, substituted amine, or heterocycle;
s is 0, 1, 2, 3, or 4;
with the proviso that at least one of R²⁴, R²⁵, and R²⁶ is not hydrogen;
or an isomer or a mixture of isomers thereof, or a pharmaceutically acceptable salt, or solvate or prodrug thereof.

21. The compound of claim 1, wherein said compound has the Formula (Ij) 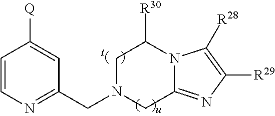
wherein
Q is selected from CO₂H, —CH═NR¹², —W, —CHR²⁰NR²¹R¹³, —CH═O and —CH(OR¹⁷)₂;
each R³ is independently selected from C₁₋₆ alkyl, C₁₋₄ fluoroalkyl, C₁₋₄ hydroxyalkyl, C₂₋₆ alkenyl, C₂₋₆ alkynyl, C₃₋₁₀ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—NR⁶—C(═O)—OR⁷, —Z—C(═O)—R⁷, —Z—OR⁷, halogen, —Z—SR⁷, —Z—SOR⁷, —Z—SO₂R⁷, —Z—SO₂NR⁶R⁷, and —Z—COOR⁷, wherein any heterocyclyl may be substituted with one or more R⁴, and wherein any heteroaryl and any aryl may be substituted with one or more R⁵;
each R⁴ is independently selected from C₁₋₆ alkyl, C₁₋₄ fluoroalkyl, C₁₋₄ hydroxyalkyl, C₁₋₄ alkoxy, C₃₋₁₀ cycloalkyl, NR⁶R⁷, C(═O)—NR⁶R⁷, NR⁶—C(═O)—R⁷, Z—C(═O)—R⁷, —Z—C(═O)—H, OR⁷, halogen, SR⁷, SOR⁷, SO₂R⁷, SO₂NR⁶R⁷ and COOR⁷ and —OH;
each R⁵ is independently selected from C₁₋₆ alkyl, C₁₋₄ fluoroalkyl, C₁₋₄ hydroxyalkyl, C₁₋₄ alkoxy, C₃₋₆ cycloalkyl, —Z—C(═O)—NR⁶R⁷, —Z—NR⁶—C(═O)—R⁷, —Z—C(═O)—R⁷, —Z—C(═O)OR⁷, —Z—NR⁶C(═O)OR⁷, OR⁷, —CN and halogen;
each of R⁶ and R⁷ is independently selected from hydrogen, C₁₋₈ alkyl, C₁₋₄ fluoroalkyl, C₁₋₄ perfluoroalkyl, C₁₋₄ hydroxyalkyl, C₂₋₈ alkenyl, C₂₋₈ alkynyl, C₃₋₁₀ cycloalkyl, —Z-heterocyclyl, —Z-heteroaryl and —Z-aryl, which alkyl, alkenyl, alkynyl, cycloalkyl, heterocyclyl, heteroaryl and aryl may optionally be substituted with one or more independently selected R⁸; or, alternatively, R⁶ and R⁷ may together with the N-atom to which they are attached form an N-heterocyclic ring optionally substituted with one or more independently selected R⁸;
each R⁸ is independently selected from C₁₋₆ alkyl, C₁₋₄ fluoroalkyl, C₁₋₄ hydroxyalkyl, C₂₋₆ alkenyl, C₂₋₆ alkynyl, C₃₋₁₀ cycloalkyl, —Z-heterocyclyl, —Z-heteroaryl, —Z-aryl, —Z—NR¹⁰R¹¹, —Z—C(═O)—NR¹⁰R¹¹, —Z—OR⁹, halogen, —CN, —Z—SR⁹, —Z—SOR⁹, —Z—SO₂R⁹ and —Z—COOR⁹, which alkyl, alkenyl, alkynyl, cycloalkyl, heterocyclics, heteroaryl and aryl may optionally be substituted with one or more selected from C₁₋₄ alkyl, C₁₋₄ fluoroalkyl, C₁₋₄ hydroxyalkyl, C₃₋₆ cycloalkyl, —Z-heterocyclyl, —Z-heteroaryl, —Z-aryl, —Z—NR¹⁰R¹¹, —Z—C(═O)—NR¹⁰R¹¹, —Z—OR⁹, halogen, —CN, —Z—SR⁹, —Z—SOR⁹, —Z—SO₂R⁹ and —Z—COOR⁹; wherein any heterocyclyl may be further substituted with one or more R⁴ as defined above, and wherein any heteroaryl and any aryl may be further substituted with one or more R⁵ as defined above;
each R⁹ is independently selected from —H, C₁₋₈ alkyl, C₁₋₄ fluoroalkyl, C₁₋₄ hydroxyalkyl, C₂₋₈ alkenyl, C₂₋₈ alkynyl, C₃₋₁₀ cycloalkyl, —Z-heterocyclyl, —Z-aryl, and —Z-heteroaryl, wherein any heterocyclyl may be substituted with one or more R⁴ as defined above, and wherein any heteroaryl and any aryl may be substituted with one or more R⁵ as defined above;
each of R¹⁰ and R¹¹ is independently selected from —H, C₁₋₆ alkyl, C₁₋₄ fluoroalkyl, C₁₋₄ hydroxyalkyl, C₂₋₈ alkenyl, C₂₋₈ alkynyl, C₃₋₁₀ cycloalkyl, heterocyclyl, heteroaryl, and aryl, wherein any heterocyclyl may be substituted with one or more R⁴ as defined above, and wherein any heteroaryl and any aryl may be substituted with one or more R⁵ as defined above, or, alternatively, R¹⁰ and R¹¹ may together with the N-atom to which they are attached form an optionally 5 to 7 membered, N-heterocyclic ring optionally substituted with one or more R⁴ as defined above;
when Q is —CH═NR¹², R¹² is selected from C₁₋₁₀ alkyl, C₂₋₁₀ alkenyl, C₂₋₁₀ alkynyl, C₃₋₁₀ cycloalkyl, —Z-heterocyclyl, —Z-aryl, —Z-heteroaryl, —Z—NR⁶R⁷, —Z—C(═O)—NR⁶R⁷, —Z—NR⁶—C(═O)—R⁷, —Z—C(═O)—R⁷, —Z—OR⁷, halogen, —Z—SR⁷, —Z—SOR⁷, —Z—SO₂R⁷ and —Z—COOR⁷, which alkyl, alkenyl, alkynyl, cycloalkyl, heterocyclyl, heteroaryl and aryl may optionally be substituted with one or more R³;
when Q is —CHR²⁰NR²¹R¹³, R¹³ is selected from hydrogen, —C(O)R⁷, —C(O)C(O)R⁷, —C(O)C(O)OR⁷, C₁₋₈ alkyl, C₁₋₄ fluoroalkyl, C₁₋₄ perfluoroalkyl, C₁₋₄ hydroxyalkyl, C₂₋₈ alkenyl, C₂₋₈ alkynyl, C₃₋₁₀ cycloalkyl, —Z-heterocyclyl, —Z-aryl and —Z-heteroaryl, which alkyl, alkenyl, alkynyl, cycloalkyl, heterocyclyl, aryl and heteroaryl may optionally be substituted with one or more independently selected R⁸, or is —CR¹⁴R¹⁵—NR⁶R⁷, —CR¹⁴R¹⁵CN, or —CR¹⁴R¹⁵OR⁷, wherein each of R¹⁴ and R¹⁵ is independently selected from —H, C₁₋₈ alkyl, C₂₋₈ alkenyl, C₂₋₈ alkynyl, C₃₋₁₀ cycloalkyl, heterocyclyl, heteroaryl and aryl, and wherein R¹⁴ and R¹⁵ together with the intervening carbon atom may designate a C₃₋₁₀ cycloalkyl or C₅₋₁₀-cycloalkenyl ring, which alkyl, alkenyl, alkynyl, cycloalkyl ring, cycloalkenyl ring, heterocyclyl, heteroaryl and aryl may optionally be substituted with one or more R³;
R²⁰ and R²¹ are hydrogen, or together form a 1,3-diaza-C₅₋₇-cycloalk-2-yl group which is N-substituted with R¹⁶ and optionally further substituted with one or more R³, and optionally containing one or two oxo groups; a 1,3-thiaza-C₅₋₇-cycloalk-2-yl group which is N-substituted with R¹⁶ and optionally further substituted with one or more R³ and optionally containing one or two oxo groups; an 1,3-oxaza-C₅₋₇-cycloalk-2-yl group which is N-substituted with R¹⁶ and optionally further substituted with one or more R³, and optionally containing one or two oxo groups, wherein in all three instances two R³'s on the same carbon atom may together form a spiro group;
when Q is W, W is selected from an 1,3-diaza-C₅₋₇-cycloalk-2-yl group which is N-substituted with R¹⁶ and optionally further substituted with one or more R³, and optionally containing one or two oxo groups; a 1,3-thiaza-C₅₋₇-cycloalk-2-yl group which is N-substituted with R¹⁶ and optionally further substituted with one or more R³ and optionally containing one or two oxo groups; an 1,3-oxaza-C₅₋₇-cycloalk-2-yl group which is N-substituted with R¹⁶ and optionally further substituted with one or more R³, and optionally containing one or two oxo groups, wherein in all three instances two R³'s on the same carbon atom may together form a spiro group;
R¹⁶ is selected from hydrogen, —C(O)R⁷, —C(O)C(O)R⁷, —C(O)C(O)OR⁷, and —C(O)C(O)NR⁶R⁷;
when Q is —CH(OR¹⁷)₂, each R¹⁷ independently is R³, or wherein two R¹⁷ substituents together with the intervening —O—CH(−)—O— may form a heterocyclyl optionally substituted with one or more R³ and containing up to two oxo groups;
R³⁰ is selected from the group consisting of hydrogen, halogen, C₁₋₆ alkyl, and aryl, wherein C₁₋₆ alkyl and aryl groups may optionally be further substituted by halogen, hydroxy, C₁₋₆ alkyl, C₁₋₆ alkoxy, or —NR⁶R⁷;
R²⁸ and R²⁹ are independently selected from the group consisting of hydrogen, halogen, and C₁₋₆ alkyl;
t is 1, 2, or 3;
u is 1, 2, or 3;
or an isomer or a mixture of isomers thereof, or a pharmaceutically acceptable salt, or solvate or prodrug thereof.

22. The compound of claim 1, wherein Q is CO₂H.

23. The compound of claim 1, wherein Q is of the formula 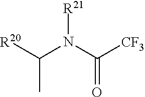
wherein R²⁰ and R²¹ are hydrogen, or together form a 1,3-diaza-C₅₋₇-cycloalk-2-yl group which is N-substituted with R¹⁶ and optionally further substituted with one or more R³, and optionally containing one or two oxo groups; a 1,3-thiaza-C₅₋₇-cycloalk-2-yl group which is N-substituted with R¹⁶ and optionally further substituted with one or more R³ and optionally containing one or two oxo groups; an 1,3-oxaza-C₅₋₇-cycloalk-2-yl group which is N-substituted with R¹⁶ and optionally further substituted with one or more R³, and optionally containing one or two oxo groups, wherein in all three instances two R³'s on the same carbon atom may together form a spiro group.

24. A compound selected from:
2-(1-{[(1S)-1-{[(3-carboxyphenyl)methyl]carbamoyl}ethyl]amino}ethyl)pyridine-4-carboxylic acid
2-[({[(3R)-2-oxo-1-[(1R)-1-phenylethyl]piperidin-3-yl]methyl}amino)methyl]pyridine-4-carboxylic acid
2-({[(1R)-1-[bis(prop-2-en-1-yl)carbamoyl]-5-(propylamino)pentyl]amino}methyl)pyridine-4-carboxylic acid
2-({[(1R)-1-[bis(prop-2-en-1-yl)carbamoyl]-5-{[(tert-butoxy)carbonyl](propyl)amino}pentyl]amino}methyl)pyridine-4-carboxylic acid
2-(1-{[(1S)-1-{[(4-nitrophenyl)methyl]carbamoyl}ethyl]amino}ethyl)pyridine-4-carboxylic acid
2-(1-{[(1S)-1-{[(2-hydroxyphenyl)methyl]carbamoyl}ethyl]amino}ethyl)pyridine-4-carboxylic acid
2-({[(1S)-3-methyl-1-({[2-(2-methylcyclopropaneamido)phenyl]methyl}carbamoyl)butyl]amino}methyl)pyridine-4-carboxylic acid
2-(1-{[(1S)-1-{[(2-nitrophenyl)methyl]carbamoyl}ethyl]amino}ethyl)pyridine-4-carboxylic acid
2-({[(1S)-1-[bis(prop-2-en-1-yl)carbamoyl]-5-[(tert-butylcarbamoyl)amino]pentyl]amino}methyl)pyridine-4-carboxylic acid
2-({[(1S)-1-[bis(prop-2-en-1-yl)carbamoyl]-2-{[3-(dimethylamino)propyl]carbamoyl}ethyl]amino}methyl)pyridine-4-carboxylic acid
2-({[(1S)-1-[bis(prop-2-en-1-yl)carbamoyl]-2-({[1-(hydroxymethyl)cyclopropyl]methyl}carbamoyl)ethyl]amino}methyl)pyridine-4-carboxylic acid
2-({[(1S)-1-({[2-(2-methoxyacetamido)phenyl]methyl}carbamoyl)-3-methylbutyl]amino}methyl)pyridine-4-carboxylic acid
2-{[({1-[(2E)-3-phenylprop-2-en-1-yl]-1H-imidazol-2-yl}methyl)amino]methyl}pyridine-4-carboxylic acid
2-[({[(3S)-2-oxo-1-[(1R)-1-phenylethyl]piperidin-3-yl]methyl}amino)methyl]pyridine-4-carboxylic acid
2-(1-{[(1S)-1-[(pyridin-4-ylmethyl)carbamoyl]ethyl]amino}ethyl)pyridine-4-carboxylic acid
2-[(1R)-1-{[(1S)-1-({[4-(hydroxymethyl)phenyl]methyl}carbamoyl)ethyl]amino}ethyl]pyridine-4-carboxylic acid
2-[({[(3S)-2-oxo-1-[(1R)-1-phenylethyl]pyrrolidin-3-yl]methyl}amino)methyl]pyridine-4-carboxylic acid
2-({[(1R)-1-[bis(prop-2-en-1-yl)carbamoyl]-2-[(cyclopropylmethyl)carbamoyl]ethyl]amino}methyl)pyridine-4-carboxylic acid
2-(1-{[(1S)-1-({[2-(hydroxymethyl)phenyl]methyl}carbamoyl)ethyl]amino}ethyl)pyridine-4-carboxylic acid
2-({[(1S)-1-[bis(prop-2-en-1-yl)carbamoyl]-5-[methyl(methylcarbamoyl)amino]pentyl]amino}methyl)pyridine-4-carboxylic acid
2-({[(1S)-1-[bis(prop-2-en-1-yl)carbamoyl]-5-(N-methylacetamido)pentyl]amino}methyl)pyridine-4-carboxylic acid
2-({[(2S)-6-{[(tert-butoxy)carbonyl]amino}-1-hydroxyhexan-2-yl]amino}methyl)pyridine-4-carboxylic acid
2-({[2-oxo-2-(piperidin-1-yl)ethyl]amino}methyl)pyrimidine-4-carboxylic acid
2-({[(1R)-1-[bis(prop-2-en-1-yl)carbamoyl]-2-(butylcarbamoyl)ethyl]amino}methyl)pyridine-4-carboxylic acid
2-({[(1R)-1-[bis(prop-2-en-1-yl)carbamoyl]-3-carbamoylpropyl]amino}methyl)pyridine-4-carboxylic acid
6-({[2-oxo-2-(piperidin-1-yl)ethyl]amino}methyl)pyridazine-4-carboxylic acid
2-({[2-(diethylcarbamoyl)ethyl](2-acetamidoethyl)amino}methyl)pyridine-4-carboxylic acid
2-(1-{[(1S)-1-(1,3-thiazol-2-yl)ethyl]amino}ethyl)pyridine-4-carboxylic acid
2-({[(1S)-1-[bis(prop-2-en-1-yl)carbamoyl]-3-methanesulfonylpropyl]amino}methyl)pyridine-4-carboxylic acid
2-(1-{[(1R)-1-(1,3-thiazol-2-yl)ethyl]amino}ethyl)pyridine-4-carboxylic acid
2-{1-[(carbamoylmethyl)[2-(diethylcarbamoyl)ethyl]amino]ethyl}pyridine-4-carboxylic acid
2-({bis[2-(diethylcarbamoyl)ethyl]amino}methyl)pyridine-4-carboxylic acid
2-(1-{[(2R)-1-hydroxy-4-methylpentan-2-yl]amino}ethyl)pyridine-4-carboxylic acid
2-{[(2-carbamoylethyl)[2-oxo-2-(piperidin-1-yl)ethyl]amino]methyl}pyridine-4-carboxylic acid
6-({[2-oxo-2-(piperidin-1-yl)ethyl]amino}methyl)pyrimidine-4-carboxylic acid
2-(1-{[(1S)-1-(benzylcarbamoyl)ethyl]amino}ethyl)pyridine-4-carboxylic acid
2-({[(1R)-1-[bis(prop-2-en-1-yl)carbamoyl]-3-methanesulfonylpropyl]amino}methyl)pyridine-4-carboxylic acid
2-({[(1S)-1-{[(1,1-dioxo-1-thiolan-3-yl)methyl]carbamoyl}-3-methylbutyl]amino}methyl)pyridine-4-carboxylic acid
2-({[(1-ethyl-2-oxopyrrolidin-3-yl)methyl]amino}methyl)pyridine-4-carboxylic acid
2-({[(1S)-1-[bis(prop-2-en-1-yl)carbamoyl]-5-{[(tert-butoxy)carbonyl]amino}pentyl]amino}methyl)pyridine-4-carboxylic acid
2-{1-[(1,3-thiazol-2-ylmethyl)amino]ethyl}pyridine-4-carboxylic acid
2-[2-(methylsulfanyl)-1-{[2-oxo-2-(piperidin-1-yl)ethyl]amino}ethyl]pyridine-4-carboxylic acid
2-({[1-(diethylcarbamoyl)propan-2-yl]amino}methyl)pyridine-4-carboxylic acid
2-({[2-(diethylcarbamoyl)ethyl](2-hydroxyethyl)amino}methyl)pyridine-4-carboxylic acid
2-(1-{[2-oxo-2-(piperidin-1-yl)ethyl]amino}butyl)pyridine-4-carboxylic acid
2-({[3-(4-methoxyphenyl)propyl][2-oxo-2-(piperidin-1-yl)ethyl]amino}methyl)pyridine-4-carboxylic acid
2-(1-{methyl[2-oxo-2-(piperidin-1-yl)ethyl]amino}ethyl)pyridine-4-carboxylic acid
2-(1-{[2-oxo-2-(piperidin-1-yl)ethyl]amino}ethyl)pyridine-4-carboxylic acid
2-({[(2S)-1-(tert-butoxy)-4-(methylsulfanyl)-1-oxobutan-2-yl]amino}methyl)pyridine-4-carboxylic acid
2-{[5-(4-fluorophenyl)-5H,6H,7H,8H,9H-imidazo[1,2-a][1,4]diazepin-8-yl]methyl}pyridine-4-carboxylic acid
2-{5H,6H,7H,8H,9H,10H-imidazo[1,2-a][1,4]diazocin-9-ylmethyl}pyridine-4-carboxylic acid
2-{5H,6H,7H,8H-imidazo[1,2-a]pyrazin-7-ylmethyl}pyridine-4-carboxylic acid
2-{5H,6H,7H,8H,9H-imidazo[1,2-a][1,4]diazepin-8-ylmethyl}pyridine-4-carboxylic acid
2-({5-[(dimethylamino)methyl]-5H,6H,7H,8H,9H-imidazo[1,2-a][1,4]diazepin-8-yl}methyl)pyridine-4-carboxylic acid
2-{[(2S)-2-(piperidine-1-carbonyl)pyrrolidin-1-yl]methyl}pyridine-4-carboxylic acid
2-{[(2R)-2-(piperidine-1-carbonyl)pyrrolidin-1-yl]methyl}pyridine-4-carboxylic acid
2-{[(2R)-2-(hydroxymethyl)pyrrolidin-1-yl]methyl}pyridine-4-carboxylic acid
2-{[(2S)-2-(hydroxymethyl)pyrrolidin-1-yl]methyl}pyridine-4-carboxylic acid
2-{[(2R,3S)-3-hydroxy-5-methyl-2-(2-methylpropyl)pyrrolidin-1-yl]methyl}pyridine-4-carboxylic acid
2-({[(1S)-3-methyl-1-(oxolan-2-yl)butyl]amino}methyl)pyridine-4-carboxylic acid
(S)-2-{[(1-hydroxy-4-methylpentan-2-yl)amino]methyl}pyridine-4-carboxylic acid
2-{[3-cyclohexyl-2-(hydroxymethyl)piperidin-1-yl]methyl}pyridine-4-carboxylic acid
2-{[2-(hydroxymethyl)-3-phenylpiperidin-1-yl]methyl}pyridine-4-carboxylic acid
2-{[(2S)-2-(hydroxymethyl)azetidin-1-yl]methyl}pyridine-4-carboxylic acid
2-{[(2S,3S)-3-ethyl-2-(hydroxymethyl)pyrrolidin-1-yl]methyl}pyridine-4-carboxylic acid
2-{[2-(hydroxymethyl)piperidin-1-yl]methyl}pyridine-4-carboxylic acid
2-({2-methyl-5H,6H,7H,8H,9H,10H-imidazo[1,2-a][1,5]diazocin-8-yl}methyl)pyridine-4-carboxylic acid
2-{[3-(ethylcarbamoyl)azetidin-1-yl]methyl}pyridine-4-carboxylic acid
2-({2-methyl-5H,6H,7H, 8H,9H-imidazo[1,2-d][1,4]diazepin-7-yl}methyl)pyridine-4-carboxylic acid
2-{[(2S)-2-[2-oxo-2-(piperidin-1-yl)ethyl]piperidin-1-yl]methyl}pyridine-4-carboxylic acid
2-{[(2S)-2-[(ethylcarbamoyl)methyl]piperidin-1-yl]methyl}pyridine-4-carboxylic acid
2-{[(2R)-2-[2-oxo-2-(piperidin-1-yl)ethyl]piperidin-1-yl]methyl}pyridine-4-carboxylic acid
2-{[(3R)-3-[(ethylcarbamoyl)methyl]pyrrolidin-1-yl]methyl}pyridine-4-carboxylic acid
2-{[3-(ethylcarbamoyl)piperidin-1-yl]methyl}pyridine-4-carboxylic acid
2-{[4-(ethylcarbamoyl)piperidin-1-yl]methyl}pyridine-4-carboxylic acid
2-{[3-(ethylcarbamoyl)pyrrolidin-1-yl]methyl}pyridine-4-carboxylic acid
2-{[(3S)-3-[(ethylcarbamoyl)methyl]pyrrolidin-1-yl]methyl}pyridine-4-carboxylic acid
2-[({[(3S)-1-[(1R)-1-(4-methoxyphenyl)ethyl]-2-oxopyrrolidin-3-yl]methyl}amino)methyl]pyridine-4-carboxylic acid
2-[({[(3R)-1-[(1R)-1-(4-methoxyphenyl)ethyl]-2-oxopyrrolidin-3-yl]methyl}amino)methyl]pyridine-4-carboxylic acid
2-[({[(3S)-1-[(1R)-1-(4-methoxyphenyl)ethyl]-2-oxopiperidin-3-yl]methyl}amino)methyl]pyridine-4-carboxylic acid
2-[({[(3R)-1-[(1R)-1-(4-methoxyphenyl)ethyl]-2-oxopiperidin-3-yl]methyl}amino)methyl]pyridine-4-carboxylic acid
2-[({[(3R)-2-oxo-1-[(1R)-1-phenylethyl]pyrrolidin-3-yl]methyl}amino)methyl]pyridine-4-carboxylic acid
2-[({[1-(4-fluorobenzyl)-1H-pyrrolo[2,3-b]pyridin-3-yl]methyl}amino)methyl]pyridine-4-carboxylic acid
2-{[(pyridin-3-ylmethyl)amino]methyl}pyridine-4-carboxylic acid
2-{[(isoquinolin-4-ylmethyl)amino]methyl}pyridine-4-carboxylic acid
2-{[({5-fluoro-1-[(4-fluorophenyl)methyl]-1H-indol-3-yl}methyl)amino]methyl}pyridine-4-carboxylic acid
2-{[(quinolin-6-ylmethyl)amino]methyl}pyridine-4-carboxylic acid
2-{[({2-tert-butylimidazo[1,2-a]pyridin-3-yl}methyl)amino]methyl}pyridine-4-carboxylic acid
6-({[(2S)-1-(benzyloxy)-4-methylpentan-2-yl]amino}methyl)pyrimidine-4-carboxylic acid
2-[({5H,6H,7H,8H-imidazo[1,2-a]pyridin-8-yl}amino)methyl]pyridine-4-carboxylic acid
2-[({4-bromo-5H,6H,7H-cyclopenta[b]pyridin-7-yl}amino)methyl]pyridine-4-carboxylic acid
2-[({4-benzyl-5H,6H,7H-cyclopenta[b]pyridin-7-yl}amino)methyl]pyridine-4-carboxylic acid
2-[({5H,6H,7H-pyrrolo[1,2-a]imidazol-7-yl}amino)methyl]pyridine-4-carboxylic acid
2-{[(5,6,7,8-tetrahydroquinolin-8-yl)amino]methyl}pyridine-4-carboxylic acid
2-({[3-(prop-2-en-1-yl)-5H,6H,7H-cyclopenta[b]pyridin-7-yl]amino}methyl)pyridine-4-carboxylic acid
2-({[4-(2-phenylethyl)-5H,6H,7H-cyclopenta[b]pyridin-7-yl]amino}methyl)pyridine-4-carboxylic acid
2-[({4-ethyl-5H,6H,7H-cyclopenta[b]pyridin-7-yl}amino)methyl]pyridine-4-carboxylic acid
2-[({5H,6H,7H-cyclopenta[b]pyridin-7-yl}amino)methyl]pyridine-4-carboxylic acid
2-({[4-(propan-2-yl)-5H,6H,7H-cyclopenta[b]pyridin-7-yl]amino}methyl)pyridine-4-carboxylic acid
2-[({4-ethenyl-5H,6H,7H-cyclopenta[b]pyridin-7-yl}amino)methyl]pyridine-4-carboxylic acid
2-[({4-methoxy-5H,6H,7H-cyclopenta[b]pyridin-7-yl}amino)methyl]pyridine-4-carboxylic acid
2-[({6,6-dimethyl-5H,6H,7H-cyclopenta[b]pyridin-7-yl}amino)methyl]pyridine-4-carboxylic acid
2-[({3-bromo-5H,6H,7H-cyclopenta[b]pyridin-7-yl}amino)methyl]pyridine-4-carboxylic acid
2-[({[(3S)-1-ethyl-2-oxopiperidin-3-yl]methyl}amino)methyl]pyridine-4-carboxylic acid
2-[({[(3S)-1-ethyl-2-oxopyrrolidin-3-yl]methyl}amino)methyl]pyridine-4-carboxylic acid
2-[({[(3R)-1-ethyl-2-oxopyrrolidin-3-yl]methyl}amino)methyl]pyridine-4-carboxylic acid
2-[({[(3R)-1-ethyl-2-oxopiperidin-3-yl]methyl}amino)methyl]pyridine-4-carboxylic acid
2,2,2-trifluoro-1-[6-(2-{5H,6H,7H,8H,9H-imidazo[1,2-a][1,4]diazepin-8-ylmethyl}pyridin-4-yl)-5-oxa-7-azaspiro[2.5]octan-7-yl]ethan-1-one
and isomers or mixtures of isomers thereof, or a pharmaceutically acceptable salt, or solvate or prodrug thereof.

25-29. (canceled)

30. A pharmaceutical composition comprising at least one compound of claim 1 and optionally one or more pharmaceutically acceptable excipients, diluents or carriers.

31-36. (canceled)

37. A method of treating a HDME dependent disease in a subject, said method comprises administering to said subject a therapeutically effective amount of at least one compound of claim 1.

38. The method according to claim 37, wherein the HDME is a member of at least one of the KDM7, KDM6, KDM5, KDM4, KDM3 or KDM2 families.

39. The method according to claim 37, wherein said HDME is at least one of PHF8, KDM6A, KDM5A, KDM5B, KDM4A, KDM4C, KDM3A, KDM2A, or KDM2B.
